(19) United States
(12) Patent Application Publication
(10) Pub. No.: US 2025/0351664 A1
(43) Pub. Date: Nov. 13, 2025

(54) LIGHT EMITTING ELEMENT, NITROGEN-CONTAINING COMPOUND FOR THE SAME AND ELECTRONIC APPARATUS INCLUDING THE SAME

(71) Applicant: Samsung Display Co., Ltd., Yongin-si (KR)

(72) Inventors: JANG YEOL BAEK, Yongin-si (KR); YOUNGJIN PARK, Yongin-si (KR); HEECHOON AHN, Yongin-si (KR); HYUNAH UM, Yongin-si (KR); JUHUI YUN, Yongin-si (KR); YESEUL LEE, Yongin-si (KR)

(21) Appl. No.: 19/196,424

(22) Filed: May 1, 2025

(30) Foreign Application Priority Data
May 9, 2024 (KR) 10-2024-0061230

(51) Int. Cl. H10K 50/12; H10K 50/16; H10K 50/17; H10K 85/30; H10K 85/40; H10K 85/60; H10K 101/00

(52) U.S. Cl. CPC H10K 50/12; H10K 50/16; H10K 50/171; H10K 85/342; H10K 85/346; H10K 85/40; H10K 85/622; H10K 85/623; H10K 85/626; H10K 85/633; H10K 85/636; H10K 85/654; H10K 85/6572; H10K 85/6574; H10K 85/6576; H10K 2101/90

(57) ABSTRACT

A light emitting element includes a first electrode, a second electrode opposite to the first electrode, and at least one functional layer disposed between the first electrode and the second electrode and including a nitrogen-containing compound represented by Formula 1.

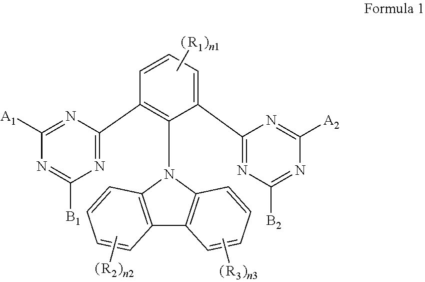

### CROSS-REFERENCE TO RELATED APPLICATION

The present application claims priority to and the benefit of Korean Patent Application No. 10-2024-0061230, filed on May 9, 2024, in the Korean Intellectual Property Office, the entire content of which is incorporated herein by reference.

### BACKGROUND

### 1. Field

One or more embodiments of the present disclosure relate to a light emitting element, a nitrogen-containing compound used for the light emitting element, and an electronic apparatus including the light emitting element.

### 2. Description of the Related Art

An electronic apparatus includes a display device that displays an image. Recently, the research and development of organic electroluminescence display devices used as image display devices have been actively conducted. The organic electroluminescence display device, which is different from a liquid crystal display device, includes a self-luminescent light emitting element. In the self-luminescent light emitting element, holes and electrons injected separately from a first electrode and a second electrode into (and recombine in) an emission layer of the light emitting element. The recombination of these charge carries (e.g., the holes and electrons) causes a light emitting material in the emission layer to emit light, thereby achieving image display (e.g., display of images).

For the application of light emitting elements to display devices, a low driving voltage, a high emission efficiency, and long lifetime of the light emitting element are desired or required. Therefore, development on materials for light emitting elements that can stably achieves such desired characteristics is being continuously and actively pursued.

### SUMMARY

One or more aspects of embodiments of the present disclosure are directed toward a light emitting element with (that has) an improved emission efficiency and element lifetime.

One or more aspects of embodiments of the present disclosure are directed toward a nitrogen-containing compound which is capable of improving the emission efficiency and lifetime of a light emitting element.

One or more aspects of embodiments of the present disclosure are directed toward an electronic apparatus with (that has) excellent or suitable display quality by including the light emitting element with (having) the improved emission efficiency and lifetime.

Additional aspects will be set forth in part in the description which follows and, in part, will be apparent from the description, or may be learned by practice of the presented embodiments.

According to one or more embodiments of the present disclosure, a light emitting element includes a first electrode, a second electrode opposite to (e.g., oppositely arranged to) the first electrode, and at least one functional layer between the first electrode and the second electrode and including a nitrogen-containing compound represented by Formula 1.

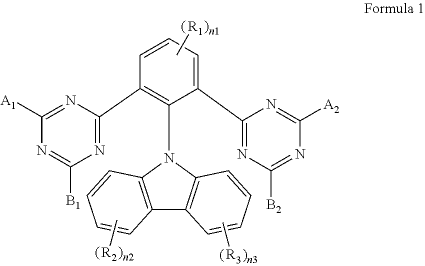

In Formula 1, R₁ to R₃ may each independently be hydrogen, deuterium, a halogen, a cyano group, a substituted or unsubstituted silyl group, a substituted or unsubstituted germanium group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, A₁, A₂, B₁, and B₂ may each independently be a substituted or unsubstituted aryl group of 6 to 30 ring-forming carbon atoms or a substituted or unsubstituted heteroaryl group of 2 to 30 ring-forming carbon atoms, at least one of A₁ or B₁, and at least one of A₂ or B₂ may each independently be a substituted or unsubstituted carbazole group, n1 may be an integer of 0 to 3, and n2 and n3 may each independently be an integer of 0 to 4.

In one or more embodiments, the nitrogen-containing compound represented by Formula 1 may be represented by Formula 2.

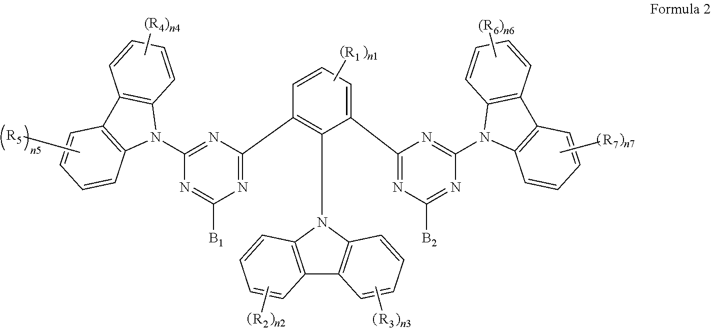

In Formula 2, R₄ to R₇ may each independently be hydrogen, deuterium, a halogen, a cyano group, a substituted or unsubstituted silyl group, a substituted or unsubstituted germanium group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and n4 to n7 may each independently be an integer of 0 to 4.

In Formula 2, the same explanation defined in Formula 1 may be applied for R₁ to R₃, n1 to n3, B₁, and B₂. In other words, R₁ to R₃, n1 to n3, B₁, and B₂ in Formula 2 may each independently be the same as defined in Formula 1.

In one or more embodiments, the nitrogen-containing compound represented by Formula 1 may be represented by Formula 3-1 or Formula 3-2.

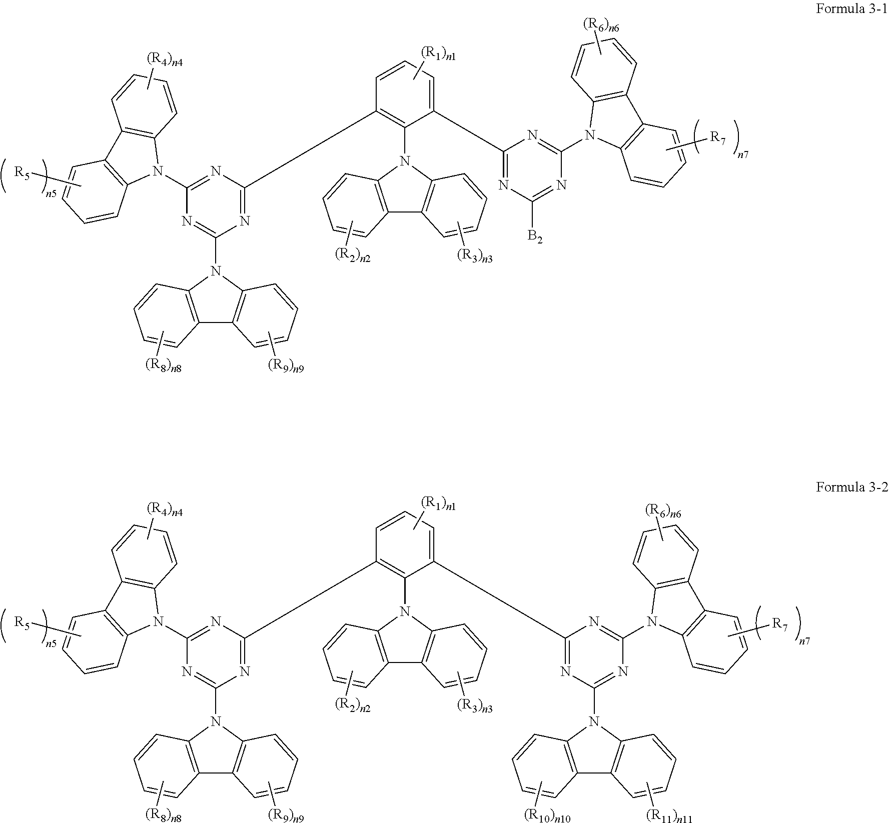

In Formula 3-1 and Formula 3-2, R₄ to R₁₁ may each independently be hydrogen, deuterium, a halogen, a cyano group, a substituted or unsubstituted silyl group, a substituted or unsubstituted germanium group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and n4 to n11 may each independently be an integer of 0 to 4.

In Formula 3-1 and Formula 3-2, the same explanation defined in Formula 1 may be applied for R₁ to R₃, n1 to n3, and B₂. In other words, R₁ to R₃, n1 to n3, and B₂ in Formula 3-1 and Formula 3-2 may each independently be the same as defined in Formula 1.

In one or more embodiments, the nitrogen-containing compound represented by Formula 1 may be represented by Formula 4-1 or Formula 4-2.

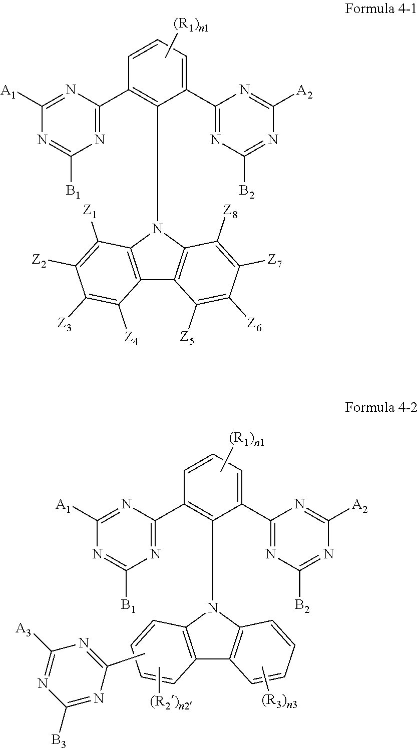

In Formula 4-1 and Formula 4-2, Z₁ to Z₈ may each independently be hydrogen or a substituted or unsubstituted aryl group of 6 to 30 ring-forming carbon atoms, R_{2′} may be hydrogen, deuterium, a halogen, a cyano group, a substituted or unsubstituted silyl group, a substituted or unsubstituted germanium group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, n2′ is an integer of 0 to 3, and A₃ and B₃ may each independently be a substituted or unsubstituted aryl group of 6 to 30 ring-forming carbon atoms or a substituted or unsubstituted heteroaryl group of 2 to 30 ring-forming carbon atoms.

In Formula 4-1 and Formula 4-2, the same explanation defined in Formula 1 may be applied for R₁, R₃, n1, n3, A₁, A₂, B₁, and B₂. In other words, R₁, R₃, n1, n3, A₁, A₂, B₁, and B₂ in Formula 4-1 and Formula 4-2 may each independently be the same as defined in Formula 1.

In one or more embodiments, in Formula 1, at least one of A₁ or B₁, and at least one of A₂ or B₂ may each independently be a substituted or unsubstituted carbazole group, and the remainder selected from among A₁, B₁, A₂, and B₂ may each independently be represented by one selected from among Formula A-1 to Formula A-4.

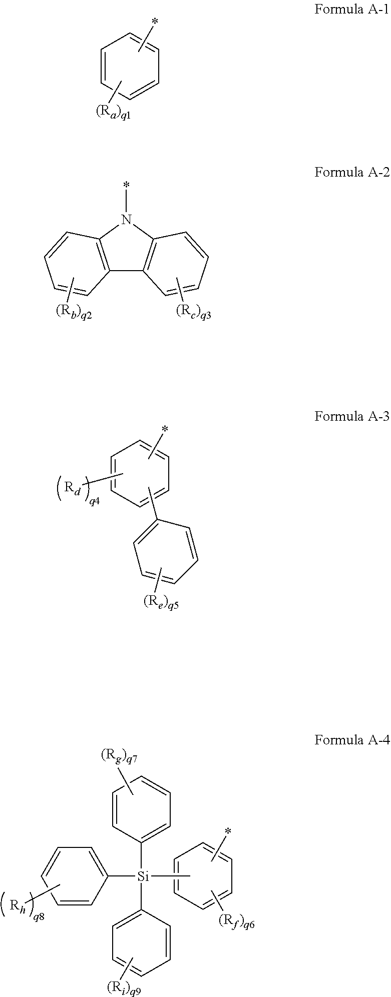

In Formula A-1 to Formula A-4, Rₐ to Rᵢ may each independently be hydrogen, deuterium, a halogen, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, q1, q5, and q7 to q9 may each independently be an integer of 0 to 5, and q2 to q4, and q6 may each independently be an integer of 0 to 4.

In one or more embodiments, the at least one functional layer may include a hole transport region on (e.g., arranged on) the first electrode, an emission layer on (e.g., arranged on) the hole transport region, and an electron transport region on (e.g., arranged on) the emission layer.

In one or more embodiments, the emission layer may include a first host and a dopant, and the first host may include the nitrogen-containing compound.

In one or more embodiments, the emission layer may be to emit thermally activated delayed fluorescence or phosphorescence.

In one or more embodiments, the dopant may be represented by Formula D-1.

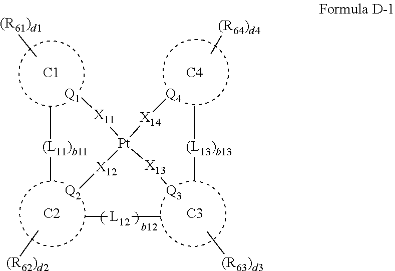

In Formula D-1, Q₁ to Q₄ may each independently be C or N, C1 to C4 may each independently be a substituted or unsubstituted hydrocarbon ring of 5 to 30 ring-forming carbon atoms, a substituted or unsubstituted aryl group of 6 to 30 ring-forming carbon atoms, or a substituted or unsubstituted heterocycle of 2 to 30 ring-forming carbon atoms, X₁₁ to X₁₄ may each independently be a direct linkage or *—O—*, L₁₁ to L₁₃ may each independently be a direct linkage,

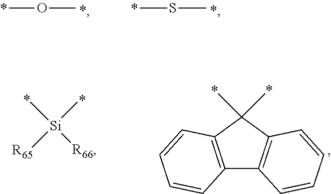

a substituted or unsubstituted alkylene group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, b11 to b13 may each independently be 0 or 1, R₆₁ to R₆₆ may each independently be hydrogen, deuterium, a halogen, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbon atoms, a substituted or unsubstituted alkenyl group of 2 to 20 carbon atoms, a substituted or unsubstituted aryl group of 6 to 60 ring-forming carbon atoms, or a substituted or unsubstituted heteroaryl group of 2 to 60 ring-forming carbon atoms, and d1 to d4 may each independently be an integer of 0 to 4.

In one or more embodiments, the emission layer may further include a second host that is different from the first host, and the second host may be represented by Formula HT.

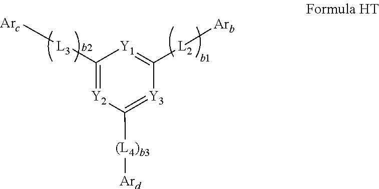

In Formula HT, at least one selected from among Y₁ to Y₃ may be N, and the remainder are CR₅₆, R₅₆ may be hydrogen, deuterium, a substituted or unsubstituted alkyl group of 1 to 20 carbon atoms, a substituted or unsubstituted aryl group of 6 to 60 ring-forming carbon atoms, or a substituted or unsubstituted heteroaryl group of 2 to 60 ring-forming carbon atoms, b1 to b3 may each independently be an integer of 0 to 10, Ar_{b} to Ar_{d} may each independently be hydrogen, deuterium, a substituted or unsubstituted alkyl group of 1 to 20 carbon atoms, a substituted or unsubstituted aryl group of 6 to 60 ring-forming carbon atoms, or a substituted or unsubstituted heteroaryl group of 2 to 60 ring-forming carbon atoms, and L₂ to L₄ may each independently be a direct linkage, a substituted or unsubstituted arylene group of 6 to 60 ring-forming carbon atoms, or a substituted or unsubstituted heteroarylene group of 2 to 60 ring-forming carbon atoms.

In one or more embodiments, the electron transport region may include an electron transport layer on (e.g., arranged on) the emission layer, and an electron injection layer on (e.g., arranged on) the electron transport layer, and the electron transport layer and/or the electron injection layer may include the nitrogen-containing compound.

According to one or more embodiments of the present disclosure, a display device includes a base layer, a circuit layer on (e.g., arranged on) the base layer, and a display element layer on (e.g., arranged on) the circuit layer and including a light emitting element, wherein the light emitting element includes a first electrode, a second electrode on (e.g., arranged on) the first electrode, and at least one functional layer between the first electrode and the second electrode and including a nitrogen-containing compound represented by Formula 1.

In one or more embodiments, the light emitting element may further include a capping layer on (e.g., arranged on) the second electrode, and a refractive index of the capping layer with respect to light in a wavelength range of about 550 nanometers (nm) to about 660 nm may be about 1.6 or more.

In one or more embodiments, the display device may further include a light controlling layer on (e.g., arranged on) the display element layer and including a quantum dot, the light emitting element may be to emit first color light, and the light controlling layer may include a first light controlling part including a first quantum dot that converts the first color light into second color light which is in a longer wavelength range than the first color light, a second light controlling part including a second quantum dot that converts the first color light into third color light which is in a longer wavelength range than both the first color light and the second color light, and a third light controlling part configured to transmit the first color light.

In one or more embodiments, the display device may further include a color filter layer on (e.g., arranged on) the light controlling layer, and the color filter layer may include a first filter configured to transmit the second color light, a second filter configured to transmit the third color light, and a third filter configured to transmit the first color light.

According to one or more embodiments of the present disclosure, a nitrogen-containing compound represented by Formula 1 is provided.

### BRIEF DESCRIPTION OF DRAWINGS

The accompanying drawings are included to provide a further understanding of the present disclosure and are incorporated in and constitute a part of this disclosure. The drawings illustrate example embodiments of the disclosure and, together with the description, serve to explain principles of the present disclosure. Above and/or other aspects of the disclosure should become apparent and appreciated from the following description of embodiments taken in conjunction with the accompanying drawings. In the drawings:

FIG. 1 is a plan view of a display device according to one or more embodiments of the present disclosure;

FIG. 2 is a cross-sectional view of a display device according to one or more embodiments of the present disclosure;

FIG. 3 is a cross-sectional view schematically showing a light emitting element according to one or more embodiments of the present disclosure;

FIG. 4 is a cross-sectional view schematically showing a light emitting element according to one or more embodiments of the present disclosure;

FIG. 5 is a cross-sectional view schematically showing a light emitting element according to one or more embodiments of the present disclosure;

FIG. 6 is a cross-sectional view schematically showing a light emitting element according to one or more embodiments of the present disclosure;

FIG. 7 and FIG. 8 are each a cross-sectional view of a display device according to one or more embodiments of the present disclosure;

FIG. 9 is a cross-sectional view showing a display device according to one or more embodiments of the present disclosure;

FIG. 10 is a cross-sectional view showing a display device according to one or more embodiments of the present disclosure; and

FIG. 11 is a diagram showing a vehicle including display devices according to one or more embodiments of the present disclosure.

### DETAILED DESCRIPTION

The present disclosure may be modified in one or more suitable manners and have many forms, and thus specific/example embodiments will be illustrated in the drawings and described in more detail in the detailed description of the present disclosure. It should be understood, however, that it is not intended to limit the disclosure to the particular forms disclosed, but rather, is intended to cover all modifications, equivalents, and alternatives falling within the spirit and scope of the disclosure.

When explaining each of drawings, like reference numbers are used for referring to like elements. In the accompanying drawings, the dimensions of each structure may be exaggeratingly illustrated for clarity of the present disclosure. It will be understood that, although the terms “first,” “second,” and/or the like, may be used herein to describe one or more suitable components, these components should not be limited by these terms. These terms are only used to distinguish one component from another. For example, a first component could be termed a second component, and, similarly, a second component could be termed a first component, without departing from the scope of example embodiments of the disclosure. As used herein, the singular forms, “a,” “an,” “one,” and “the” are intended to include the plural forms as well, unless the context clearly indicates otherwise. Further, the utilization of “may” when describing embodiments of the present disclosure refers to “one or more embodiments of the present disclosure”.

In the present disclosure, it will be understood that the terms “comprise(s)/comprising, “include(s)/including,” “has(have)/having,” and/or the like specify the presence of features, numbers, steps, operations, component, parts, and/or one or more (e.g., any suitable) combinations thereof disclosed in the disclosure, but do not exclude the possibility of presence or addition of one or more other features, numbers, steps, operations, component, parts, and/or one or more (e.g., any suitable) combinations thereof. As used herein, the terms “and,” “or,” and “and/or” may include any and all combinations of one or more of the associated listed items. Expressions such as “at least one of,” “one of,” and “selected from,” when preceding a list of elements, modify the entire list of elements and do not modify the individual elements of the list. For example, “at least one of a, b, or c”, “at least one selected from a, b, and c”, “at least one selected from among a to c”, etc., may indicate only a, only b, only c, both (e.g., simultaneously) a and b, both (e.g., simultaneously) a and c, both (e.g., simultaneously) b and c, all of a, b, and c, or variations thereof. The “/” utilized herein may be interpreted as “and” or as “or” depending on the situation.

In the present disclosure, if (e.g., when) a layer, a film, a region, or a plate is referred to as being “on” or “in an upper portion of” another layer, film, region, or plate, it may be not only “directly on” the layer, film, region, or plate, but one or more intervening layers, films, regions, or plates may also be present therebetween. On the contrary to this, if (e.g., when) a layer, a film, a region, or a plate is referred to as being “below”, “in a lower portion of” another layer, film, region, or plate, it can be not only directly under the layer, film, region, or plate, but one or more intervening layers, films, regions, or plates may also be present therebetween. In addition, it will be understood that if (e.g., when) a part is referred to as being “on” another part, the part may be arranged above the other part, or arranged under the other part as well. In the present disclosure, “directly on” may refer to that there are no additional layers, films, regions, plates, and/or like, between a layer, a film, a region, a plate, etc. and the other part. For example, “directly on” may refer to two layers or two members are arranged without utilizing an additional member such as an adhesive member therebetween.

In the present disclosure, the term “substituted or unsubstituted” may refer to substituted or unsubstituted with at least one substituent selected from the group consisting of deuterium, a halogen, a cyano group, a nitro group, an amine group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In addition, each of the substituents exemplified above may be substituted or unsubstituted. For example, a biphenyl group may be interpreted as an aryl group or a phenyl group substituted with a phenyl group.

In the present disclosure, the phrase “bonded to an adjacent group to form a ring” may refer to that a group is bonded to an adjacent group to form a substituted or unsubstituted hydrocarbon ring or a substituted or unsubstituted heterocycle. The hydrocarbon ring may include an aliphatic hydrocarbon ring and/or an aromatic hydrocarbon ring. The heterocycle may include an aliphatic heterocycle and/or an aromatic heterocycle. The hydrocarbon ring and the heterocycle may each be monocyclic or polycyclic. In addition, the rings formed by adjacent groups being bonded to each other may be connected to another ring to form a spiro structure.

In the present disclosure, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other, and two ethyl groups in 1,1-diethylcyclopentane may be interpreted as “adjacent groups” to each other. In addition, two methyl groups in 4,5-dimethylphenanthrene may be interpreted as “adjacent groups” to each other.

In the present disclosure, examples of a halogen may include fluorine, chlorine, bromine, or iodine.

In the present disclosure, an alkyl group may be linear or branched. The number of carbons in the alkyl group may be 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, and/or the like, but embodiments of the present disclosure are not limited thereto.

In the present disclosure, a cycloalkyl group may refer to a cyclic alkyl group. The number of carbons in the cycloalkyl group may be 3 to 50, 3 to 30, 3 to 20, or 3 to 10. Examples of the cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, and/or the like, but embodiments of the present disclosure are not limited thereto.

In the present disclosure, an alkenyl group refers to a hydrocarbon group including at least one carbon-carbon double bond in the middle or terminal of an alkyl group having 2 or more carbon atoms. The alkenyl group may be linear or branched. The number of carbon atoms in the alkenyl group is not specifically limited, for example, may be 2 to 30, 2 to 20, or 2 to 10. Examples of the alkenyl group may include a vinyl group, a 1-butenyl group, a 1-pentenyl group, a 1,3-butadienyl aryl group, a styrenyl group, a styryl vinyl group, and/or the like, but embodiments of the present disclosure are not limited thereto.

In the present disclosure, an alkynyl group refers to a hydrocarbon group including at least one carbon-carbon triple bond in the middle or terminal of an alkyl group having 2 or more carbon atoms. The alkynyl group may be linear or branched. Although the number of carbon atoms is not specifically limited, it may be 2 to 30, 2 to 20, or 2 to 10. Examples of the alkynyl group may include an ethynyl group, a propynyl group, and/or the like, but embodiments of the present disclosure are not limited thereto.

In the present disclosure, a hydrocarbon ring group refers to any functional group or substituent derived from an aliphatic hydrocarbon ring. The hydrocarbon ring group may be a saturated hydrocarbon ring group having 5 to 20 ring-forming carbon atoms.

In the present disclosure, an aryl group refers to any functional group or substituent derived from an aromatic hydrocarbon ring. The aryl group may be a monocyclic aryl group or a polycyclic aryl group. The number of ring-forming carbon atoms in the aryl group may be 6 to 60, 6 to 50, 6 to 40, 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, and/or the like, but embodiments of the present disclosure are not limited thereto.

In the present disclosure, a fluorenyl group may be substituted, and two substituents may be bonded to each other to form a spiro structure. Examples of the substituted fluorenyl group are as follows. However, embodiments of the present disclosure are not limited thereto.

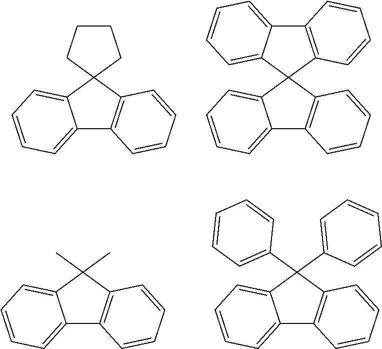

A heterocyclic group as used herein refers to any functional group or substituent derived from a ring containing at least one of B, O, N, P, Si, or Se as a heteroatom. The heterocyclic group includes an aliphatic heterocyclic group and/or an aromatic heterocyclic group. The aromatic heterocyclic group may be a heteroaryl group. The aliphatic heterocycle and the aromatic heterocycle may each be monocyclic or polycyclic.

In the present disclosure, the heterocyclic group may contain at least one of B, O, N, P, Si, or S as a heteroatom. If (e.g., when) the heterocyclic group contains two or more heteroatoms, the two or more heteroatoms may be the same as or different from each other. The heterocyclic group may be a monocyclic heterocyclic group or a polycyclic heterocyclic group, and may include a heteroaryl group. The number of ring-forming carbon atoms in the heterocyclic group may be 2 to 60, 2 to 50, 2 to 40, 2 to 30, 2 to 20, or 2 to 10.

In the present disclosure, the aliphatic heterocyclic group may include at least one of B, O, N, P, Si, or S as a heteroatom. The number of ring-forming carbon atoms in the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, and/or the like, but embodiments of the present disclosure are not limited thereto.

In the present disclosure, the heteroaryl group may contain at least one of B, O, N, P, Si, or S as a heteroatom. If (e.g., when) the heteroaryl group contains two or more heteroatoms, the two or more heteroatoms may be the same as or different from each other. The heteroaryl group may be a monocyclic heterocyclic group or a polycyclic heterocyclic group. The number of ring-forming carbon atoms in the heteroaryl group may be 2 to 60, 2 to 50, 2 to 40, 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, and/or the like, but embodiments of the present disclosure are not limited thereto.

In the present disclosure, the above description of the aryl group may be applied to an arylene group except that the arylene group is a divalent group. The above description of the heteroaryl group may be applied to a heteroarylene group except that the heteroarylene group is a divalent group.

In the present disclosure, a silyl group may include an alkylsilyl group and/or an arylsilyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and/or the like, but embodiments of the present disclosure are not limited thereto.

In the present disclosure, a germanium group may include an alkylgermanium group and/or an arylgermanium group. Examples of germanium group may include a trimethylgermanium group, triethylgermanium group, a t-butyldimethylgermanium group, a vinyldimethylgermanium group, a propyldimethylgermanium group, a triphenylgermanium group, a tri(biphenyl)germanium group, a diphenylgermanium group, a phenyl germanium group, and/or the like, but embodiments of the present disclosure are not limited thereto.

In the present disclosure, the number of carbon atoms in a carbonyl group is not specifically limited, for example, may be 1 to 40, 1 to 30, or 1 to 20. For example, the carbonyl group may have the following structures, but embodiments of the present disclosure are not limited thereto.

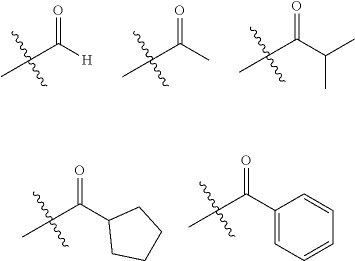

In the present disclosure, the number of carbon atoms in a sulfinyl group or a sulfonyl group is not particularly limited, for example, may be 1 to 30. The sulfinyl group may include an alkyl sulfinyl group and/or an aryl sulfinyl group. The sulfonyl group may include an alkyl sulfonyl group and/or an aryl sulfonyl group.

In the present disclosure, a thio group may include an alkylthio group and/or an arylthio group. The thio group may refer to that a sulfur atom is bonded to the alkyl group or the aryl group defined above. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, and a naphthylthio group, but embodiments of the present disclosure are not limited thereto.

In the present disclosure, an oxy group may refer to that an oxygen atom is bonded to the alkyl group or the aryl group defined above. The oxy group may include an alkoxy group and/or an aryl oxy group. The alkoxy group may be a linear chain, a branched chain, or a ring. The number of carbon atoms in the alkoxy group is not specifically limited, but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, and/or the like, but embodiments of the present disclosure are not limited thereto.

A boron group as used herein may refer to that a boron atom is bonded to the alkyl group or the aryl group defined above. The boron group may include an alkyl boron group and/or an aryl boron group. Examples of the boron group may include a dimethylboron group, a trimethylboron group, a t-butyldimethylboron group, a diphenylboron group, a phenylboron group, and/or the like, but embodiments of the present disclosure are not limited thereto.

In the present disclosure, the number of carbon atoms in an amine group is not specifically limited, for example, may be 1 to 30. The amine group may include an alkyl amine group and/or an aryl amine group. Examples of the amine group may include a methylamine group, a dimethylamine group, a phenylamine group, a diphenylamine group, a naphthylamine group, a 9-methyl-anthracenylamine group, and/or the like, but embodiments of the present disclosure are not limited thereto.

In the present disclosure, the alkyl group among an alkylthio group, an alkylsulfoxy group, an alkylaryl group, an alkylamino group, an alkyl boron group, an alkyl silyl group, and an alkyl amine group may be the same as the examples of the alkyl group described above.

In the present disclosure, the aryl group among an aryloxy group, an arylthio group, an arylsulfoxy group, an arylamino group, an arylboron group, an arylsilyl group, an arylamine group is the same as the examples of the aryl group described above.

In the present disclosure, a direct linkage may refer to a single bond.

In the present disclosure,

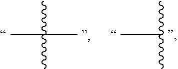

and “ [symbol] ” each refer to a position to be connected.

Hereinafter, embodiments of the disclosure will be described in more detail with reference to the accompanying drawings.

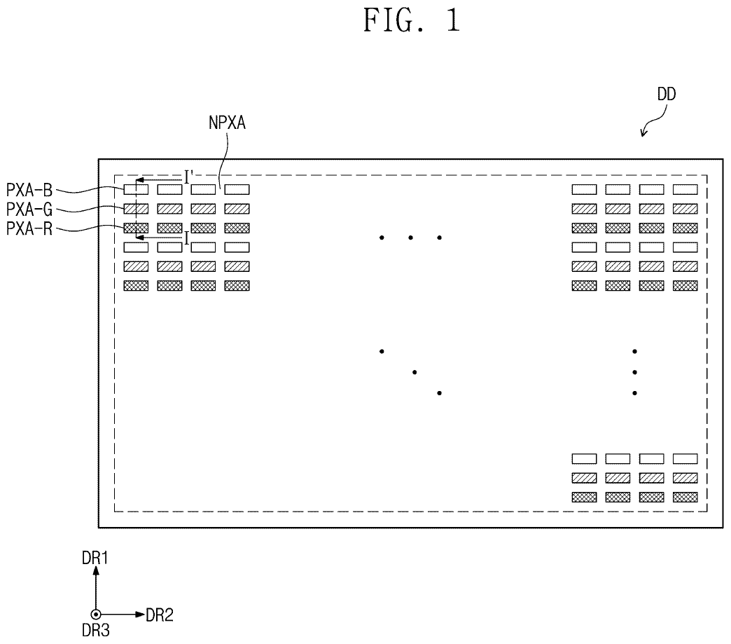

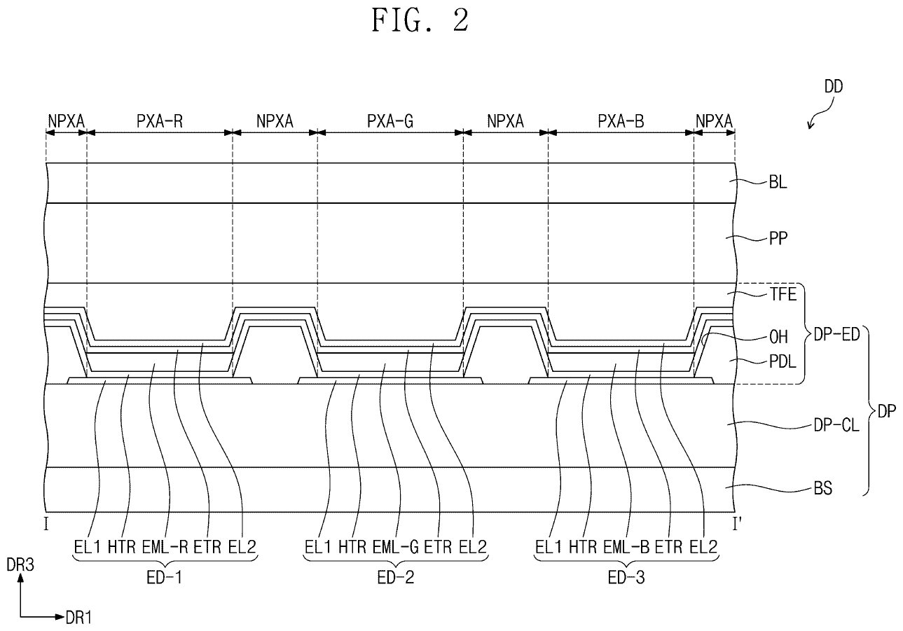

FIG. 1 is a plan view illustrating a display device DD according to one or more embodiments of the present disclosure. FIG. 2 is a cross-sectional view of the display device DD. FIG. 2 is a cross-sectional view illustrating a part taken along the line I-I′ of the display device DD of FIG. 1.

The display device DD may include a display panel DP and an optical layer PP arranged on the display panel DP. The display panel DP may include light emitting elements ED-1, ED-2, and ED-3. The display device DD may include a plurality of light emitting elements ED-1, ED-2, and ED-3. The optical layer PP may be arranged on the display panel DP to control reflected light in the display panel DP due to external light. The optical layer PP may include, for example, a polarization layer and/or a color filter layer. In one or more embodiments, the optical layer PP may not be provided in the display device DD.

A base substrate BL may be arranged on the optical layer PP. The base substrate BL may be a member which provides a base surface on which the optical layer PP arranged. The base substrate BL may be a glass substrate, a metal substrate, a plastic substrate, and/or the like. However, embodiments of the present disclosure are not limited thereto, and the base substrate BL may be an inorganic layer, an organic layer, or a composite material layer. In one or more embodiments, the base substrate BL may not be provided.

The display device DD according to one or more embodiments may further include a filling layer. The filling layer may be arranged between a display device layer DP-ED and the base substrate BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and a display device layer DP-ED. The display device layer DP-ED may include a pixel defining film PDL, the light emitting elements ED-1, ED-2, and ED-3 arranged between respective portions of the pixel defining film PDL, and an encapsulation layer TFE arranged on the light emitting elements ED-1, ED-2, and ED-3.

The base layer BS may be a member which provides a base surface on which the display device layer DP-ED is arranged. The base layer BS may be a glass substrate, a metal substrate, a plastic substrate, and/or the like. However, embodiments of the present disclosure are not limited thereto, and the base layer BS may be an inorganic layer, an organic layer, or a composite material layer.

In one or more embodiments, the circuit layer DP-CL may be arranged on the base layer BS, and the circuit layer DP-CL may include a plurality of transistors. Each of the transistors may include a control electrode, an input electrode, and an output electrode. For example, in one or more embodiments, the circuit layer DP-CL may include switching transistor(s) and driving transistor(s) for driving the light emitting elements ED-1, ED-2, and ED-3 of the display device layer DP-ED.

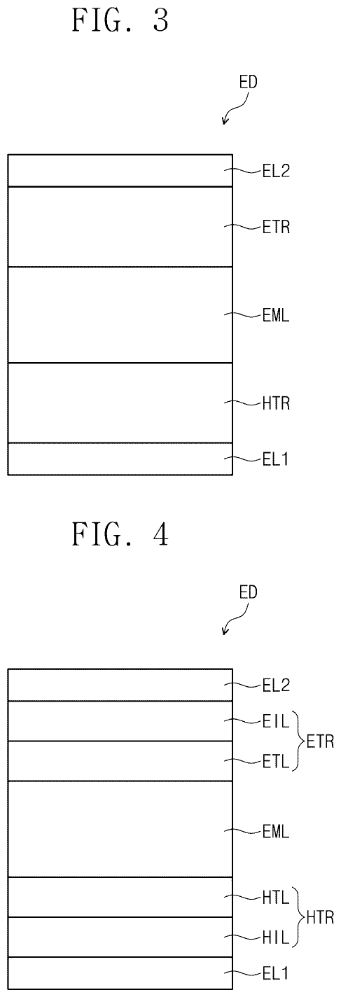

Each of the light emitting elements ED-1, ED-2, and ED-3 may have a structure of one of light emitting elements ED of embodiments according to FIGS. 3 to 6, which will be described in more detail later. Each of the light emitting elements ED-1, ED-2, and ED-3 may include a first electrode EL1, a hole transport region HTR, respective emission layers EML-R, EML-G, and EML-B, an electron transport region ETR, and a second electrode EL2.

FIG. 2 illustrates one or more embodiments in which the respective emission layers EML-R, EML-G, and EML-B of the light emitting elements ED-1, ED-2, and ED-3 are arranged in openings OH defined in the pixel defining film PDL, and the hole transport region HTR, the electron transport region ETR, and the second electrode EL2 are each provided as a common layer across the entire light emitting elements ED-1, ED-2, and ED-3. However, embodiments of the present disclosure are not limited thereto, for example, in one or more embodiments, the hole transport region HTR and the electron transport region ETR may each be provided by being patterned inside the openings OH defined in the pixel defining film PDL. For example, the hole transport region HTR, the respective emission layers EML-R, EML-G, and EML-B, and the electron transport region ETR of the light emitting elements ED-1, ED-2, and ED-3 in one or more embodiments may be provided by being patterned in an inkjet printing method.

The encapsulation layer TFE may cover the light emitting elements ED-1, ED-2 and ED-3. The encapsulation layer TFE may seal the display device layer DP-ED. The encapsulation layer TFE may be a thin film encapsulation layer. The encapsulation layer TFE may be formed by laminating one layer or a plurality of layers. The encapsulation layer TFE may include at least one insulation layer. The encapsulation layer TFE according to one or more embodiments may include at least one inorganic film (hereinafter, an encapsulation-inorganic film). The encapsulation layer TFE according to one or more embodiments may include at least one organic film (hereinafter, an encapsulation-organic film) and at least one encapsulation-inorganic film.

The encapsulation-inorganic film protects the display device layer DP-ED from moisture/oxygen, and the encapsulation-organic film protects the display device layer DP-ED from foreign substances such as dust particles. The encapsulation-inorganic film may include silicon nitride, silicon oxynitride, silicon oxide, titanium oxide, aluminum oxide, and/or the like, but embodiments of the present disclosure are not particularly limited thereto. The encapsulation-organic film may include an acrylic-based compound, an epoxy-based compound, and/or the like. In one or more embodiments, the encapsulation-organic film may include a photopolymerizable organic material, but embodiments of the present disclosure are not particularly limited thereto.

The encapsulation layer TFE may be arranged on the second electrode EL2 and may be arranged filling the opening OH.

Referring to FIG. 1 and FIG. 2, the display device DD may include a non-light emitting region NPXA and light emitting regions PXA-R, PXA-G, and PXA-B. The light emitting regions PXA-R, PXA-G, and PXA-B may be regions in which light generated by the respective light emitting elements ED-1, ED-2, and ED-3 is emitted. The light emitting regions PXA-R, PXA-G, and PXA-B may be spaced and/or apart (e.g., spaced apart or separated) from each other on a plane (e.g., in a plan view).

Each of the light emitting regions PXA-R, PXA-G, and PXA-B may be a region divided by the pixel defining film PDL. The non-light emitting areas NPXA may be areas between adjacent light emitting regions PXA-R, PXA-G, and PXA-B, which correspond to the pixel defining film PDL. In one or more embodiments, the light emitting regions PXA-R, PXA-G, and PXA-B may respectively correspond to pixels. The pixel defining film PDL may divide the light emitting elements ED-1, ED-2, and ED-3. The respective emission layers EML-R, EML-G, and EML-B of the light emitting elements ED-1, ED-2, and ED-3 may be arranged in openings OH defined in the pixel defining film PDL and separated from each other.

The light emitting regions PXA-R, PXA-G, and PXA-B may be divided into a plurality of groups according to the color of light generated from the light emitting elements ED-1, ED-2, and ED-3. In the display device DD of one or more embodiments illustrated in FIG. 1 and FIG. 2, three light emitting regions PXA-R, PXA-G, and PXA-B, which emit red light, green light, and blue light, respectively, are exemplarily illustrated. For example, the display device DD of one or more embodiments may include a red light emitting region PXA-R, a green light emitting region PXA-G, and a blue light emitting region PXA-B that are separated from each other.

In the display device DD according to one or more embodiments, the plurality of light emitting elements ED-1, ED-2 and ED-3 may be to emit light beams having wavelengths different from each other. For example, in one or more embodiments, the display device DD may include a first light emitting element ED-1 that emits red light, a second light emitting element ED-2 that emits green light, and a third light emitting element ED-3 that emits blue light. For example, the red light emitting region PXA-R, the green light emitting region PXA-G, and the blue light emitting region PXA-B of the display device DD may correspond to the first light emitting element ED-1, the second light emitting element ED-2, and the third light emitting element ED-3, respectively.

However, embodiments of the present disclosure are not limited thereto, and the first to third light emitting elements ED-1, ED-2, and ED-3 may be to emit light beams in substantially the same wavelength range or at least one light emitting element may be to emit a light beam in a wavelength range different from the others. For example, in one or more embodiments, the first to third light emitting elements ED-1, ED-2, and ED-3 may all emit blue light.

The light emitting regions PXA-R, PXA-G, and PXA-B in the display device DD according to one or more embodiments may be arranged in a stripe form. Referring to FIG. 1, a plurality of red light emitting regions PXA-R may be arranged with each other along a second direction axis DR2, a plurality of green light emitting regions PXA-G may be arranged with each other along the second direction axis DR2, and a plurality of blue light emitting regions PXA-B may be arranged with each other along the second direction axis DR2. In addition, the red light emitting region PXA-R, the green light emitting region PXA-G, and the blue light emitting region PXA-B may be alternately arranged in this order along a first direction axis DR1.

FIG. 1 and FIG. 2 illustrate that all the light emitting regions PXA-R, PXA-G, and PXA-B have similar area, but embodiments of the present disclosure are not limited thereto. Thus, in one or more embodiments, the light emitting regions PXA-R, PXA-G, and PXA-B may have different areas from each other according to the wavelength range of the emitted light. The areas of the light emitting regions PXA-R, PXA-G, and PXA-B may refer to areas if (e.g., when) viewed on a plane defined by the first direction axis DR1 and the second direction axis DR2 (e.g., the areas in a plan view of the light emitting regions PXA-R, PXA-G, and PXA-B).

In one or more embodiments, an arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in one or more suitable combinations according to the characteristics of display quality desired or required in the display device DD. For example, in one or more embodiments, the arrangement form of the light emitting regions PXA-R, PXA-G, and PXA-B may be a pentile (PENTILE©) arrangement form (for example, an RGBG matrix, an RGBG structure, or an RGBG matrix structure) or a diamond (Diamond Pixel™) arrangement form (e.g., a display (e.g., an OLED display) containing red, blue, and green (RGB) light-emitting regions arranged in the shape of diamonds). PENTILE© is a duly registered trademark of Samsung Display Co., Ltd. Diamond Pixel™ is a trademark of Samsung Display Co., Ltd.

In one or more embodiments, the areas of the light emitting regions PXA-R, PXA-G, and PXA-B may be different from each other. For example, in one or more embodiments, the area of the green light emitting region PXA-G may be smaller than that of the blue light emitting region PXA-B, but embodiments of the present disclosure are not limited thereto.

Hereinafter, FIG. 3 to FIG. 6 are each a cross-sectional view schematically showing a light emitting element according to one or more embodiments of the present disclosure. A light emitting element ED according to one or more embodiments may include a first electrode EL1, a second electrode EL2 opposite to (e.g., oppositely arranged to) the first electrode EL1, and at least one functional layer between the first electrode EL1 and the second electrode EL2. The light emitting element ED of one or more embodiments may include a nitrogen-containing compound of one or more embodiments of the present disclosure, which will be explained later, in the at least one functional layer.

In one or more embodiments, the light emitting element ED may include a hole transport region HTR, an emission layer EML, an electron transport region ETR, and/or the like, stacked in order (e.g., in the stated order) as the at least one functional layer. For example, the light emitting element ED of one or more embodiments may include a first electrode EL1, a hole transport region HTR, an emission layer EML, an electron transport region ETR, and a second electrode EL2, stacked in the stated order.

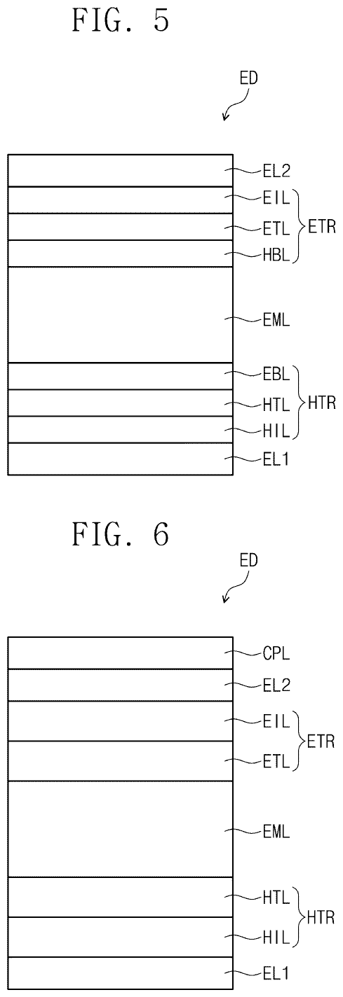

Compared to FIG. 3, FIG. 4 represents a cross-sectional view of a light emitting element ED of one or more embodiments, in which a hole transport region HTR includes a hole injection layer HIL and a hole transport layer HTL, and an electron transport region ETR includes an electron injection layer EIL and an electron transport layer ETL. In addition, compared to FIG. 3, FIG. 5 represents a cross-sectional view of a light emitting element ED of one or more embodiments, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared to FIG. 4, FIG. 6 represents a cross-sectional view of a light emitting element ED of one or more embodiments further including a capping layer CPL arranged on a second electrode EL2.

The light emitting element ED of one or more embodiments may include the nitrogen-containing compound of one or more embodiments represented by Formula 1, which will be explained later, in at least one functional layer included in the light emitting element ED. For example, in one or more embodiments, the light emitting element ED may include the nitrogen-containing compound of one or more embodiments represented by Formula 1, which will be explained later, in an emission layer EML or an electron transport region ETR. However, embodiments of the present disclosure are not limited thereto, and the light emitting element ED of one or more embodiments may include the nitrogen-containing compound of one or more embodiments represented by Formula 1, which will be explained later, in a hole transport region HTR that corresponds to multiple functional layers arranged between the first electrode EL1 and the second electrode EL2, or in a capping layer CPL arranged on a second electrode EL2.

The first electrode EL1 has conductivity (e.g., is a conductor). The first electrode EL1 may be formed of a metal material, a metal alloy, and/or a conductive compound. The first electrode EL1 may be an anode or a cathode. However, embodiments of the present disclosure are not limited thereto. In one or more embodiments, the first electrode EL1 may be a pixel electrode. The first electrode EL1 may be a transmissive electrode, a transflective electrode, or a reflective electrode. The first electrode EL1 may include at least one selected from among silver (Ag), magnesium (Mg), copper (Cu), aluminum (Al), platinum (Pt), palladium (Pd), gold (Au), nickel (Ni), neodymium (Nd), iridium (Ir), chromium (Cr), lithium (Li), calcium (Ca), lithium fluoride (LiF), molybdenum (Mo), titanium (Ti), tungsten (W), indium (In), tin (Sn), and zinc (Zn), a compound of two or more selected therefrom, a mixture of two or more selected therefrom, and/or an oxide thereof.

If (e.g., when) the first electrode EL1 is a transmissive electrode, the first electrode EL1 may include a transparent metal oxide such as indium tin oxide (ITO), indium zinc oxide (IZO), zinc oxide (ZnO), and/or indium tin zinc oxide (ITZO). If (e.g., when) the first electrode EL1 is a transflective electrode or a reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound thereof, or a mixture thereof (e.g., a mixture of Ag and Mg). In one or more embodiments, the first electrode EL1 may have a multilayer structure including a reflective film or a transflective film formed of one or more of the above-described materials, and a transparent conductive film formed of ITO, IZO, ZnO, ITZO, and/or the like. For example, in one or more embodiments, the first electrode EL1 may have a three-layer structure of ITO/Ag/ITO, but embodiments of the present disclosure are not limited thereto. In one or more embodiments, the first electrode EL1 may include one of the above-described metal materials, one or more combinations of at least two metal materials of the above-described metal materials, any oxide of the above-described metal materials, and/or the like. A thickness of the first electrode EL1 may be from about 700 ångström (Å) to about 10,000 Å. For example, in one or more embodiments, the thickness of the first electrode EL1 may be from about 1,000 Å to about 3,000 Å.

The hole transport region HTR may be provided on the first electrode EL1. The hole transport region HTR may include at least one of a hole injection layer HIL, a hole transport layer HTL, a buffer layer or an emission-auxiliary layer, or an electron blocking layer EBL. A thickness of the hole transport region HTR may be, for example, from about 50 Å to about 15,000 Å.

The hole transport region HTR may have a single layer formed of a single material, a single layer formed of a plurality of different materials, or a multilayer structure including a plurality of layers formed of a plurality of different materials.

For example, in one or more embodiments, the hole transport region HTR may have a single layer structure of a hole injection layer HIL or a hole transport layer HTL, or may have a single layer structure formed of a hole injection material and/or a hole transport material. In one or more embodiments, the hole transport region HTR may have a single layer structure formed of a plurality of different materials, or a structure in which a hole injection layer HIL/hole transport layer HTL, a hole injection layer HIL/hole transport layer HTL/buffer layer, a hole injection layer HIL/buffer layer, a hole transport layer HTL/buffer layer, or a hole injection layer HIL/hole transport layer HTL/electron blocking layer EBL are stacked in order (e.g., in the stated order) from the first electrode EL1, but embodiments of the present disclosure are not limited thereto.

The hole transport region HTR may be formed using one or more suitable methods such as a vacuum deposition method, a spin coating method, a casting method, a Langmuir-Blodgett (LB) method, an inkjet printing method, a laser printing method, and/or a laser induced thermal imaging (LITI) method.

In one or more embodiments, the hole transport region HTR may include a compound represented by Formula H-1:

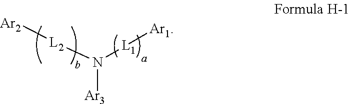

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. a and b may each independently be an integer of 0 to 10. In one or more embodiments, if (e.g., when) a and/or b are each an integer of 2 or greater, a plurality of L₁'s and/or a plurality of L₂'s may each independently be a substituted or unsubstituted arylene group having 6 to 30 ring-forming carbon atoms or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms or a substituted or unsubstituted heteroaryl group having 2 to 30 ring-forming carbon atoms. In addition, in Formula H-1, Ar₃ may be a substituted or unsubstituted aryl group having 6 to 30 ring-forming carbon atoms or a substituted or unsubstituted heteroaryl group having 2 to 30 ring-forming carbon atoms.

In one or more embodiments, the compound represented by Formula H-1 may be a monoamine compound. In one or more embodiments, the compound represented by Formula H-1 may be a diamine compound in which at least one selected from among Ar₁ to Ar₃ includes an amine group as a substituent. In one or more embodiments, the compound represented by Formula H-1 may be a carbazole-based compound including a substituted or unsubstituted carbazole group in at least one of Ar₁ or Ar₂, or a fluorene-based compound including a substituted or unsubstituted fluorene group in at least one of Ar₁ or Ar₂.

The compound represented by Formula H-1 may be any one selected from among compounds in Compound Group H. However, the compounds listed in Compound Group H are mere examples, and the compounds represented by Formula H-1 are not limited to those represented in Compound Group H:

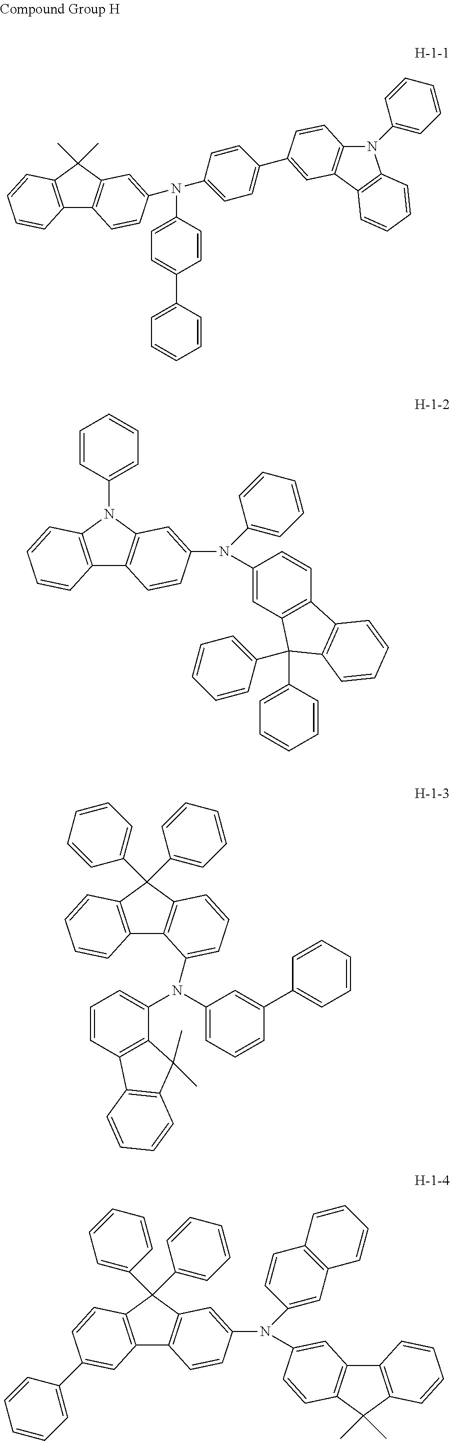 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

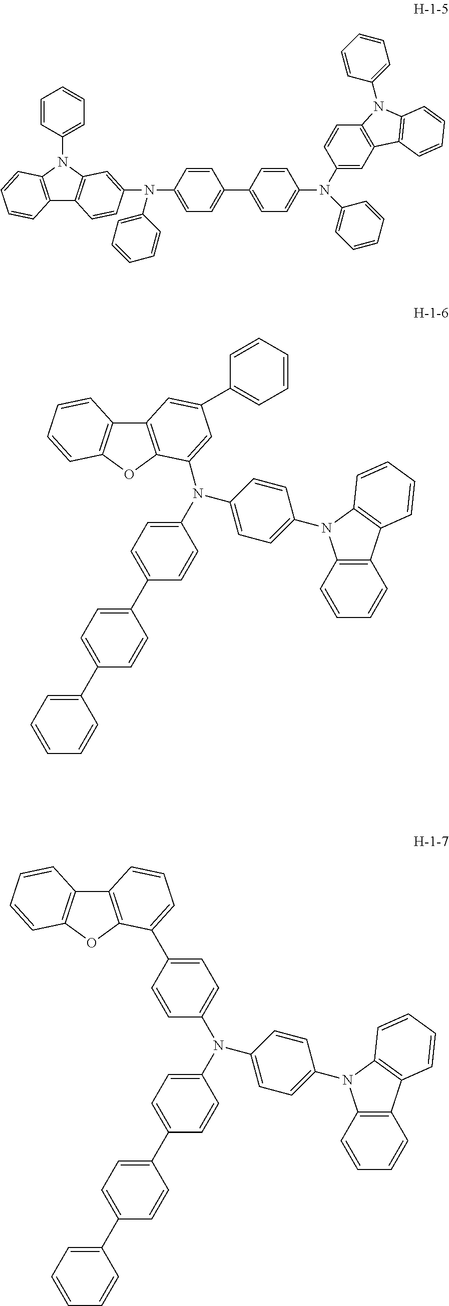 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

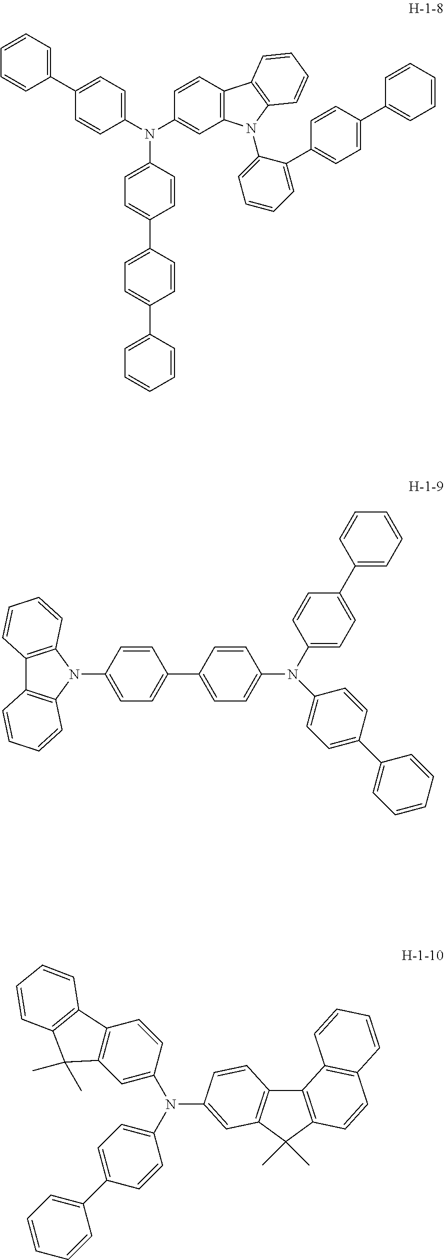 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

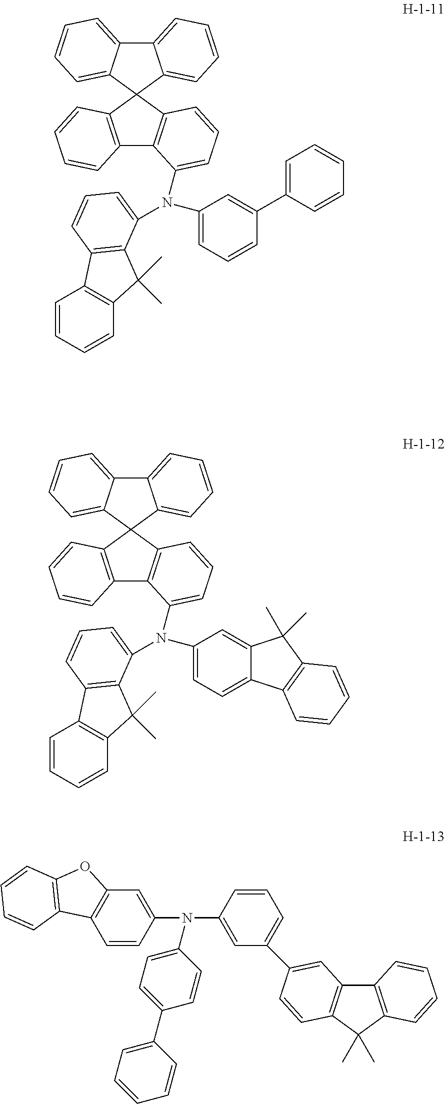 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

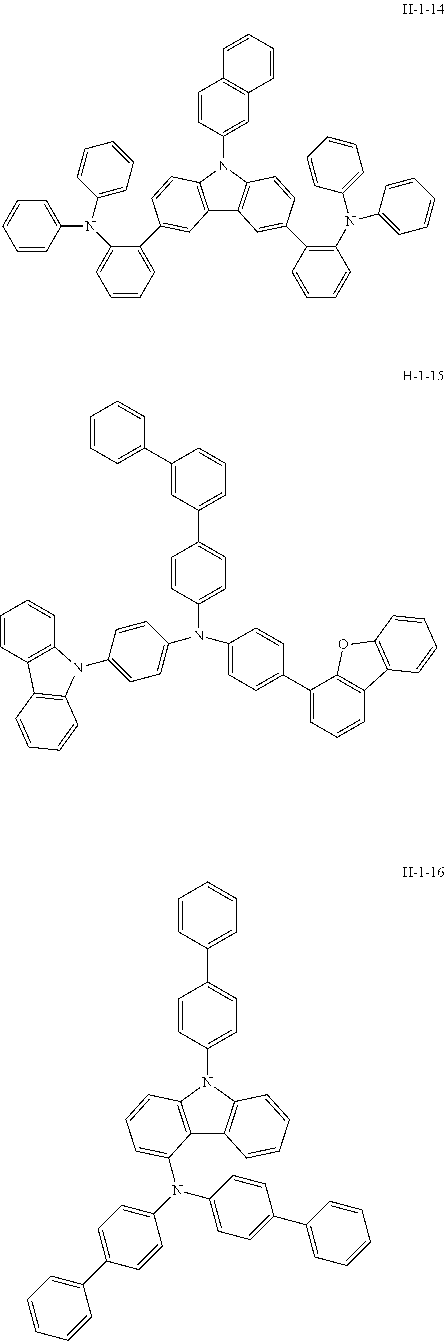 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

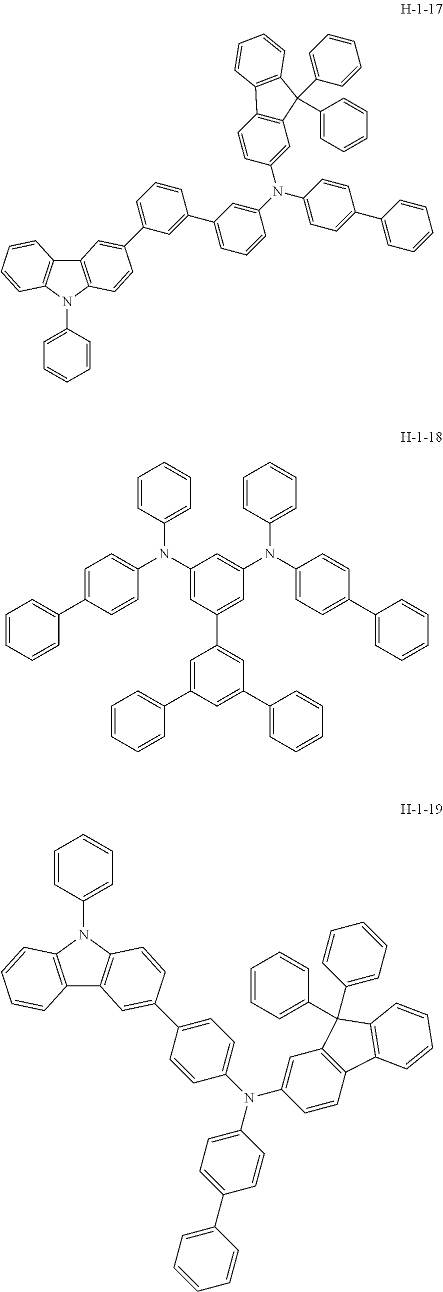 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

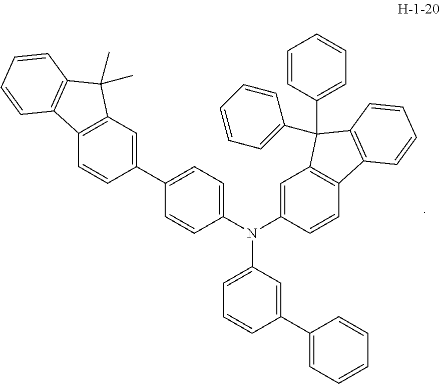 [SMILES: CC1(C)c2ccccc2-c2ccc(-c3ccc(N(c4cccc(-c5ccccc5)c4)c4ccc5c(c4)C(c4ccccc4)(c4ccccc4)c4ccccc4-5)cc3)cc21]

In one or more embodiments, the hole transport region HTR may include one or more selected from among a phthalocyanine compound (such as copper phthalocyanine), N¹,N^{1′}-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine (m-MTDATA), 4,4′,4″-tris(N,N-diphenylamino)triphenylamine (TDATA), 4,4′,4″-tris[N-(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalen-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HATCN), and/or the like.

In one or more embodiments, the hole transport region HTR may include one or more selected from among a carbazole-based derivative (such as N-phenyl carbazole and/or polyvinyl carbazole), a fluorene-based derivative, a triphenylamine-based derivative (such as N,N′-bis(3-methylphenyl)-N,N′-diphenyl-[1,1-biphenyl]-4,4′-diamine (TPD) and/or 4,4′,4″-tris(N-carbazolyl)triphenylamine (TCTA)), N,N′-di(naphthalen-1-yl)-N,N′-diphenyl-benzidine (NPB), 4,4′-cyclohexylidene bis[N,N-bis(4-methylphenyl]benzenamine] (TAPC), 4,4′-bis[N,N′-(3-tolyl)amino]-3,3′-dimethylbiphenyl (HMTPD), 1,3-bis(N-carbazolyl)benzene (mCP), and/or the like.

In one or more embodiments, the hole transport region HTR may include one or more selected from among 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), and/or the like.

In one or more embodiments, the hole transport region HTR may include any one selected from among compounds in Compound Group 4.

The hole transport region HTR may include one or more of the above-described compounds of the hole transport region in at least one of a hole injection layer HIL, a hole transport layer HTL, or an electron blocking layer EBL.

A thickness of the hole transport region HTR may be from about 100 Å to about 10,000 Å, for example, from about 100 Å to about 5,000 Å. If (e.g., when) the hole transport region HTR includes a hole injection layer HIL, the hole injection layer HIL may have, for example, a thickness of about 30 Å to about 1,000 Å. If (e.g., when) the hole transport region HTR includes a hole transport layer HTL, the hole transport layer HTL may have a thickness of about 250 Å to about 1,000 Å. For example, if (e.g., when) the hole transport region HTR includes an electron blocking layer EBL, the electron blocking layer EBL may have a thickness of about 10 Å to about 1,000 Å. If (e.g., when) the thicknesses of the hole transport region HTR, the hole injection layer HIL, the hole transport layer HTL and the electron blocking layer EBL satisfy the above-described ranges, satisfactory hole transport properties may be achieved without a substantial increase in driving voltage.

In one or more embodiments, the hole transport region HTR may further include a charge generating material to increase conductivity (e.g., electric conductivity) in addition to the above-described materials. The charge generating material may be dispersed uniformly (e.g., substantially uniformly) or non-uniformly in the hole transport region HTR. The charge generating material may be, for example, a p-dopant. The p-dopant may include at least one of a halogenated metal compound (e.g., a metal halide compound), a quinone derivative, a metal oxide, or a cyano group-containing compound, but embodiments of the present disclosure are not limited thereto. For example, in one or more embodiments, the p-dopant may include a metal halide compound such as CuI and/or RbI, a quinone derivative such as tetracyanoquinodimethane (TCNQ) and/or 2,3,5,6-tetrafluoro-7,7,8,8-tetracyanoquinodimethane (F4-TCNQ), a metal oxide such as tungsten oxide and/or molybdenum oxide, a cyano group-containing compound such as dipyrazino[2,3-f: 2′,3′-h]quinoxaline-2,3,6,7,10,11-hexacarbonitrile (HATCN) and/or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), and/or the like, but embodiments of the present disclosure are not limited thereto.

As described above, the hole transport region HTR may further include at least one of the buffer layer or the electron blocking layer EBL in addition to the hole injection layer HIL and the hole transport layer HTL. The buffer layer may compensate for a resonance distance according to the wavelength of light emitted from the emission layer EML and may thus increase light emission efficiency. A material that may be included in the hole transport region HTR may be used as a material to be included in the buffer layer. The electron blocking layer EBL is a layer that serves to prevent or reduce the electron injection from the electron transport region ETR to the hole transport region HTR.

The emission layer EML may be provided on the hole transport region HTR. The emission layer EML may have a thickness of, for example, about 100 Å to about 1,000 Å or about 100 Å to about 300 Å. The emission layer EML may have a single layer formed using a single material, a single layer formed using multiple different materials, or a multilayer structure having multiple layers formed using multiple different materials.

The light emitting element ED of one or more embodiments may include a nitrogen-containing compound represented by Formula 1 in at least one functional layer arranged between the first electrode EL1 and the second electrode EL2. In the light emitting element ED of one or more embodiments, the emission layer EML may include the nitrogen-containing compound of one or more embodiments. In one or more embodiments, the emission layer EML may include the nitrogen-containing compound of one or more embodiments as a host. The nitrogen-containing compound of one or more embodiments may be a host material of the emission layer EML. In one or more embodiments of the present disclosure, the nitrogen-containing compound of one or more embodiments may be referred to as a first host.

The nitrogen-containing compound of one or more embodiments includes a carbazole moiety and two triazine moieties. In the nitrogen-containing compound of one or more embodiments, the carbazole moiety and the two triazine moieties are combined via one linker and connected from one another. The linker may be a substituted or unsubstituted trivalent phenyl group. In the present disclosure, the linker connecting the carbazole moiety and the two triazine moieties may be referred to as a “phenyl linker.”

The nitrogen-containing compound of one or more embodiments may include a carbazole moiety, a first triazine moiety, and a second triazine moiety, and may have a structure in which the carbazole moiety, the first triazine moiety, and the second triazine moiety are connected from one another via a linker (e.g., a phenyl linker). The nitrogen at position 9 of the carbazole moiety may be combined with the phenyl linker, and the first and second triazine moieties may be combined with (e.g., bonded to) two carbon atoms at the phenyl linker at ortho positions each with respect to the nitrogen at position 9. For example, the first triazine moiety and the second triazine moiety may be each connected with an ortho position with respect to the carbazole moiety.

Each of the first triazine moiety and the second triazine moiety may be substituted with at least one carbazole group. The phenyl linker is connected with one carbon among carbon atoms at positions 2, 4, and 6 of each of the first triazine moiety and the second triazine moiety, and a substituted or unsubstituted carbazole group is substituted in at least one selected from among two remaining carbon atoms of each of the first triazine moiety and the second triazine moiety. For example, the phenyl linker may be connected with carbon at position 2 selected from among carbons at positions 2, 4, and 6 of the first triazine moiety, and a substituted or unsubstituted carbazole group may be substituted in at least one selected from among carbon atoms at positions 4 and 6 of the first triazine moiety. In addition, the phenyl linker may be connected with carbon at position 2 selected from among carbons at positions 2, 4, and 6 of the second triazine moiety, and a substituted or unsubstituted carbazole group may be substituted in at least one selected from among carbon atoms at positions 4 and 6 of the second triazine moiety. In the present disclosure, the numbering of the ring-forming atoms of each of the first triazine moiety and the second triazine moiety is as follows.

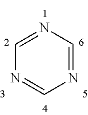

The nitrogen-containing compound of one or more embodiments may be represented by Formula 1.

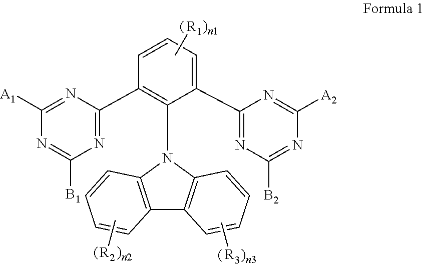

In Formula 1, R₁ to R₃ may each independently be hydrogen, deuterium, a halogen, a cyano group, a substituted or unsubstituted silyl group, a substituted or unsubstituted germanium group, a substituted or unsubstituted amine group, a substituted or unsubstituted oxy group, a substituted or unsubstituted thio group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms. In one or more embodiments, one or more selected from among R₁ to R₃ may be combined with an adjacent group to form a ring.

In one or more embodiments, R₁ to R₃ may each independently be hydrogen, deuterium, a halogen, a cyano group, a substituted or unsubstituted silyl group, a substituted or unsubstituted germanium group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, in one or more embodiments, R₁ may be hydrogen, a substituted or unsubstituted t-butyl group, or a substituted or unsubstituted phenyl group, and R₂ and R₃ may each independently be hydrogen, a substituted or unsubstituted triazine group, or a substituted or unsubstituted phenyl group.

In Formula 1, n1 is an integer of 0 to 3. If (e.g., when) n1 is 0, the nitrogen-containing compound of one or more embodiments may be unsubstituted with R₁. In Formula 1, an embodiment in which n1 is 3, and R₁ are all hydrogens, may be the same as an embodiment in which n1 is 0. If (e.g., when) n1 is an integer of 2 or more, multiple R₁'s may be all the same, or at least one selected from among multiple R₁'s may be different.

In Formula 1, n2 and n3 may each independently be an integer of 0 to 4. If (e.g., when) n2 and n3 are each 0, the nitrogen-containing compound of one or more embodiments may be unsubstituted with R₂ and R₃, respectively. In Formula 1, an embodiment in which each of n2 and n3 is 4, and each of R₂'s and R₃'s is hydrogen, may be the same as an embodiment in which each of n2 and n3 is 0. If each of n2 and n3 is an integer of 2 or more, each of multiple R₂'s and R₃'s may be all the same, or at least one selected from among multiple R₂'s and R₃'s may be different.

In Formula 1, A₁, A₂, B₁, and B₂ may each independently be a substituted or unsubstituted aryl group of 6 to 30 ring-forming carbon atoms or a substituted or unsubstituted heteroaryl group of 2 to 30 ring-forming carbon atoms. For example, in one or more embodiments, A₁, A₂, B₁, and B₂ may each independently be a substituted or unsubstituted phenyl group, a substituted or unsubstituted biphenyl group, or a substituted or unsubstituted carbazole group.

In Formula 1, at least one of A₁ or B₁, and at least one of A₂ or B₂ may each independently be a substituted or unsubstituted carbazole group. For example, in one or more embodiments, one of A₁ and B₁, and one of A₂ and B₂ may each independently be a substituted or unsubstituted carbazole group. In one or more embodiments, A₁ and B₁, and one of A₂ and B₂ may each independently be a substituted or unsubstituted carbazole group. In one or more embodiments, A₁, B₁, A₂, and B₂ may each independently be a substituted or unsubstituted carbazole group.

In one or more embodiments, at least one of A₁ or B₁, and at least one of A₂ or B₂ may each independently be represented by Formula Z.

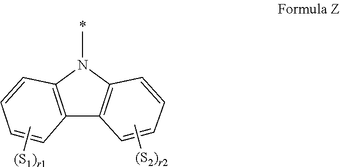

In Formula Z, S₁ and S₂ may each independently be hydrogen, deuterium, a halogen, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula Z, r1 and r2 may each independently be an integer of 0 to 4. If (e.g., when) r1 and r2 are each 0, the nitrogen-containing compound of one or more embodiments may be unsubstituted with S₁ and S₂, respectively. In Formula Z, an embodiment in which each of r1 and r2 is 4, and each of S₁'s and S₂'s is hydrogen, may be the same as an embodiment of Formula Z in which each of r1 and r2 is 0. If (e.g., when) each of r1 and r2 is an integer of 2 or more, each of multiple S₁'s and S₂'s may be all the same, or at least one selected from among multiple S₁'s and S₂'s may be different.

In one or more embodiments, at least one of A₁ or B₁, and at least one of A₂ or B₂ may each independently be a substituted or unsubstituted carbazole group, and the remainder selected from among A₁, B₁, A₂, and B₂ may each independently be represented by one selected from among Formula A-1 to Formula A-4.

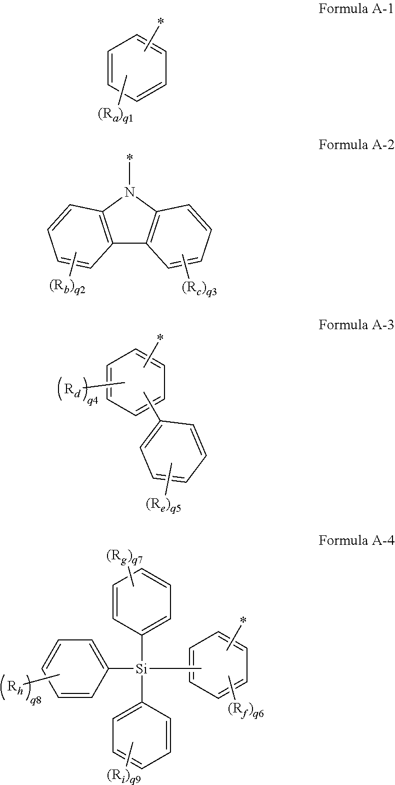

In Formula A-1 to Formula A-4, Rₐ to Rᵢ may each independently be hydrogen, deuterium, a halogen, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, in one or more embodiments, Rₐ to Rᵢ may each independently be hydrogen, a substituted or unsubstituted t-butyl group, or a substituted or unsubstituted phenyl group.

In Formula A-1 to Formula A-4, q1, q5, and q7 to q9 may each independently be an integer of 0 to 5, and q2 to q4, and q6 may each independently be an integer of 0 to 4. If (e.g., when) q1 to q9 are each 0, the nitrogen-containing compound of one or more embodiments may be unsubstituted with Rₐ to Rᵢ, respectively. An embodiment in which each of q1, q5, and q7 to q9 is 5, and each of Rₐ's, Rₑ's, and R_{g}'s to Rᵢ's is hydrogen, may be the same as an embodiment in which each of q1, q5, and q7 to q9 is 0. An embodiment in which each of q2 to q4, and q6 is 4, and each of Rb's to Rd's, and Rt is hydrogen, may be the same as an embodiment in which each of q2 to q4, and q6 is 0. If each of q1 to q9 is an integer of 2 or more, each of multiple Rₐ's to Rᵢ's may be all the same, or at least one selected from among multiple Rₐ's to Rᵢ's may be different.

In one or more embodiments, at least one of A₁ or B₁, and at least one of A₂ or B₂ may each independently be a substituted or unsubstituted carbazole group, and the remainder may each independently be a substituted or unsubstituted aryl group of 6 to 30 ring-forming carbon atoms. For example, in one or more embodiments, at least one of A₁ or B₁, and at least one of A₂ or B₂ may each independently be a substituted or unsubstituted carbazole group, and the remainder may each independently be a substituted or unsubstituted phenyl group or a substituted or unsubstituted biphenyl group.

In one or more embodiments, the nitrogen-containing compound represented by Formula 1 may be represented by Formula 2.

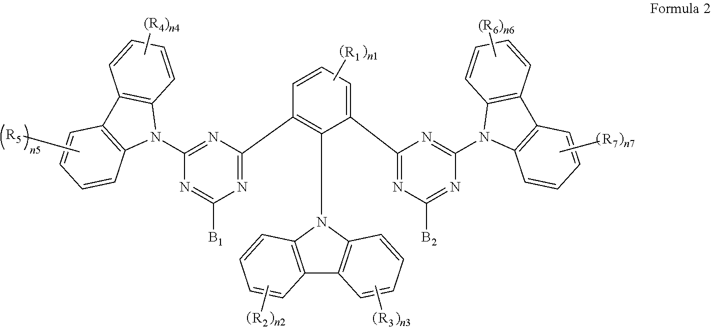

Formula 2 represents embodiments of Formula 1 in which the types (kinds) of A₁ and A₂ are specified. Formula 2 represents embodiments of Formula 1 in which the types (kinds) of A₁ and A₂ are each a substituted or unsubstituted carbazole group.

In Formula 2, R₄ to R₇ may each independently be hydrogen, deuterium, a halogen, a cyano group, a substituted or unsubstituted silyl group, a substituted or unsubstituted germanium group, a substituted or unsubstituted amine group, a substituted or unsubstituted oxy group, a substituted or unsubstituted thio group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms. In one or more embodiments, one or more selected from among R₄ to R₇ may be combined with an adjacent group to form a ring.

In one or more embodiments, R₄ to R₇ may each independently be hydrogen, deuterium, a halogen, a cyano group, a substituted or unsubstituted silyl group, a substituted or unsubstituted germanium group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, in one or more embodiments, R₄ to R₇ may each independently be hydrogen, a substituted or unsubstituted t-butyl group, or a substituted or unsubstituted phenyl group.

In Formula 2, n4 to n7 may each independently be an integer of 0 to 4. If (e.g., when) n4 to n7 are each 0, the nitrogen-containing compound of one or more embodiments may be unsubstituted with R₄ to R₇, respectively. An embodiment in which each of n4 to n7 is 4, and each of R₄'s to R₇'s is hydrogen, may be the same as an embodiment in which each of n4 to n7 is 0. If (e.g., when) each of n4 to n7 is an integer of 2 or more, each of multiple R₄'s to R₇'s may be all the same, or at least one selected from among multiple R₄'s to R₇'s may be different.

In Formula 2, the same contents as those explained in Formula 1 may be applied for R₁ to R₃, n1 to n3, B₁, and B₂.

In one or more embodiments, the nitrogen-containing compound represented by Formula 1 may be represented by Formula 3-1 or Formula 3-2.

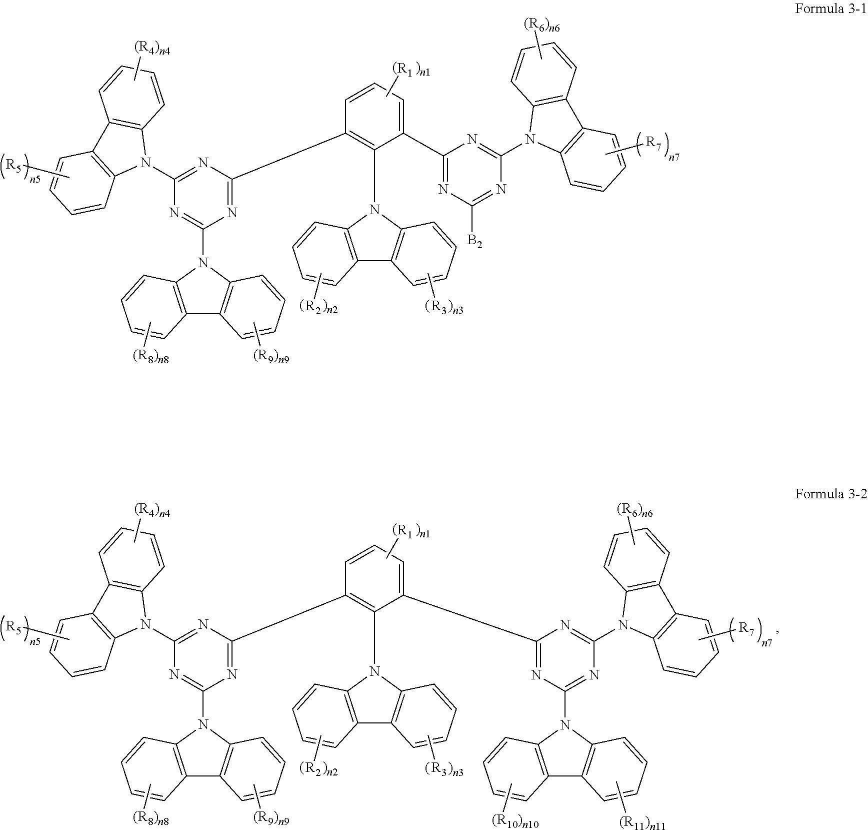

Formula 3-1 and Formula 3-2 represent embodiments of Formula 1 in which three or four types (kinds) selected from among A₁, B₁, A₂, and B₂ are specified. Formula 3-1 corresponds to embodiments of Formula 1 in which A₁, B₁, and A₂ are substituted or unsubstituted carbazole groups. Formula 3-2 corresponds to embodiments of Formula 1 in which A₁, B₁, A₂, and B₂ are substituted or unsubstituted carbazole groups.

In Formula 3-1 and Formula 3-2, R₄ to R₁₁ may each independently be hydrogen, deuterium, a halogen, a cyano group, a substituted or unsubstituted silyl group, a substituted or unsubstituted germanium group, a substituted or unsubstituted amine group, a substituted or unsubstituted oxy group, a substituted or unsubstituted thio group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms. In one or more embodiments, one or more selected from among R₄ to R₁₁ may be combined with an adjacent group to form a ring.

In one or more embodiments, R₄ to R₁₁ may each independently be hydrogen, deuterium, a halogen, a cyano group, a substituted or unsubstituted silyl group, a substituted or unsubstituted germanium group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, in one or more embodiments, R₄ to R₁₁ may each independently be hydrogen, a substituted or unsubstituted t-butyl group, or a substituted or unsubstituted phenyl group.

In Formula 3-1 and Formula 3-2, n4 to n11 may each independently be an integer of 0 to 4. If (e.g., when) n4 to n11 are each 0, the nitrogen-containing compound of one or more embodiments may be unsubstituted with R₄ to R₁₁, respectively. An embodiment in which each of n4 to n11 is 4, and each of R₄'s to R₁₁'s is hydrogen, may be the same as an embodiment in which each of n4 to n11 is 0. If (e.g., when) each of n4 to n11 is an integer of 2 or more, each of multiple R₄'s to R₁₁'s may be all the same, or at least one selected from among multiple R₄'s to R₁₁'s may be different.

In Formula 3-1 and Formula 3-2, the same contents as those explained in Formula 1 may be applied for R₁ to R₃, n1 to n3, and B₂.

In one or more embodiments, the nitrogen-containing compound represented by Formula 1 may be represented by Formula 4-1 or Formula 4-2.

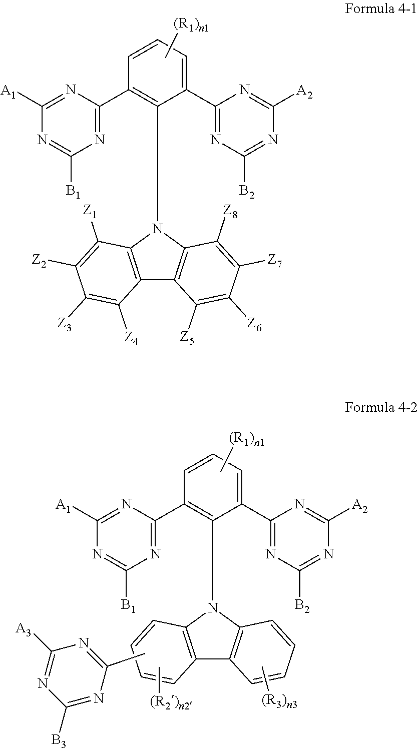

In Formula 4-1, Z₁ to Z₈ may each independently be hydrogen or a substituted or unsubstituted aryl group of 6 to 30 ring-forming carbon atoms. For example, in one or more embodiments, Z₁ to Z₈ may each independently be hydrogen or a substituted or unsubstituted phenyl group.

In Formula 4-2, R_{2′} may be hydrogen, deuterium, a halogen, a cyano group, a substituted or unsubstituted silyl group, a substituted or unsubstituted germanium group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula 4-2, n2′ is an integer of 0 to 3. If (e.g., when) n2′ is 0, the nitrogen-containing compound of one or more embodiments may be unsubstituted with R_{2′}. An embodiment in which n2′ is 3, and all R_{2′} are hydrogens, may be the same as an embodiment in which n2′ is 0. If (e.g., when) n2′ is an integer of 2 or more, each of multiple R_{2′}(s) may be all the same, or at least one selected from among multiple R_{2′}(s) may be different.

In Formula 4-2, A₃ and B₃ may each independently be a substituted or unsubstituted aryl group of 6 to 30 ring-forming carbon atoms or a substituted or unsubstituted heteroaryl group of 2 to 30 ring-forming carbon atoms. For example, in one or more embodiments, A₃ and B₃ may each independently be a substituted or unsubstituted phenyl group.

In Formula 4-1 and Formula 4-2, the same contents as those explained in Formula 1 may be applied for R₁, R₃, n1, n3, A₁, A₂, B₁, and B₂.

In one or more embodiments, the nitrogen-containing compound represented by Formula 1 may be represented by one selected from among Formula 5-1 to Formula 5-3.

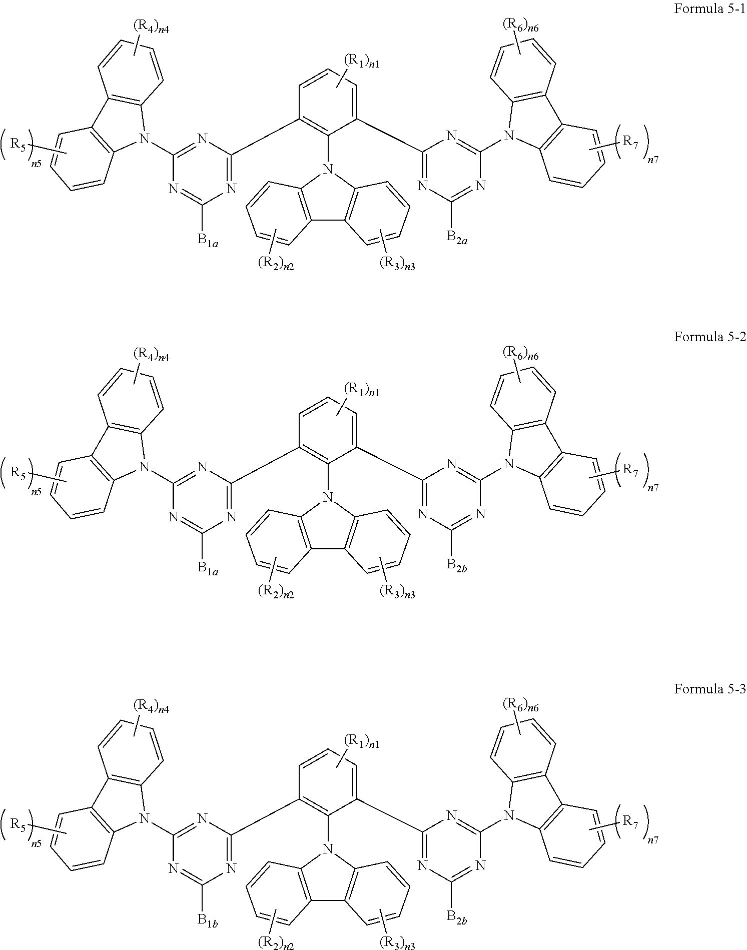

In Formula 5-1 and Formula 5-2, Bia and B₃₂a may each independently be a substituted or unsubstituted aryl group of 6 to 30 ring-forming carbon atoms. For example, in one or more embodiments, Bia and B₂a may each independently be a substituted or unsubstituted phenyl group.

In Formula 5-2 and Formula 5-3, B_{1b} and B_{2b} may each independently be a substituted or unsubstituted heteroaryl group of 2 to 30 ring-forming carbon atoms. For example, in one or more embodiments, B_{1b} and B_{2b} may each independently be a substituted or unsubstituted carbazole group.

In Formula 5-1 to Formula 5-3, the same contents as those explained in Formula 1 may be applied for R₁ to R₃, and n1 to n3.

In Formula 5-1 to Formula 5-3, the same contents as those explained in Formula 2 may be applied for R₄ to R₇, and n4 to n7.

In one or more embodiments, the nitrogen-containing compound represented by Formula 1 may be represented by Formula 6.

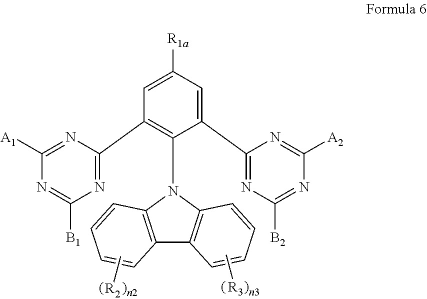

In Formula 6, R₁ₐ may be hydrogen, a substituted or unsubstituted alkyl group of 1 to 10 carbon atoms, or a substituted or unsubstituted aryl group of 6 to 15 ring-forming carbon atoms. For example, in one or more embodiments, R₁ₐ may be hydrogen, a substituted or unsubstituted t-butyl group, or a substituted or unsubstituted phenyl group.

In Formula 6, the same contents as those explained in Formula 1 may be applied for R₂, R₃, n2, n3, A₁, A₂, B₁, and B₂.

The nitrogen-containing compound of one or more embodiments may be any one selected from among compounds represented in Compound Group 1. At least one functional layer included in the light emitting element ED of one or more embodiments may include at least one nitrogen-containing compound selected from among the compounds represented in Compound Group 1. The light emitting element ED of one or more embodiments may include at least one nitrogen-containing compound selected from among the compounds represented in Compound Group 1 in an emission layer EML. In one or more embodiments, the light emitting element ED of one or more embodiments may include at least one nitrogen-containing compound selected from among the compounds represented in Compound Group 1 in an electron transport region.

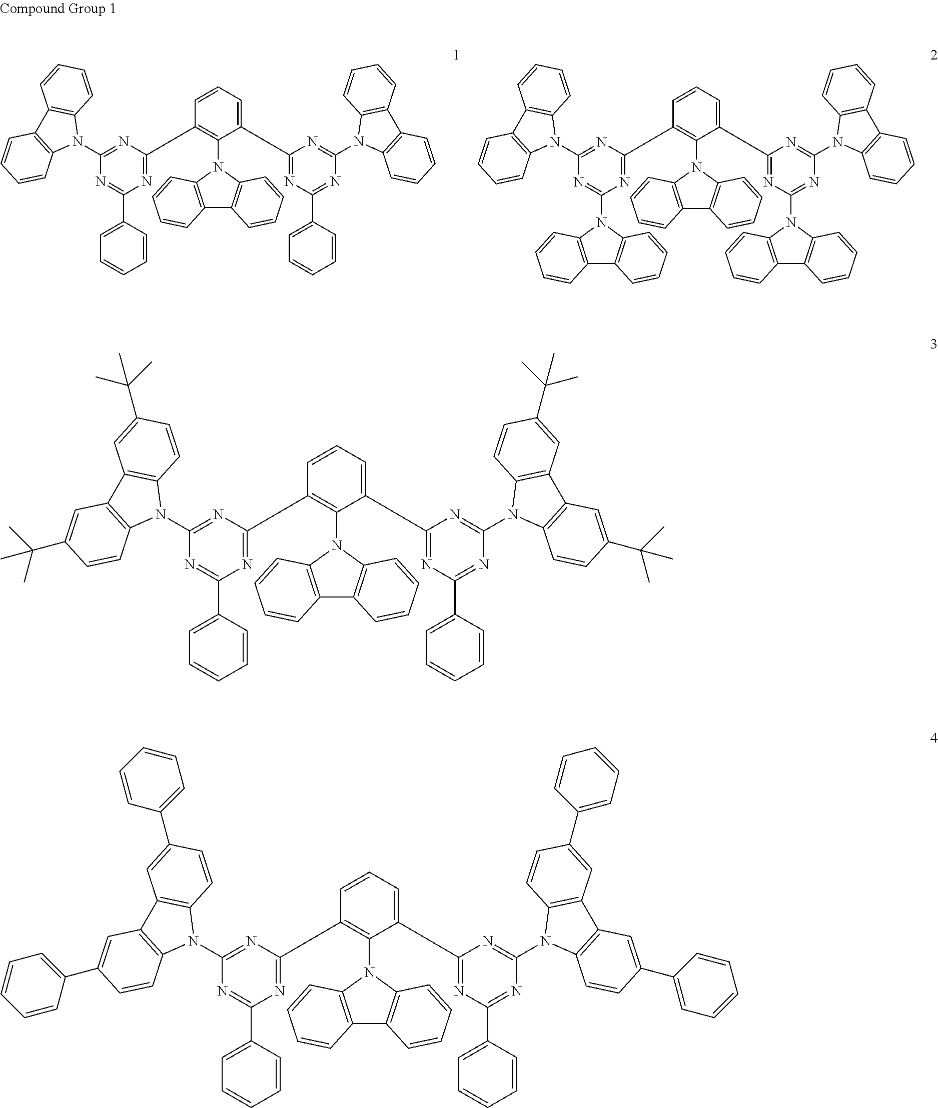

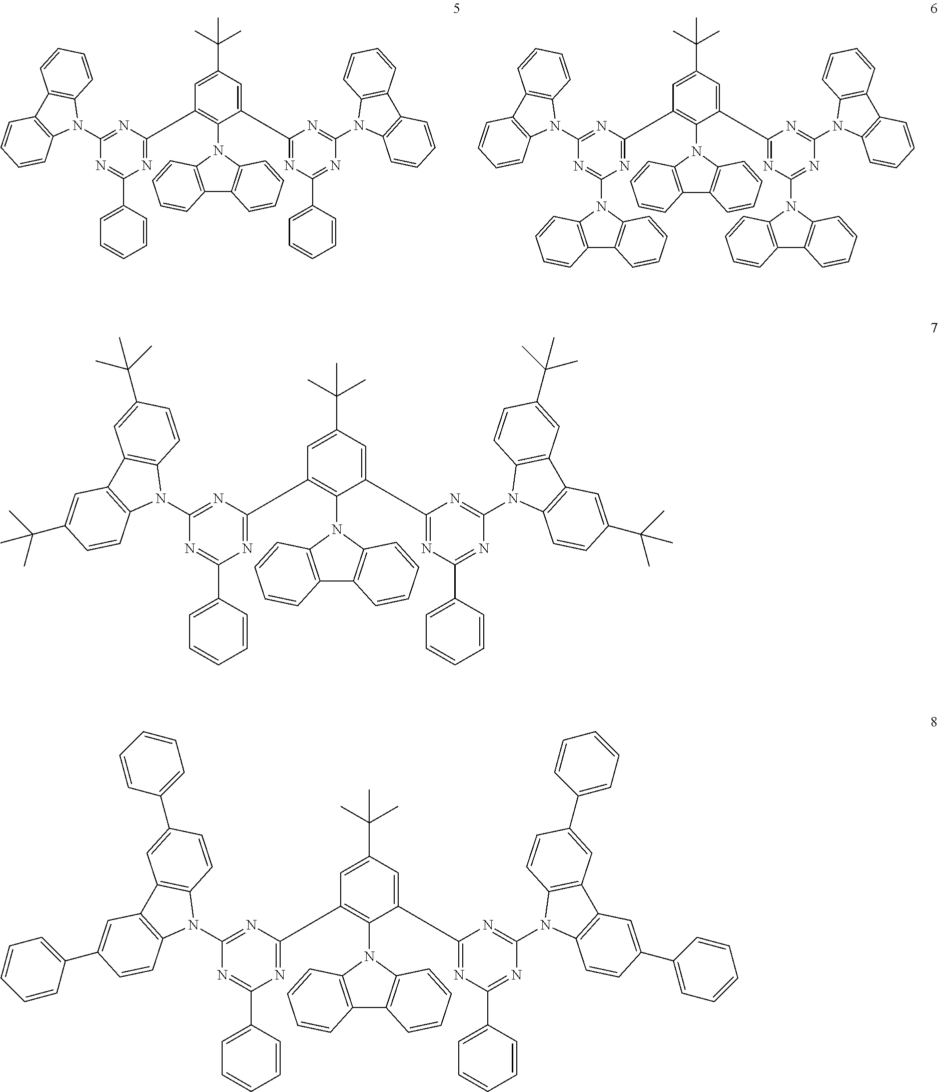

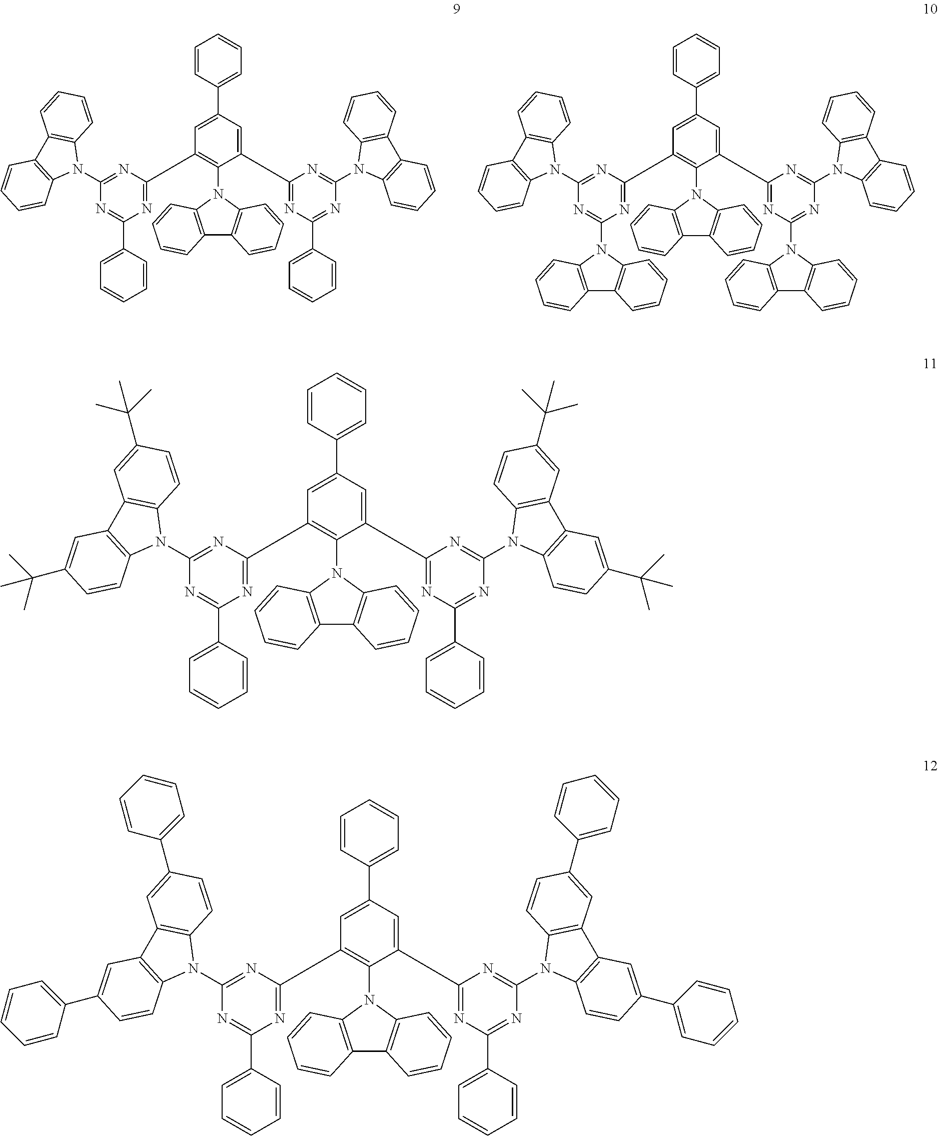

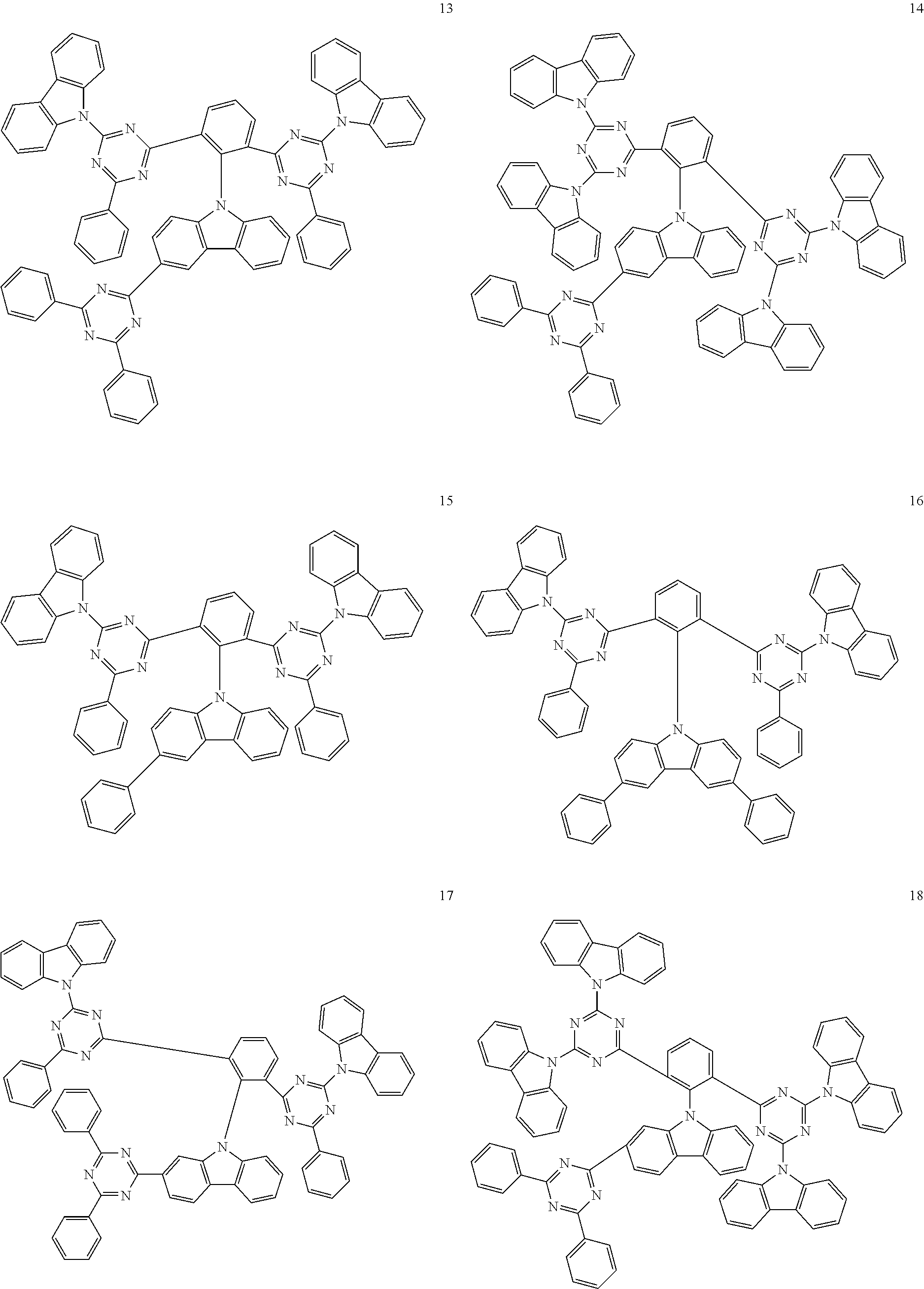

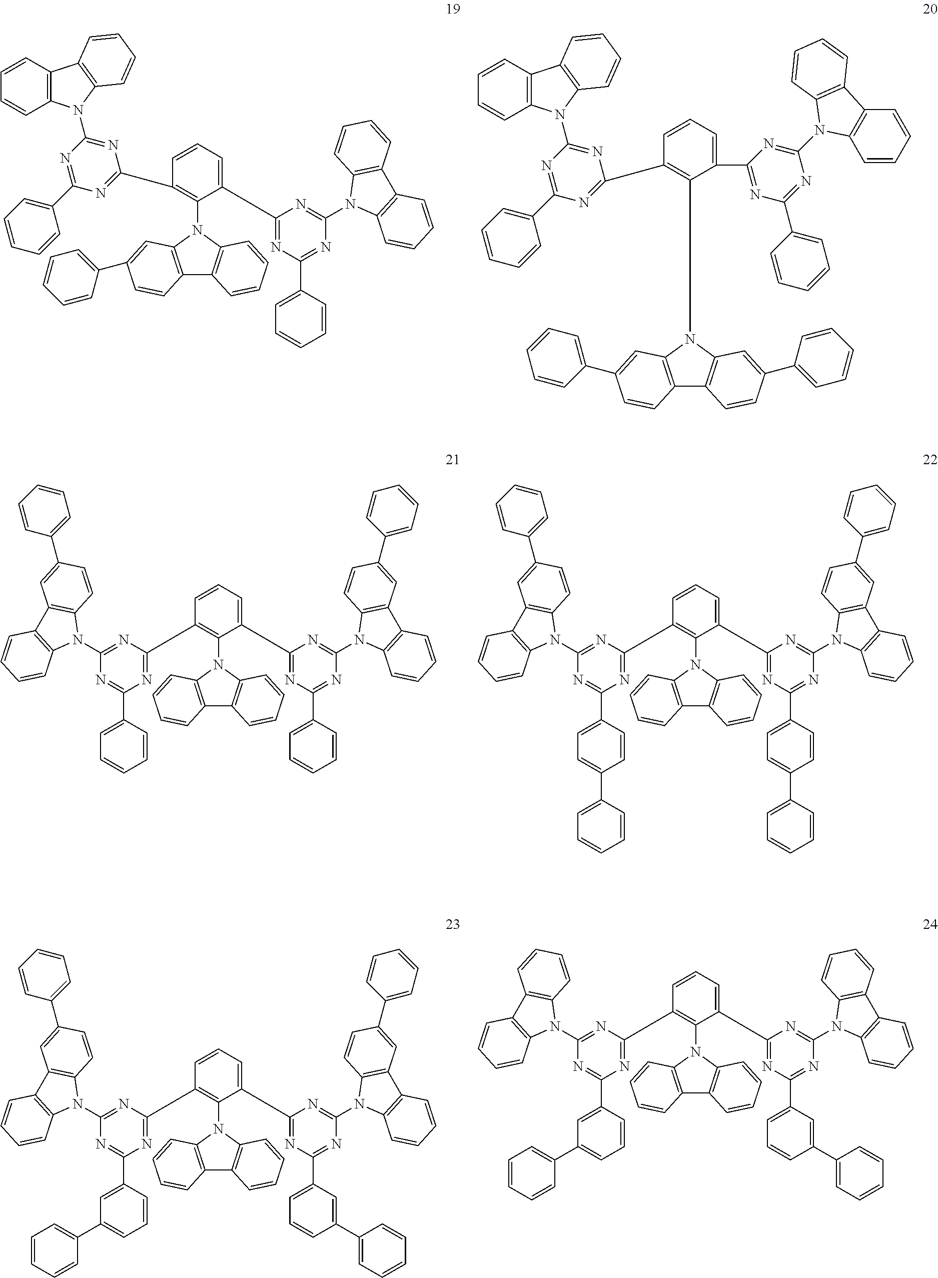

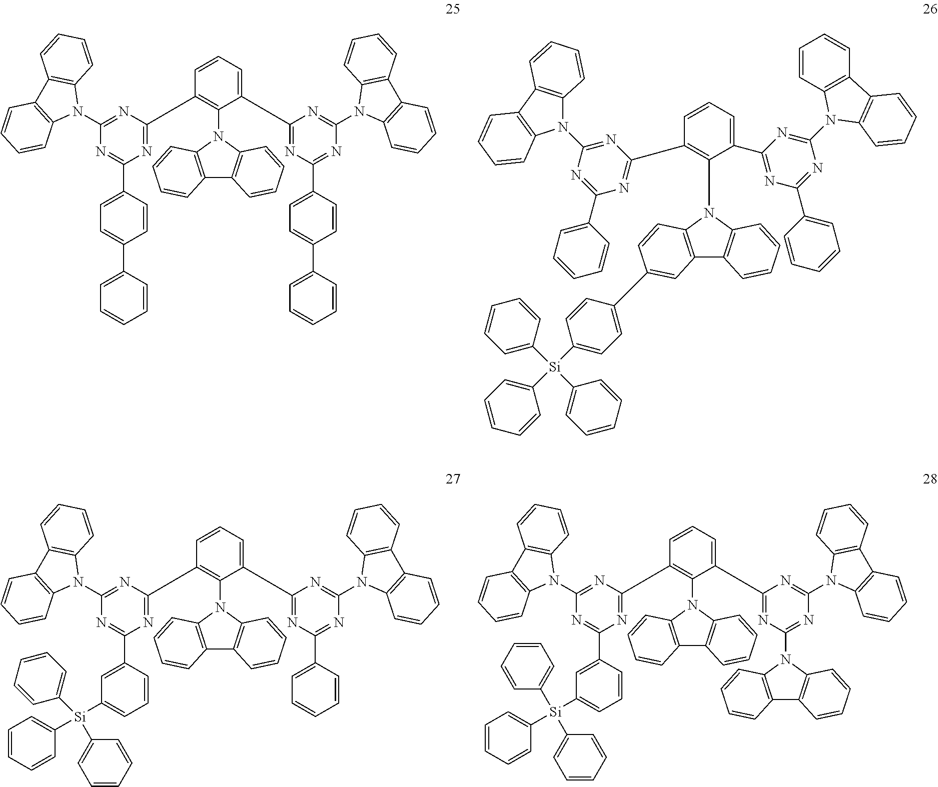

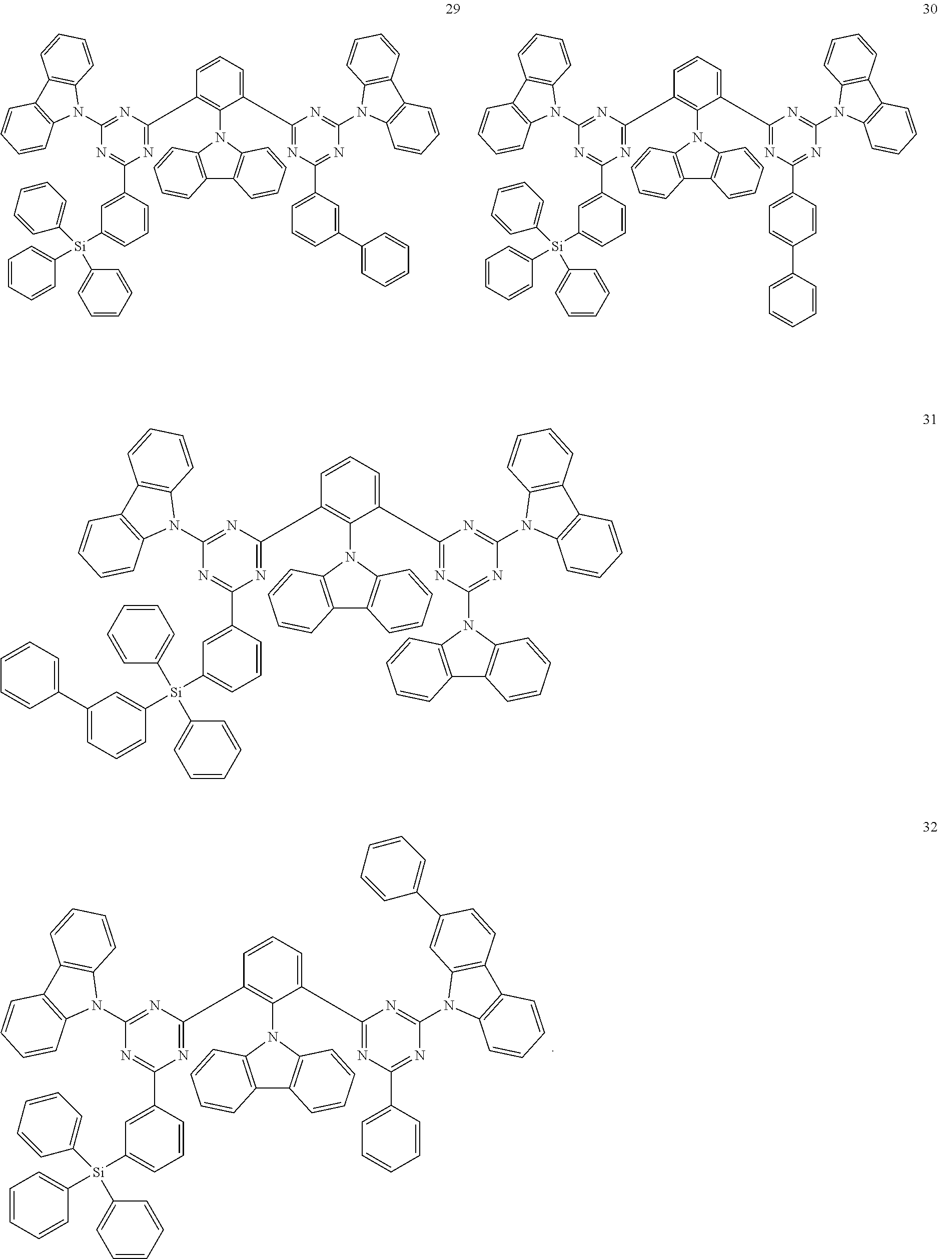

In the light emitting elements ED of embodiments, shown in FIG. 3 to FIG. 6, the emission layer EML may include a host and a dopant, and the emission layer EML may include the nitrogen-containing compound represented by Formula 1 as a host material. However, embodiments of the present disclosure are not limited thereto. For example, in the light emitting element ED of one or more embodiments, the electron transport region ETR may include the nitrogen-containing compound represented by Formula 1.

The nitrogen-containing compound of one or more embodiments may be a thermally activated delayed fluorescence host or a phosphorescence host. The emission layer EML including the nitrogen-containing compound of one or more embodiments may be to emit phosphorescence or thermally activated delayed fluorescence. For example, in one or more embodiments, the emission layer EML may be to emit phosphorescence.

The nitrogen-containing compound of one or more embodiments may have a relatively high lowest excited triplet energy level (T1 level) of about 2.8 eV or higher. The lowest excited triplet energy level is appropriate or suitable for the application as the host material of the emission layer EML of a light emitting element, and the emission layer is to emit thermally activated delayed fluorescence or phosphorescence.

The nitrogen-containing compound of one or more embodiments may have a structure in which a carbazole moiety and first and second triazine moieties are connected with (e.g., bonded to) one phenyl linker, and the first triazine moiety and the second triazine moiety are each substituted at an ortho position with respect to the carbazole moiety. Accordingly, torsion occurs between the carbazole moiety and the first and second triazine moieties, which may result in the high lowest triplet excited energy level. In addition, in the nitrogen-containing compound of one or more embodiments, at least one substituted or unsubstituted carbazole is substituted at the first triazine moiety and the second triazine moieties, respectively. For example, in one or more embodiments, at least one substituted or unsubstituted carbazole is attached to each of the first and second triazine moieties. By introducing a carbazole group that is a donor to the triazine moiety, holes and electrons in the triazine may be balanced (e.g., the carbazole group, acts as a donor to the triazine moiety to balance of hole and electron characteristics in the triazine), and by introducing two or more carbazole groups in a molecule, the controlling effect of charge mobility may be obtained (e.g., incorporating two or more carbazole groups within a molecule enhances the control of charge mobility).

Accordingly, the light emitting element ED of one or more embodiments may show high efficiency and at the same time, long lifetime and low driving voltage properties by including the nitrogen-containing compound of the disclosure in the emission layer EML as a blue phosphorescence host or a thermally activated delayed fluorescence host.

In one or more embodiments, the emission layer EML may include one or two or more types (kinds) of the nitrogen-containing compounds of Formula 1.

In one or more embodiments, the emission layer EML of the light emitting element ED may be to emit blue light. For example, the emission layer EML of the light emitting element ED of one or more embodiments may be to emit blue light in a wavelength range of about 440 nm to about 500 nm. However, embodiments of the present disclosure are not limited thereto, for example, in one or more embodiments, the emission layer EML may be to emit green light or red light.

In one or more embodiments, the nitrogen-containing compound of one or more embodiments may be included in the emission layer EML. The nitrogen-containing compound of one or more embodiments may be included in the emission layer EML as a host material. For example, the emission layer EML in the light emitting element ED of one or more embodiments may include at least one selected from among the nitrogen-containing compounds represented in Compound Group 1 as a host material. However, the use of the nitrogen-containing compound of one or more embodiments is not limited thereto.

In one or more embodiments, the emission layer EML may include multiple compounds. The emission layer EML of one or more embodiments may include first and second hosts, which are different, and a dopant. For example, the emission layer EML of one or more embodiments may include first and second hosts, which are different, and a phosphorescent dopant. In one or more embodiments, the emission layer EML may include first and second hosts, which are different, and a thermally activated delayed fluorescence dopant. In one or more embodiments, the first host may be represented by the above-described Formula 1, the second host may be represented by Formula HT, and the dopant may be represented by Formula D-1. The dopant represented by Formula D-1 may be a phosphorescent dopant.

In one or more embodiments, the emission layer EML may further include a second host represented by Formula HT.

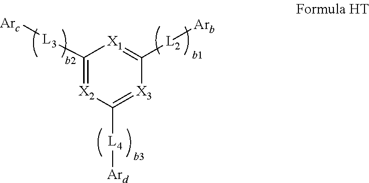

In Formula HT, at least one selected from among X₁ to X₃ may be N, and the remainder may be CR₅₆. For example, in one or more embodiments, any one selected from among X₁ to X₃ may be N, and the remainder two may each independently be CR₅₆. In these embodiments, the second host represented by Formula HT may include a pyridine moiety. In one or more embodiments, two selected from among X₁ to X₃ may be N, and the remainder one may be CR₅₆. In these embodiments, the second host represented by Formula HT may include a pyrimidine moiety. In one or more embodiments, X₁ to X₃ may be all N. In these embodiments, the second host represented by Formula HT may include a triazine moiety.

In Formula HT, R₅₆ may be hydrogen, deuterium, a substituted or unsubstituted alkyl group of 1 to 20 carbon atoms, a substituted or unsubstituted aryl group of 6 to 60 ring-forming carbon atoms, or a substituted or unsubstituted heteroaryl group of 2 to 60 ring-forming carbon atoms.

In Formula HT, b1 to b3 may each independently be an integer of 0 to 10.

In Formula HT, Ar_{b} to Ar_{d} may each independently be hydrogen, deuterium, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, in one or more embodiments, Ar_{b} to Ar_{d} may each independently be a substituted or unsubstituted phenyl group or a substituted or unsubstituted carbazole group.

In Formula HT, L₂ to L₄ may each independently be a direct linkage, a substituted or unsubstituted arylene group of 6 to 30 ring-forming carbon atoms, or a substituted or unsubstituted heteroarylene group of 2 to 30 ring-forming carbon atoms. In one or more embodiments, if (e.g., when) each of b1 to b3 is an integer of 2 or more, L₂'s to L₄'s may each independently be a substituted or unsubstituted arylene group of 6 to 30 ring-forming carbon atoms or a substituted or unsubstituted heteroarylene group of 2 to 30 ring-forming carbon atoms.

In one or more embodiments, the second host represented by Formula HT may be any one selected from among compounds in Compound Group 2. The light emitting element ED of one or more embodiments may include at least one (e.g., any one) selected from among the compounds in Compound Group 2.

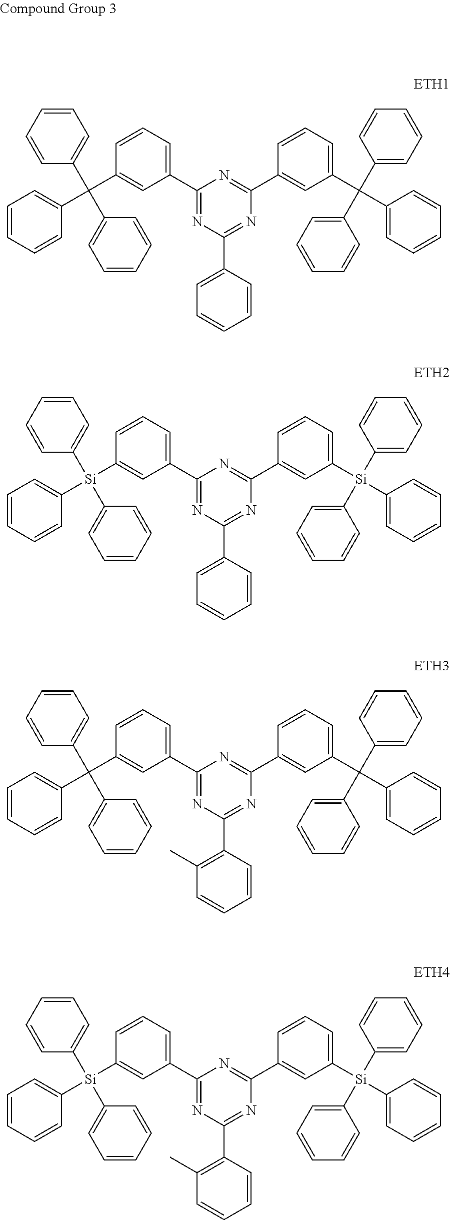 [SMILES: c1ccc(-c2nc(-c3cccc(C(c4ccccc4)(c4ccccc4)c4ccccc4)c3)nc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)nc(-c2cccc([Si](c3ccccc3)(c3ccccc3)c3ccccc3)c2)n1]

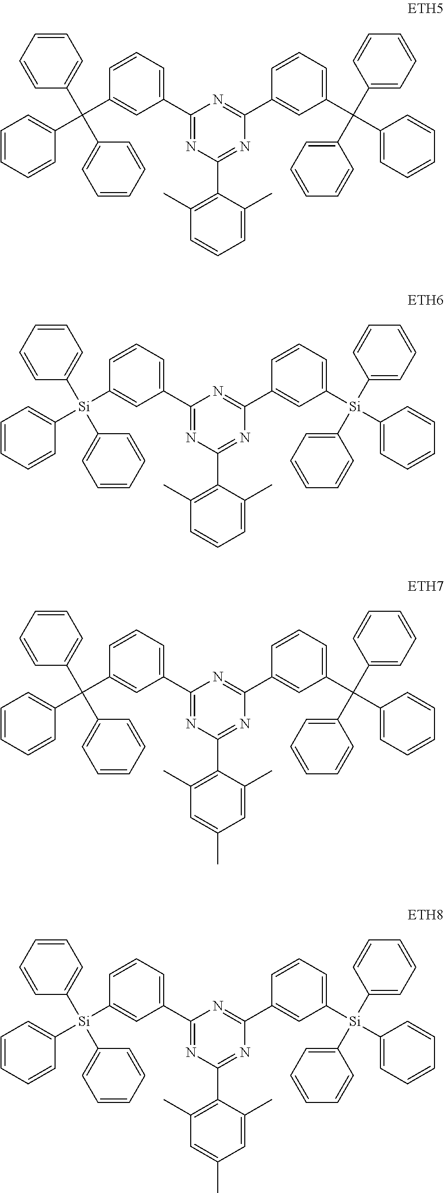 [SMILES: Cc1cccc(C)c1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)nc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)nc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)nc(-c3cccc([Si](c4ccccc4)(c4ccccc4)c4ccccc4)c3)n2)c(C)c1]

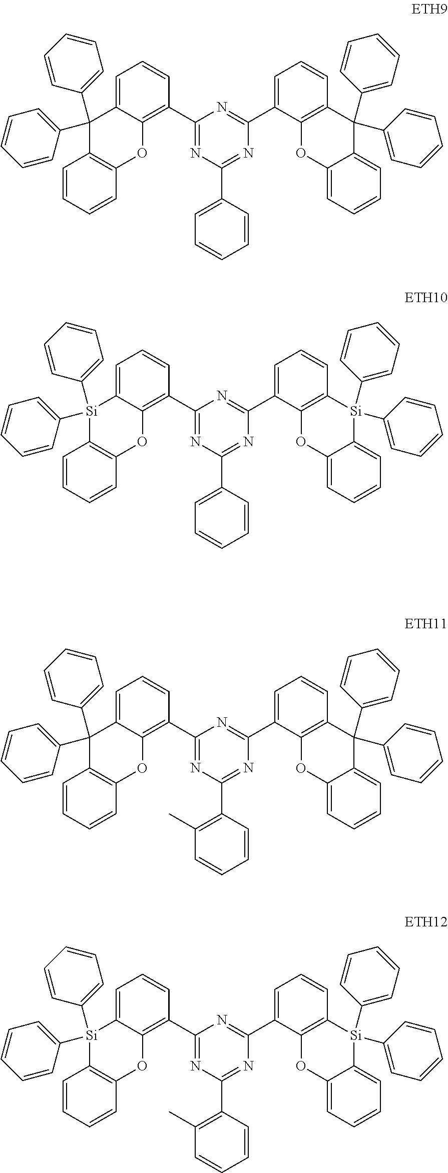 [SMILES: c1ccc(-c2nc(-c3cccc4c3Oc3ccccc3C4(c3ccccc3)c3ccccc3)nc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1]

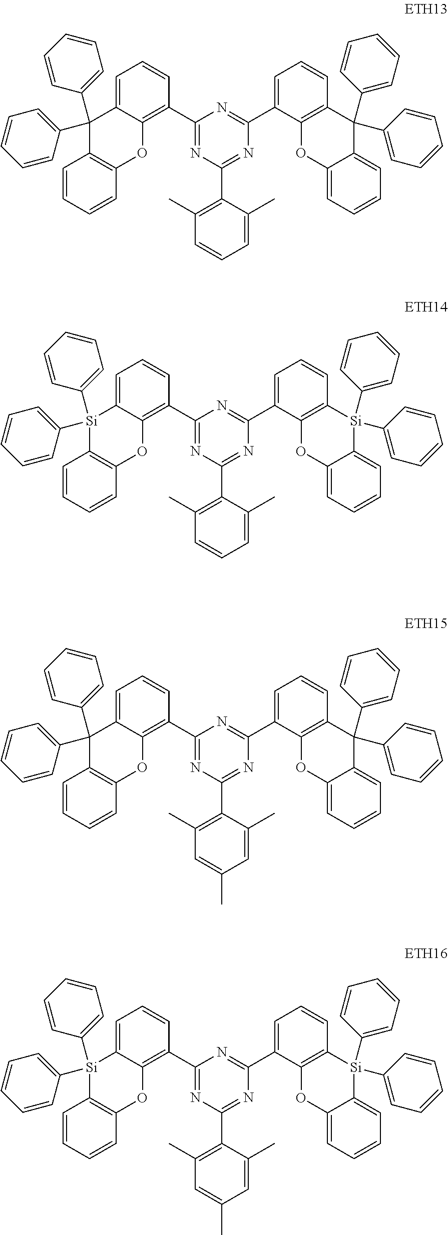 [SMILES: Cc1cccc(C)c1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)nc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)c(C)c1]

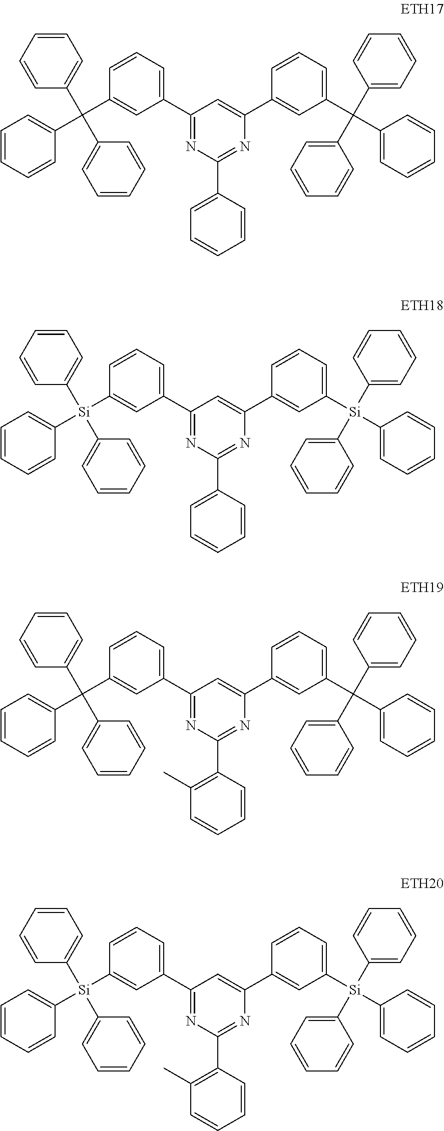 [SMILES: c1ccc(-c2nc(-c3cccc(C(c4ccccc4)(c4ccccc4)c4ccccc4)c3)cc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)cc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)cc(-c2cccc([Si](c3ccccc3)(c3ccccc3)c3ccccc3)c2)n1]

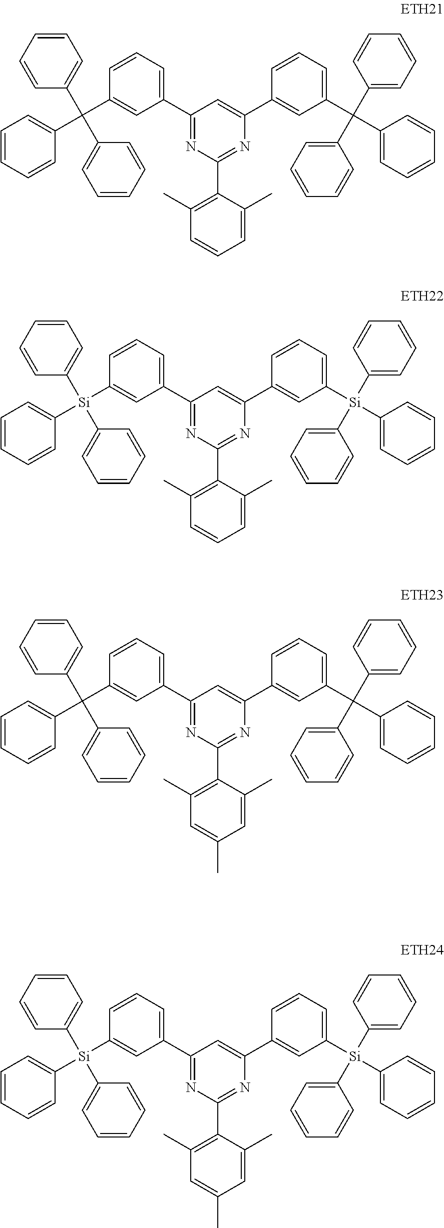 [SMILES: Cc1cccc(C)c1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)cc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)cc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)cc(-c3cccc([Si](c4ccccc4)(c4ccccc4)c4ccccc4)c3)n2)c(C)c1]

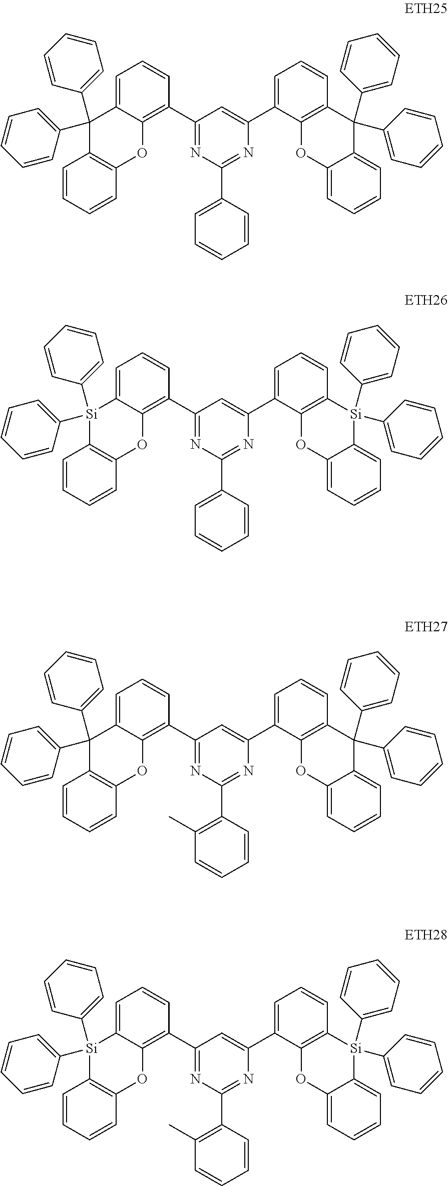 [SMILES: c1ccc(-c2nc(-c3cccc4c3Oc3ccccc3C4(c3ccccc3)c3ccccc3)cc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1]

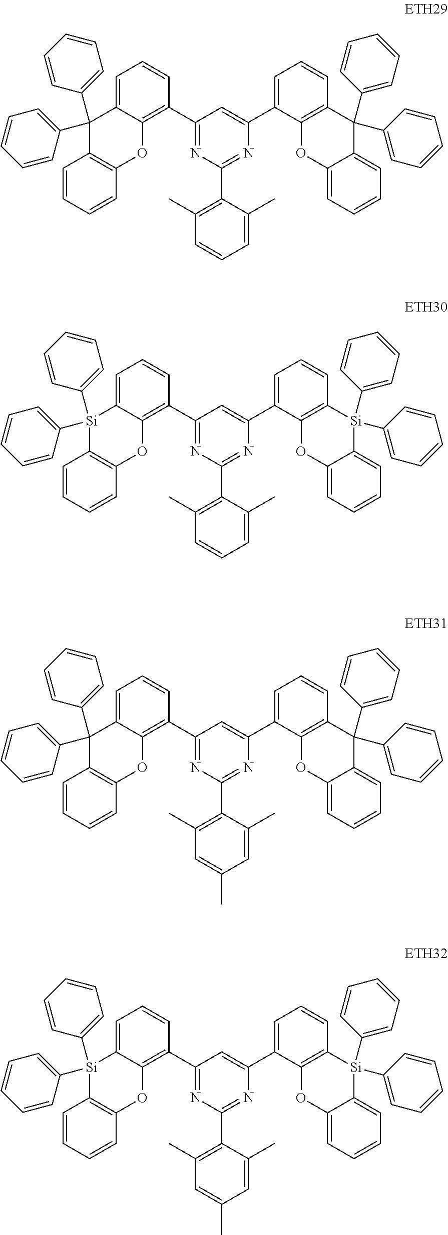 [SMILES: Cc1cccc(C)c1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)cc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)c(C)c1]

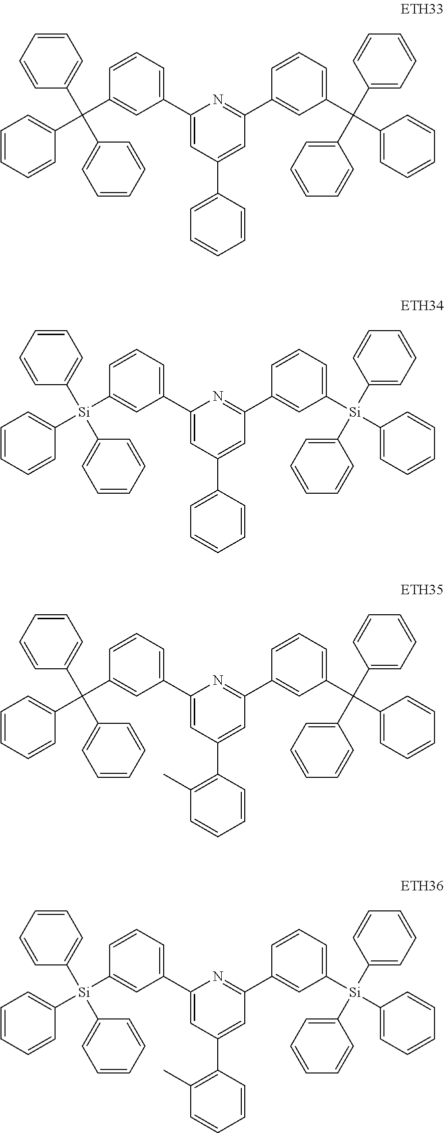 [SMILES: c1ccc(-c2cc(-c3cccc(C(c4ccccc4)(c4ccccc4)c4ccccc4)c3)nc(-c3cccc(C(c4ccccc4)(c4ccccc4)c4ccccc4)c3)c2)cc1 ; c1ccc(-c2cc(-c3cccc([Si](c4ccccc4)(c4ccccc4)c4ccccc4)c3)nc(-c3cccc([Si](c4ccccc4)(c4ccccc4)c4ccccc4)c3)c2)cc1 ; Cc1ccccc1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1ccccc1-c1cc(-c2cccc([Si](c3ccccc3)(c3ccccc3)c3ccccc3)c2)nc(-c2cccc([Si](c3ccccc3)(c3ccccc3)c3ccccc3)c2)c1]

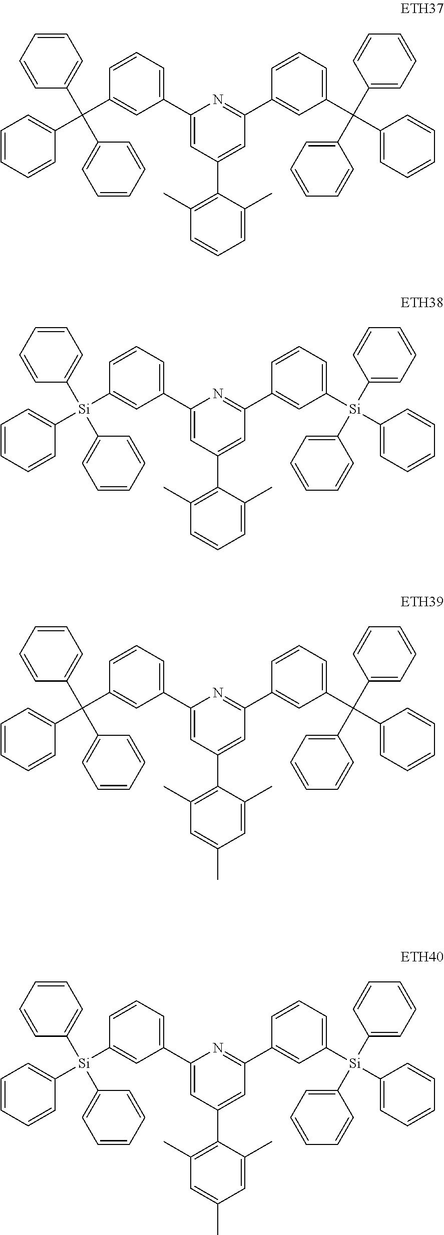 [SMILES: Cc1cccc(C)c1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1cccc(C)c1-c1cc(-c2cccc([Si](c3ccccc3)(c3ccccc3)c3ccccc3)c2)nc(-c2cccc([Si](c3ccccc3)(c3ccccc3)c3ccccc3)c2)c1 ; Cc1cc(C)c(-c2cc(-c3cccc(C(c4ccccc4)(c4ccccc4)c4ccccc4)c3)nc(-c3cccc(C(c4ccccc4)(c4ccccc4)c4ccccc4)c3)c2)c(C)c1 ; Cc1cc(C)c(-c2cc(-c3cccc([Si](c4ccccc4)(c4ccccc4)c4ccccc4)c3)nc(-c3cccc([Si](c4ccccc4)(c4ccccc4)c4ccccc4)c3)c2)c(C)c1]

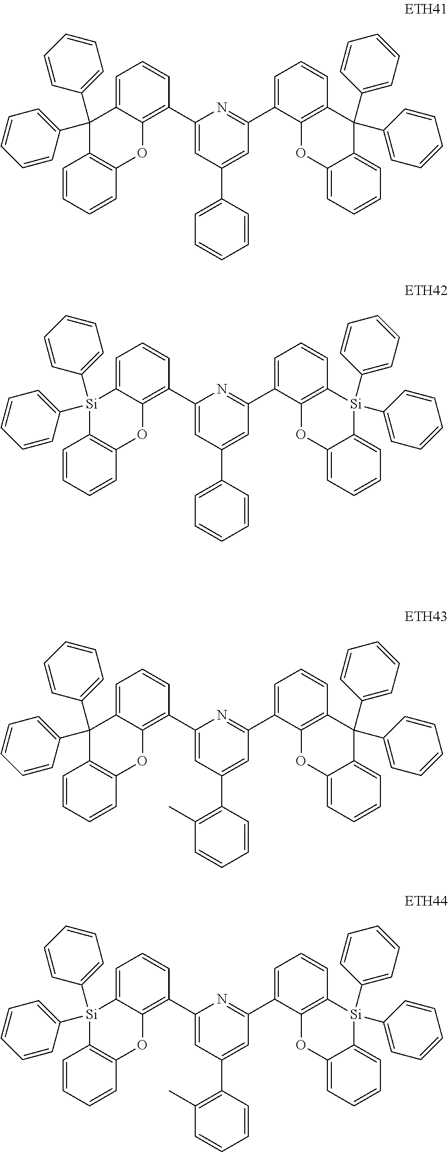 [SMILES: c1ccc(-c2cc(-c3cccc4c3Oc3ccccc3C4(c3ccccc3)c3ccccc3)nc(-c3cccc4c3Oc3ccccc3C4(c3ccccc3)c3ccccc3)c2)cc1 ; c1ccc(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)cc1 ; Cc1ccccc1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1ccccc1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1]

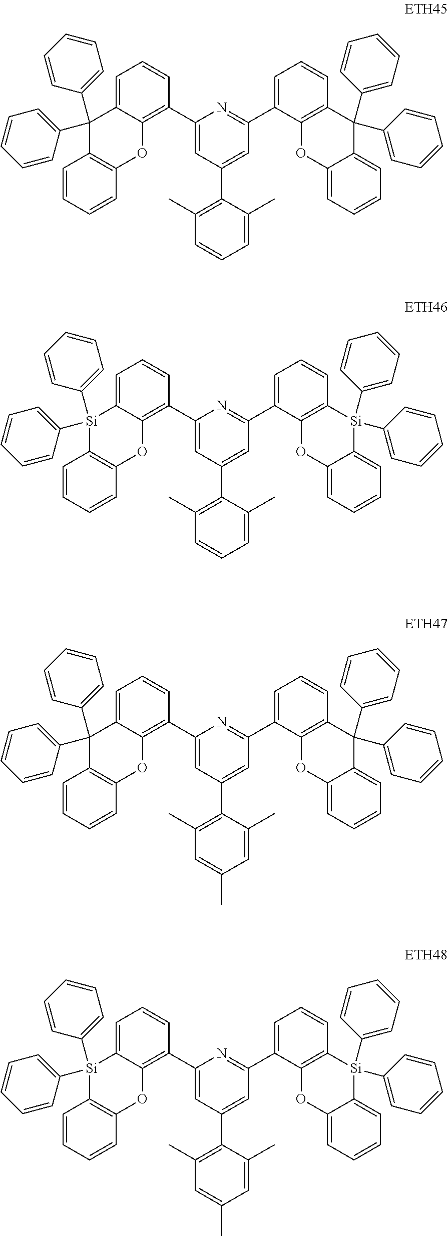 [SMILES: Cc1cccc(C)c1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1cccc(C)c1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1 ; Cc1cc(C)c(-c2cc(-c3cccc4c3Oc3ccccc3C4(c3ccccc3)c3ccccc3)nc(-c3cccc4c3Oc3ccccc3C4(c3ccccc3)c3ccccc3)c2)c(C)c1 ; Cc1cc(C)c(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)c(C)c1]

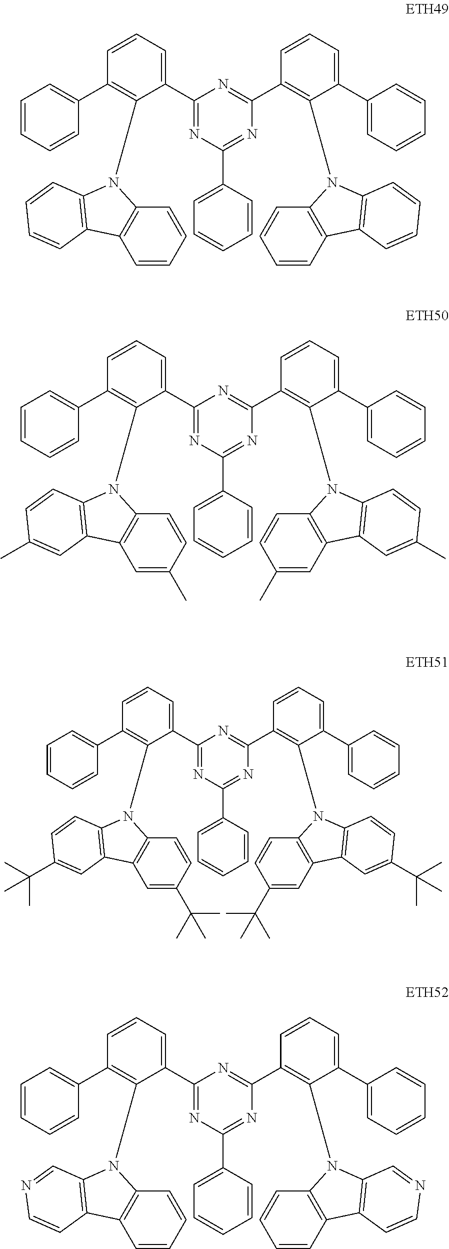

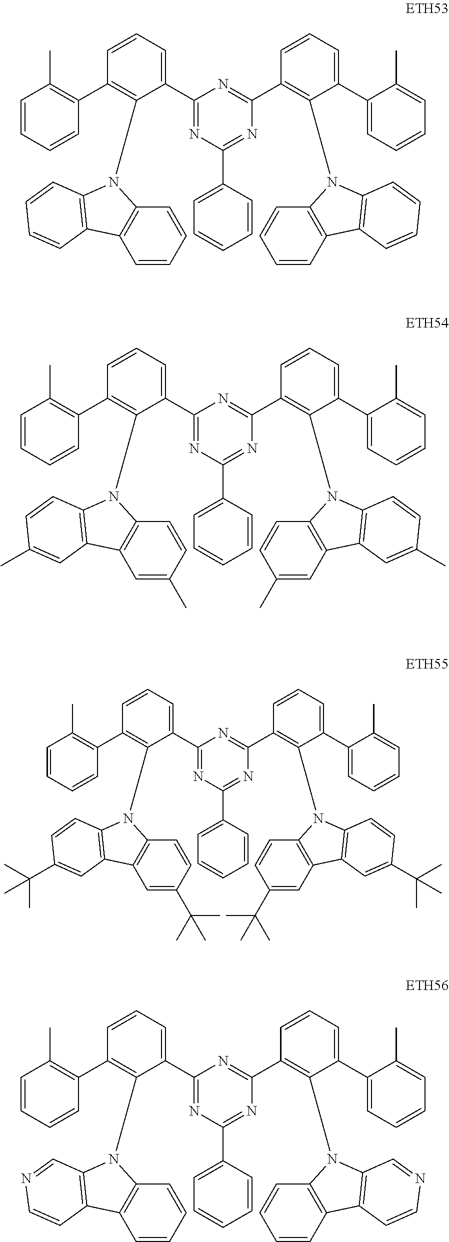

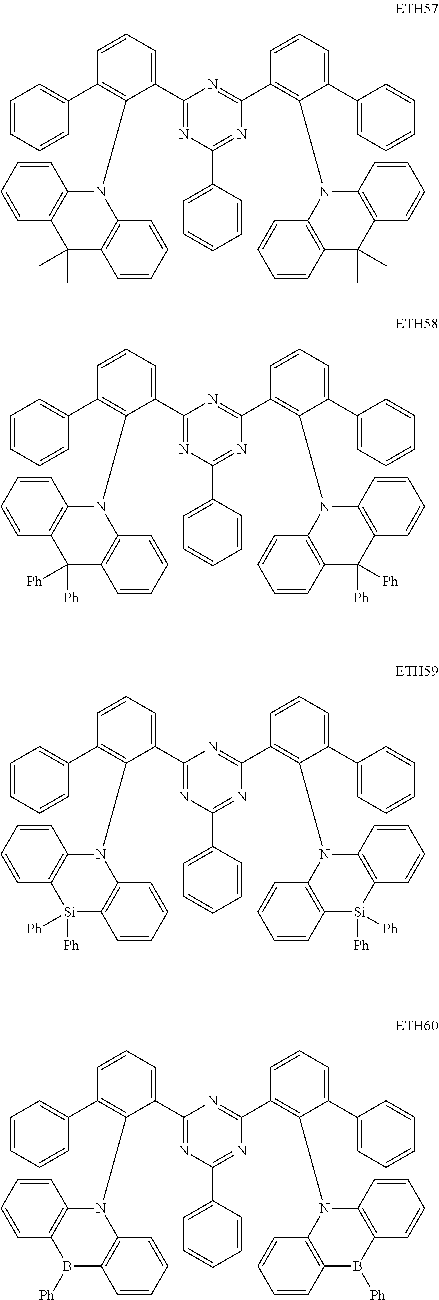

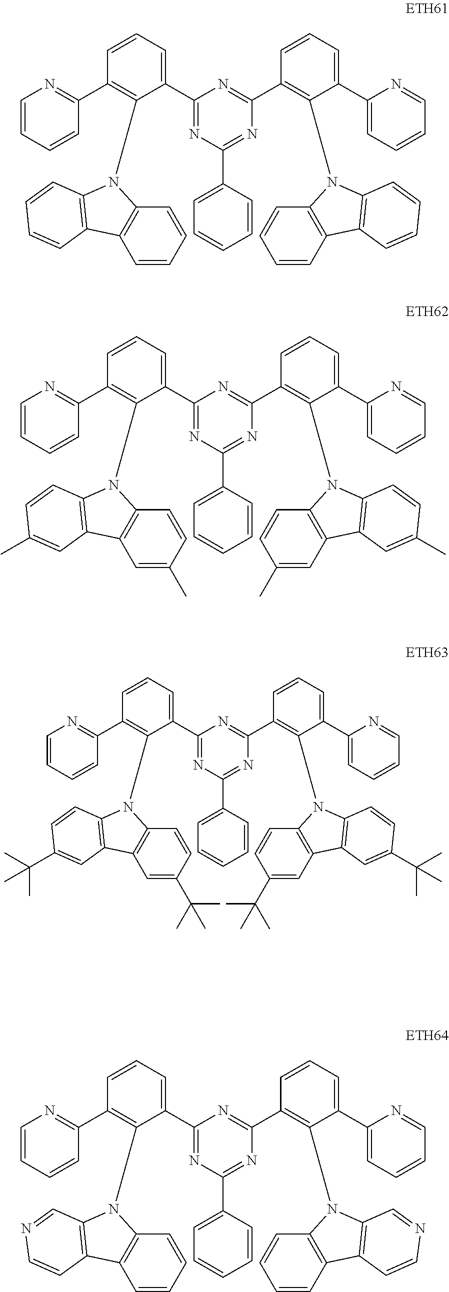

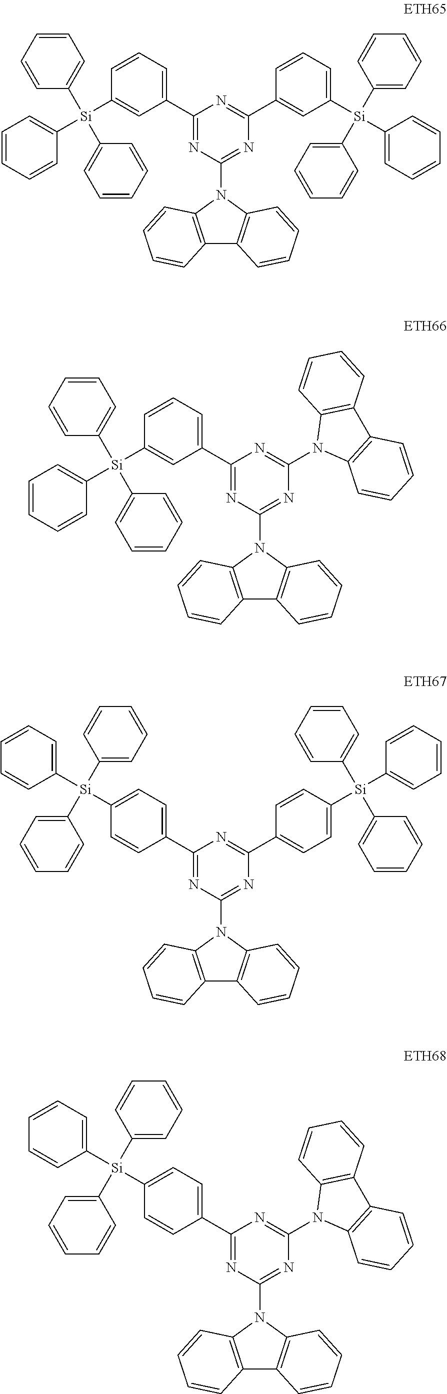 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5ccccc54)n3)cc2)cc1 ; c1ccc([Si](c2ccccc2)(c2ccccc2)c2ccc(-c3nc(-n4c5ccccc5c5ccccc54)nc(-n4c5ccccc5c5ccccc54)n3)cc2)cc1]

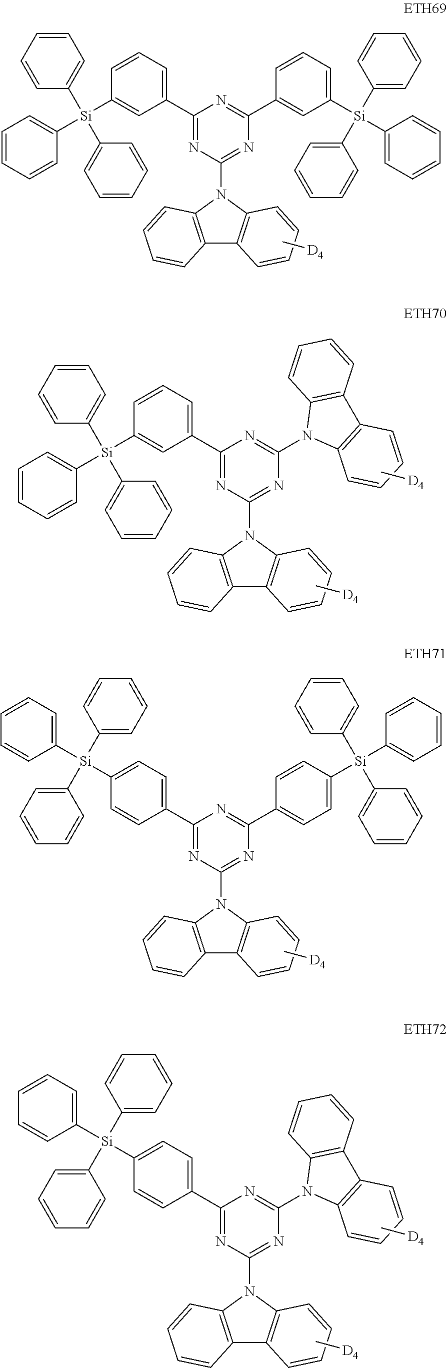

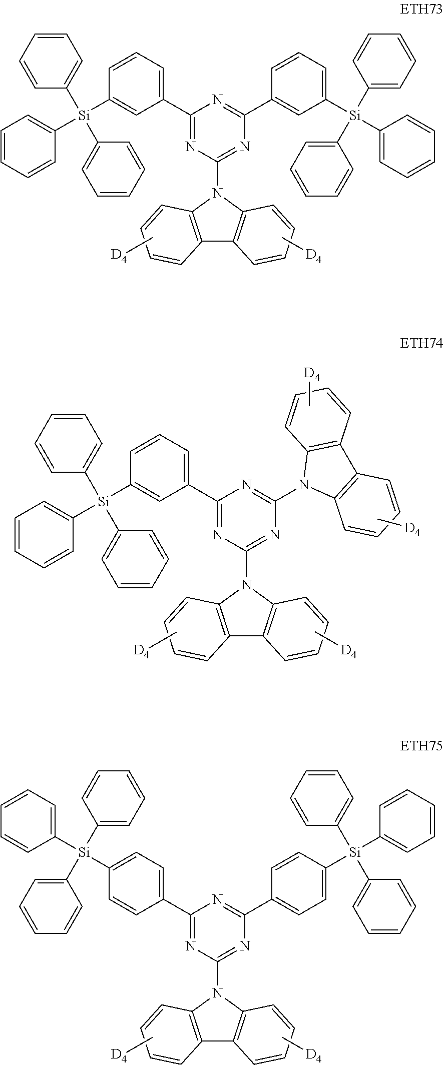

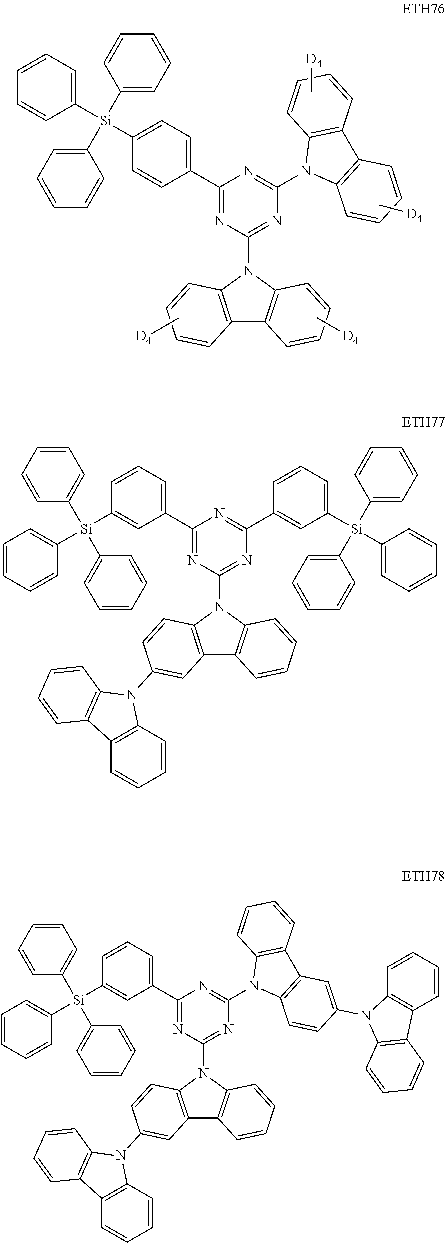

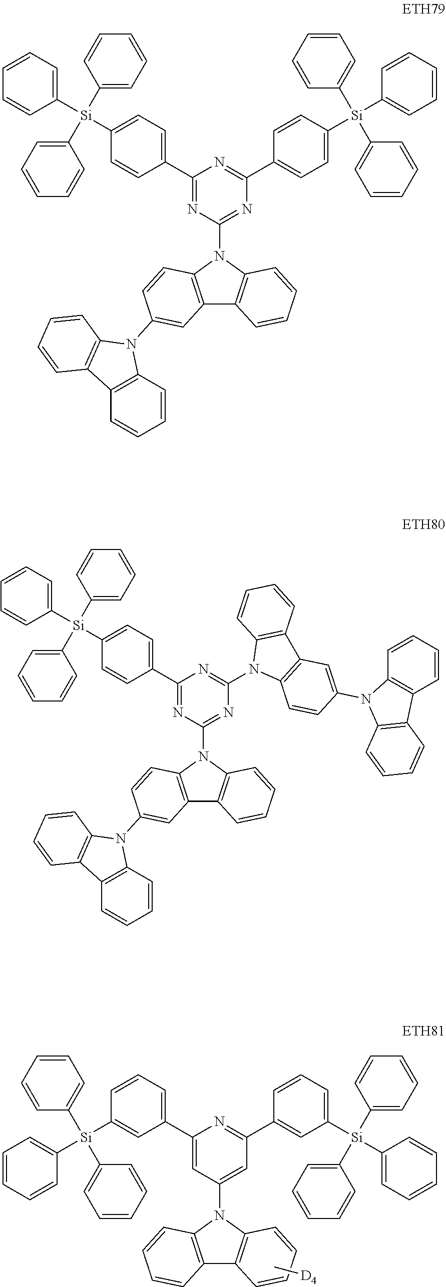

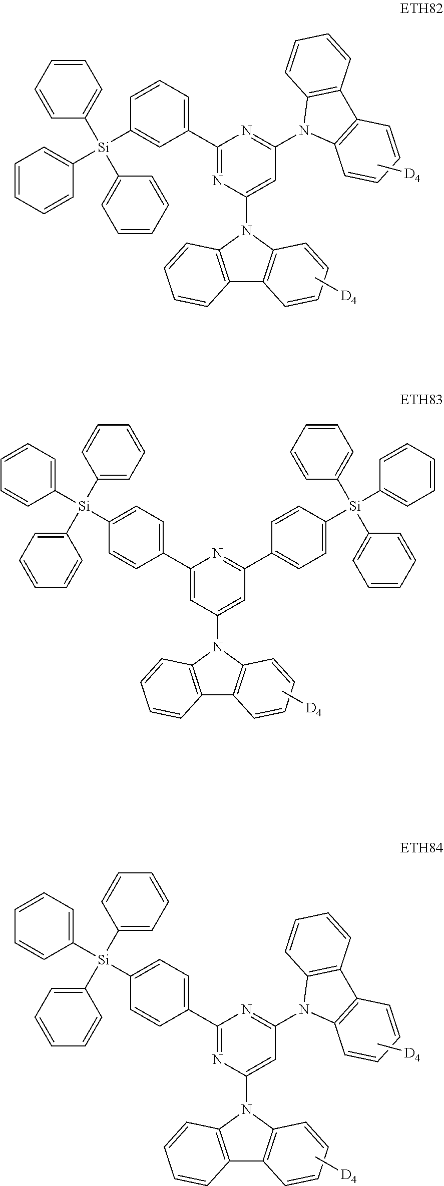

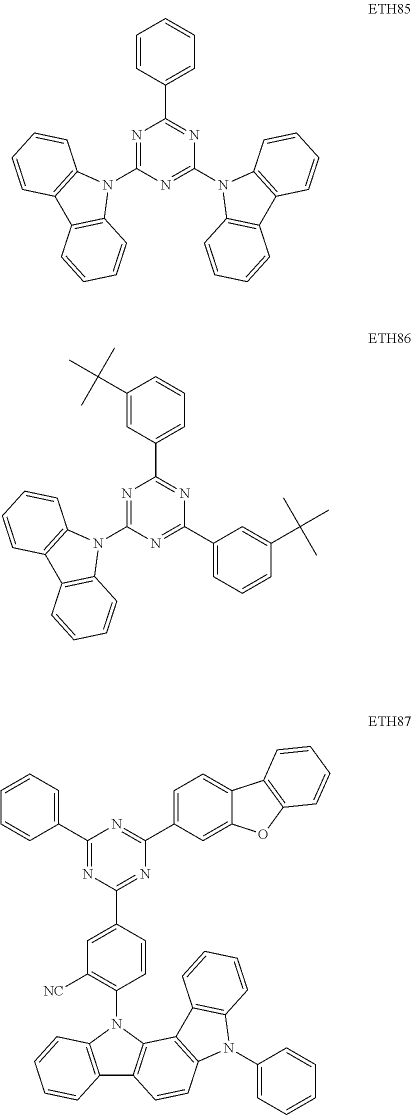 [SMILES: c1ccc(-c2nc(-n3c4ccccc4c4ccccc43)nc(-n3c4ccccc4c4ccccc43)n2)cc1 ; CC(C)(C)c1cccc(-c2nc(-c3cccc(C(C)(C)C)c3)nc(-n3c4ccccc4c4ccccc43)n2)c1 ; N#Cc1cc(-c2nc(-c3ccccc3)nc(-c3ccc4c(c3)oc3ccccc34)n2)ccc1-n1c2ccccc2c2ccc3c(c4ccccc4n3-c3ccccc3)c21]

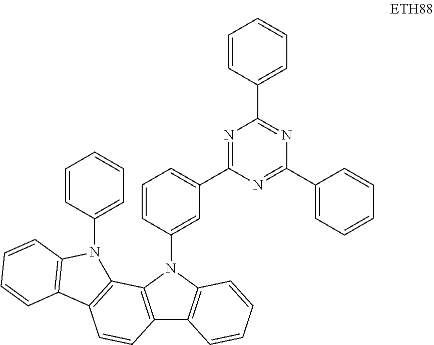 [SMILES: c1ccc(-c2nc(-c3ccccc3)nc(-c3cccc(-n4c5ccccc5c5ccc6c7ccccc7n(-c7ccccc7)c6c54)c3)n2)cc1]

In the example compounds suggested in Compound Group 2, “D” refers to deuterium, and “Ph” refers to an unsubstituted phenyl group.

In one or more embodiments, the emission layer EML may further include a dopant represented by Formula D-1. For example, the emission layer EML may include an organometallic complex including platinum (Pt) as a central metal atom and ligands bonded to the central metal atom, as the dopant. In the light emitting element ED of one or more embodiments, the emission layer EML may include a compound represented by Formula D-1 as the phosphorescent dopant.

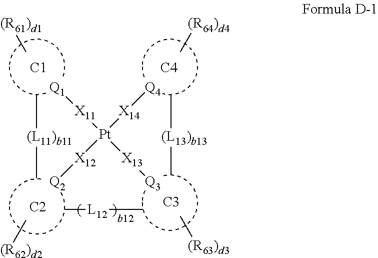

In Formula D-1, Q₁ to Q₄ may each independently be C or N.

In Formula D-1, C1 to C4 may each independently be a substituted or unsubstituted hydrocarbon ring of 5 to 30 ring-forming carbon atoms or a substituted or unsubstituted heterocycle of 2 to 30 ring-forming carbon atoms.

In Formula D-1, X₁₁ to X₁₄ may each independently be a direct linkage or *—O—*. For example, any one selected from among X₁₁ to X₁₄ may be *—O—*, and the others may each be a direct linkage.

In Formula D-1, L₁₁ to L₁₃ may each independently be a direct linkage,

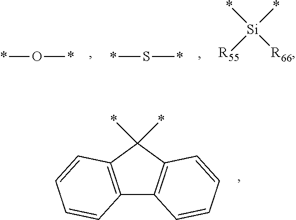

a substituted or unsubstituted alkylene group of 1 to 20 carbon atoms, a substituted or unsubstituted arylene group of 6 to 30 ring-forming carbon atoms, or a substituted or unsubstituted heteroarylene group of 2 to 30 ring-forming carbon atoms. In L₁₁ to L₁₃, “ [symbol] ” refers to a part connected with C1 to C4.

In Formula D-1, b11 to b13 may each independently be 0 or 1. If (e.g., when) b11 is 0, C1 and C2 may be unconnected. If (e.g., when) b12 is 0, C2 and C3 may be unconnected. If (e.g., when) b13 is 0, C3 and C4 may be unconnected.

In Formula D-1, R₆₁ to R₆₆ may each independently be hydrogen, deuterium, a halogen, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbon atoms, a substituted or unsubstituted alkenyl group of 2 to 20 carbon atoms, a substituted or unsubstituted aryl group of 6 to 60 ring-forming carbon atoms, or a substituted or unsubstituted heteroaryl group of 2 to 60 ring-forming carbon atoms. In one or more embodiments, one or more selected from among R₆₁ to R₆₆ may be combined with an adjacent group to form a ring. In one or more embodiments, R₆₁ to R₆₆ may each independently be a substituted or unsubstituted methyl group or a substituted or unsubstituted t-butyl group.

In Formula D-1, d1 to d4 may each independently be an integer of 0 to 4. In Formula D-1, if (e.g., when) d1 to d4 are each 0, the compound represented by Formula D-1 may be unsubstituted with R₆₁ to R₆₄, respectively. An embodiment in which d1 to d4 are each 4, and R₆₁ to R₆₄ are all hydrogens, may be the same as an embodiment in which d1 to d4 are each 0. If (e.g., when) d1 to d4 are integers of 2 or more, each of multiple R₆₁'s to R₆₄'s may be all the same, or at least one selected from among multiple R₆₁'s to R₆₄'s may be different.

In Formula D-1, C1 to C4 may each independently be a substituted or unsubstituted hydrocarbon ring or a substituted or unsubstituted heterocycle, represented by any one selected from among C-1 to C-5.

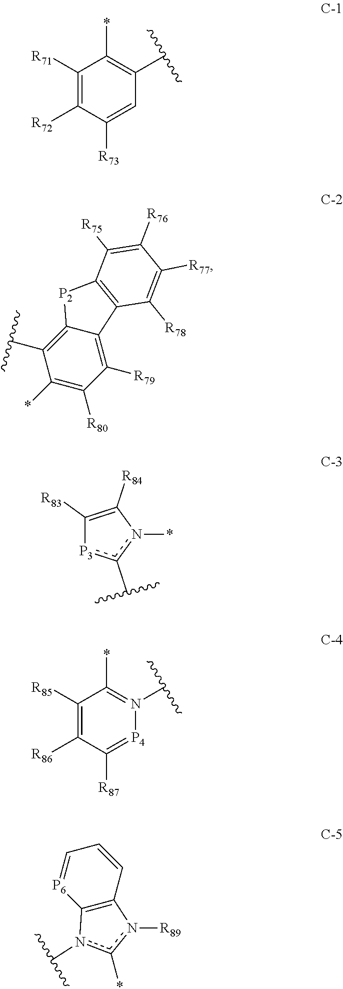

In C-1 to C-5, P₁ may be C—* or CR₇₄, P₂ may be N—* or NR₈₁, P₃ may be N—* or NR₈₂, P₄ may be C—* or CR₈₈, and P₆ may be C—* or CR₉₀. R₇₁ to R₉₀ may each independently be a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring.

In addition, in C-1 to C-5,

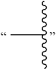

is a part connected with a central metal atom of Pt, and “ [symbol] ” corresponds to a part connected with an adjacent ring group (C1 to C4) or an adjacent linker (L₁₁ to L₁₃).

The light emitting element ED of one or more embodiments includes all of the first host, the second host, and the dopant, and the emission layer EML may include the combination of two host materials and one dopant material. In the light emitting element ED of one or more embodiments, the emission layer EML may include first host and second host, which are two different hosts, and the phosphorescent dopant including an organometallic complex, concurrently (e.g., simultaneously), and may show excellent or suitable emission efficiency properties.

In one or more embodiments, the dopant represented by Formula D-1 may include (e.g., be) at least one (e.g., any one) selected from among compounds represented in Compound Group 3. The emission layer EML may include at least one selected from among the compounds represented in Compound Group 3 as a phosphorescent dopant material.

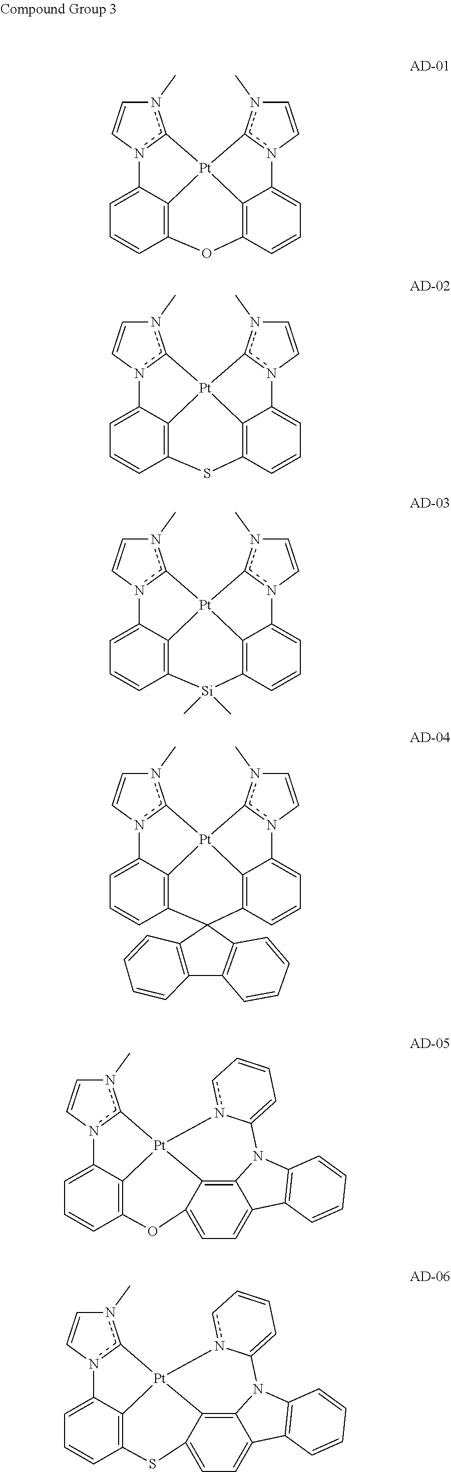

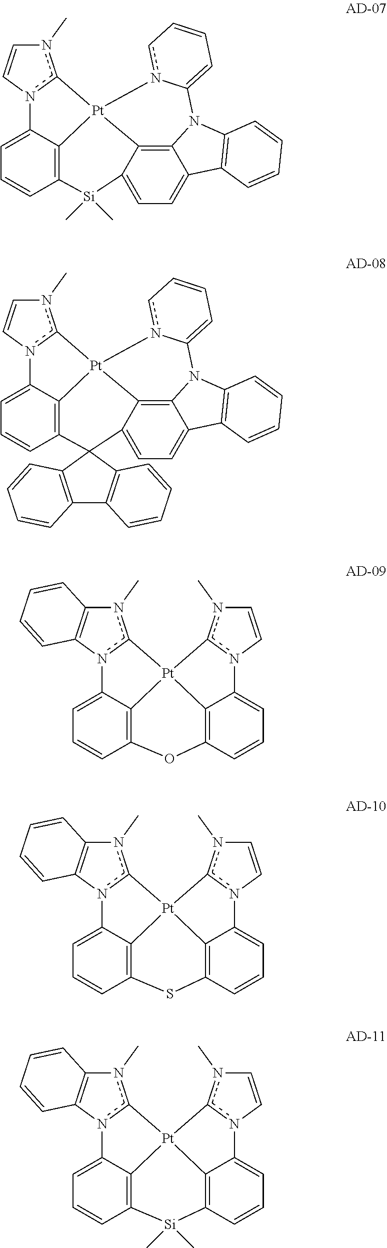

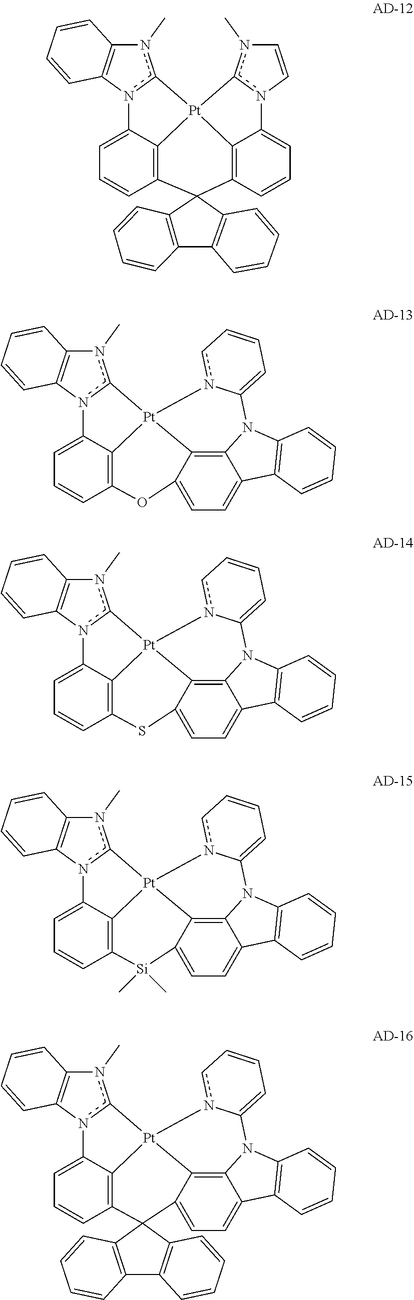

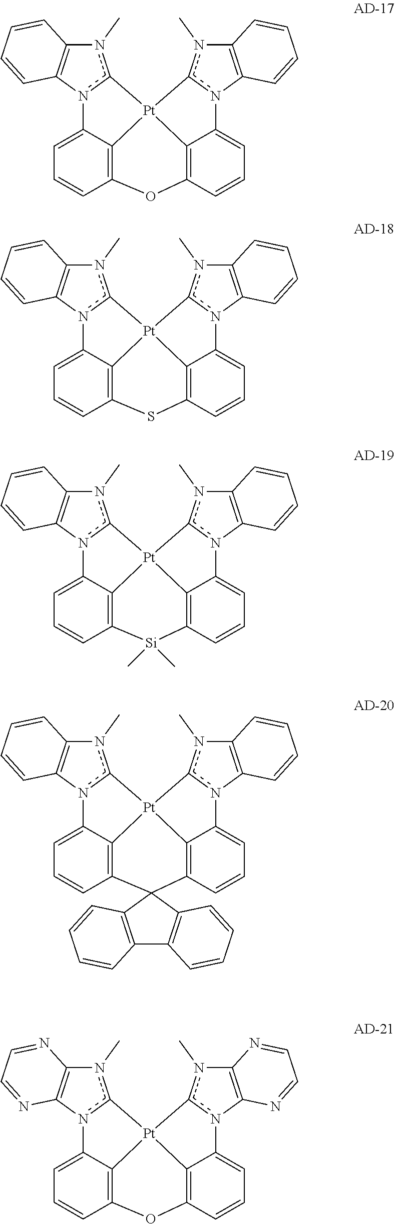

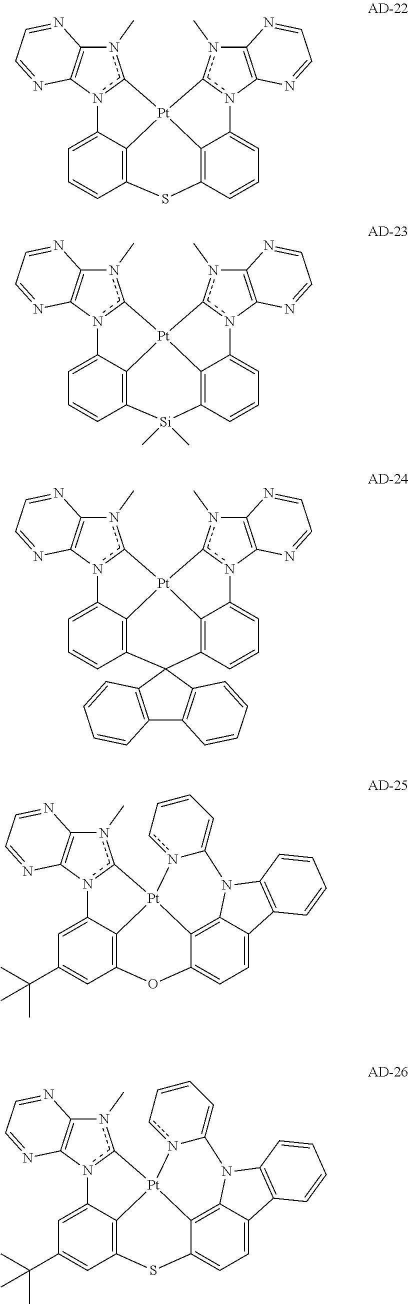

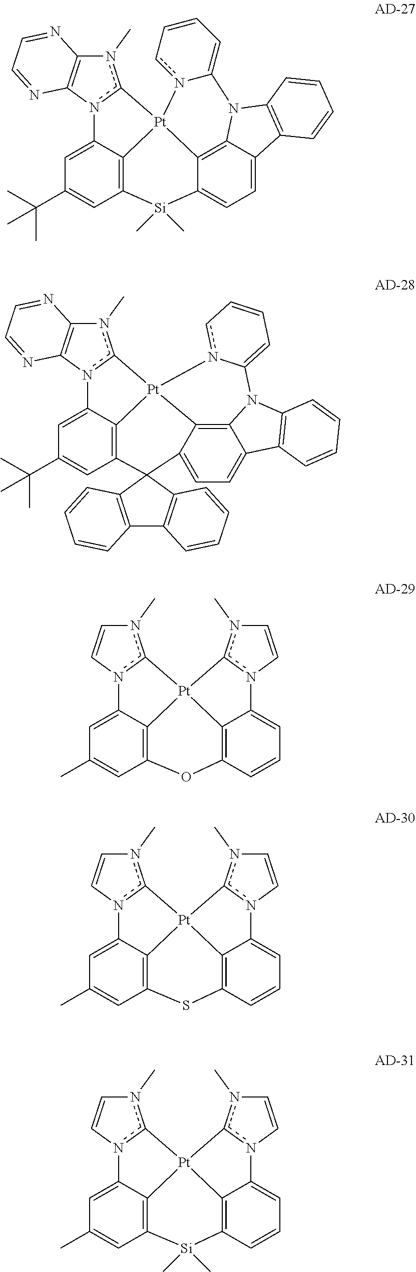

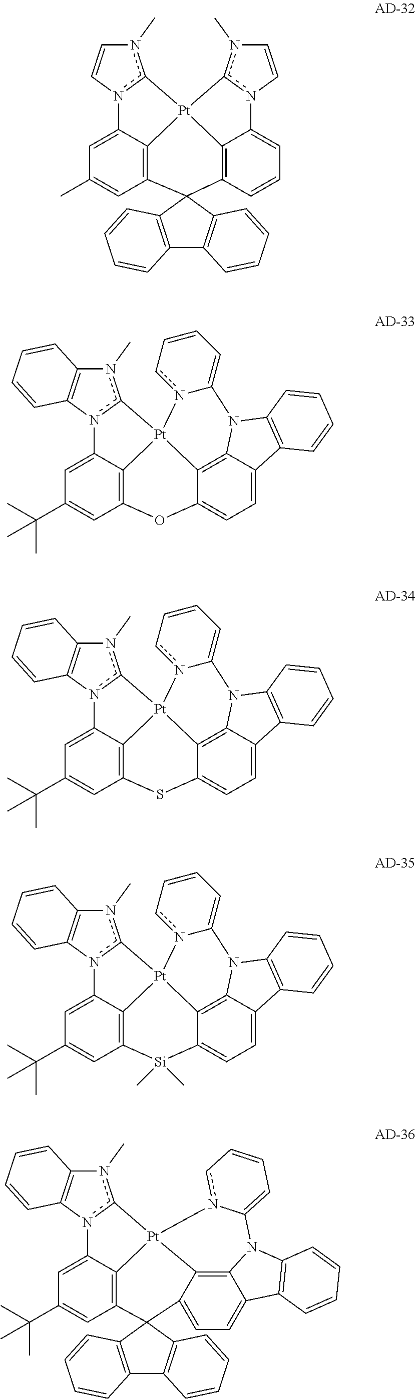

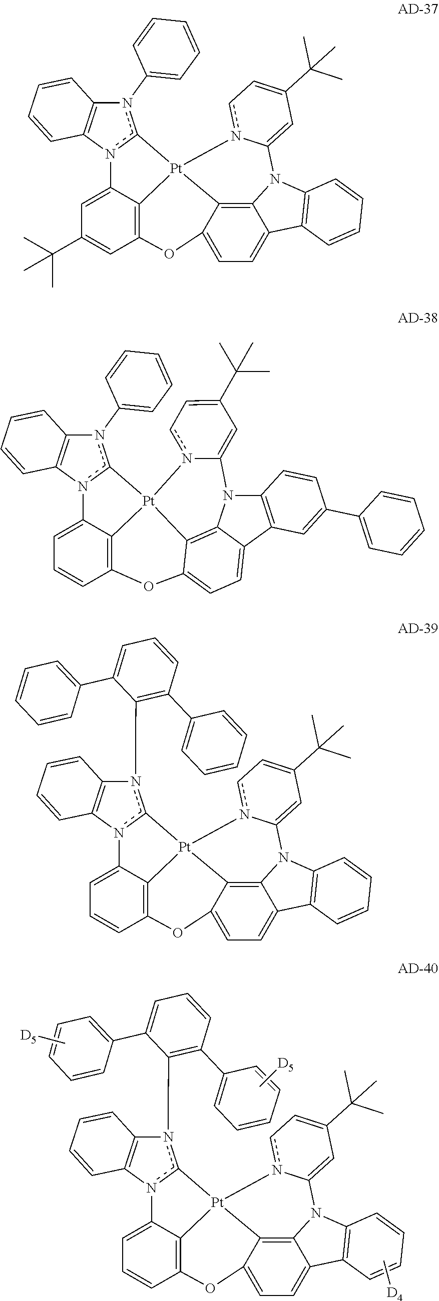

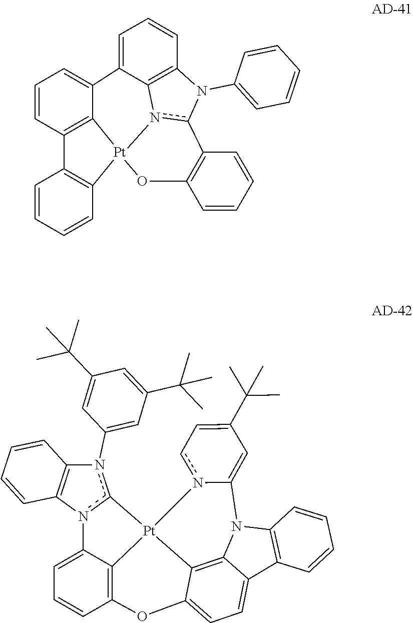

In the example compounds suggested in Compound Group 3, “D” refers to deuterium.

In the light emitting element ED of one or more embodiments, the emission layer EML may further include a material well-suitable in the art in addition to the above-described nitrogen-containing compound.

In the light emitting elements ED of embodiments, shown in FIG. 3 to FIG. 6, the emission layer EML may include a host and a dopant, and the emission layer EML may include a compound represented by Formula H-2. The compound represented by Formula H-2 may be used as a thermally activated delayed fluorescence host material.

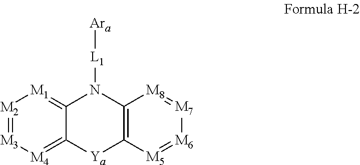

In Formula H-2, M₁ to M₈ may each independently be N or CR₅₁. For example, in one or more embodiments, all M₁ to M₈ may be CR₅₁. In one or more embodiments, any one selected from among M₁ to M₈ may be N, and the remainder may be CR₅₁.

In Formula H-2, L₁ may be a direct linkage, a substituted or unsubstituted arylene group of 6 to 30 ring-forming carbon atoms, or a substituted or unsubstituted heteroarylene group of 2 to 30 ring-forming carbon atoms. For example, in one or more embodiments, L₁ may be a direct linkage, a substituted or unsubstituted phenylene group, a substituted or unsubstituted divalent biphenyl group, a substituted or unsubstituted divalent carbazole group, and/or the like, but embodiments of the present disclosure are not limited thereto.

In Formula H-2, Ya may be a direct linkage, CR₅₂R₅₃, or SiR₅₄R₅₅. For example, it may refer to that two six-membered rings (e.g., two benzene rings) connected with the nitrogen atom of Formula H-2 may be connected via a direct linkage,

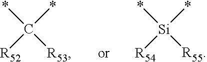

In Formula H-2, if (e.g., when) Ya is a direct linkage, the substituent represented by Formula H-2 may include a carbazole moiety.

In Formula H-2, Ara may be a substituted or unsubstituted aryl group of 6 to 30 ring-forming carbon atoms or a substituted or unsubstituted heteroaryl group of 2 to 30 ring-forming carbon atoms. For example, in one or more embodiments, Ara may be a substituted or unsubstituted carbazole group, a substituted or unsubstituted dibenzofuran group, a substituted or unsubstituted dibenzothiophene group, a substituted or unsubstituted biphenyl group, and/or the like, but embodiments of the present disclosure are not limited thereto.

In Formula H-2, R₅₁ to R₅₅ may each independently be hydrogen, deuterium, a halogen, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbon atoms, a substituted or unsubstituted alkenyl group of 2 to 20 carbon atoms, a substituted or unsubstituted aryl group of 6 to 60 ring-forming carbon atoms, or a substituted or unsubstituted heteroaryl group of 2 to 60 ring-forming carbon atoms. In one or more embodiments, one or more selected from among R₅₁ to R₅₅ may be combined with an adjacent group to form a ring. For example, in one or more embodiments, R₅₁ to R₅₅ may each independently be hydrogen or deuterium. In one or more embodiments, R₅₁ to R₅₅ may each independently be an unsubstituted methyl group or an unsubstituted phenyl group.

In one or more embodiments, the compound represented by Formula H-2 may be any one selected from among compounds represented in Compound Group 4. The emission layer EML may include at least one selected from among the compounds represented in Compound Group 4 as a fluorescence host material or a thermally activated delayed fluorescence host material.

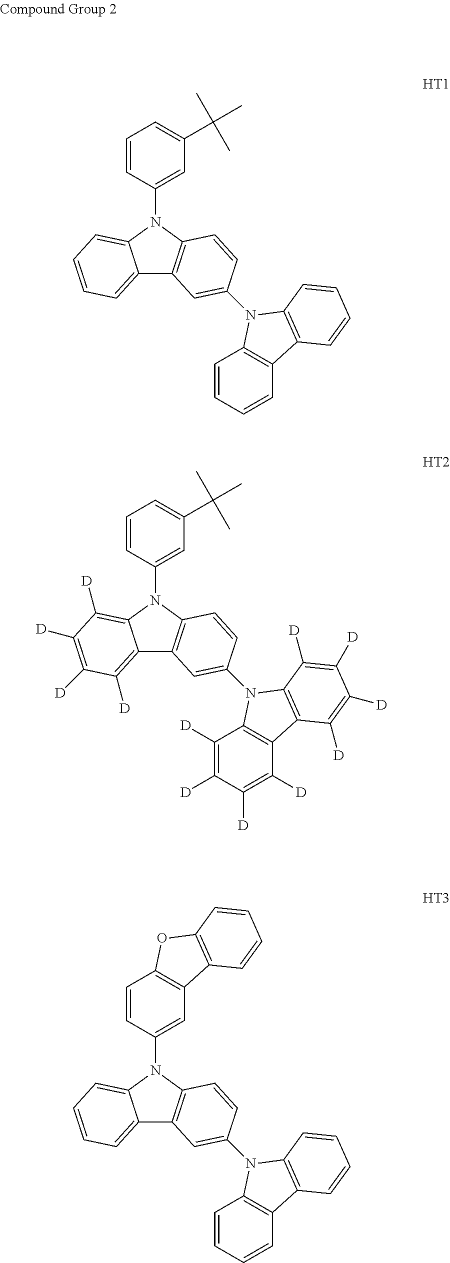 [SMILES: CC(C)(C)c1cccc(-n2c3ccccc3c3cc(-n4c5ccccc5c5ccccc54)ccc32)c1 ; [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)oc1ccc(-n3c4ccccc4c4cc(-n5c6ccccc6c6ccccc65)ccc43)cc12]

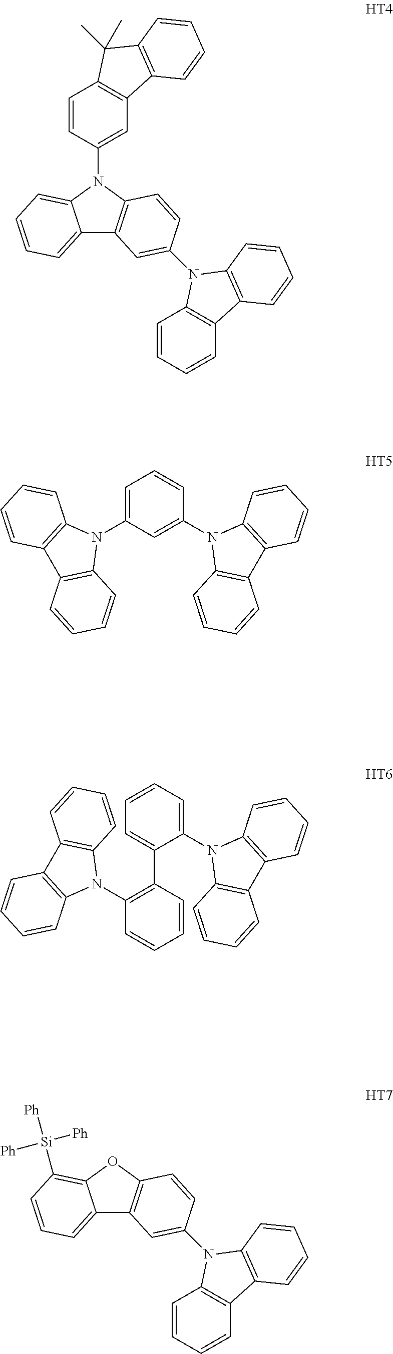 [SMILES: CC1(C)c2ccccc2-c2cc(-n3c4ccccc4c4cc(-n5c6ccccc6c6ccccc65)ccc43)ccc21 ; c1cc(-n2c3ccccc3c3ccccc32)cc(-n2c3ccccc3c3ccccc32)c1 ; c1ccc(-n2c3ccccc3c3ccccc32)c(-c2ccccc2-n2c3ccccc3c3ccccc32)c1 ; c1ccc([Si](c2ccccc2)(c2ccccc2)c2cccc3c2oc2ccc(-n4c5ccccc5c5ccccc54)cc23)cc1]

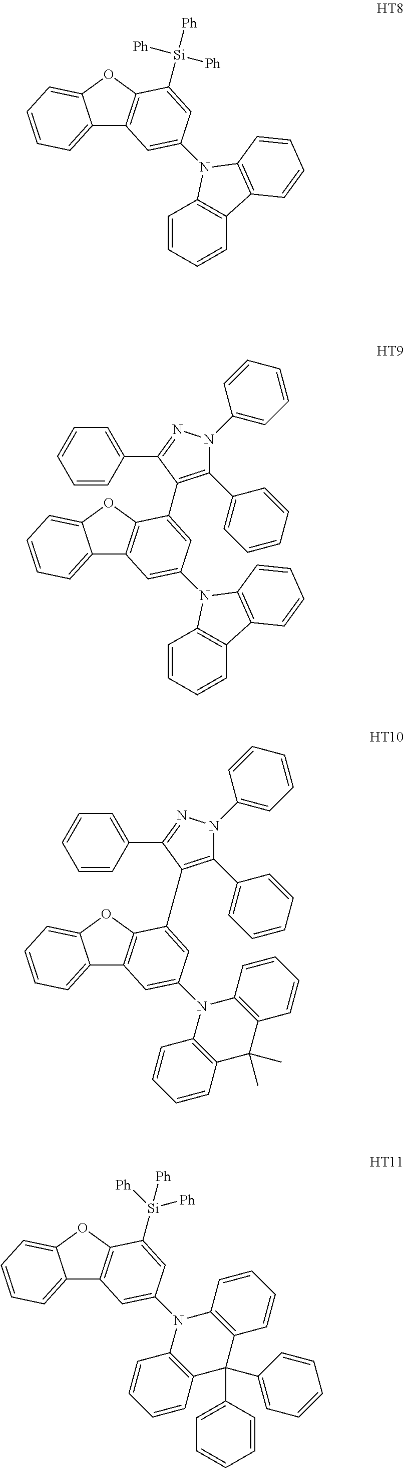 [SMILES: c1ccc([Si](c2ccccc2)(c2ccccc2)c2cc(-n3c4ccccc4c4ccccc43)cc3c2oc2ccccc23)cc1 ; c1ccc(-c2nn(-c3ccccc3)c(-c3ccccc3)c2-c2cc(-n3c4ccccc4c4ccccc43)cc3c2oc2ccccc23)cc1 ; CC1(C)c2ccccc2N(c2cc(-c3c(-c4ccccc4)nn(-c4ccccc4)c3-c3ccccc3)c3oc4ccccc4c3c2)c2ccccc21 ; c1ccc(C2(c3ccccc3)c3ccccc3N(c3cc([Si](c4ccccc4)(c4ccccc4)c4ccccc4)c4oc5ccccc5c4c3)c3ccccc32)cc1]

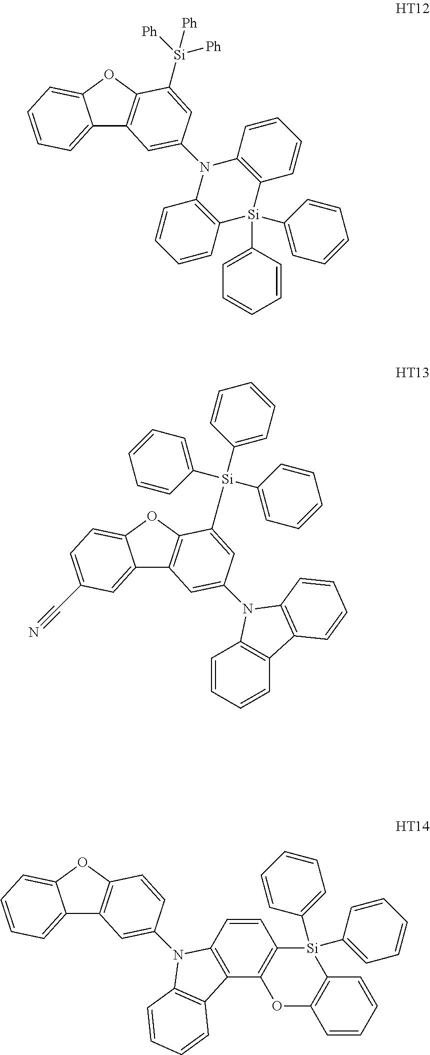 [SMILES: c1ccc([Si]2(c3ccccc3)c3ccccc3N(c3cc([Si](c4ccccc4)(c4ccccc4)c4ccccc4)c4oc5ccccc5c4c3)c3ccccc32)cc1 ; N#Cc1ccc2oc3c([Si](c4ccccc4)(c4ccccc4)c4ccccc4)cc(-n4c5ccccc5c5ccccc54)cc3c2c1 ; c1ccc([Si]2(c3ccccc3)c3ccccc3Oc3c2ccc2c3c3ccccc3n2-c2ccc3oc4ccccc4c3c2)cc1]

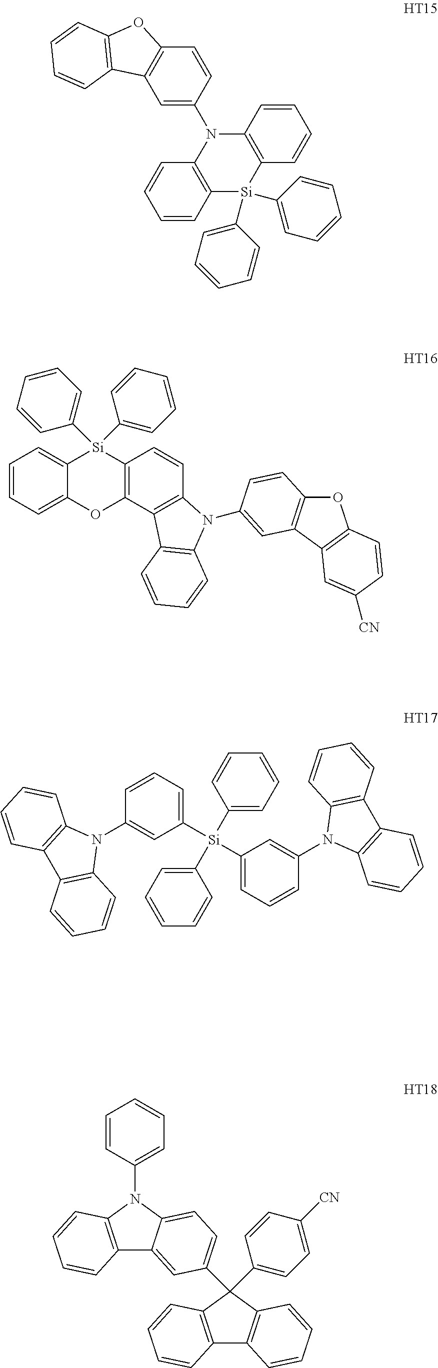 [SMILES: c1ccc([Si]2(c3ccccc3)c3ccccc3N(c3ccc4oc5ccccc5c4c3)c3ccccc32)cc1 ; N#Cc1ccc2oc3ccc(-n4c5ccccc5c5c6c(ccc54)[Si](c4ccccc4)(c4ccccc4)c4ccccc4O6)cc3c2c1 ; c1ccc([Si](c2ccccc2)(c2cccc(-n3c4ccccc4c4ccccc43)c2)c2cccc(-n3c4ccccc4c4ccccc43)c2)cc1 ; N#Cc1ccc(C2(c3ccc4c(c3)c3ccccc3n4-c3ccccc3)c3ccccc3-c3ccccc32)cc1]

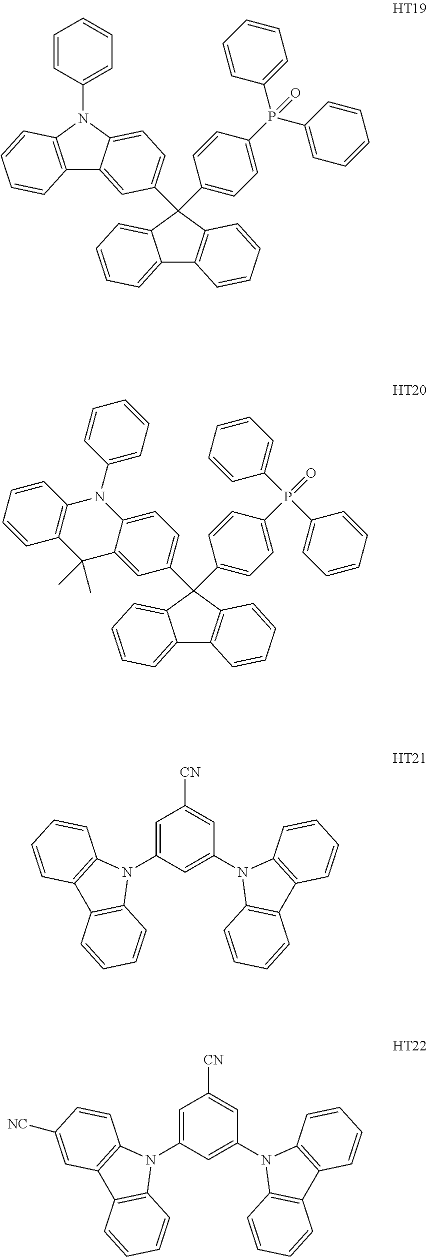 [SMILES: O=P(c1ccccc1)(c1ccccc1)c1ccc(C2(c3ccc4c(c3)c3ccccc3n4-c3ccccc3)c3ccccc3-c3ccccc32)cc1 ; CC1(C)c2ccccc2N(c2ccccc2)c2ccc(C3(c4ccc(P(=O)(c5ccccc5)c5ccccc5)cc4)c4ccccc4-c4ccccc43)cc21 ; N#Cc1cc(-n2c3ccccc3c3ccccc32)cc(-n2c3ccccc3c3ccccc32)c1 ; N#Cc1cc(-n2c3ccccc3c3ccccc32)cc(-n2c3ccccc3c3cc(C#N)ccc32)c1]

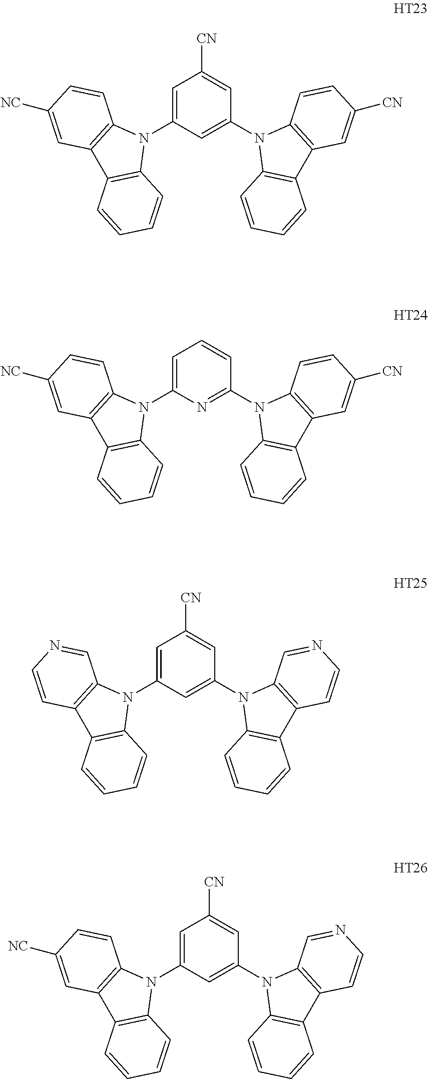 [SMILES: N#Cc1cc(-n2c3ccccc3c3cc(C#N)ccc32)cc(-n2c3ccccc3c3cc(C#N)ccc32)c1 ; N#Cc1ccc2c(c1)c1ccccc1n2-c1cccc(-n2c3ccccc3c3cc(C#N)ccc32)n1 ; N#Cc1cc(-n2c3ccccc3c3ccncc32)cc(-n2c3ccccc3c3ccncc32)c1 ; N#Cc1cc(-n2c3ccccc3c3cc(C#N)ccc32)cc(-n2c3ccccc3c3ccncc32)c1]

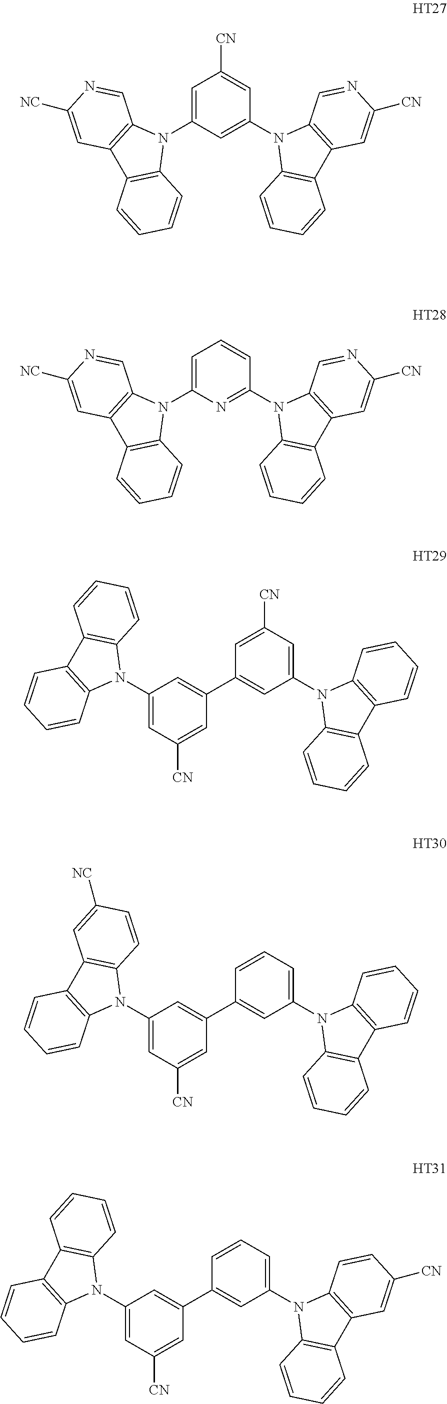 [SMILES: N#Cc1cc(-n2c3ccccc3c3cc(C#N)ncc32)cc(-n2c3ccccc3c3cc(C#N)ncc32)c1 ; N#Cc1cc2c3ccccc3n(-c3cccc(-n4c5ccccc5c5cc(C#N)ncc54)n3)c2cn1 ; N#Cc1cc(-c2cc(C#N)cc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1cc(-c2cccc(-n3c4ccccc4c4cc(C#N)ccc43)c2)cc(-n2c3ccccc3c3ccccc32)c1]

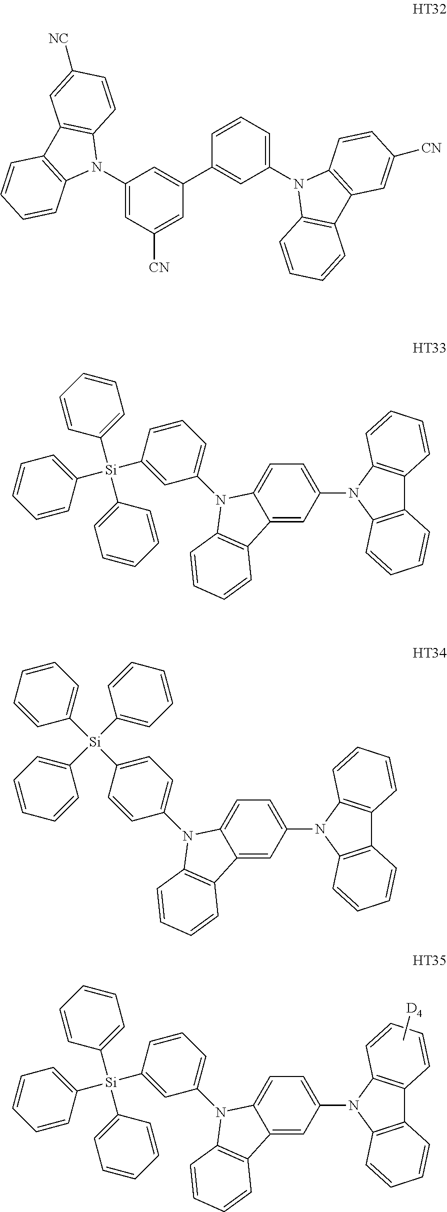

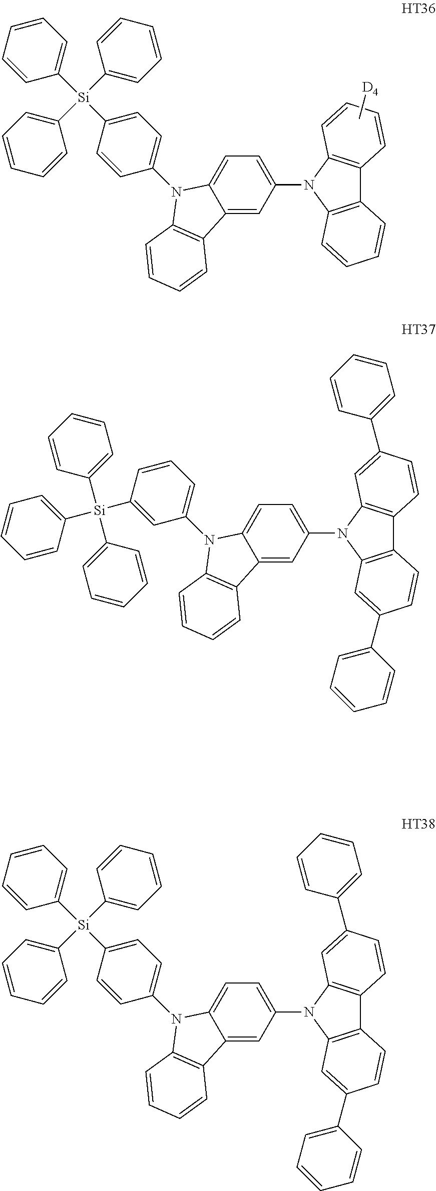

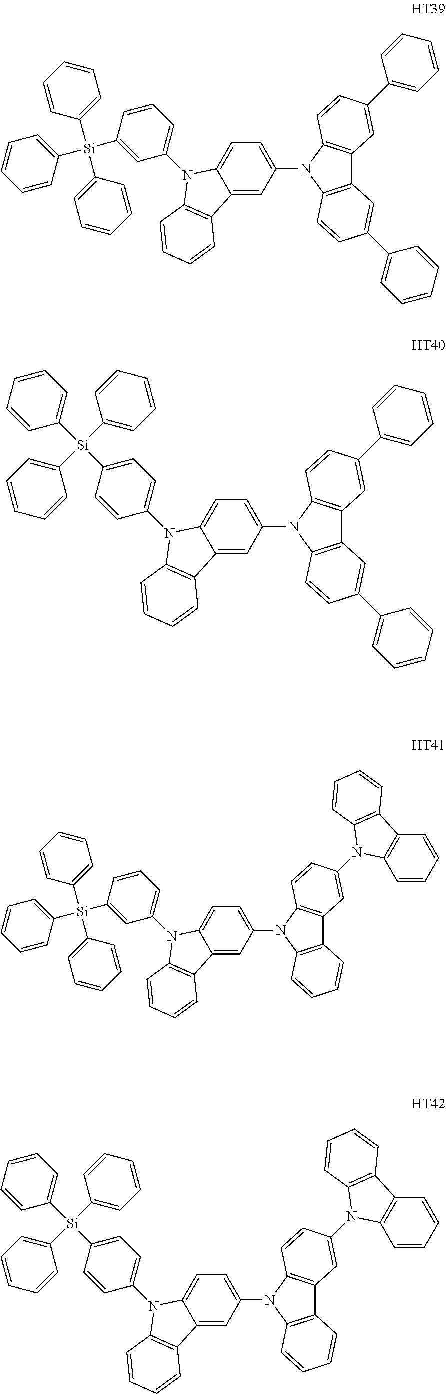 [SMILES: c1ccc(-c2ccc3c(c2)c2cc(-c4ccccc4)ccc2n3-c2ccc3c(c2)c2ccccc2n3-c2cccc([Si](c3ccccc3)(c3ccccc3)c3ccccc3)c2)cc1 ; c1ccc(-c2ccc3c(c2)c2cc(-c4ccccc4)ccc2n3-c2ccc3c(c2)c2ccccc2n3-c2ccc([Si](c3ccccc3)(c3ccccc3)c3ccccc3)cc2)cc1 ; c1ccc([Si](c2ccccc2)(c2ccccc2)c2cccc(-n3c4ccccc4c4cc(-n5c6ccccc6c6cc(-n7c8ccccc8c8ccccc87)ccc65)ccc43)c2)cc1 ; c1ccc([Si](c2ccccc2)(c2ccccc2)c2ccc(-n3c4ccccc4c4cc(-n5c6ccccc6c6cc(-n7c8ccccc8c8ccccc87)ccc65)ccc43)cc2)cc1]

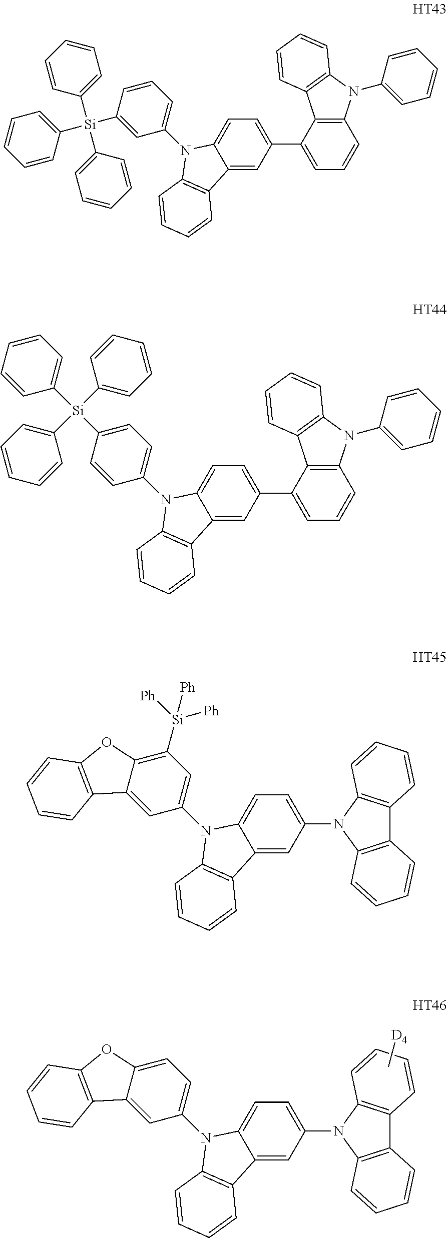

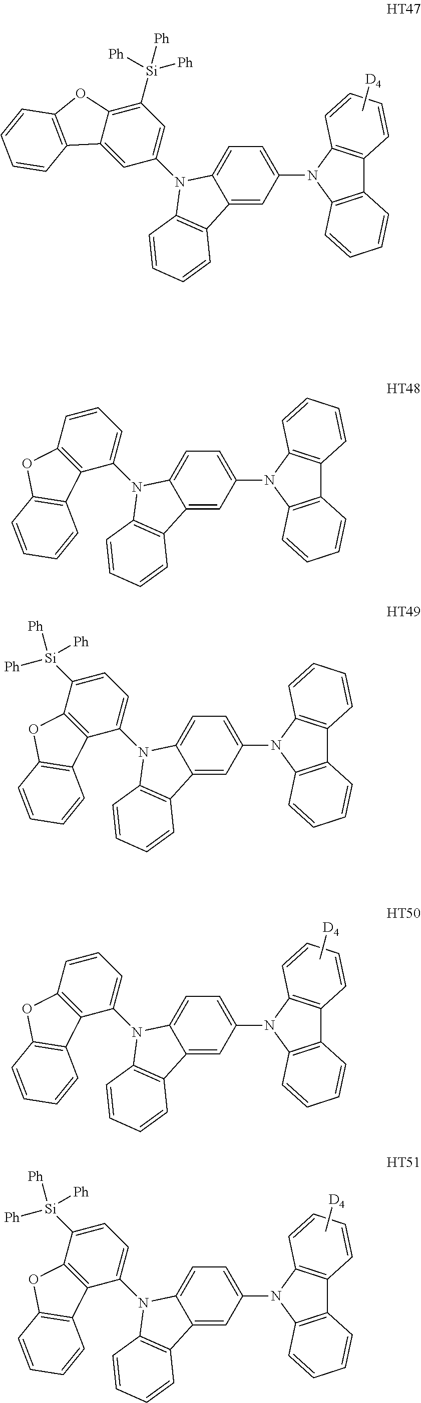

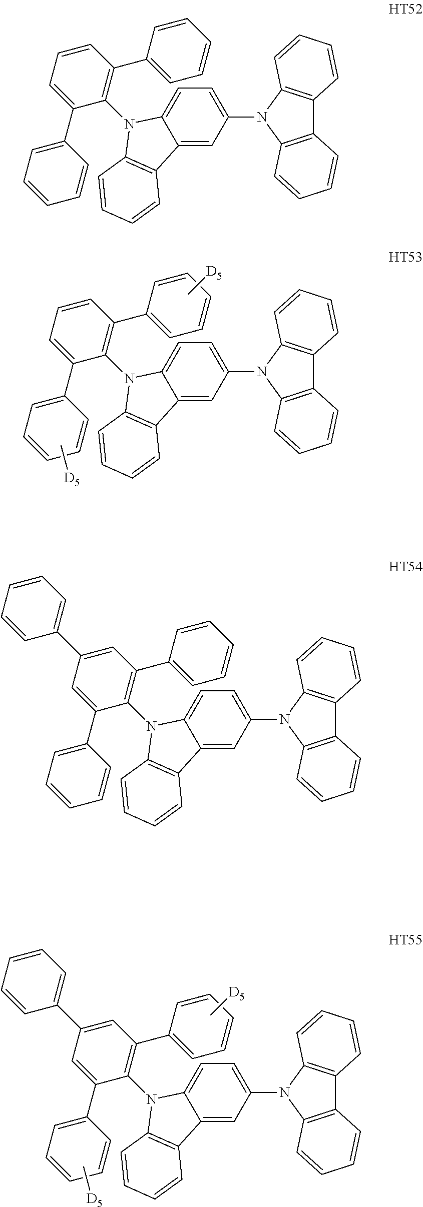

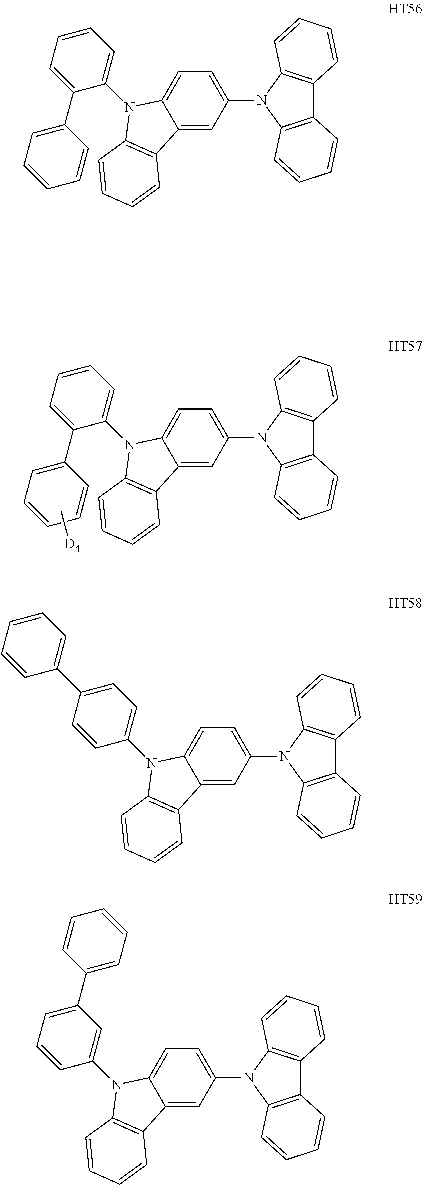

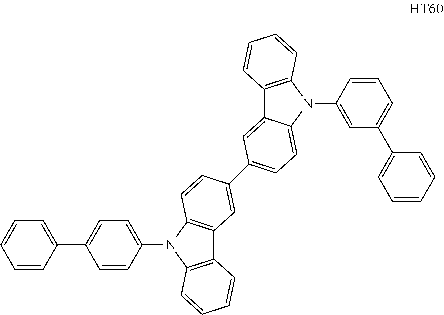 [SMILES: c1ccc(-c2ccc(-n3c4ccccc4c4cc(-c5ccc6c(c5)c5ccccc5n6-c5cccc(-c6ccccc6)c5)ccc43)cc2)cc1]

In the example compounds suggested in Compound Group 4, “D” refers to deuterium, and “Ph” may be an unsubstituted phenyl group.

In the light emitting element ED of one or more embodiments, the emission layer EML may further include one or more selected from among anthracene derivatives, pyrene derivatives, fluoranthene derivatives, chrysene derivatives, dihydrobenzanthracene derivatives, and triphenylene derivatives. For example, in one or more embodiments, the emission layer EML may include one or more anthracene derivatives and/or one or more pyrene derivatives.

In the light emitting element ED of embodiments, shown in FIG. 3 to FIG. 6, the emission layer EML may include a host and a dopant, and the emission layer EML may include a compound represented by Formula E-1. The compound represented by Formula E-1 may be used as a fluorescence host material or a thermally activated delayed fluorescence host material.

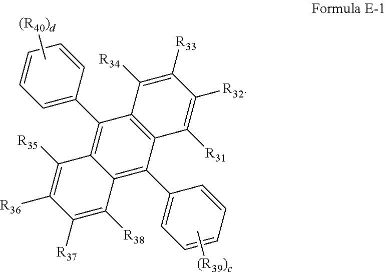

In Formula E-1, R₃₁ to R₄₀ may each independently be hydrogen, deuterium, a halogen, a substituted or unsubstituted silyl group, a substituted or unsubstituted thiol group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring. In one or more embodiments, one or more selected from among R₃₁ to R₄₀ may be combined with an adjacent group to form a saturated hydrocarbon ring, an unsaturated hydrocarbon ring, a saturated heterocycle, or an unsaturated heterocycle.

In Formula E-1, “c” and “d” may each independently be an integer of 0 to 5.

The compound represented by Formula E-1 may be any one selected from among Compound E1 to Compound E21.

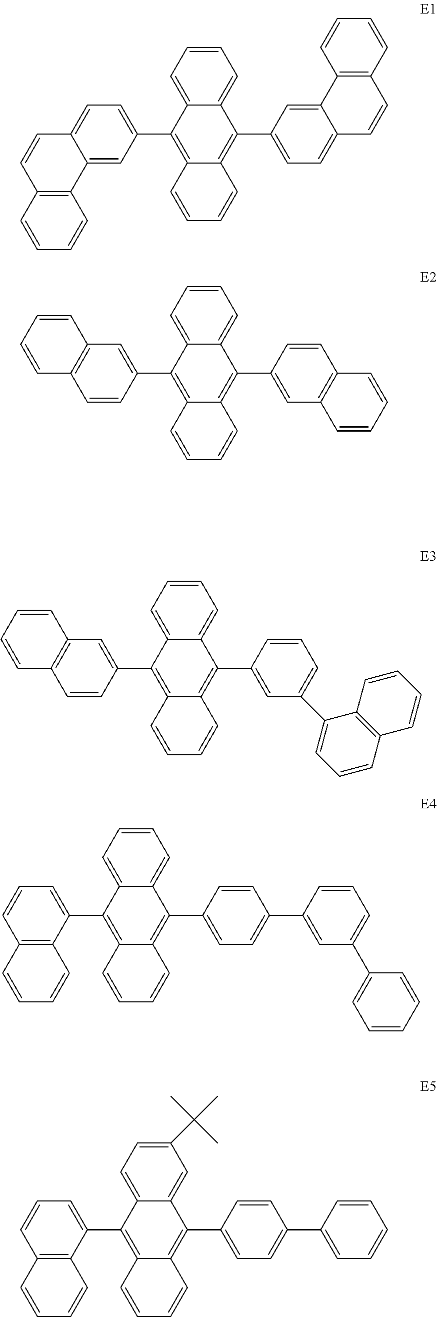

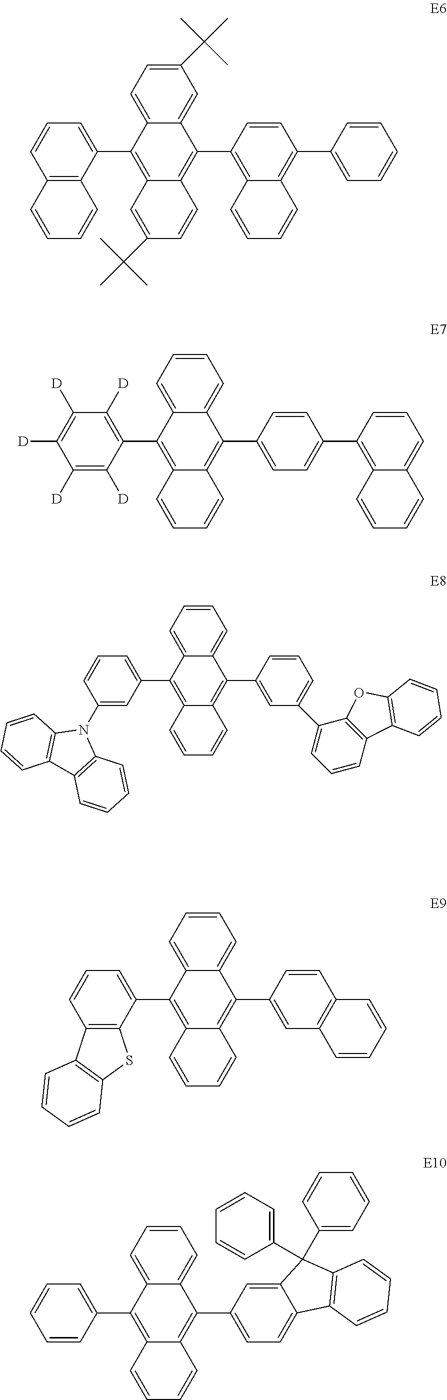

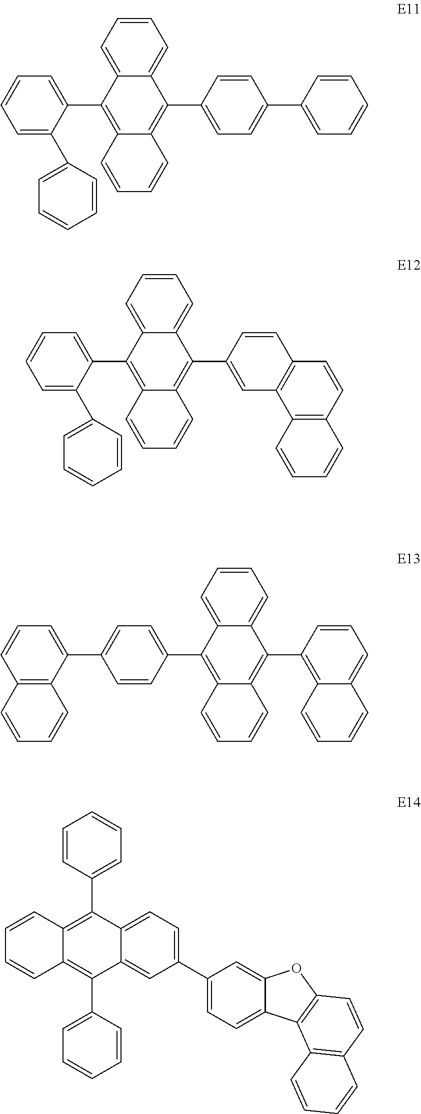

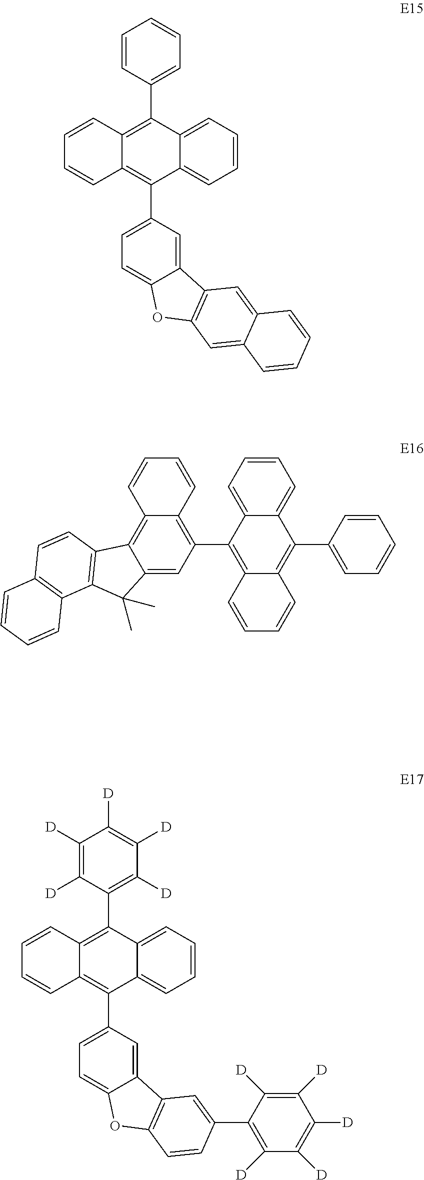

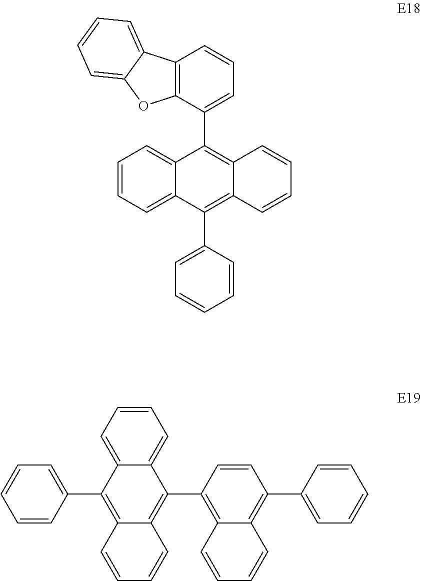

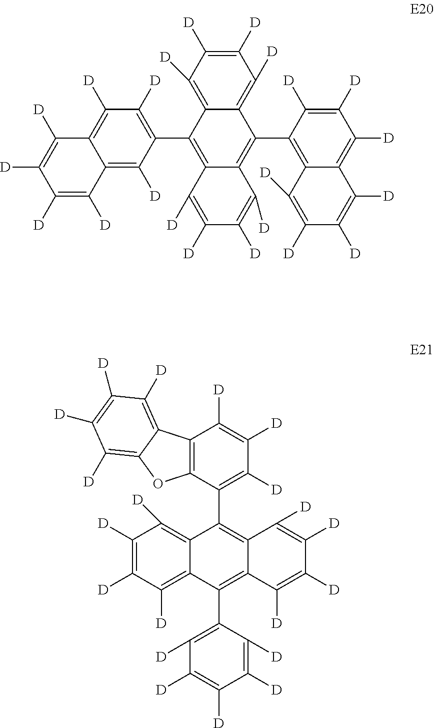 [SMILES: [2H]c1c([2H])c([2H])c2c([2H])c(-c3c4c([2H])c([2H])c([2H])c([2H])c4c(-c4c([2H])c([2H])c([2H])c5c([2H])c([2H])c([2H])c([2H])c45)c4c([2H])c([2H])c([2H])c([2H])c34)c([2H])c([2H])c2c1[2H] ; [2H]c1c([2H])c([2H])c(-c2c3c([2H])c([2H])c([2H])c([2H])c3c(-c3c([2H])c([2H])c([2H])c4c3oc3c([2H])c([2H])c([2H])c([2H])c34)c3c([2H])c([2H])c([2H])c([2H])c23)c([2H])c1[2H]]

In one or more embodiments, the emission layer EML may include a compound represented by Formula E-2a or Formula E-2b. The compound represented by Formula E-2a or Formula E-2b may be used as a phosphorescence host material or a thermally activated delayed fluorescence host material.

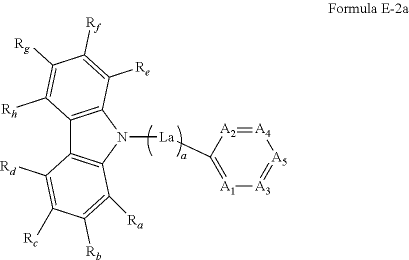

In Formula E-2a, “a” may be an integer of 0 to 10, La may be a direct linkage, a substituted or unsubstituted arylene group of 6 to 30 ring-forming carbon atoms, or a substituted or unsubstituted heteroarylene group of 2 to 30 ring-forming carbon atoms. In one or more embodiments, if (e.g., when) “a” is an integer of 2 or more, multiple La's may each independently be a substituted or unsubstituted arylene group of 6 to 30 ring-forming carbon atoms or a substituted or unsubstituted heteroarylene group of 2 to 30 ring-forming carbon atoms.

In addition, in Formula E-2a, A₁ to A₅ may each independently be N or CRᵢ. Rₐ to Rᵢ may each independently be hydrogen, deuterium, a substituted or unsubstituted amine group, a substituted or unsubstituted thiol group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be independently combined with an adjacent group to form a ring. In one or more embodiments, one or more selected from among Rₐ to Rᵢ may be combined with an adjacent group to form a hydrocarbon ring or a heterocycle including N, O, S, and/or the like, as a ring-forming atom.

In one or more embodiments, in Formula E-2a, two or three selected from among A₁ to A₅ may be N, and the remainder may be CRᵢ.

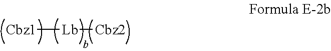

In Formula E-2b, Cbz1 and Cbz2 may each independently be an unsubstituted carbazole group or a carbazole group substituted with an aryl group of 6 to 30 ring-forming carbon atoms. L_{b} may be a direct linkage, a substituted or unsubstituted arylene group of 6 to 30 ring-forming carbon atoms, or a substituted or unsubstituted heteroarylene group of 2 to 30 ring-forming carbon atoms. “b” may be an integer of 0 to 10, and if (e.g., when) “b” is an integer of 2 or more, multiple L_{b}'s may each independently be a substituted or unsubstituted arylene group of 6 to 30 ring-forming carbon atoms or a substituted or unsubstituted heteroarylene group of 2 to 30 ring-forming carbon atoms.

The compound represented by Formula E-2a or Formula E-2b may be any one selected from among compounds in Compound Group E-2. However, the compounds shown in Compound Group E-2 are mere examples, and the compound represented by Formula E-2a or Formula E-2b is not limited to the compounds represented in Compound Group E-2.

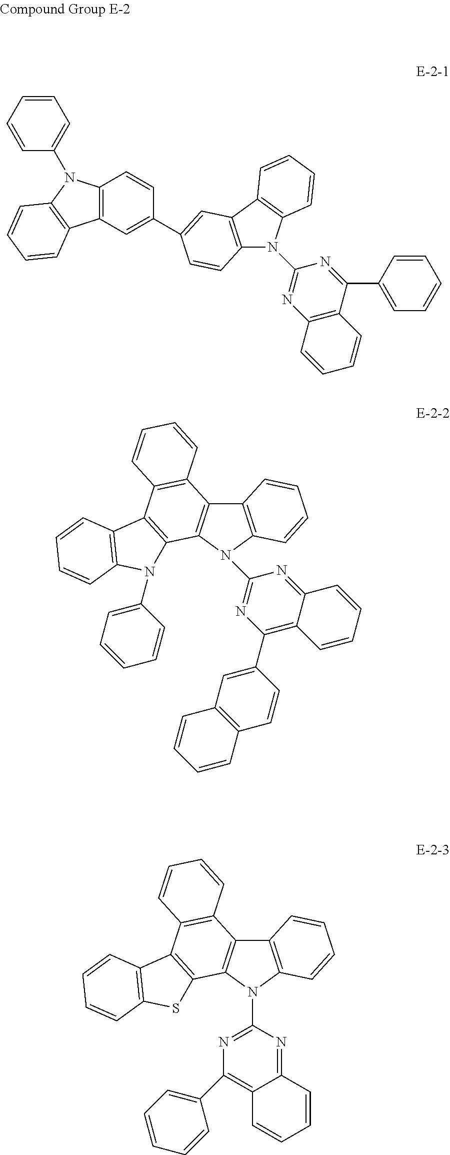 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

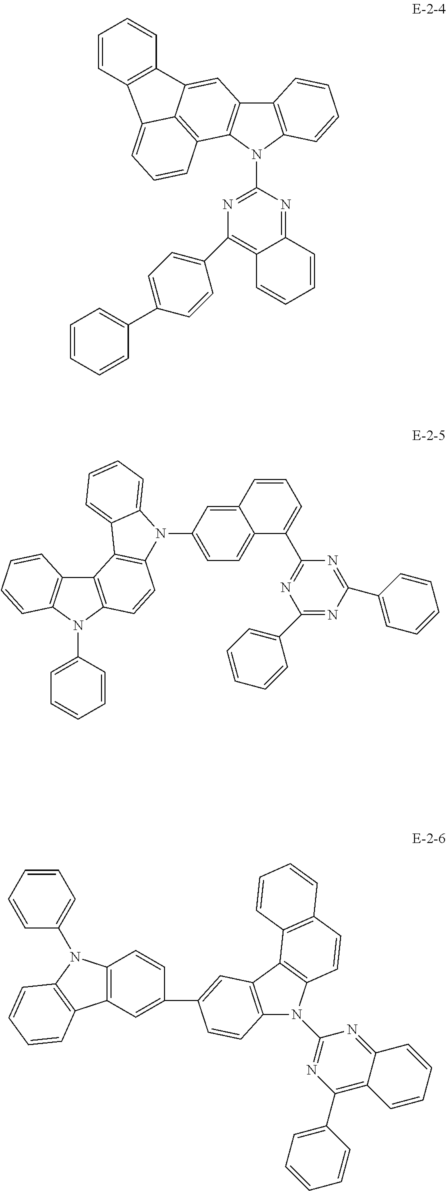 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

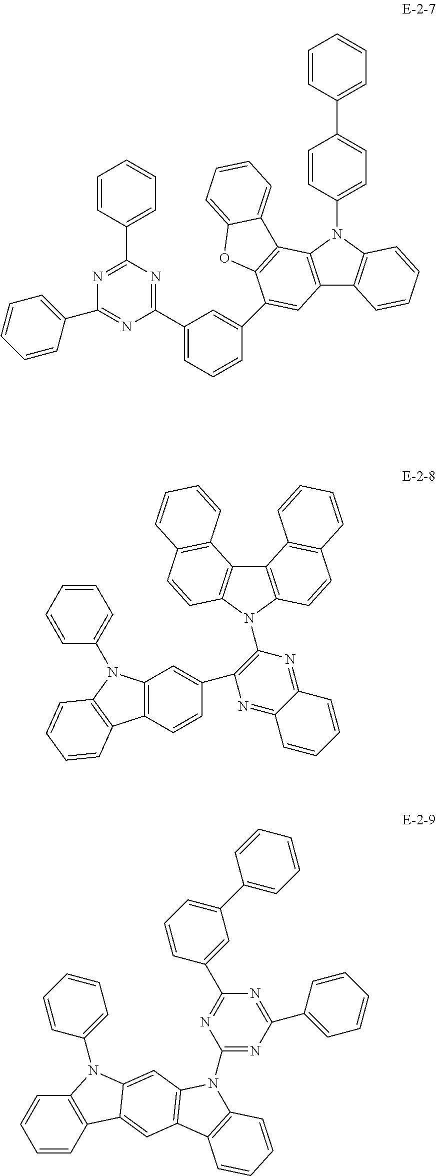 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1]

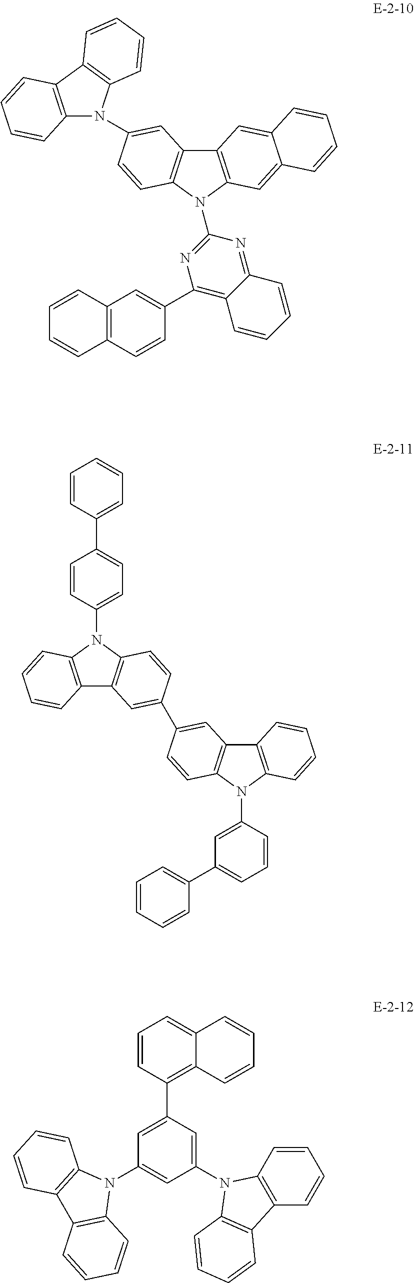 [SMILES: c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1]

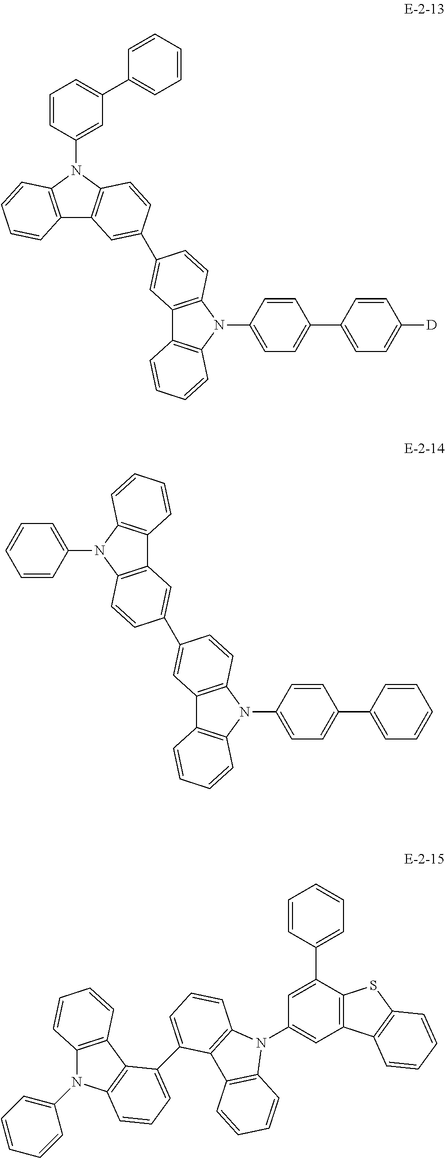 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

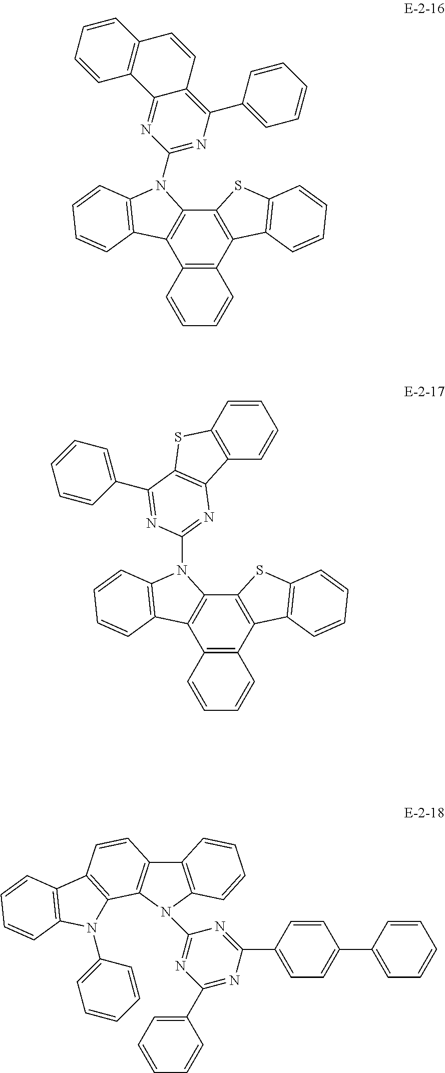 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1]

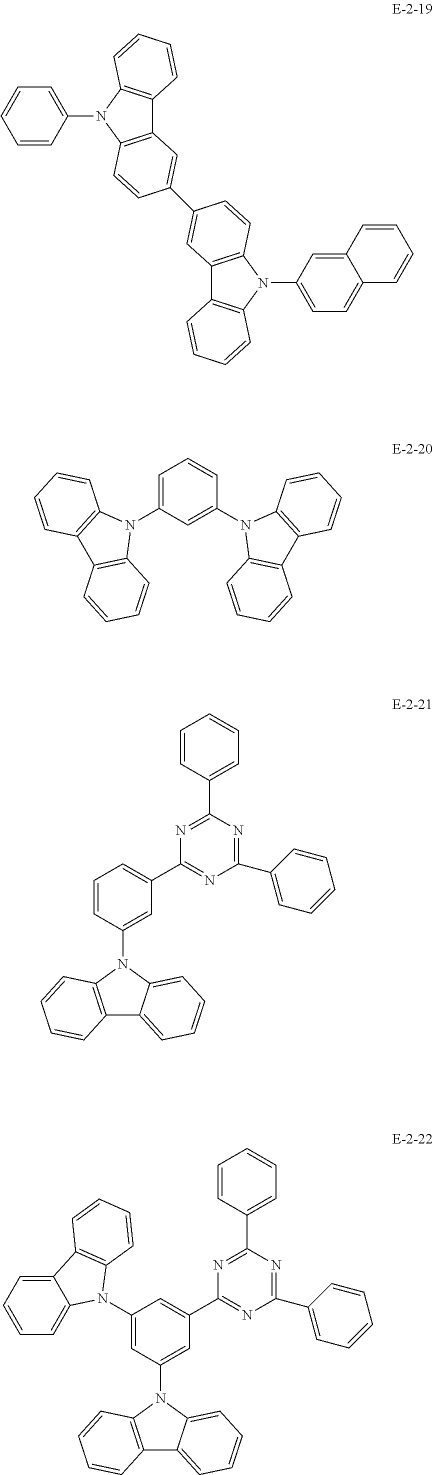 [SMILES: c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1]

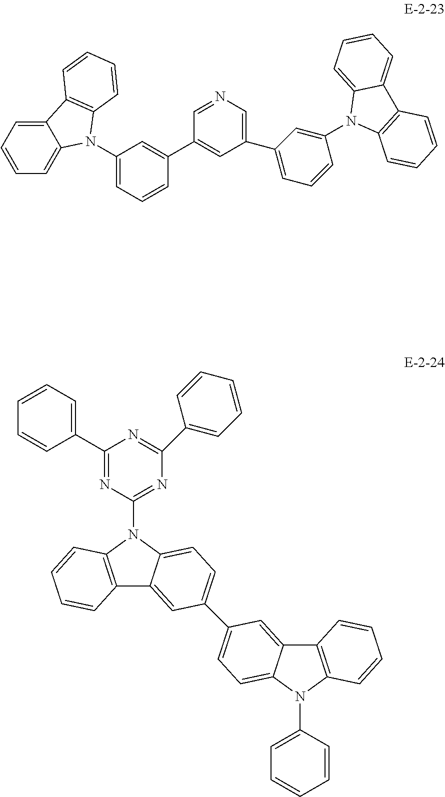 [SMILES: c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

In one or more embodiments, the emission layer EML may further include a material well-suitable in the art. For example, the emission layer EML may include as a host material, at least one of bis(4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazol-2-yl)benzene (TPBi). However, embodiments of the present disclosure are not limited thereto. For example, tris(8-hydroxyquinolinato)aluminum (Alq3), 9,10-di(naphthalen-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), and/or the like, may be used as the host material.

In one or more embodiments, the emission layer EML may include a compound represented by Formula M-a. The compound represented by Formula M-a may be used as a phosphorescence dopant material.

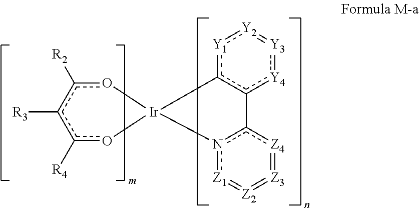

In Formula M-a, Y₁ to Y₄, and Z₁ to Z₄ may each independently be CR₁ or N, and R₁ to R₄ may each independently be hydrogen, deuterium, a substituted or unsubstituted amine group, a substituted or unsubstituted thiol group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be independently combined with an adjacent group to form a ring. In Formula M-a, “m” is 0 or 1, and “n” is 2 or 3. In Formula M-a, if (e.g., when) “m” is 0, “n” is 3, and if (e.g., when) “m” is 1, “n” is 2.

The compound represented by Formula M-a may be used as a phosphorescence dopant.

The compound represented by Formula M-a may be any one selected from among Compounds M-a1 to M-a25. However, Compounds M-a1 to M-a25 are mere examples, and the compound represented by Formula M-a is not limited to the compounds represented by Compounds M-a1 to M-a25.

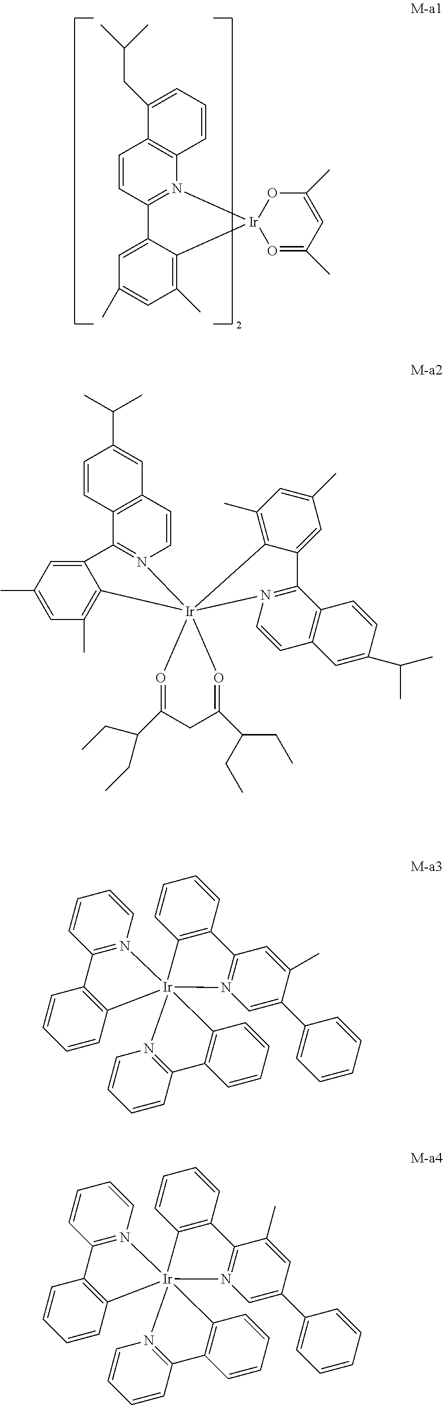

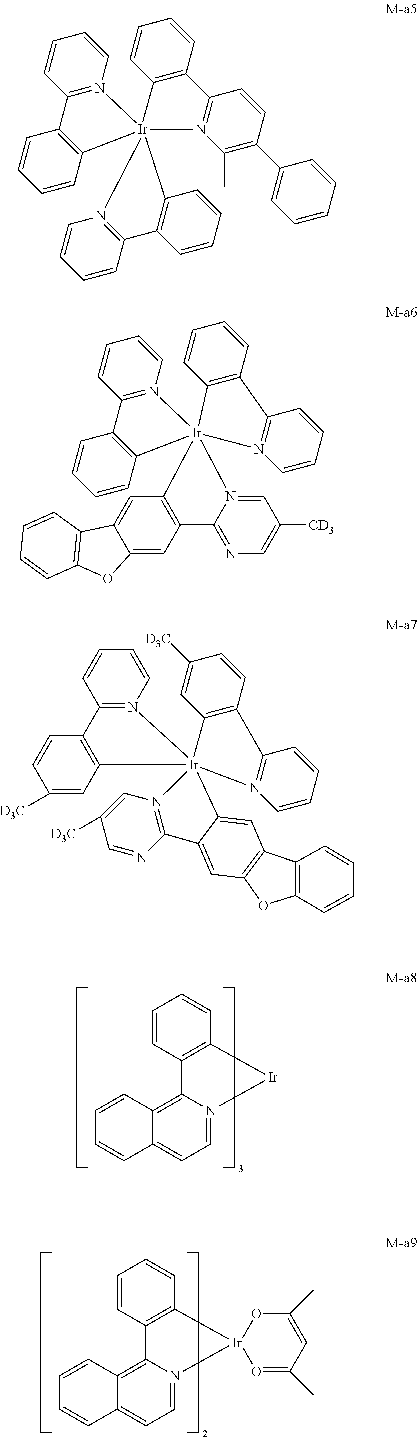

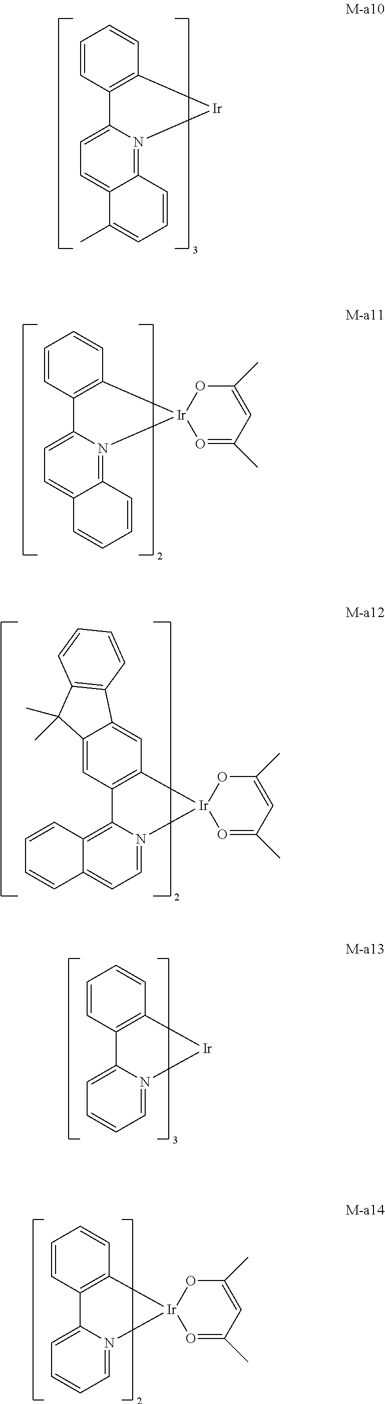

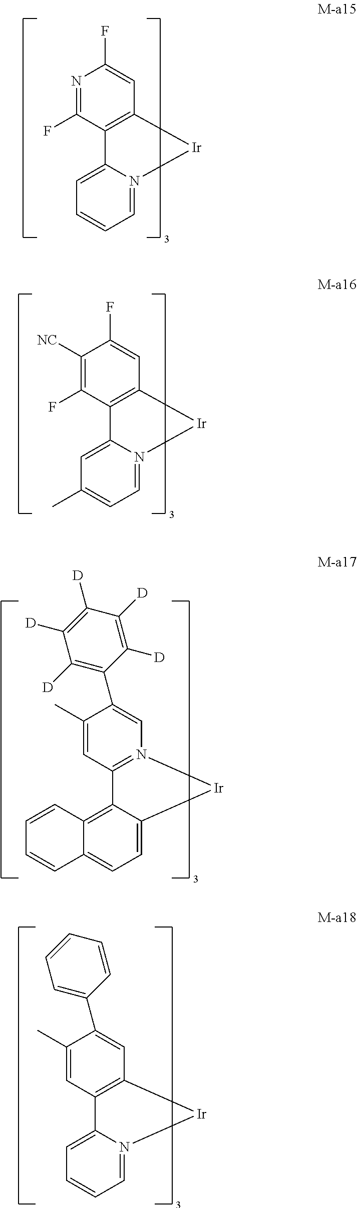

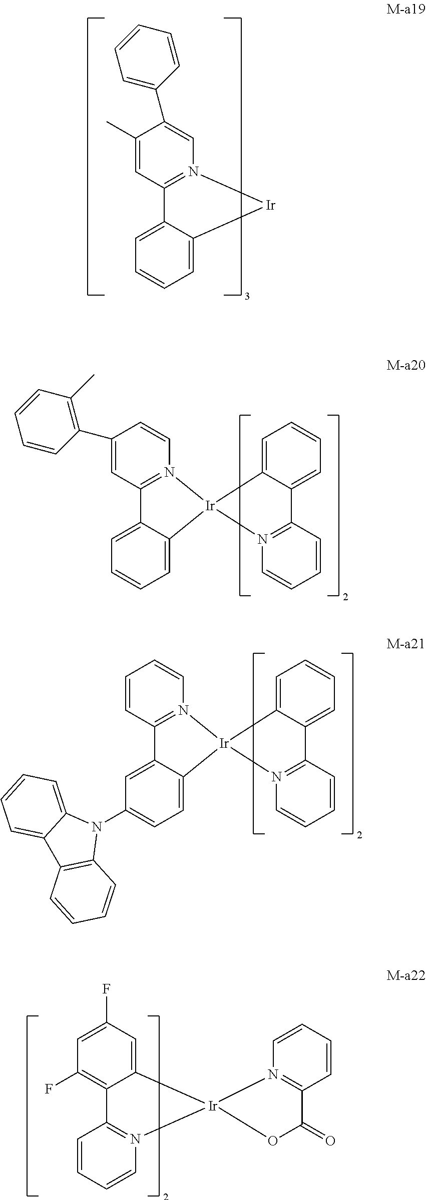

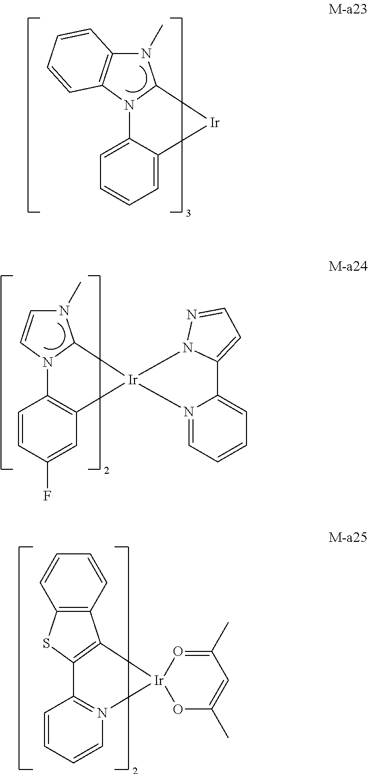

In one or more embodiments, the emission layer EML may further include a compound represented by any one selected from among Formula F-a to Formula F-c. The compounds represented by Formula F-a to Formula F-c may be used as fluorescence dopant materials or a thermally activated delayed fluorescence dopant material.

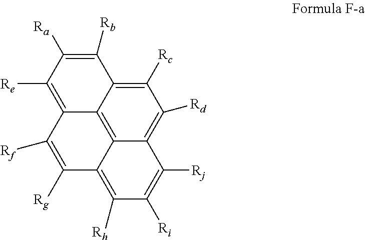

In Formula F-a, two selected from among Rₐ to Rⱼ may each independently be substituted with *—NAr₁Ar₂. The remainder not substituted with *—NAr₁Ar₂ among Rₐ to Rⱼ may each independently be hydrogen, deuterium, a halogen, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. In *—NAr₁Ar₂, Ar₁ and Ar₂ may each independently be a substituted or unsubstituted aryl group of 6 to 30 ring-forming carbon atoms or a substituted or unsubstituted heteroaryl group of 2 to 30 ring-forming carbon atoms. For example, in one or more embodiments, at least one selected from among Ar₁ and Ar₂ may be a heteroaryl group including O or S as a ring-forming atom.

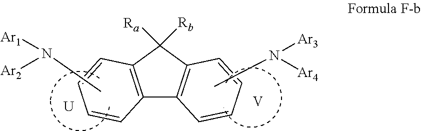

In Formula F-b, Rₐ and Rb may each independently be hydrogen, deuterium, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be independently combined with an adjacent group to form a ring. Ar₁ to Ar₄ may each independently be a substituted or unsubstituted aryl group of 6 to 30 ring-forming carbon atoms or a substituted or unsubstituted heteroaryl group of 2 to 30 ring-forming carbon atoms.

In Formula F-b, U and V may each independently be a substituted or unsubstituted hydrocarbon ring of 5 to 30 ring-forming carbon atoms or a substituted or unsubstituted heterocycle of 2 to 30 ring-forming carbon atoms. At least one selected from among Ar₁ to Ar₄ may be a heteroaryl group including O or S as a ring-forming atom.

In Formula F-b, the number of rings represented by U and V may each independently be 0 or 1. For example, in Formula F-b, if (e.g., when) the number of U or V is 1, one ring forms a part of a fused ring at the designated part by U or V, and if (e.g., when) the number of U or V is 0, a ring is not present at the designated part by U or V. For example, if (e.g., when) the number of U is 0, and the number of V is 1, or if (e.g., when) the number of U is 1, and the number of V is 0, a fused ring having the fluorene core of Formula F-b may be a ring compound with four rings. In one or more embodiments, if (e.g., when) the number of both (e.g., simultaneously) U and V is 0, the fused ring of Formula F-b may be a ring compound with three rings. In one or more embodiments, if (e.g., when) the number of both (e.g., simultaneously) U and V is 1, a fused ring having the fluorene core of Formula F-b may be a ring compound with five rings.

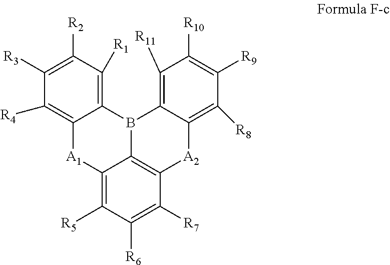

In Formula F-c, A₁ and A₂ may each independently be O, S, Se, or NRₘ, and Rₘ may be hydrogen, deuterium, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ may each independently be hydrogen, deuterium, a halogen, a cyano group, a substituted or unsubstituted amine group, a substituted or unsubstituted boryl group, a substituted or unsubstituted oxy group, a substituted or unsubstituted thiol group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or independently combined with an adjacent group to form a ring.

In Formula F-c, A₁ and A₂ may each independently be combined with the substituents of an adjacent ring to form a fused ring. For example, if (e.g., when) A₁ and A₂ may each independently be NRₘ, A₁ may be combined with R₄ or R₅ to form a ring. In addition, A₂ may be combined with R₇ or R₈ to form a ring.

In one or more embodiments, the emission layer EML may include as a suitable dopant material, one or more selected from among styryl derivatives (for example, 1,4-bis[2-(3-N-ethylcarbazolyl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and 4,4′-bis[2-(4-(N,N-diphenylamino)phenyl)vinyl]biphenyl (DPAVBi)), perylene and the derivatives thereof (for example, 2,5,8,11-tetra-t-butylperylene (TBP)), pyrene and the derivatives thereof (for example, 1,1-dipyrene, 1,4-dipyrenylbenzene, and 1,4-bis(N,N-diphenylamino)pyrene), and/or the like.

In one or more embodiments, the emission layer EML may include a suitable phosphorescence dopant material. For example, the phosphorescence dopant may use a metal complex including iridium (Ir), platinum (Pt), osmium (Os), gold (Au), titanium (Ti), zirconium (Zr), hafnium (Hf), europium (Eu), terbium (Tb), or thulium (Tm). For example, in one or more embodiments, iridium(III) bis(4,6-difluorophenylpyridinato-N,C2′)picolinate (Flrpic), bis(2,4-difluorophenylpyridinato)-tetrakis(1-pyrazolyl)borate iridium(III) (Fir6), or platinum octaethyl porphyrin (PtOEP) may be used as the phosphorescence dopant. However, embodiments of the present disclosure are not limited thereto.

In one or more embodiments, the emission layer may include a quantum dot.

In the present disclosure, the quantum dot refers to the crystal of a semiconductor compound. The quantum dot may be to emit light in one or more suitable emission wavelengths according to the size of the crystal. The quantum dot may be to emit light in one or more suitable emission wavelengths by controlling an element ratio in the quantum dot compound.

A diameter of the quantum dot may be, for example, about 1 nm to about 10 nm. In the present disclosure, when dot, dots, or dot particles are spherical, “diameter” indicates a particle diameter or an average particle diameter, and when the particles are non-spherical, the “diameter” indicates a major axis length or an average major axis length. The diameter of the particles may be measured utilizing a scanning electron microscope or a particle size analyzer. As the particle size analyzer, for example, HORIBA, LA-950 laser particle size analyzer, may be utilized. When the size of the particles is measured utilizing a particle size analyzer, the average particle diameter is referred to as D50. D50 refers to the average diameter of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size.

The quantum dot may be synthesized by a chemical bath deposition, a metal organic chemical vapor deposition, a molecular beam epitaxy, or a similar process therewith.

The chemical bath deposition is a method of mixing an organic solvent and a precursor material of a quantum dot and then, growing a quantum dot particle crystal. During growing the crystal, the organic solvent may naturally play the role of a dispersant which is coordinated on the surface of the quantum dot crystal and may control the growth of the crystal. Accordingly, the chemical bath deposition is more advantageous and beneficial if (e.g., when) compared to a vapor deposition method including a metal organic chemical vapor deposition (MOCVD) and/or molecular beam epitaxy (MBE), and the growth of the quantum dot particle may be controlled or selected through a low-cost process.

In one or more embodiments, the emission layer EML may include a quantum dot material. The quantum dot may have a core/shell structure. The core of the quantum dot may be selected from among a II-VI group compound, a III-VI group compound, a I-III-VI group compound, a III-V group compound, a III-II-V group compound, a IV-VI group compound, a IV group element, a IV group compound, and/or one or more (e.g., any suitable) combinations thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and a (e.g., any suitable) mixture thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a (e.g., any suitable) mixture thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and a (e.g., any suitable) mixture thereof. In one or more embodiments, the Group II-VI compound may further include a Group I metal and/or a Group IV element. The Group I-II-VI compound may be selected from among CuSnS and CuZnS, and the Group II-IV-VI compound may be selected from among ZnSnS and/or the like. The Group I-II-IV-VI compound may be selected from among quaternary compounds selected from the group consisting of Cu₂ZnSnS₂, Cu₂ZnSnS₄, Cu₂ZnSnSe₄, Ag₂ZnSnS₂, and a (e.g., any suitable) mixture thereof.

The III-VI group compound may include a binary compound such as In₂S₃ and/or In₂Se₃, a ternary compound such as InGaS₃ and/or InGaSes, or a (e.g., any suitable) combination thereof.

The I-III-VI group compound may be selected from among a ternary compound selected from the group consisting of AgInS, AgInS₂, CuInS, CuInS₂, AgGaS₂, CuGaS₂, CuGaO₂, AgGaO₂, AgAlO₂, and mixtures thereof, and/or a quaternary compound such as AgInGaS₂ and CuInGaS₂.

The III-V group compound may be selected from the group consisting of a binary compound selected from the group consisting of GaN, GaP, GaAs, GaSb, AlN, AlP, AlAs, AlSb, InN, InP, InAs, InSb, and mixtures thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and mixtures (e.g., combinations) thereof. In one or more embodiments, the III-V group compound may further include a II group metal. For example, InZnP, and/or the like, may be selected as a III-II-V group compound.

The Group IV-VI compound may be selected from the group consisting of a binary compound selected from the group consisting of SnS, SnSe, SnTe, PbS, PbSe, PbTe, and a (e.g., any suitable) mixture thereof, a ternary compound selected from the group consisting of SnSeS, SnSeTe, SnSTe, PbSeS, PbSeTe, PbSTe, SnPbS, SnPbSe, SnPbTe, and a (e.g., any suitable) mixture (e.g., combination) thereof, and a quaternary compound selected from the group consisting of SnPbSSe, SnPbSeTe, SnPbSTe, and a (e.g., any suitable) mixture thereof.

The Group II-IV-V compound may be a ternary compound selected from the group consisting of ZnSnP, ZnSnP₂, ZnSnAs₂, ZnGeP₂, ZnGeAs₂, CdSnP₂, CdGeP₂, and a (e.g., any suitable) mixture thereof.

The Group IV element may be selected from the group consisting of Si, Ge, and a (e.g., any suitable) mixture thereof. The Group IV compound may be a binary compound selected from the group consisting of SiC, SiGe, and a (e.g., any suitable) mixture thereof.

Each element included in a multi-element compound such as the binary compound, ternary compound, or quaternary compound may be present in particles at a substantially uniform concentration or a non-uniform concentration. For example, Formula above indicates the types (kinds) of elements included in a compound, and element ratios in the compound may be different. For example, AgInGaS₂ may indicate AgInₓGa₁₋ₓS₂ (x is a real number between 0 and 1).

In one or more embodiments, the binary compound, the ternary compound, or the quaternary compound may be present at substantially uniform concentration in a particle or may be present at a partially different concentration distribution state in the same particle. In one or more embodiments, a core/shell structure in which one quantum dot wraps another quantum dot may be desired. The interface of the core and the shell may have a concentration gradient in which the concentration of an element present in the shell is decreased toward the center.

In one or more embodiments, the quantum dot may have the above-described core-shell structure including a core including a nanocrystal and a shell wrapping the core. The shell of the quantum dot may play the role of a protection layer for preventing or reducing the chemical deformation of the core to maintain semiconductor properties and/or a charging layer for imparting the quantum dot with electrophoretic properties. The shell may have a single layer or a multilayer. Examples of the shell of the quantum dot may include a metal or non-metal oxide, a semiconductor compound, and/or one or more (e.g., any suitable) combinations thereof.

For example, the metal or non-metal oxide for the shell may include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, and/or NiO, or a ternary compound such as MgAl₂O₄, CoFe₂O₄, NiFe₂O₄, and/or CoMn₂O₄, but embodiments of the present disclosure are not limited thereto.

Also, the semiconductor compound suitable as a shell may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, and/or the like, but embodiments of the present disclosure are not limited thereto.

The quantum dot may have a full width of half maximum (FWHM) of emission spectrum of about 45 nm or less, about 40 nm or less, or about 30 nm or less. Within this range, color purity or color reproducibility of the quantum dot may be improved. In addition, light emitted via such quantum dot is emitted in all directions, and light view angle properties may be improved.

In addition, the shape of the quantum dot may be a generally used shape in the art, without specific limitation. For example, spherical nanoparticles, pyramidal nanoparticles, multi-arm nanoparticles, cubic nanoparticles, nanotubes, nanowires, nanofibers, nanoplates, and/or the like, may be used.

As the size of the quantum dot or the ratio of elements in the quantum dot compound is regulated, the energy band gap of the quantum dot may be accordingly controlled or selected to obtain light of one or more suitable wavelengths from a quantum dot emission layer. Therefore, by using the quantum dots as described above (using quantum dots of different sizes or having different element ratios in the quantum dot compound), a light emitting element emitting light of one or more suitable wavelengths may be obtained. For example, the size of the quantum dots or the ratio of elements in the quantum dot compound may be regulated to enable the quantum dots to emit red, green, and/or blue light. In one or more embodiments, the quantum dots may be configured to emit white light by combining light of one or more suitable colors.

In the light emitting element ED of embodiments, as shown in FIG. 3 to FIG. 6, the electron transport region ETR may be provided on the emission layer EML. The electron transport region ETR may include at least one of a hole blocking layer HBL, an electron transport layer ETL, or an electron injection layer EIL. However, embodiments of the present disclosure are not limited thereto.

The electron transport region ETR may have a single layer formed using a single material, a single layer formed using multiple different materials, or a multilayer structure having multiple layers formed using multiple different materials.

In one or more embodiments, the electron transport region ETR may include the nitrogen-containing compound represented by Formula 1. For example, the nitrogen-containing compound of one or more embodiments may be included in a layer in contact with the emission layer EML among the above-described multiple functional layers. For example, the nitrogen-containing compound of one or more embodiments may be included in the electron transport layer ETL. However, embodiments of the present disclosure are not limited thereto. The nitrogen-containing compound of one or more embodiments may be included in the hole blocking layer HBL or the electron injection layer EIL in addition to the electron transport layer ETL. As for the nitrogen-containing compound, the explanation for the nitrogen-containing compound included in the emission layer EML may be applied in substantially the same way, so detailed explanation will not be provided for conciseness.

Because the nitrogen-containing compound of one or more embodiments has a structure in which a carbazole moiety and first and second triazine moieties, each substituted with at least one carbazole group, are connected with one phenyl linker, and the first triazine moiety and the second triazine moiety are each substituted at an ortho position with respect to the carbazole moiety, excellent or suitable electron transport properties may be shown, and the high lowest excited triplet energy level may be obtained.

Accordingly, the light emitting element ED of one or more embodiments may achieve high efficiency and long lifetime by including the nitrogen-containing compound of one or more embodiments in the electron transport region ETR. For example, the light emitting element ED of one or more embodiments may achieve high efficiency and long lifetime by including the nitrogen-containing compound of one or more embodiments in the electron transport layer ETL. However, embodiments of the present disclosure are not limited thereto, and the multiple functional layers of the electron transport region ETR may independently include the nitrogen-containing compound of one or more embodiments, without limitation.

The electron transport region ETR may have a single layer formed using a single material, a single layer formed using multiple different materials, or a multilayer structure having multiple layers formed using multiple different materials.

For example, in one or more embodiments, the electron transport region ETR may have a single layer structure of an electron injection layer EIL or an electron transport layer ETL, or a single layer structure formed using an electron injection material and/or an electron transport material. Further, in one or more embodiments, the electron transport region ETR may have a single layer structure formed using multiple different materials, or a structure stacked from the emission layer EML of electron transport layer ETL/electron injection layer EIL, or hole blocking layer HBL/electron transport layer ETL/electron injection layer EIL, without limitation. A thickness of the electron transport region ETR may be, for example, from about 1,000 Å to about 1,500 Å. At least one selected from among the multiple layers of the electron transport region ETR may include the nitrogen-containing compound of one or more embodiments.

The electron transport region ETR may include one or two or more types (kinds) of the nitrogen-containing compounds of Formula 1. For example, in one or more embodiments, the electron transport region ETR may include at least one nitrogen-containing compound selected from among the compounds represented in the above-described Compound Group 1.

The electron transport region ETR may be formed using one or more suitable methods such as a vacuum deposition method, a spin coating method, a casting method, a Langmuir-Blodgett (LB) method, an inkjet printing method, a laser printing method, and/or a laser induced thermal imaging (LITI) method.

In one or more embodiments, the electron transport region ETR may include a compound represented by Formula ET-2.

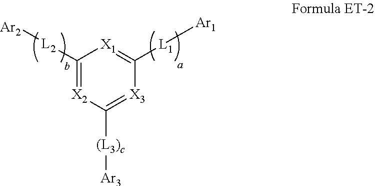

In Formula ET-2, at least one selected from among X₁ to X₃ may be N, and the remainder are CRₐ. Rₐ may be hydrogen, deuterium, a substituted or unsubstituted alkyl of 1 to 20 carbon atoms, a substituted or unsubstituted aryl group of 6 to 30 ring-forming carbon atoms, or a substituted or unsubstituted heteroaryl group of 2 to 30 ring-forming carbon atoms. Ar₁ to Ar₃ may each independently be hydrogen, deuterium, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-2, “a” to “c” may each independently be an integer of 0 to 10. In Formula ET-2, L₁ to L₃ may each independently be a direct linkage, a substituted or unsubstituted arylene group of 6 to 30 ring-forming carbon atoms, or a substituted or unsubstituted heteroarylene group of 2 to 30 ring-forming carbon atoms. In one or more embodiments, if (e.g., when) “a” to “c” are each an integer of 2 or more, L₁'s to L₃'s may each independently be a substituted or unsubstituted arylene group of 6 to 30 ring-forming carbon atoms or a substituted or unsubstituted heteroarylene group of 2 to 30 ring-forming carbon atoms.

In one or more embodiments, the electron transport region ETR may include an anthracene-based compound. However, embodiments of the present disclosure are not limited thereto, and the electron transport region ETR may include, for example, tris(8-hydroxyquinolinato)aluminum (Alq3), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzimidazolyl-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(biphenyl-4-yl)-4-phenyl-5-tert-butylphenyl-1,2,4-triazole (TAZ), 4-(naphthalen-1-yl)-3,5-diphenyl-4H-1,2,4-triazole (NTAZ), 2-(biphenyl-4-yl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalen-2-yl)anthracene (ADN), 1,3-bis[3,5-di(pyridin-3-yl)phenyl]benzene (BmPyPhB), CNNPTRZ (4′-(4-(4-(4,6-diphenyl-1,3,5-triazin-2-yl)phenyl)naphthalen-1-yl)-[1,1′-biphenyl]-4-carbonitrile), and/or a (e.g., any suitable) mixture thereof, without limitation.

In one or more embodiments, electron transport region ETR may include any one selected from among the compounds in Compound Group 2.

In one or more embodiments, the electron transport region ETR may include at least one selected from among Compounds ET1 to ET38.

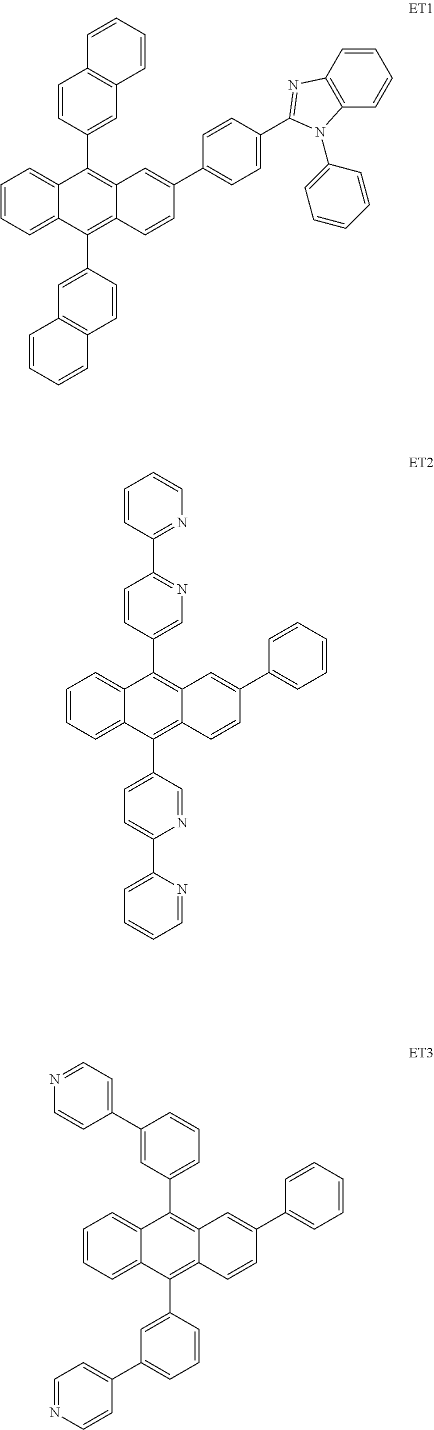 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

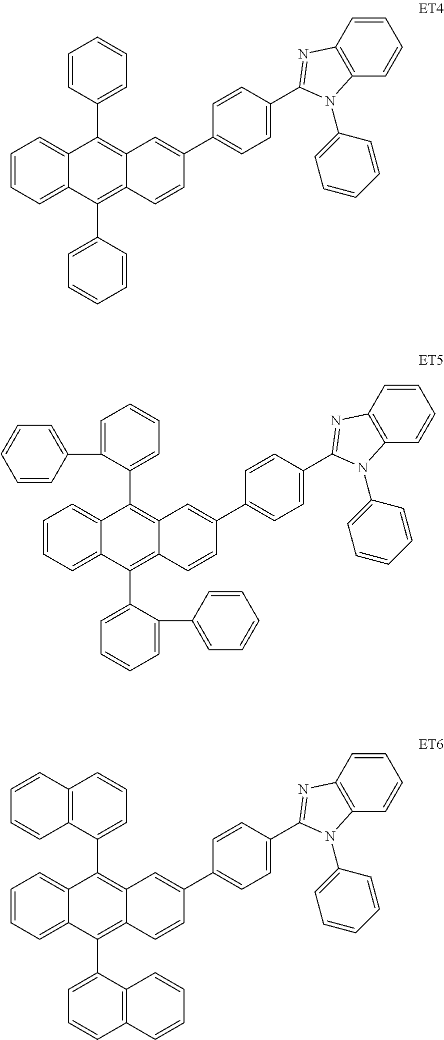 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

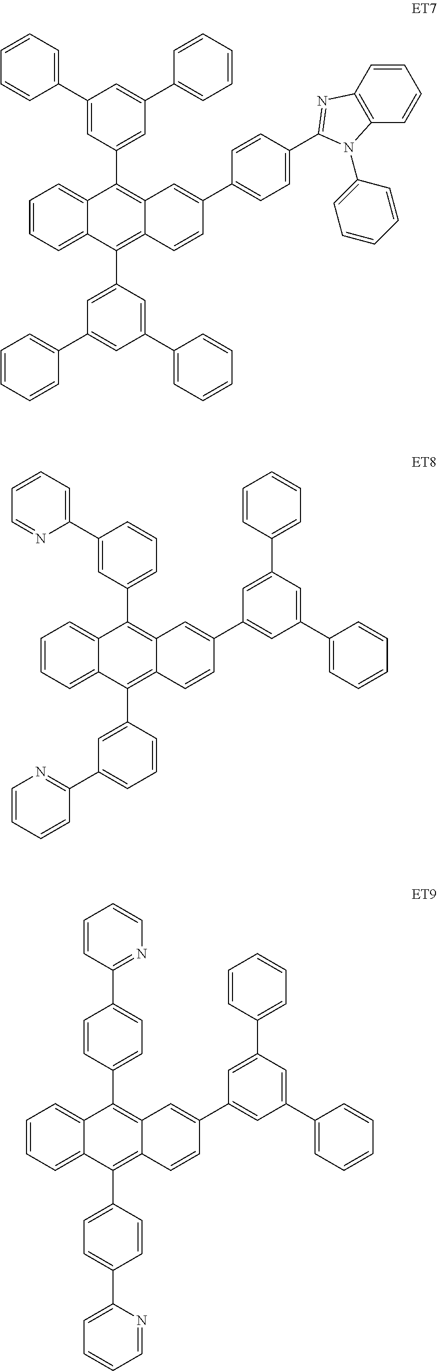 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

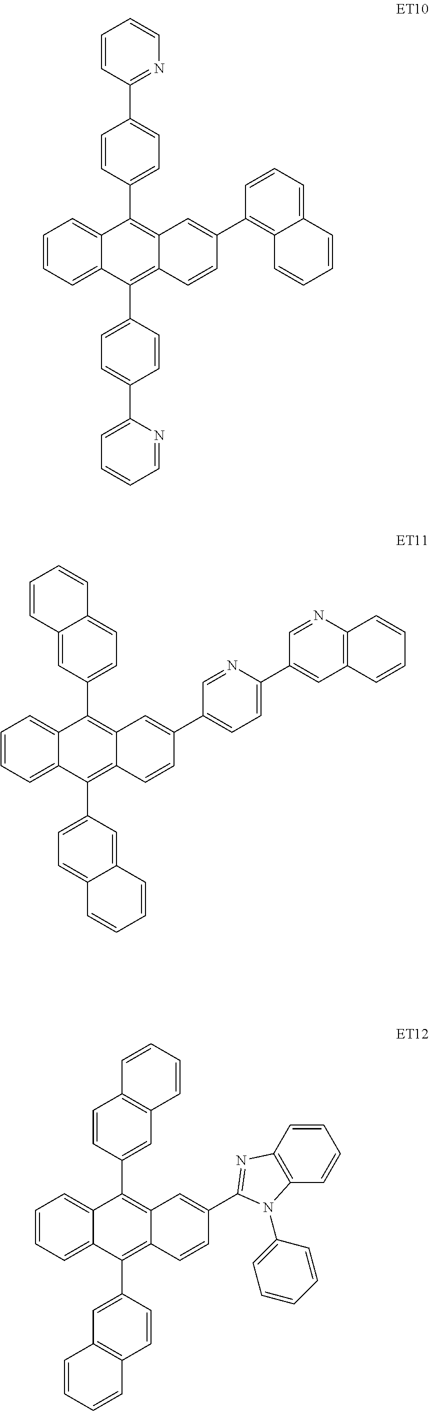 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

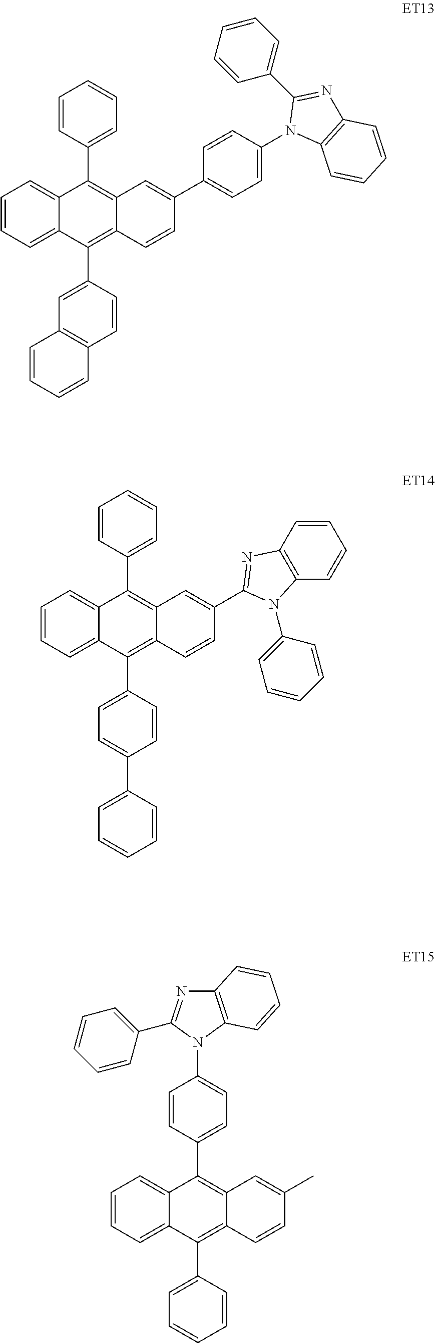 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

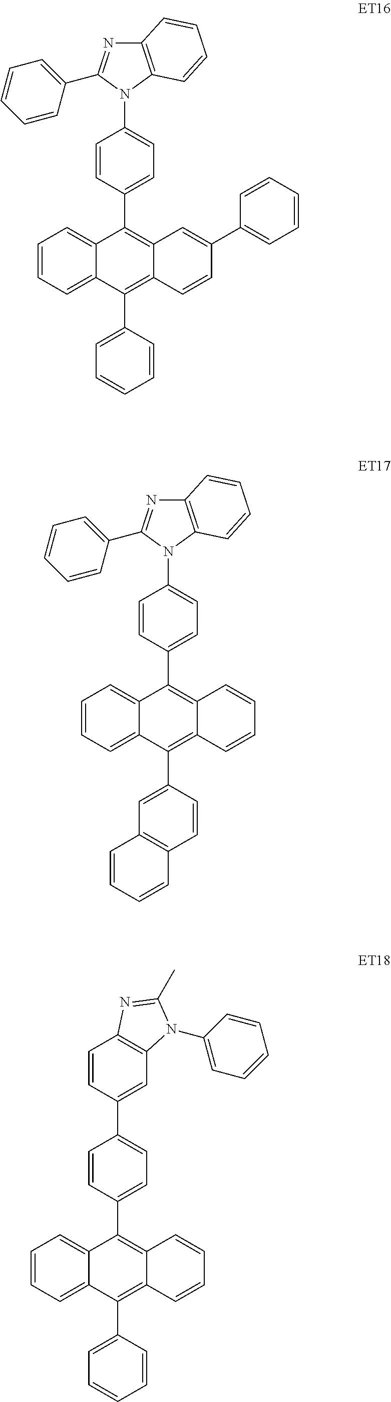 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

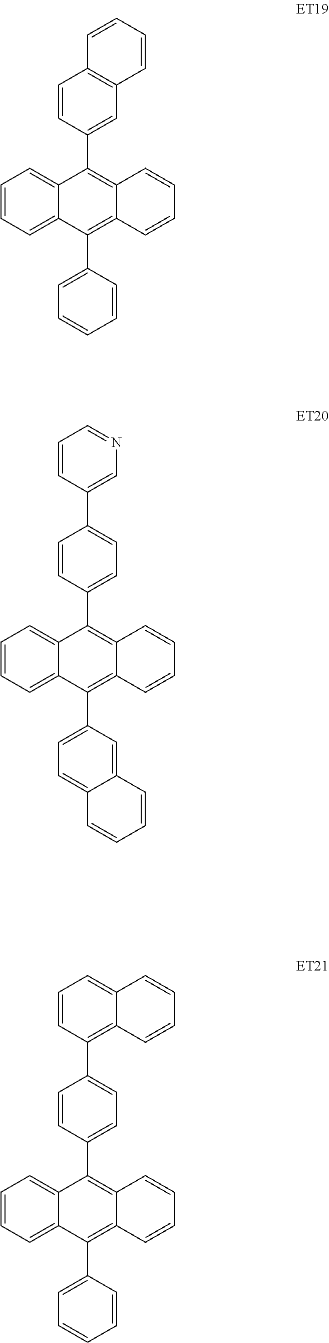

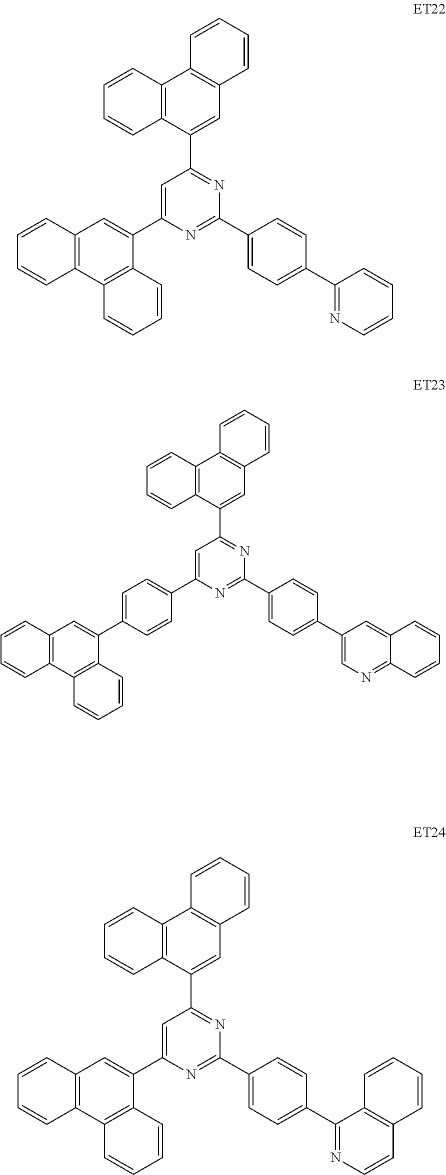 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

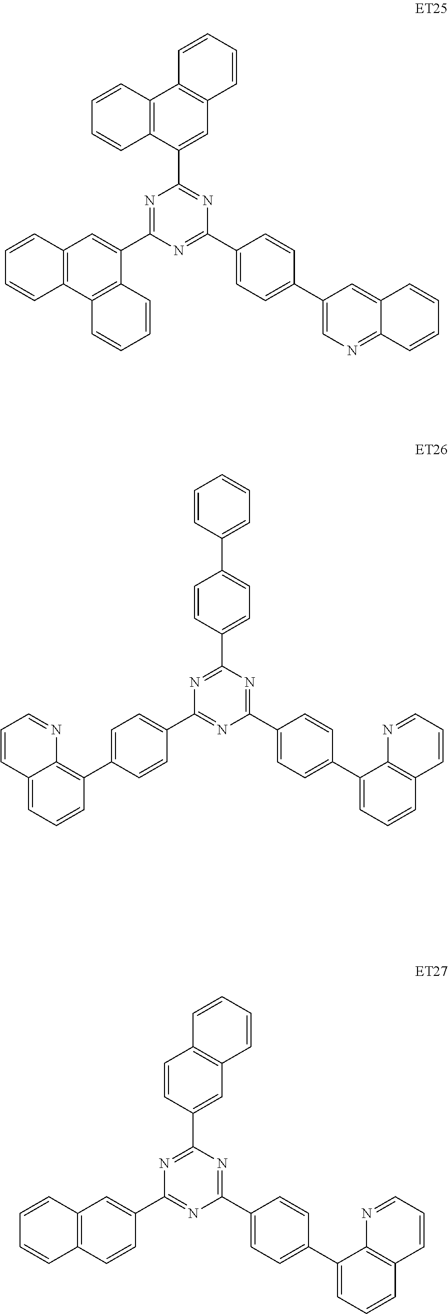 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

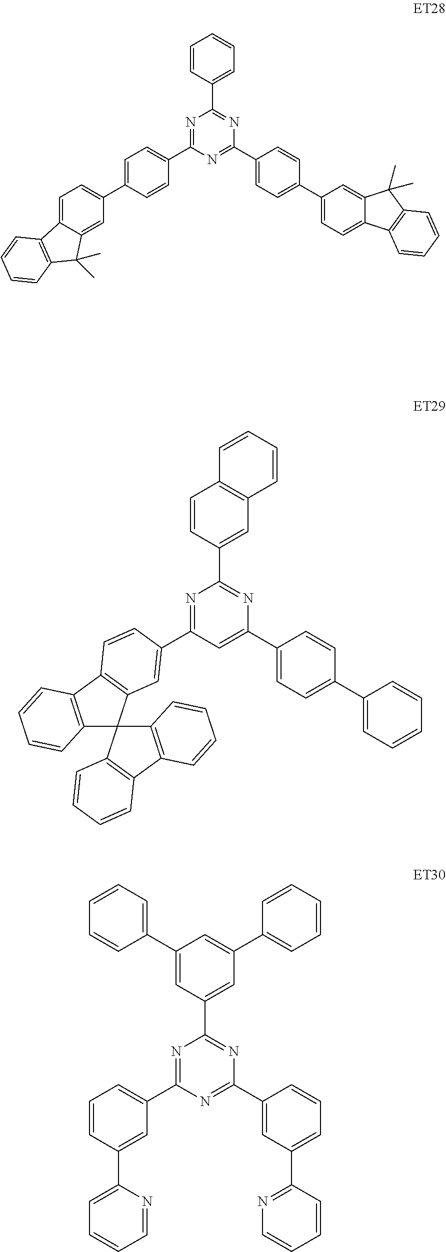 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

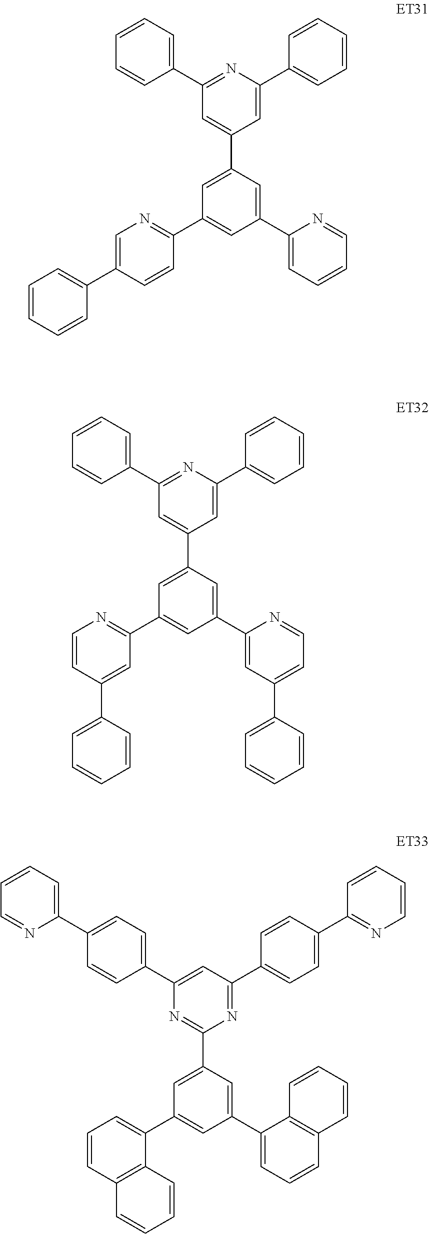 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

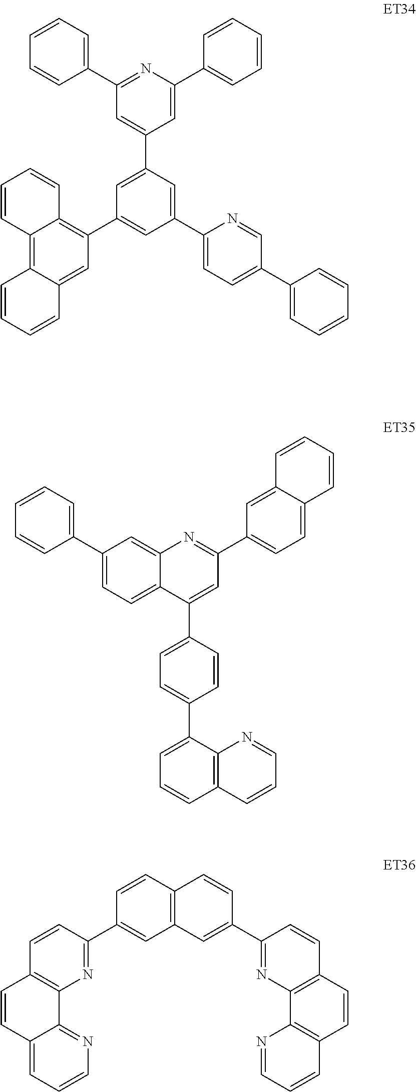 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

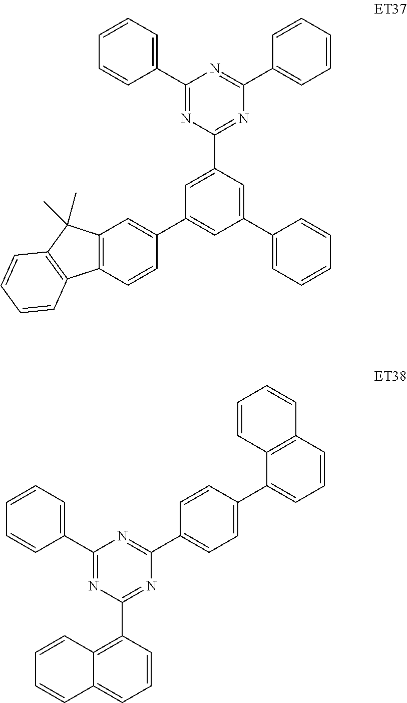 [SMILES: CC1(C)c2ccccc2-c2ccc(-c3cc(-c4ccccc4)cc(-c4nc(-c5ccccc5)nc(-c5ccccc5)n4)c3)cc21 ; c1ccc(-c2nc(-c3ccc(-c4cccc5ccccc45)cc3)nc(-c3cccc4ccccc34)n2)cc1]

In one or more embodiments, the electron transport region ETR may include a metal halide such as LiF, NaCl, CsF, RbCl, RbI, CuI, and/or KI, a lanthanide metal such as Yb, or a co-depositing material of the metal halide and the lanthanide metal. For example, in one or more embodiments, the electron transport region ETR may include KI:Yb, RbI:Yb, LiF:Yb, and/or the like, as the co-depositing material. In one or more embodiments, the electron transport region ETR may use a metal oxide such as Li₂O and/or BaO, or lithium 8-hydroxyquinolinolate (LiQ). However, embodiments of the present disclosure are not limited thereto. The electron transport region ETR also may be formed using a mixture material of an electron transport material and an insulating organo metal salt. The organo metal salt may be a material having an energy band gap of about 4 eV or more. For example, the organo metal salt may include, for example, one or more of (e.g., at least one of) metal acetates, metal benzoates, metal acetoacetates, metal acetylacetonates, or metal stearates.

In one or more embodiments, the electron transport region ETR may include at least one of 2,9-dimethyl-4,7-diphenyl-1,10-phenanthroline (BCP), diphenyl(4-(triphenylsilyl)phenyl)phosphine oxide (TSPO1), or 4,7-diphenyl-1,10-phenanthroline (Bphen) in addition to one or more of the aforementioned materials. However, embodiments of the present disclosure are not limited thereto.

The electron transport region ETR may include one or more of the compounds of the electron transport region in at least one selected from among an electron injection layer EIL, an electron transport layer ETL, and a hole blocking layer HBL.

If (e.g., when) the electron transport region ETR includes an electron transport layer ETL, a thickness of the electron transport layer ETL may be from about 100 Å to about 1,000 Å, for example, from about 150 Å to about 500 Å. If (e.g., when) the thickness of the electron transport layer ETL satisfies the above-described range, satisfactory electron transport properties may be obtained without substantial increase of a driving voltage. If (e.g., when) the electron transport region ETR includes an electron injection layer EIL, a thickness of the electron injection layer EIL may be from about 1 Å to about 100 Å, or from about 3 Å to about 90 Å. If (e.g., when) the thickness of the electron injection layer EIL satisfies the above described range, satisfactory electron injection properties may be obtained without inducing substantial increase of a driving voltage.

The second electrode EL2 may be provided on the electron transport region ETR. The second electrode EL2 may be a common electrode. The second electrode EL2 may be a cathode or an anode, but embodiments of the present disclosure are not limited thereto. For example, if (e.g., when) the first electrode EL1 is an anode, the second cathode EL2 may be a cathode, and if (e.g., when) the first electrode EL1 is a cathode, the second electrode EL2 may be an anode.

The second electrode EL2 may be a transmissive electrode, a transflective electrode, or a reflective electrode. If (e.g., when) the second electrode EL2 is the transmissive electrode, the second electrode EL2 may include a transparent metal oxide, for example, ITO, IZO, ZnO, ITZO, and/or the like.

If (e.g., when) the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, a (e.g., any suitable) compound thereof, or a (e.g., any suitable) mixture thereof (for example, AgMg, AgYb, or MgYb). In one or more embodiments, the second electrode EL2 may have a multilayered structure including a reflective layer or a transflective layer formed using one or more of the above-described materials and a transparent conductive layer formed using ITO, IZO, ZnO, ITZO, and/or the like. For example, in one or more embodiments, the second electrode EL2 may include the one of aforementioned metal materials, a (e.g., any suitable) combination of two or more metal materials selected therefrom, or an oxide of the aforementioned metal materials.

In one or more embodiments, the second electrode EL2 may be connected with an auxiliary electrode. If (e.g., when) the second electrode EL2 is connected with the auxiliary electrode, the resistance of the second electrode EL2 may decrease.

In one or more embodiments, on the second electrode EL2 in the light emitting element ED, a capping layer CPL may be further arranged. The capping layer CPL may include a multilayer or a single layer.

In one or more embodiments, the capping layer CPL may be an organic layer or an inorganic layer. For example, if (e.g., when) the capping layer CPL includes an inorganic material, the inorganic material may include an alkali metal compound such as LiF, an alkaline earth metal compound such as MgF₂, SiON, SiNₓ, SiO_{y}, and/or the like.

In one or more embodiments, if (e.g., when) the capping layer CPL includes an organic material, the organic material may include 2,2′-dimethyl-N,N′-di-[(1-naphthyl)-N,N′-diphenyl]-1,1′-biphenyl-4,4′-diamine(α-NPD), NPB, TPD, m-MTDATA, Alq3, CuPc, N4,N4,N4′,N4′-tetra(biphenyl-4-yl) biphenyl-4,4′-diamine (TPD15), 4,4′,4″-tris(carbazol-9-yl) triphenylamine (TCTA), and/or the like, or may include an epoxy-based resin or an acrylate-based resin such as poly(methacrylate). In one or more embodiments, the capping layer CPL may include at least one selected from among Compounds P1 to P5, but embodiments of the present disclosure are not limited thereto.

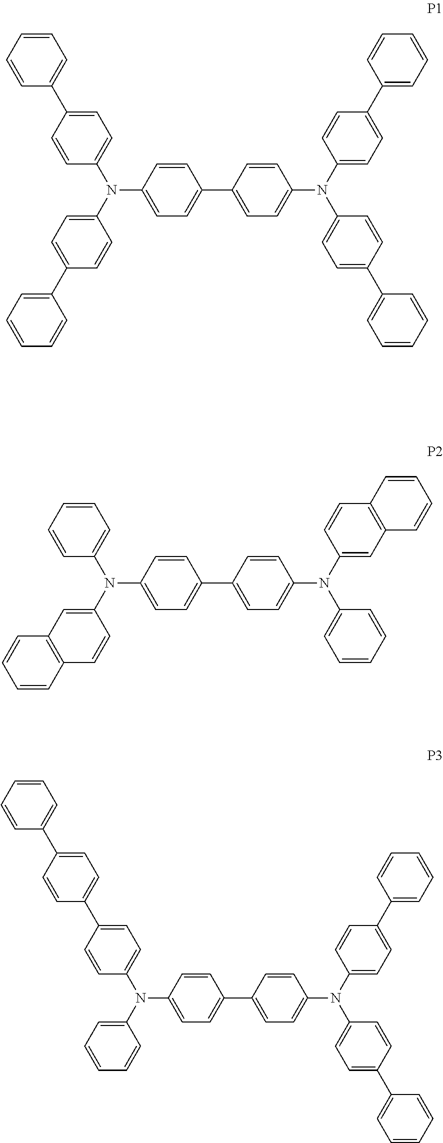 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

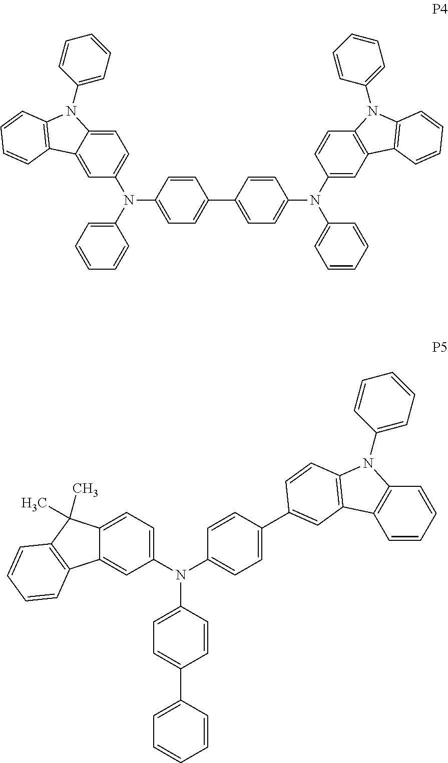 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In one or more embodiments, the refractive index of the capping layer CPL may be about 1.6 or more. For example, in one or more embodiments, the refractive index of the capping layer CPL with respect to light in a wavelength range of about 550 nm to about 660 nm may be about 1.6 or more.

In the light emitting element ED, according to the application of a voltage to each of the first electrode EL1 and the second electrode EL2, holes injected from the first electrode EL1 may move via the hole transport region HTR to the emission layer EML, and electrons injected from the second electrode EL2 may move via the electron transport region ETR to the emission layer EML. The electrons and holes recombine in the emission layer EML to produce excitons, light is generated and emitted through the transition of the excitons from an excited state to a ground state.

The nitrogen-containing compound according to one or more embodiments of the disclosure has a structure in which a carbazole moiety and first and second triazine moieties each substituted with at least one carbazole group, are connected with one phenyl linker, and the first triazine moiety and the second triazine moiety are each substituted at an ortho position with respect to the carbazole moiety, and may have excellent or suitable electron transport properties and the high lowest excited triplet energy level (T1 level).

The nitrogen-containing compound of one or more embodiments may have the high lowest excited triplet energy level (T1 level) of about 2.8 eV or higher. Accordingly, the light emitting element ED of one or more embodiments may be to emit blue phosphorescence or blue thermally activated delayed light by using the nitrogen-containing compound of one or more embodiments in the emission layer EML as a host. In addition, the nitrogen-containing compound of one or more embodiments exhibits high electron mobility, and if (e.g., when) included in the electron transport region ETR of the light emitting element ED, an excellent or suitable role of electron transport may be shown. Accordingly, the light emitting element ED of one or more embodiments may have high efficiency and long-life characteristics.

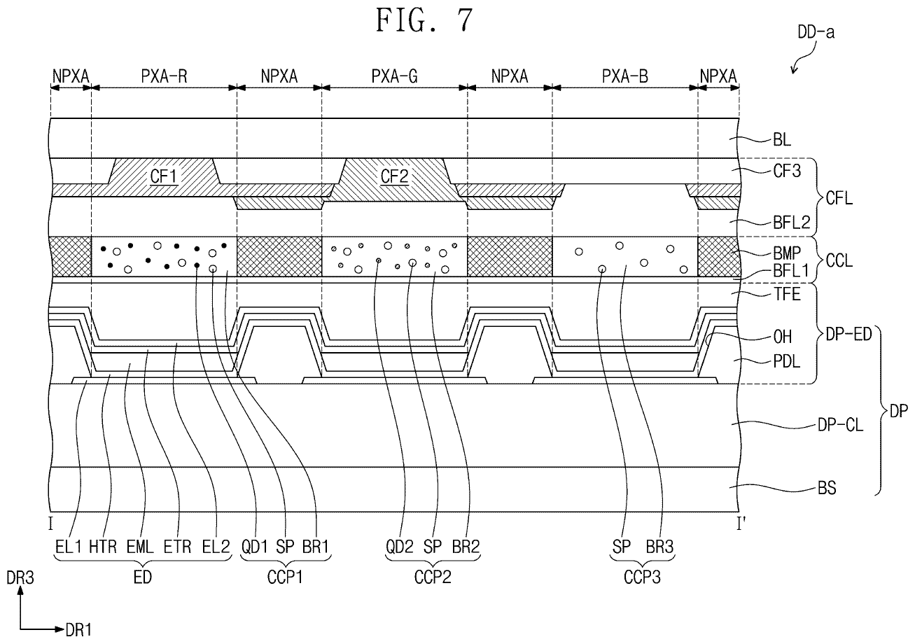

FIG. 7 to FIG. 10 are each a cross-sectional view of a display device according to one or more embodiments of the present disclosure. In the explanation on the display devices of embodiments referring to FIG. 7 to FIG. 10, the overlapping parts with the explanation on FIG. 1 to FIG. 6 will not be explained again, and only different features will be explained chiefly.

Referring to FIG. 7, the display device DD-a according to one or more embodiments may include a display panel DP including a display device layer DP-ED, a light controlling layer CCL arranged on the display panel DP, and a color filter layer CFL. In one or more embodiments, as shown in FIG. 7, the display panel DP may include a base layer BS, a circuit layer DP-CL provided on the base layer BS, and the display device layer DP-ED, and the display device layer DP-ED may include a light emitting element ED.

The light emitting element ED may include a first electrode EL1, a hole transport region HTR arranged on the first electrode EL1, an emission layer EML arranged on the hole transport region HTR, an electron transport region ETR arranged on the emission layer EML, and a second electrode EL2 arranged on the electron transport region ETR. In one or more embodiments, the same structure as any one of the light emitting elements of FIG. 3 to FIG. 6 may be applied to the structure of the light emitting element ED shown in FIG. 7.

The emission layer EML of the light emitting element ED included in the display device DD-a according to one or more embodiments may include the nitrogen-containing compound of one or more embodiments described above. In one or more embodiments, the electron transport region ETR of the light emitting element ED included in the display device DD-a according to one or more embodiments may include the nitrogen-containing compound of one or more embodiments described above.

Referring to FIG. 7, the emission layer EML may be arranged in an opening part OH defined in a pixel definition layer PDL. For example, the emission layer EML divided by the pixel definition layer PDL and correspondingly provided to each of luminous areas PXA-R, PXA-G, and PXA-B may be to emit light in substantially the same wavelength region. In the display device DD-a of one or more embodiments, the emission layer EML may be to emit blue light. In one or more embodiments, the emission layer EML may be provided as a common layer for all luminous areas PXA-R, PXA-G, and PXA-B.

The light controlling layer CCL may be arranged on the display panel DP. The light controlling layer CCL may include a light converter. The light converter may be a quantum dot or a phosphor. The light converter may transform the wavelength of light provided and then emit the transformed light. For example, the light controlling layer CCL may be a layer including a quantum dot or a layer including a phosphor.

The light controlling layer CCL may include multiple light controlling parts CCP1, CCP2, and CCP3. The light controlling parts CCP1, CCP2, and CCP3 may be separated from one another.

Referring to FIG. 7, a partition pattern BMP may be arranged between the separated light controlling parts CCP1, CCP2, and CCP3, but embodiments of the disclosure are not limited thereto. In FIG. 7, the partition pattern BMP is shown not to be overlapped with the light controlling parts CCP1, CCP2, and CCP3, but, in one or more embodiments, at least a portion of the edges of the light controlling parts CCP1, CCP2, and CCP3 may be overlapped with the partition pattern BMP.

The light controlling layer CCL may include a first light controlling part CCP1 including a first quantum dot QD1 that converts first color light provided from the light emitting element ED into second color light, a second light controlling part CCP2 including a second quantum dot QD2 that converts the first color light into third color light, and a third light controlling part CCP3 transmitting the first color light. In one or more embodiments, the first light controlling part CCP1 may provide red light which is the second color light, and the second light controlling part CCP2 may provide green light which is the third color light. The third color controlling part CCP3 may be to transmit and provide blue light which is the first color light provided from the light emitting element ED. For example, in one or more embodiments, the first quantum dot QD1 may be a red quantum dot to emit red light, and the second quantum dot QD2 may be a green quantum dot to emit green light. On the quantum dots QD1 and QD2, the same contents as those described above on the quantum dot may be applied.

In one or more embodiments, the light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include a (e.g., may exclude any) quantum dot but include the scatterer SP.

The scatterer SP may be an inorganic particle. For example, the scatterer SP may include at least one selected from among TiO₂, ZnO, Al₂O₃, SiO₂, and hollow silica. In one or more embodiments, the scatterer SP may include at least one selected from among TiO₂, ZnO, Al₂O₃, SiO₂, and hollow silica, or may be a mixture of two or more materials selected from among TiO₂, ZnO, Al₂O₃, SiO₂, and hollow silica.

The first light controlling part CCP1, the second light controlling part CCP2, and the third light controlling part CCP3 may respectively include base resins BR1, BR2, and BR3 in which the quantum dots QD1 and QD2 and the scatterer SP are dispersed accordingly. In one or more embodiments, the first light controlling part CCP1 may include the first quantum dot QD1 and the scatterer SP dispersed in a first base resin BR1, the second light controlling part CCP2 may include the second quantum dot QD2 and the scatterer SP dispersed in a second base resin BR2, and the third light controlling part CCP3 may include the scatterer particle SP dispersed in a third base resin BR3.

The base resins BR1, BR2, and BR3 are media in which the quantum dots QD1 and QD2 and the scatterer SP are dispersed accordingly, and may each be composed of one or more suitable resin compositions which may be generally referred to as a binder. For example, the base resins BR1, BR2, and BR3 may each independently be an acrylic-based resin, a urethane-based resin, a silicone-based resin, an epoxy-based resin, and/or the like. The base resins BR1, BR2, and BR3 may each be a transparent resin. In one or more embodiments, the first base resin BR1, the second base resin BR2, and the third base resin BR3 may be the same or different from one another.

In one or more embodiments, the light controlling layer CCL may include a barrier layer BFL1. The barrier layer BFL1 may play the role of blocking the penetration of moisture and/or oxygen (hereinafter, will be referred to as “humidity/oxygen”). The barrier layer BFL1 may block the exposure of the light controlling parts CCP1, CCP2, and CCP3 to humidity/oxygen. In one or more embodiments, the barrier layer BFL1 may cover the light controlling parts CCP1, CCP2, and CCP3. In one or more embodiments, a color filter layer CFL, which will be explained later, may include a barrier layer BFL2 arranged on the light controlling parts CCP1, CCP2, and CCP3.

The barrier layers BFL1 and BFL2 may each include at least one inorganic layer. For example, in one or more embodiments, the barrier layers BFL1 and BFL2 may each be formed by including an inorganic material. For example, the barrier layers BFL1 and BFL2 may be each independently formed by including silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, or a metal thin film securing light transmittance. In one or more embodiments, the barrier layers BFL1 and BFL2 may each independently further include an organic layer. The barrier layers BFL1 and BFL2 may each independently be composed of a single layer of multiple layers.

In the display device DD-a of one or more embodiments, the color filter layer CFL may be arranged on the light controlling layer CCL. For example, in one or more embodiments, the color filter layer CFL may be arranged directly on the light controlling layer CCL. In these embodiments, the barrier layer BFL2 may not be provided.

The color filter layer CFL may include filters CF1, CF2, and CF3. The first to third filters CF1, CF2 and CF3 may be arranged corresponding to a red luminous area PXA-R, a green luminous area PXA-G, and a blue luminous area PXA-B, respectively.

The color filter layer CFL may include a first filter CF1 configured to transmit the second color light, a second filter CF2 configured to transmit the third color light, and a third filter CF3 configured to transmit the first color light. For example, in one or more embodiments, the first filter CF1 may be a red filter, the second filter CF2 may be a green filter, and the third filter CF3 may be a blue filter. Each of the filters CF1, CF2 and CF3 may include a polymer photosensitive resin and a pigment and/or a dye. For example, the first filter CF1 may include a red pigment and/or a red dye, the second filter CF2 may include a green pigment and/or a green dye, and the third filter CF3 may include a blue pigment and/or a blue dye.

Embodiments of the present disclosure are not limited thereto, for example, the third filter CF3 may not include (e.g., may exclude) any pigment or any dye. The third filter CF3 may include a polymer photosensitive resin and not include a (e.g., any) pigment or dye. The third filter CF3 may be transparent. The third filter CF3 may be formed using a transparent photosensitive resin.

In one or more embodiments, the first filter CF1 and the second filter CF2 may each be a yellow filter. The first filter CF1 and the second filter CF2 may be provided in one body without distinction.

In one or more embodiments, the color filter layer CFL may further include a light blocking part. The light blocking part may be a black matrix. The light blocking part may be formed by including an organic light blocking material and/or an inorganic light blocking material, each including a black pigment and/or a black dye. The light blocking part may prevent or reduce light leakage phenomenon and divide the boundaries among adjacent filters CF1, CF2, and CF3.

On the color filter layer CFL, a base substrate BL may be arranged. The base substrate BL may be a member providing a base surface on which the color filter layer CFL, the light controlling layer CCL, and/or the like are arranged. The base substrate BL may be a glass substrate, a metal substrate, a plastic substrate, and/or the like. However, embodiments of the present disclosure are not limited thereto, and the base substrate BL may be an inorganic layer, an organic layer, or a composite material layer. In one or more embodiments, the base substrate BL may not be provided.

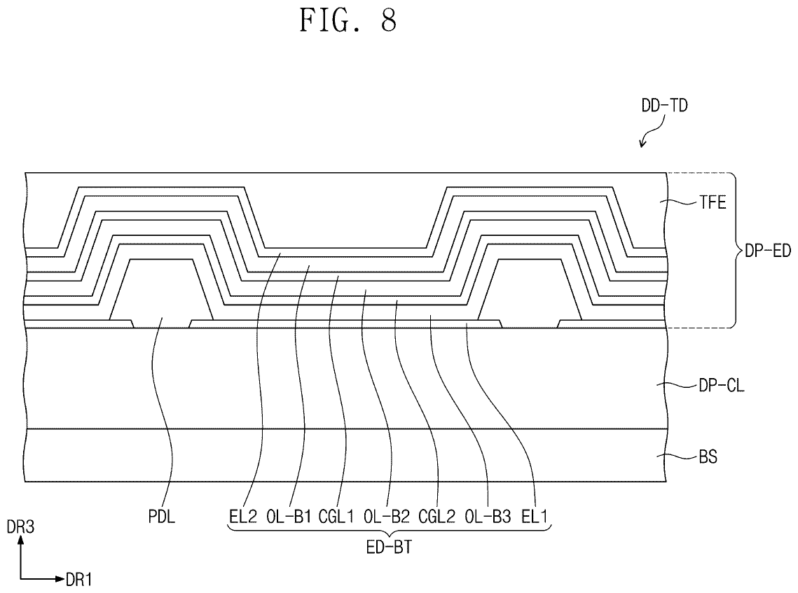

FIG. 8 is a cross-sectional view showing a portion of a display device according to one or more embodiments. In a display device DD-TD of one or more embodiments, the light emitting element ED-BT may include multiple light emitting structures OL-B1, OL-B2, and OL-B3. The light emitting element ED-BT may include oppositely arranged first electrode EL1 and second electrode EL2, and the multiple light emitting structures OL-B1, OL-B2, and OL-B3 stacked in order in a thickness direction and provided between the first electrode EL1 and the second electrode EL2. Each of the light emitting structures OL-B1, OL-B2, and OL-B3 may include an emission layer EML (FIG. 7), and a hole transport region HTR and an electron transport region ETR arranged with the emission layer EML (FIG. 7) therebetween.

For example, the light emitting element ED-BT included in the display device DD-TD of one or more embodiments may be a light emitting element of a tandem structure including multiple emission layers.

In one or more embodiments shown in FIG. 8, light emitted from the light emitting structures OL-B1, OL-B2, and OL-B3 may be all blue light. However, embodiments of the present disclosure are not limited thereto, for example, in one or more embodiments, the wavelength regions of light emitted from the light emitting structures OL-B1, OL-B2, and OL-B3 may be different from one another. For example, in one or more embodiments, the light emitting element ED-BT including the multiple light emitting structures OL-B1, OL-B2, and OL-B3 each emitting light in different wavelength regions may be to emit white light (e.g., combined white light).

Between neighboring light emitting structures OL-B1, OL-B2, and OL-B3, charge generating layers CGL1 and CGL2 may be respectively arranged. The charge generating layers CGL1 and CGL2 may independently include a p-type (kind) charge (e.g., P-charge) generating layer and/or an n-type (kind) charge (e.g., N-charge) generating layer.

In at least one selected from among the light emitting structures OL-B1, OL-B2, and OL-B3, included in the display device DD-TD of one or more embodiments, the nitrogen-containing compound of one or more embodiments may be included. For example, at least one selected from among multiple emission layers included in the light emitting element ED-BT may include the nitrogen-containing compound of one or more embodiments. In one or more embodiments, the electron transport region ETR included in the light emitting element ED-BT may include the nitrogen-containing compound of one or more embodiments.

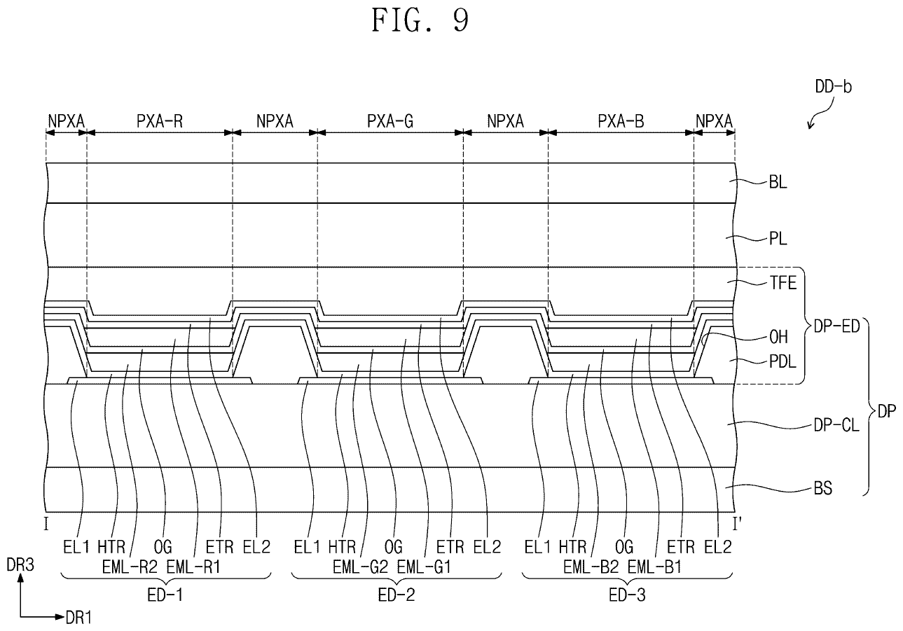

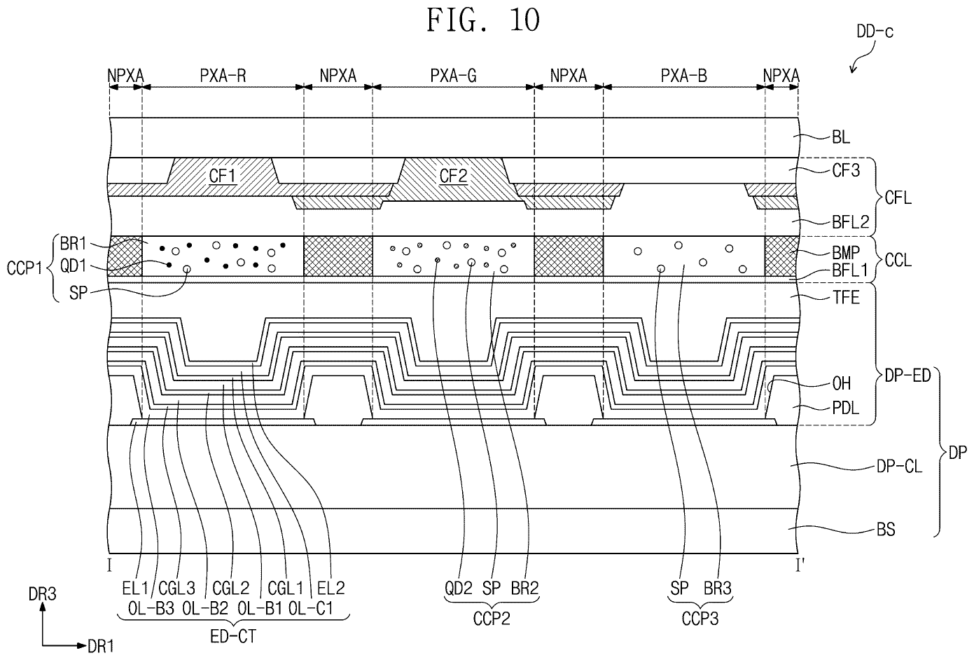

FIG. 9 is a cross-sectional view showing a display device according to one or more embodiments of the disclosure. FIG. 10 is a cross-sectional view showing a display device according to one or more embodiments of the disclosure.

Referring to FIG. 9, a display device DD-b according to one or more embodiments may include light emitting elements ED-1, ED-2, and ED-3, each formed by stacking two emission layers. Compared to the display device DD shown in FIG. 2, the display device DD-b shown in FIG. 9 is different in that first to third light emitting elements ED-1, ED-2, and ED-3 each include two emission layers stacked in a thickness direction. In each of the first to third light emitting elements ED-1, ED-2, and ED-3, two emission layers may be to emit light in substantially the same wavelength region.

In one or more embodiments, the first light emitting element ED-1 may include a first red emission layer EML-R1 and a second red emission layer EML-R2. The second light emitting element ED-2 may include a first green emission layer EML-G1 and a second green emission layer EML-G2. In addition, the third light emitting element ED-3 may include a first blue emission layer EML-B1 and a second blue emission layer EML-B2. Between the first red emission layer EML-R1 and the second red emission layer EML-R2, between the first green emission layer EML-G1 and the second green emission layer EML-G2, and between the first blue emission layer EML-B1 and the second blue emission layer EML-B2, an emission auxiliary part OG may be arranged.

The emission auxiliary part OG may include a single layer or a multilayer. The emission auxiliary part OG may include a charge generating layer. In one or more embodiments, the emission auxiliary part OG may include an electron transport region, a charge generating layer, and a hole transport region stacked in order (e.g., in the stated order). The emission auxiliary part OG may be provided as a common layer across all of the first to third light emitting elements ED-1, ED-2, and ED-3. However, embodiments of the present disclosure are not limited thereto, and the emission auxiliary part OG may be patterned and provided in an opening part OH defined in a pixel definition layer PDL.

The first red emission layer EML-R1, the first green emission layer EML-G1, and the first blue emission layer EML-B1 may each be arranged between the electron transport region ETR and the emission auxiliary part OG. The second red emission layer EML-R2, the second green emission layer EML-G2, and the second blue emission layer EML-B2 may each be arranged between the emission auxiliary part OG and the hole transport region HTR.

For example, in one or more embodiments, the first light emitting element ED-1 may include a first electrode EL1, a hole transport region HTR, a second red emission layer EML-R2, an emission auxiliary part OG, a first red emission layer EML-R1, an electron transport region ETR, and a second electrode EL2, stacked in order (e.g., in the stated order). The second light emitting element ED-2 may include a first electrode EL1, a hole transport region HTR, a second green emission layer EML-G2, an emission auxiliary part OG, a first green emission layer EML-G1, an electron transport region ETR, and a second electrode EL2, stacked in order (e.g., in the stated order). The third light emitting element ED-3 may include a first electrode EL1, a hole transport region HTR, a second blue emission layer EML-B2, an emission auxiliary part OG, a first blue emission layer EML-B1, an electron transport region ETR, and a second electrode EL2, stacked in order (e.g., in the stated order).

In one or more embodiments, an optical auxiliary layer PL may be arranged on the display device layer DP-ED. The optical auxiliary layer PL may include a polarization layer. The optical auxiliary layer PL may be arranged on the display panel DP and may control reflected light at the display panel DP by external light. In one or more embodiments, the optical auxiliary layer PL may not be provided in the display device.

In at least one emission layer included in the display device DD-b of one or more embodiments, shown in FIG. 9, the above-described nitrogen-containing compound of one or more embodiments may be included. For example, in one or more embodiments, at least one selected from among the first blue emission layer EML-B1 and the second blue emission layer EML-B2 may include the nitrogen-containing compound of one or more embodiments. In one or more embodiments, the electron transport region ETR included in the display device DD-b may include the nitrogen-containing compound of one or more embodiments.

Different from FIG. 8 and FIG. 9, a display device DD-c in FIG. 10 is shown to include four light emitting structures OL-B1, OL-B2, OL-B3, and OL-C1. A light emitting element ED-CT may include oppositely arranged first electrode EL1 and second electrode EL2, and first to fourth light emitting structures OL-B1, OL-B2, OL-B3, and OL-C1 stacked in a thickness direction between the first electrode EL1 and the second electrode EL2. The third light emitting structures OL-B3, the second light emitting structures OL-B2, the first light emitting structures OL-B1, and the fourth light emitting structures OL-C1 are stacked in order (e.g., in the stated order) in the thickness direction. Between the first to fourth light emitting structures OL-B1, OL-B2, OL-B3, and OL-C1, charge generating layers CGL1, CGL2, and CGL3 may each be separately arranged. For example, a first charge generating layer CGL1 is arranged between the first light emitting structures OL-B1 and the fourth light emitting structures OL-C1. A second charge generating layer CGL2 is arranged between the first light emitting structures OL-B1 and the second light emitting structures OL-B2. A third charge generating layer CGL3 is arranged between the second light emitting structures OL-B2 and the third light emitting structures OL-B3.

In one or more embodiments, among the four light emitting structures, the first to third light emitting structures OL-B1, OL-B2 and OL-B3 may be to emit blue light, and the fourth light emitting structure OL-C1 may be to emit green light. However, embodiments of the present disclosure are not limited thereto, and the first to fourth light emitting structures OL-B1, OL-B2, OL-B3, and OL-C1 may be to emit different wavelengths of light (respectively).

Between neighboring light emitting structures OL-C1, OL-B1, OL-B2, and OL-B3, charge generating layers CGL1, CGL2, and CGL3 may be arranged. The charge generating layers CGL1, CGL2, and CGL3 may each include a p-type (kind) charge (e.g., P-charge) generating layer and/or an n-type (kind) charge (e.g., N-charge) generating layer.

In at least one selected from among the light emitting structures OL-B1, OL-B2, OL-B3, and OL-C1 included in the display device DD-c of one or more embodiments, the nitrogen-containing compound of one or more embodiments may be included. For example, in one or more embodiments, at least one selected from among the first to third light emitting structures OL-B1, OL-B2, and OL-B3 may include the nitrogen-containing compound of one or more embodiments.

The light emitting element ED according to one or more embodiments of the disclosure may include the nitrogen-containing compound of one or more embodiments, represented by Formula 1 in at least one functional layer arranged between the first electrode EL1 and the second electrode EL2 to show excellent or suitable emission efficiency and improved lifetime characteristics. For example, the nitrogen-containing compound according to one or more embodiments may be included in the emission layer EML of the light emitting element ED of one or more embodiments, and the light emitting element of one or more embodiments may show high efficiency and long-life characteristics.

In one or more embodiments, an electronic apparatus may include a display device including multiple light emitting elements and a control part controlling the display device. The electronic apparatus of one or more embodiments may be an apparatus activated according to electrical signals. The electronic apparatus may include display devices of one or more suitable embodiments. For example, the electronic apparatus may include one or more selected from among televisions, monitors, large-size display devices such as outside billboards, personal computers, laptop computers, personal digital terminals, display devices for automobiles, game consoles, portable electronic devices, Internet of Things (IoT) devices, medium- and small-size display devices such as cameras, mobile phones, smartphones, tablet computers, mobile communication terminals, electronic notebooks, electronic books, portable multimedia players (PMPs), navigation devices, ultra-mobile personal computers (UMPCs), smartwatches, watch phones, and/or head-mounted display devices (HMDs) for implementing virtual reality and/or augmented reality.

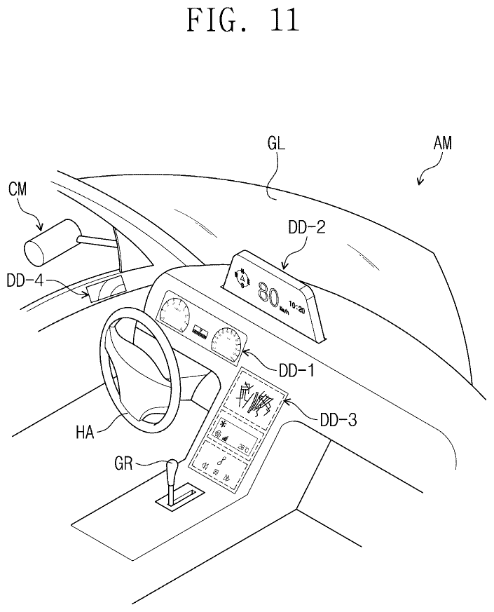

FIG. 11 is a diagram showing an automobile AM in which first to fourth display devices DD-1, DD-2, DD-3, and DD-4 are arranged. At least one selected from among the first to fourth display devices DD-1, DD-2, DD-3, and DD-4 may include the same configuration as one of the display devices DD, DD-TD, DD-a, DD-b, and DD-c, explained referring to FIGS. 1, 2, and 7 to 10.

In FIG. 11, a vehicle is shown as an automobile AM, but this is a mere example, for example, the first to fourth display devices DD-1, DD-2, DD-3, and DD-4 may be arranged on other transport apparatus such as one or more selected from among bicycles, motorcycles, trains, ships, and airplanes. In addition, at least one selected from among the first to fourth display devices DD-1, DD-2, DD-3 and DD-4 including the same configurations as one of the display devices DD, DD-TD, DD-a, DD-b, and DD-c may be introduced in personal computers, laptop computers, personal digital terminals, game consoles, portable electronic devices, televisions, monitors, external billboards, and/or the like. These are suggested as mere examples, and the display device may be introduced in other electronic devices as long as not deviated from the disclosure.

In one or more embodiments, at least one selected from among the first to fourth display devices DD-1, DD-2, DD-3, and DD-4 may include the light emitting element ED of one or more embodiments as described with reference to FIGS. 3 to 6. The light emitting element ED of one or more embodiments may include a nitrogen-containing compound of one or more embodiments. At least one selected from among the first to fourth display devices DD-1, DD-2, DD-3, and DD-4 may include the light emitting element ED including the nitrogen-containing compound of one or more embodiments, thereby improving a display service life.

Referring to FIG. 11, an automobile AM may include a steering wheel HA for the operation of the automobile AM and a gear GR. In addition, the automobile AM may include a front window GL arranged to face a driver.

A first display device DD-1 may be arranged in a first region overlapping with the steering wheel HA. For example, the first display device DD-1 may be a digital cluster displaying first information of the automobile AM. The first information may include a first graduation showing a driving speed of the automobile AM, a second graduation showing the number of revolution of an engine (i.e., revolutions per minute (RPM)), and an image showing a fuel state. The first graduation and the second graduation may each be represented by a digital image.

A second display device DD-2 may be arranged in a second region opposite to (e.g., facing) a driver seat and overlapping with the front window GL. The driver seat may be a seat where the steering wheel HA faces. For example, the second display device DD-2 may be a head up display (HUD) showing second information of the automobile AM. The second display device DD-2 may be optically clear. The second information may include digital numbers showing the driving speed of the automobile AM and may further include information including the current time. In one or more embodiments, the second information of the second display device DD-2 may be projected and displayed on the front window GL.

A third display device DD-3 may be arranged in a third region adjacent to the gear GR. For example, the third display device DD-3 may be a center information display (CID) for the automobile, arranged between the driver seat and a passenger seat and showing third information. The passenger seat may be a seat separated from the driver seat with the gear GR therebetween. The third information may include information on road conditions (for example, navigation information), on playing music or radio, on playing a dynamic image (or image), on the temperature in the automobile AM, and/or the like.

A fourth display device DD-4 may be arranged in a fourth region separated from the steering wheel HA and the gear GR and adjacent to a side of the automobile AM. For example, the fourth display device DD-4 may be a digital wing mirror displaying fourth information. The fourth display device DD-4 may display an external image of the automobile AM, taken by a camera module CM arranged at the outside of the automobile AM. The fourth information may include the external image of the automobile AM.

The above-described first to fourth information is for illustration, and the first to fourth display devices DD-1, DD-2, DD-3, and DD-4 may further display information on the inside and outside of the automobile. The first to fourth information may include different information from one another. However, embodiments of the present disclosure are not limited thereto, and a portion of the first to fourth information may include the same information as one another.

Hereinafter, nitrogen-containing compounds according to one or more embodiments and light emitting elements according to one or more embodiments of the disclosure will be explained in more detail by referring to the Examples and Comparative Examples. In addition, the Examples are only illustrative to assist the understanding of the disclosure, and the scope of the disclosure is not limited thereto.

### Examples

### 1. Synthesis of Nitrogen-Containing Compound

First, the synthetic method of the nitrogen-containing compound according to one or more embodiments will be explained in illustrating the synthetic methods of Compounds 1, 2, 13, 16, 21, 22, and 28. In addition, the synthetic methods of the nitrogen-containing compounds explained hereinafter are illustrative embodiments, and the synthetic method of the nitrogen-containing compound according to one or more embodiments of the disclosure is not limited to the Examples.

### (1) Synthesis of Compound 1

Nitrogen-containing Compound 1 according to one or more embodiments may be synthesized, for example, according to the following reaction scheme.

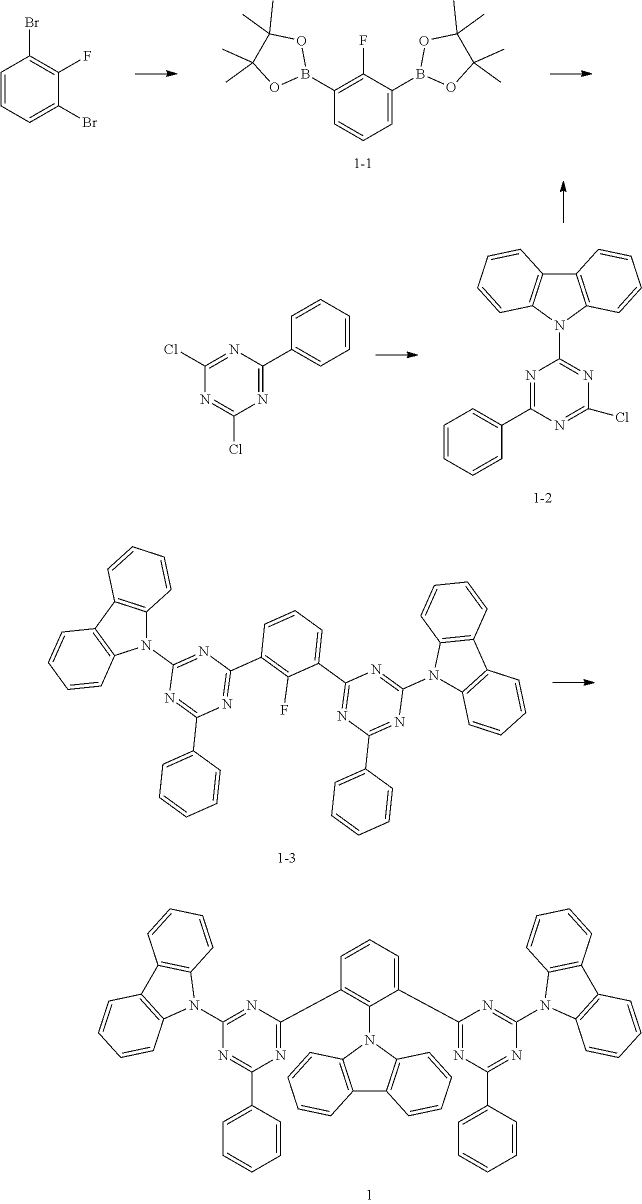

### Synthesis of Intermediate 1-1

1,3-Dibromo-2-fluorobenzene (20 g, 78.77 mmol), bis(pinacolato)diboron (80 g, 315 mmol), potassium acetate (KOAc) (46.38 g, 472.6 mmol), and 1,1′-bis(diphenylphosphino)ferrocene]palladium(II) dichloride (Pd(dppf)Cl₂) (4.42 g, 6.30 mmol) were dissolved in 1,4-dioxane (300 mL) and stirred at about 80° C. for about 12 hours. After finishing the reaction, a reaction solution was extracted, and an organic layer obtained was dried. After purifying the residual material, Intermediate 1-1 (16 g, yield: 58%) was obtained. Intermediate 1-1 was identified by a liquid chromatography mass analysis method (LC-MS) (C₁₈H₂₇B₂FO₄: M+1 348.21).

### Synthesis of Intermediate 1-2

2,4-Dichloro-6-phenyl-1,3,5-triazine (20 g, 88.47 mmol), 9H-carbazole (14.8 g, 88.47 mmol), Pd(PPh₃)₄ (2.04 g, 1.76 mmol), and 2 M K₂CO₃ aqueous solution (100 mL) were dissolved in toluene (300 mL) and stirred at about 90° C. for about 12 hours. After finishing the reaction, a reaction solution was extracted, and an organic layer obtained was dried. After purifying the residual material, Intermediate 1-2 (15 g, yield: 48%) was obtained. Intermediate 1-2 was identified by LC-MS (C₂₁H₁₃ClN₄: M+1 356.08).

### Synthesis of Intermediate 1-3

Intermediate 1-1 (7 g, 20.11 mmol), Intermediate 1-2 (14.35 g, 40.22 mmol), Pd(PPh₃)₄ (1.16 g, 1.00 mmol), and 2 M K₂CO₃ aqueous solution (50 mL) were dissolved in xylene (150 mL) and stirred at about 130° C. for about 12 hours. After finishing the reaction, a reaction solution was extracted, and an organic layer obtained was dried. After purifying the residual material, Intermediate 1-3 (5 g, yield 34%) was obtained. Intermediate 1-3 was identified by LC-MS (C₄₈H₂₉FN₈: M+1 736.25).

### Synthesis of Compound 1

Intermediate 1-3 (5 g, 6.78 mmol), 9H-carbazole (2.3 g, 13.52 mmol), and K₃PO₄ (5.75 g, 27.14 mmol) were dissolved in dimethylformamide (DMF) (150 mL) and stirred at about 160° C. for about 12 hours. After finishing the reaction, a reaction solution was extracted, and an organic layer obtained was dried. After purifying the residual material, Compound 1 (2 g, yield 33%) was obtained. Compound 1 was identified by LC-MS (C₆₀H₃₇N₉: M+1 883.32).

### (2) Synthesis of Compound 2

Nitrogen-containing Compound 2 according to one or more embodiments may be synthesized, for example, according to the following reaction scheme.

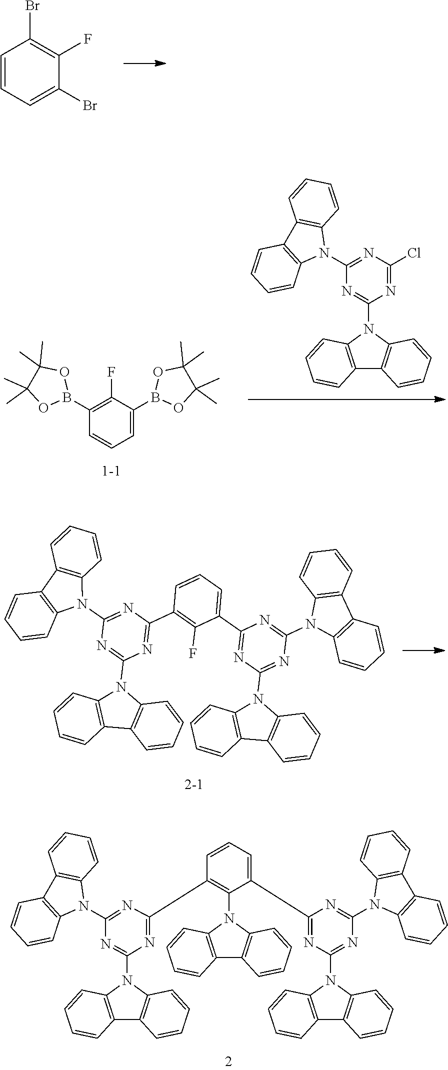

### Synthesis of Intermediate 2-1

Intermediate 2-1 was synthesized by substantially the same method as the synthesis process of Intermediate 1-3 except for using 9,9′-(6-chloro-1,3,5-triazin-2,4-diyl)bis(9H-carbazole) (CAS No.=877615-05-9) instead of Intermediate 1-2. The M+1 peak value of Intermediate 2-1 was confirmed by using LC-MS (C₆₀H₃₅FN₁₀: M+1 914.30).

### Synthesis of Compound 2

Compound 2 was synthesized by substantially the same method as the synthesis process of Compound 1 except for using Intermediate 2-1 instead of Intermediate 1-3. 1.5 g (yield: 32%) of Compound 2 was obtained. Compound 2 was identified by LC-MS (C₇₂H₄₃N₁₁: M+1 1061.37).

### (3) Synthesis of Compound 13

Nitrogen-containing Compound 13 according to one or more embodiments may be synthesized, for example, according to the following reaction scheme.

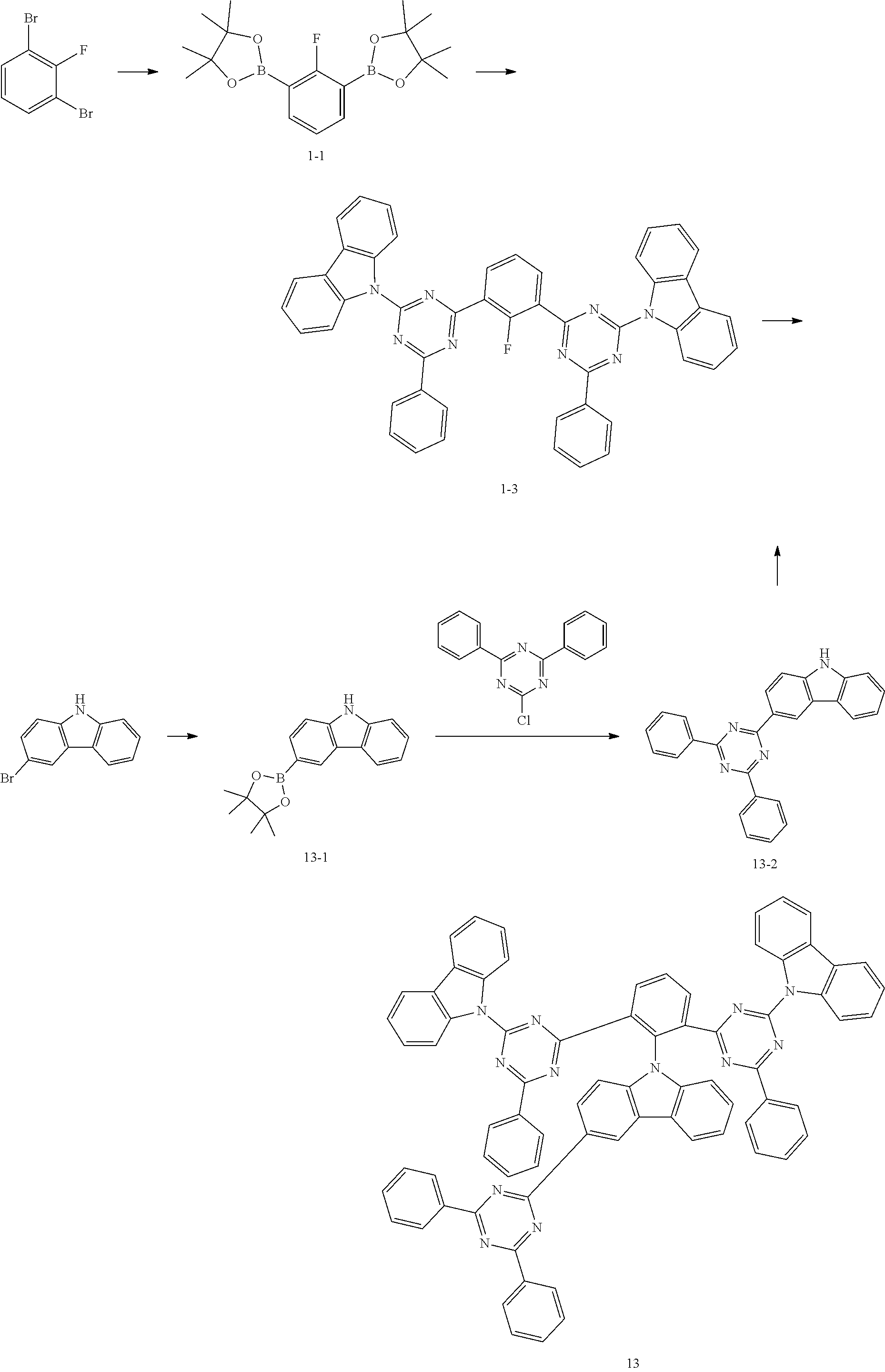

### Synthesis of Intermediate 13-1

3-Bromo-9H-carbazole (25 g, 101.58 mmol), bis(pinacolato)diboron (51.6 g, 203.16 mmol), KOAc (39.9 g, 406.3 mmol), and Pd(dppf)Cl₂ (7.13 g, 10.15 mmol) were dissolved in 1,4-dioxane (500 mL) and stirred at about 110° C. for about 12 hours. After finishing the reaction, a reaction solution was extracted, and an organic layer obtained was dried. After purifying the residual material, Intermediate 13-1 (17 g, yield 57%) was obtained. Intermediate 13-1 was identified by LC-MS (C₁₈H₂₀BNO₂: M+1 293.16).

### Synthesis of Intermediate 13-2

Intermediate 13-2 was synthesized by substantially the same method as the synthesis process of Intermediate 1-3 except for using Intermediate 13-1 instead of Intermediate 1-1 and using 2-chloro-4,6-diphenyl-1,3,5-triazine (CAS No.=3842-55-5) instead of Intermediate 1-2. The M+1 peak value of Intermediate 13-2 was confirmed by using LC-MS (C₂₇H₁₈N₄: M+1 398.15).

### Synthesis of Compound 13

Compound 13 was synthesized by substantially the same method as the synthesis process of Compound 1 except for using Intermediate 13-2 instead of 9H-carbazole. 3.0 g (yield: 40%) of Compound 13 was obtained. Compound 13 was identified by LC-MS (C₇₅H₄₆N₁₂: M+1 1114.40).

### (4) Synthesis of Compound 16

Nitrogen-containing Compound 16 according to one or more embodiments may be synthesized, for example, according to the following reaction scheme.

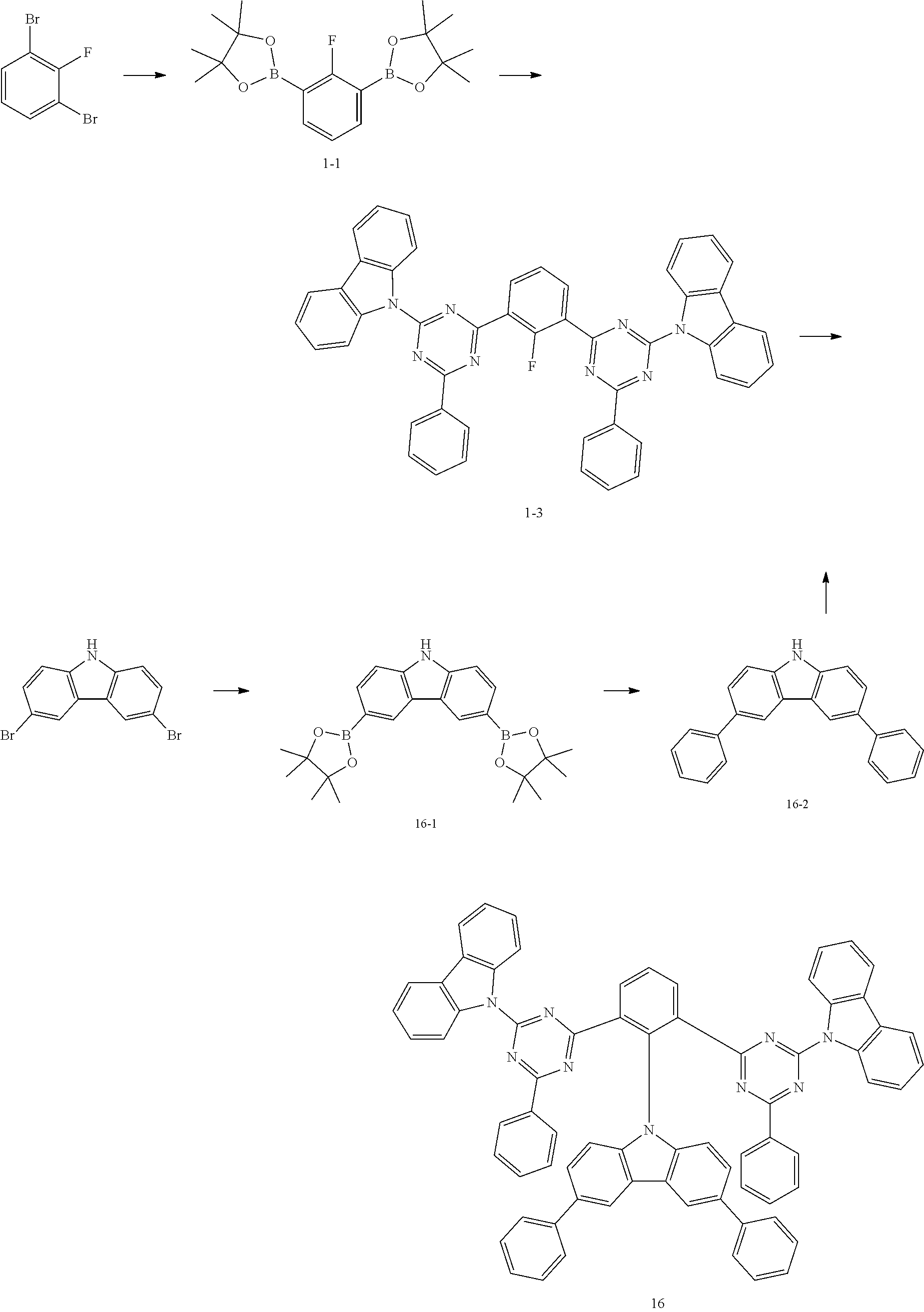

### Synthesis of Intermediate 16-1

Intermediate 16-1 was synthesized by substantially the same method as the synthesis process of Intermediate 13-1 except for using 3,6-dibromo-9H-carbazole instead of 3-bromo-9H-carbazole. The M+1 peak value of Intermediate 16-1 was confirmed by using LC-MS (C₂₄H₃₁B₂NO₄: M+1 419.24).

### Synthesis of Intermediate 16-2

Intermediate 16-2 was synthesized by substantially the same method as the synthesis process of Intermediate 13-2 except for using Intermediate 16-1 instead of Intermediate 13-1 and using bromobenzene instead of 2-chloro-4,6-diphenyl-1,3,5-triazine. The M+1 peak value of Intermediate 16-2 was confirmed by using LC-MS (C₂₄H₁₇N: M+1 319.14).

### Synthesis of Compound 16

Compound 16 was synthesized by substantially the same method as the synthesis process of Compound 1 except for using Intermediate 16-2 instead of 9H-carbazole. 4.1 g (yield: 53%) of Compound 16 was obtained. Compound 16 was identified by LC-MS (C₇₂H₄₅N₉: M+1 1035.38).

### (5) Synthesis of Compound 21

Nitrogen-containing Compound 21 according to one or more embodiments may be synthesized, for example, according to the following reaction scheme.

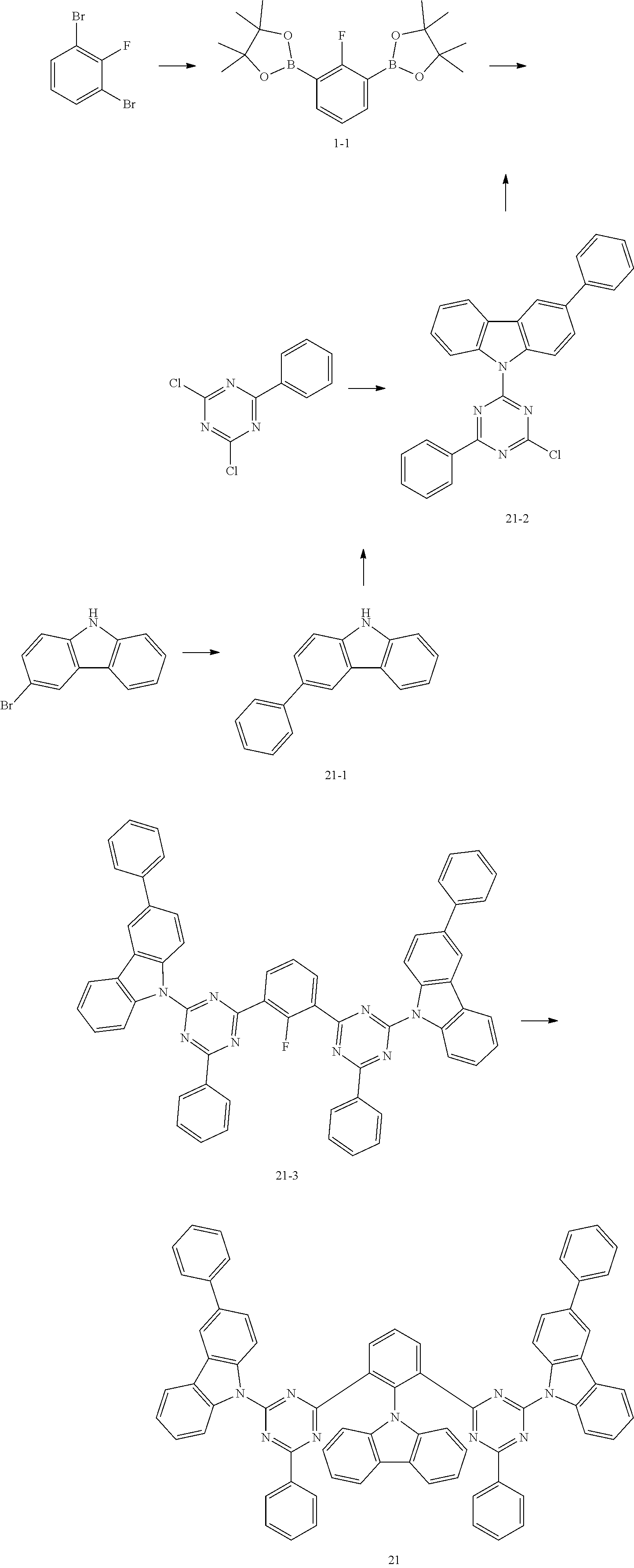

### Synthesis of Intermediate 21-1

Intermediate 21-1 was synthesized by substantially the same method as the synthesis process of Intermediate 1-3 except for using 3-bromo-9H-carbazole instead of Intermediate 1-1 and using phenylboronic acid instead of Intermediate 1-2. The M+1 peak value of Intermediate 21-1 was confirmed by using LC-MS (C₁₈H₁₃N: M+1 243.10).

### Synthesis of Intermediate 21-2

Intermediate 21-2 was synthesized by substantially the same method as the synthesis process of Intermediate 1-2 except for using Intermediate 21-1 instead of 9H-carbazole. The M+1 peak value of Intermediate 21-2 was confirmed by using LC-MS (C₂₇H₁₇ClN₄: M+1 432.11).

### Synthesis of Intermediate 21-3

Intermediate 21-3 was synthesized by substantially the same method as the synthesis process of Intermediate 1-3 except for using Intermediate 21-2 instead of Intermediate 1-2. The M+1 peak value of Intermediate 21-3 was confirmed by using LC-MS (C₆₀H₃₇FN₈: M+1 888.31).

### Synthesis of Compound 21

Compound 21 was synthesized by substantially the same method as the synthesis process of Compound 1 except for using Intermediate 21-3 instead of Intermediate 1-3. 2.7 g (yield: 46%) of Compound 21 was obtained. Compound 21 was identified by LC-MS (C₇₂H₄₅N₉: M+1 1035.38).

### (6) Synthesis of Compound 22

Nitrogen-containing Compound 22 according to one or more embodiments may be synthesized, for example, according to the following reaction scheme.

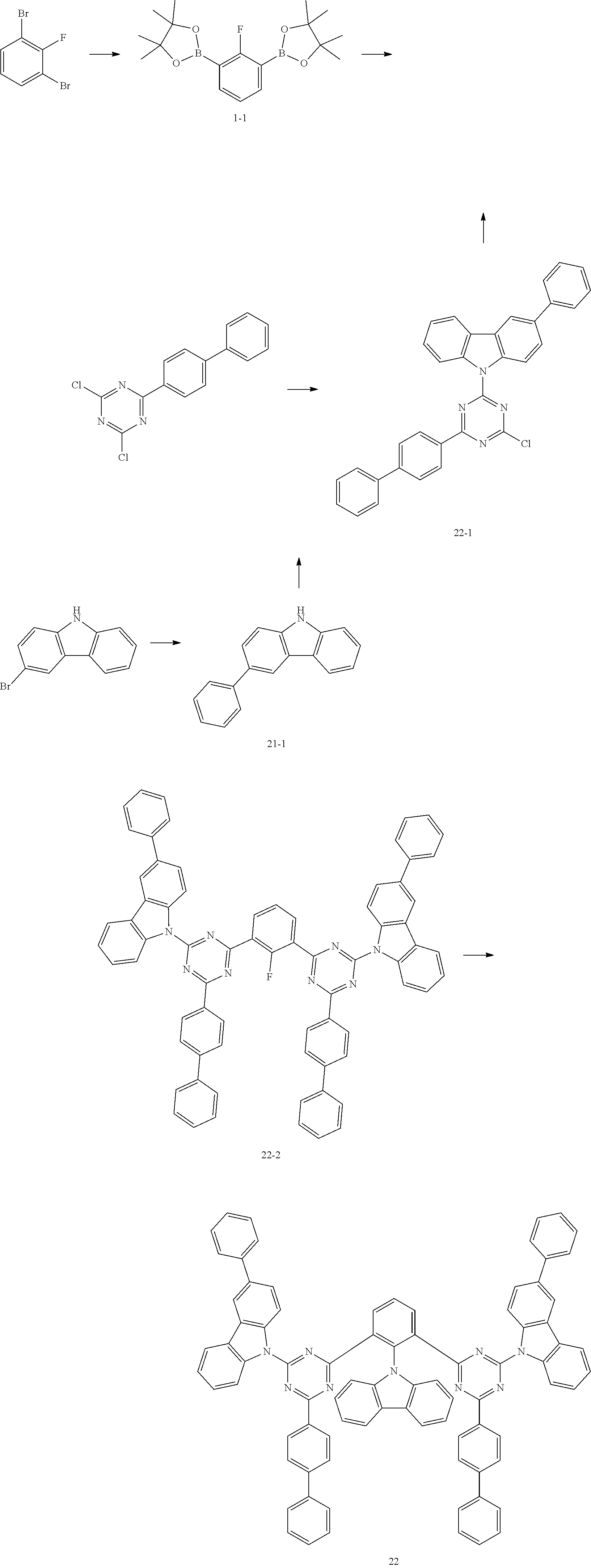

### Synthesis of Intermediate 22-1

Intermediate 22-1 was synthesized by substantially the same method as the synthesis process of Intermediate 21-2 except for using 2-([1,1′-biphenyl]-4-yl)-4,6-dichloro-1,3,5-triazine (CAS No.=10202-45-6) instead of 2,4-dichloro-6-phenyl-1,3,5-triazine. The M+1 peak value of Intermediate 22-1 was confirmed by using LC-MS (C₃₃H₂₁ClN₄: M+1 508.15).

### Synthesis of Intermediate 22-2

Intermediate 22-2 was synthesized by substantially the same method as the synthesis process of Intermediate 1-3 except for using Intermediate 22-1 instead of Intermediate 1-2. The M+1 peak value of Intermediate 22-2 was confirmed by using LC-MS (C₇₂H₄₅FN₈: M+1 1040.38).

### Synthesis of Compound 22

Compound 22 was synthesized by substantially the same method as the synthesis process of Compound 1 except for using Intermediate 22-2 instead of Intermediate 1-3. 1.5 g (yield: 26%) of Compound 22 was obtained. Compound 22 was identified by LC-MS (C₈₄H₅₃N₉: M+1 1187.44).

### (7) Synthesis of Compound 28

Nitrogen-containing Compound 28 according to one or more embodiments may be synthesized, for example, according to the following reaction scheme.

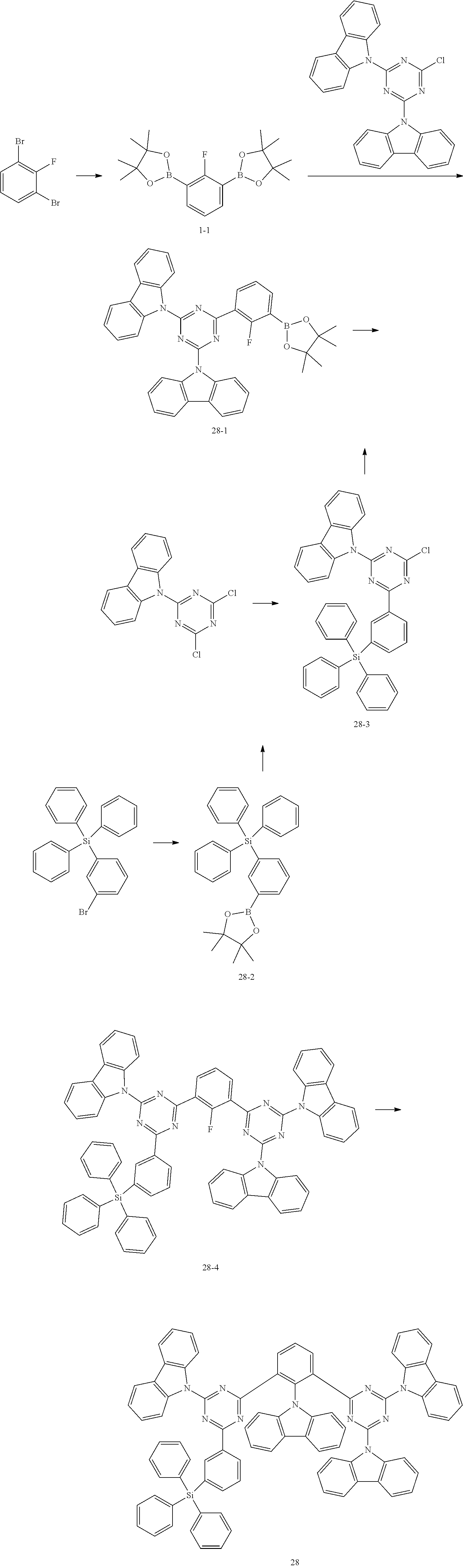

### Synthesis of Intermediate 28-1

Intermediate 28-1 was synthesized by substantially the same method as the synthesis process of Intermediate 1-3 except for using 9,9′-(6-chloro-1,3,5-triazin-2,4-diyl)bis(9H-carbazole) (CAS No.=877615-05-9) instead of Intermediate 1-2. The M+1 peak value of Intermediate 28-1 was confirmed by using LC-MS (C₃₉H₃₁BFN₅O₂: M+1 631.26).

### Synthesis of Intermediate 28-2

Intermediate 28-2 was synthesized by substantially the same method as the synthesis process of Intermediate 1-1 except for using (3-bromophenyl)triphenylsilane (CAS No.=185626-73-7) instead of 1,3-dibromo-2-fluorobenzene. The M+1 peak value of Intermediate 28-2 was confirmed by using LC-MS (C₃₀H₃₁BO₂Si: M+1 462.22).

### Synthesis of Intermediate 28-3

Intermediate 28-3 was synthesized by substantially the same method as the synthesis process of Intermediate 1-3 except for using Intermediate 28-2 instead of Intermediate 1-1 and using 9-(4,6-dichloro-1,3,5-triazin-2-yl)-9H-carbazole (CAS No.=24209-95-8) instead of Intermediate 1-2. The M+1 peak value of Intermediate 28-3 was confirmed by using LC-MS (C₃₉H₂₇ClN₄Si: M+1 614.17).

### Synthesis of Intermediate 28-4

Intermediate 28-4 was synthesized by substantially the same method as the synthesis process of Intermediate 1-3 except for using Intermediate 28-1 instead of Intermediate 1-1 and using Intermediate 28-3 instead of Intermediate 1-2. The M+1 peak value of Intermediate 28-4 was confirmed by using LC-MS (C₇₂H₄₆FN₉Si: M+1 1083.36).

### (Synthesis of Compound 28)

Compound 28 was synthesized by substantially the same method as the synthesis process of Compound 1 except for using Intermediate 28-4 instead of Intermediate 1-3. 3.2 g (yield: 56%) of Compound 28 was obtained. Compound 28 was identified by LC-MS (C₈₄H₅₄N₁₀Si: M+1 1230.43).

### 2. Manufacture and Evaluation of Light Emitting Element

A light emitting element of one or more embodiments, including the nitrogen-containing compound of one or more embodiments in an emission layer was manufactured by a method described herein. Light emitting elements of Examples 1 to 8 were each manufactured by respectively using the nitrogen-containing compounds of Example Compounds 1, 2, 7, 13, 16, 21, 22, and 28 as a host material of an emission layer. Comparative Examples 1 to 3 correspond to light emitting elements manufactured by respectively using Comparative Compounds C₁ to C₃ as a host material of an emission layer.

### Example Compounds

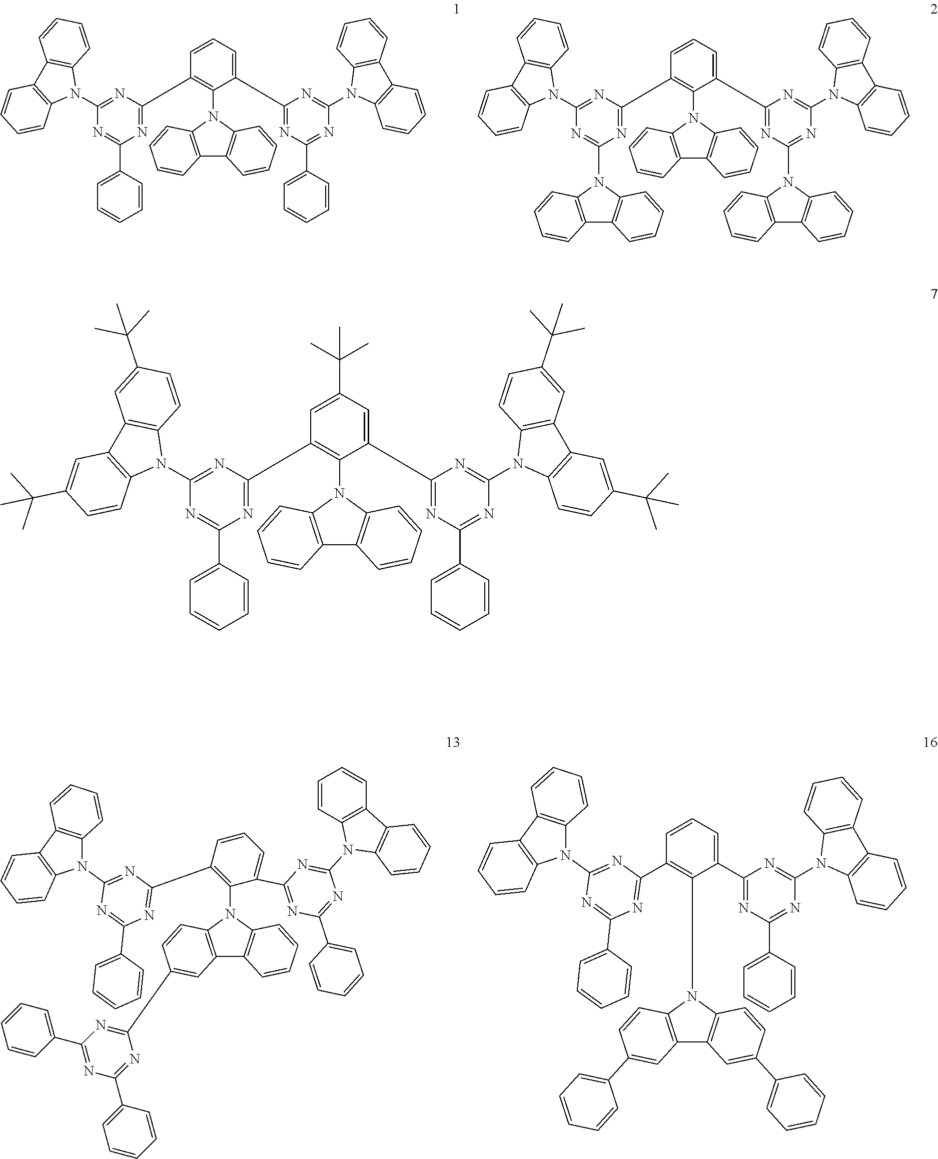

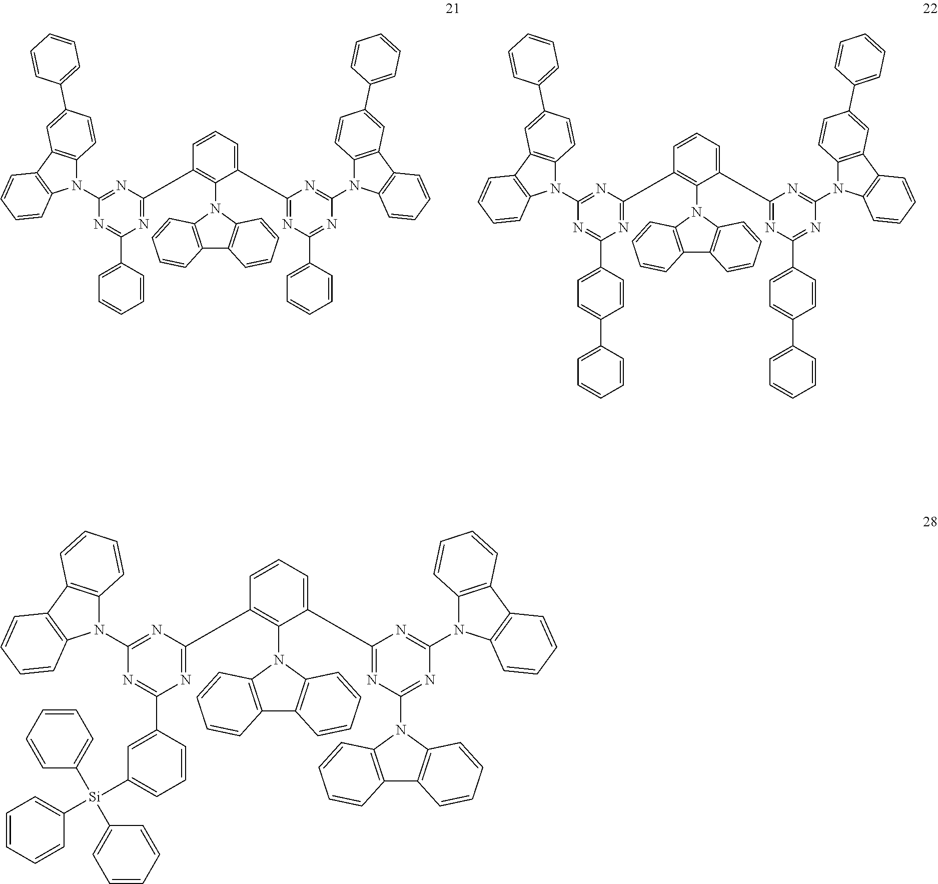 [SMILES: c1ccc(-c2ccc3c(c2)c2ccccc2n3-c2nc(-c3ccccc3)nc(-c3cccc(-c4nc(-c5ccccc5)nc(-n5c6ccccc6c6cc(-c7ccccc7)ccc65)n4)c3-n3c4ccccc4c4ccccc43)n2)cc1 ; c1ccc(-c2ccc(-c3nc(-c4cccc(-c5nc(-c6ccc(-c7ccccc7)cc6)nc(-n6c7ccccc7c7cc(-c8ccccc8)ccc76)n5)c4-n4c5ccccc5c5ccccc54)nc(-n4c5ccccc5c5cc(-c6ccccc6)ccc54)n3)cc2)cc1 ; c1ccc([Si](c2ccccc2)(c2ccccc2)c2cccc(-c3nc(-c4cccc(-c5nc(-n6c7ccccc7c7ccccc76)nc(-n6c7ccccc7c7ccccc76)n5)c4-n4c5ccccc5c5ccccc54)nc(-n4c5ccccc5c5ccccc54)n3)c2)cc1]

### Comparative Compounds

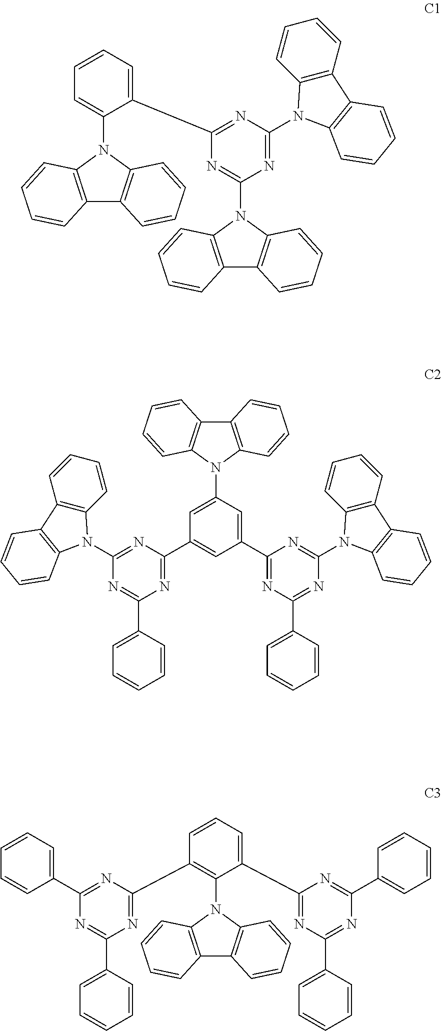 [SMILES: c1ccc(-n2c3ccccc3c3ccccc32)c(-c2nc(-n3c4ccccc4c4ccccc43)nc(-n3c4ccccc4c4ccccc43)n2)c1 ; c1ccc(-c2nc(-c3cc(-c4nc(-c5ccccc5)nc(-n5c6ccccc6c6ccccc65)n4)cc(-n4c5ccccc5c5ccccc54)c3)nc(-n3c4ccccc4c4ccccc43)n2)cc1 ; c1ccc(-c2nc(-c3ccccc3)nc(-c3cccc(-c4nc(-c5ccccc5)nc(-c5ccccc5)n4)c3-n3c4ccccc4c4ccccc43)n2)cc1]

### Manufacture of Light Emitting Elements

A light emitting element of one or more embodiments including a nitrogen-containing compound of one or more embodiments in an emission layer was manufactured by a method described herein. Light emitting elements of Examples 1 to 8 were each manufactured by respectively using Compounds 1, 2, 7, 13, 16, 21, 22, and 28 as the host material of an emission layer. Light emitting elements of Comparative Examples 1 to 3 were each manufactured by respectively using Comparative Compounds C1, C2, and C3 as the host material of an emission layer. The dopant material of the emission layer used a phosphorescence dopant material.

For the manufacture of each of the light emitting elements of the Examples and Comparative Examples, a glass substrate on which an ITO electrode was formed (a product of Corning Co.) was cut to a size of about 50 mm×50 mm×0.5 mm, cleansed with ultrasonic waves in isopropyl alcohol and then pure water for about 5 minutes each, exposed to ultraviolet for about 30 minutes, cleansed by exposing to ozone to form an anode, and the anode was installed in a vacuum evaporation apparatus.

On the anode, HATCN was deposited to form a hole injection layer with a thickness of about 100 Å, H-1-1 was vacuum deposited on the hole injection layer to form a hole transport layer with a thickness of about 600 Å, and HT33 was vacuum deposited to form an electron blocking layer with a thickness of about 50 Å.

On the electron blocking layer, the Example Compound or Comparative Compound, ETH66, and a PtON-TBBI phosphorescence dopant (AD-42) were co-deposited in a weight ratio of about 60:27:13 to form an emission layer with a thickness of about 350 Å.

After that, on the emission layer, ETH2 was deposited to form a hole blocking layer with a thickness of about 50 Å, and ETH2 and LiQ were co-deposited in a weight ratio of about 1:1 to form an electron transport layer with a thickness of about 350 Å. On the electron transport layer, an alkali metal halide of LiF was deposited to form an electron injection layer with a thickness of about 15 Å. After that, aluminum (Al) was deposited to a thickness of about 80 Å to form a LiF/A₁ electrode and to manufacture a light emitting element. All layers were formed by a vacuum deposition method.

The compounds used for the manufacture of each of the light emitting elements of the Examples and Comparative Examples are shown as follows.

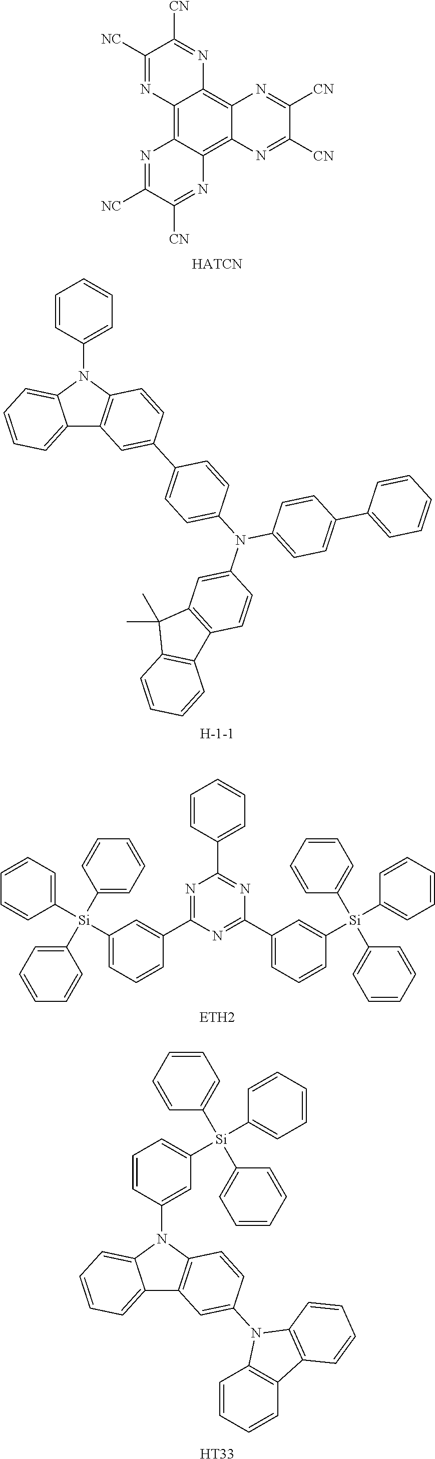

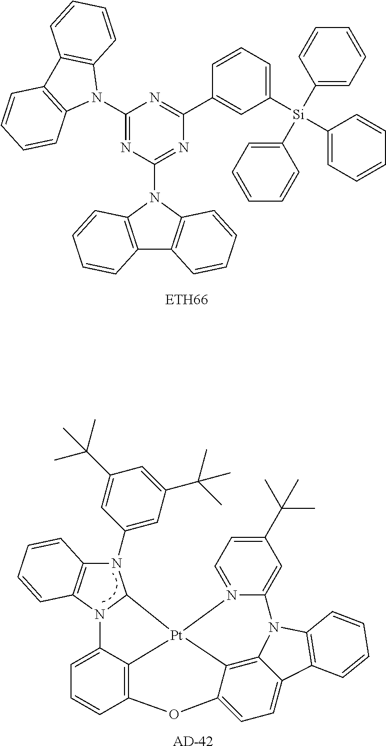

### Evaluation of Properties of Light Emitting Elements

In order to evaluate the properties of each of the light emitting elements manufactured by using Example Compounds 1, 2, 7, 13, 16, 21, 22, and 28 and Comparative Compounds C1, C2 and C3, driving voltage, current density, and maximum quantum efficiency at a current density of about 10 mA/cm² were each measured. The driving voltage and current density of the light emitting elements were measured using a source meter (Keithley Instrument Co., 2400 series), and the maximum quantum efficiency was measured using an external quantum efficiency measurement apparatus C9920-2-12 of Hamamatsu Photonics Co. In evaluating the maximum quantum efficiency, the luminance/current density was measured using a luminance meter calibrated for wavelength sensitivity, and the maximum quantum efficiency was converted assuming angular luminance distribution (Lambertian) assuming a fully diffuse reflective surface. The evaluation results of the properties of each of the organic light emitting elements are shown in Table 1. Lifetime (T95) was evaluated by comparing the time from an initial luminance value to about 95% luminance deterioration when continuously driven at a current density of about 10 mA/cm², and expressed as a relative element lifetime with respect to a value of Example 4 (i.e., a lifetime ratio (%) (T95)).

TABLE 1

| | | | Maximum | | |
|---|---|---|---|---|---|
| | | Driving | quantum | Lifetime | |
| | Emission | voltage | efficiency | ratio (%) | Emission |
| Division | layer host | (V) | (%) | (T95) | color |
| Example 1 | Compound 1 | 5.2 | 22.1 | 131 | Blue |
| Example 2 | Compound 2 | 5.1 | 23.3 | 121 | Blue |
| Example 3 | Compound 7 | 5.4 | 24.4 | 105 | Blue |
| Example 4 | Compound | 5.3 | 23.3 | 100 | Blue |
| | 13 | | | | |
| Example 5 | Compound | 5.2 | 22.3 | 112 | Blue |
| | 16 | | | | |
| Example 6 | Compound | 5.1 | 22.7 | 142 | Blue |
| | 21 | | | | |
| Example 7 | Compound | 5.1 | 23.6 | 132 | Blue |
| | 22 | | | | |
| Example 8 | Compound | 5.3 | 23.4 | 127 | Blue |
| | 28 | | | | |
| Comparativ | Comparative | 5.9 | 20.9 | 82 | Blue |
| e Example 1 | Compound | | | | |
| | C1 | | | | |
| Comparativ | Comparative | 6.1 | 17.4 | 76 | Blue |
| e Example 2 | Compound | | | | |
| | C2 | | | | |
| Comparativ | Comparative | 5.7 | 19.1 | 88 | Blue |
| e Example 3 | Compound | | | | |
| | C3 | | | | |

Referring to the results of Table 1, if (e.g., when) the nitrogen-containing compound according to one or more embodiments of the present disclosure is used as the host material of the emission layer of a light emitting element, high efficiency and long lifetime may be achieved. For example, it can be confirmed that Examples 1 to 8 each exhibit higher efficiency and longer lifetime characteristics compared to Comparative Examples 1 to 3. The nitrogen-containing compound of the present disclosure has a structure in which a carbazole moiety and first and second triazine moieties each substituted with at least one carbazole group are connected with one phenyl linker, and the first triazine moiety and the second triazine moiety are substituted at the ortho positions with respect to the carbazole moiety, and may have excellent or suitable electron transport properties and the high lowest excited triplet energy level (T1 level). Accordingly, the light emitting element that includes the nitrogen-containing compound of one or more embodiments in the emission layer may show high efficiency and long-life characteristics concurrently (e.g., simultaneously).

Comparative Compound C1 included in Comparative Example 1 corresponds to a compound including only one triazine moiety in a phenyl linker that is connected with a carbazole moiety, compared to other Example Compounds. Accordingly, it can be confirmed that Comparative Compound C1 has degraded electron transport properties compared to the Example Compounds, and if (e.g., when) applied to a light emitting element, emission efficiency and lifetime characteristics were deteriorated compared to the Examples.

Comparative Example 2 showed degraded emission efficiency and lifetime characteristics compared to the Examples. If Comparing Example 1 with Comparative Example 2, Comparative Compound C2 included in Comparative Example 2 has a structure in which triazine moieties each substituted with a carbazole group are substituted at the meta positions with respect to the carbazole moiety bonded to the linker phenyl group compared to Example Compound 1. Accordingly, it can be confirmed that the electron transport properties of Comparative Compound C2 were degraded compared to Example Compound 1, and if (e.g., when) applied to a light emitting element, emission efficiency and lifetime characteristics were degraded compared to Example 1.

Comparative Example 3 showed degraded results of emission efficiency and lifetime characteristics compared to the Examples. If Comparing Example 1 with Comparative Example 3, Comparative Compound C3 included in Comparative Example 3 has a structure in which two triazine moieties are not substituted with a carbazole group compared to Example Compound 1. Accordingly, it can be confirmed that Comparative Compound C3 has degraded electron transport properties compared to Example Compound 1, and if (e.g., when) applied to a light emitting element, emission efficiency and lifetime characteristics were degraded compared to Example 1. In contrast, because the Example Compounds each have a structure in which carbazole groups are substituted at two triazine moieties, respectively, holes and electrons in the triazine may be balanced, and excellent or suitable emission efficiency and improved element properties may be exhibited. For example, the nitrogen-containing compound of one or more embodiments introduces two or more carbazole groups in a molecule and may show the controlling effect of charge mobility. Accordingly, if (e.g., when) the nitrogen-containing compound of one or more embodiments is applied to a light emitting element, high efficiency and long-life characteristics may be shown concurrently (e.g., simultaneously).

The light emitting element of one or more embodiments includes the nitrogen-containing compound of one or more embodiments as a host of the emission layer in the light emitting element, and high element efficiency and improved lifetime characteristic may be accomplished.

The light emitting element of one or more embodiments may show improved element properties of high efficiency and long lifetime.

The nitrogen-containing compound of one or more embodiments may be included in the emission layer of a light emitting element and may contribute to the increase of the efficiency and lifetime of the light emitting element.

The display device of one or more embodiments may show excellent or suitable display quality by including the light emitting element of one or more embodiments of the disclosure.

As utilized herein, the terms “substantially,” “about,” or similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, or 5% of the stated value.

In the context of the present application and unless otherwise defined, the terms “use,” “using,” and “used” may be considered synonymous with the terms “utilize,” “utilizing,” and “utilized,” respectively.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The light-emitting element, the display device, the electronic device/apparatus, a device of manufacturing the same, and/or any other relevant apparatuses/devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

In the present disclosure, each suitable feature of the various embodiments of the disclosure may be combined or combined with each other, partially or entirely, and may be technically interlocked and operated in various suitable ways, and each embodiment may be implemented independently of each other or in conjunction with each other in any suitable manner unless otherwise stated or implied.

Although one or more embodiments of disclosure have been described, it is understood that disclosure should not be limited to these embodiments, but one or more suitable changes and modifications can be made by one ordinary skilled in the art within the spirit and scope of disclosure as hereinafter claimed and equivalents thereof.

What is claimed is:

1. A light emitting element, comprising:
a first electrode;
a second electrode opposite to the first electrode; and
at least one functional layer between the first electrode and the second electrode and comprising a nitrogen-containing compound represented by Formula 1: 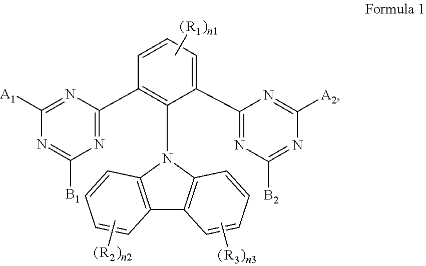
wherein, in Formula 1,
R₁ to R₃ are each independently hydrogen, deuterium, a halogen, a cyano group, a substituted or unsubstituted silyl group, a substituted or unsubstituted germanium group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
A₁, A₂, B₁, and B₂ are each independently a substituted or unsubstituted aryl group of 6 to 30 ring-forming carbon atoms or a substituted or unsubstituted heteroaryl group of 2 to 30 ring-forming carbon atoms,
at least one of A₁ or B₁, and at least one of A₂ or B₂ are each independently a substituted or unsubstituted carbazole group,
n1 is an integer of 0 to 3, and
n2 and n3 are each independently an integer of 0 to 4.

2. The light emitting element of claim 1, wherein the nitrogen-containing compound represented by Formula 1 is represented by Formula 2: 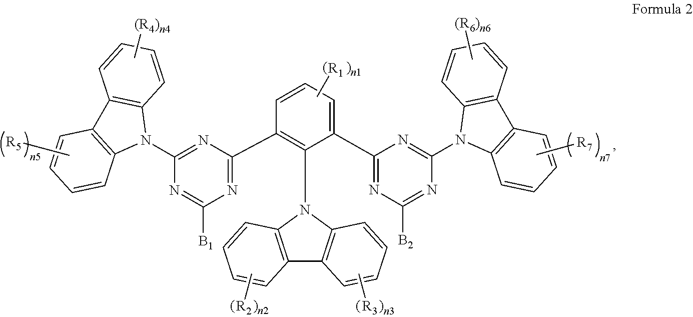
in Formula 2,
R₄ to R₇ being each independently hydrogen, deuterium, a halogen, a cyano group, a substituted or unsubstituted silyl group, a substituted or unsubstituted germanium group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
n4 to n7 being each independently an integer of 0 to 4, and
R₁ to R₃, n1 to n3, B₁, and B₂ being each the same as defined in Formula 1.

3. The light emitting element of claim 1, wherein the nitrogen-containing compound represented by Formula 1 is represented by Formula 3-1 or Formula 3-2: 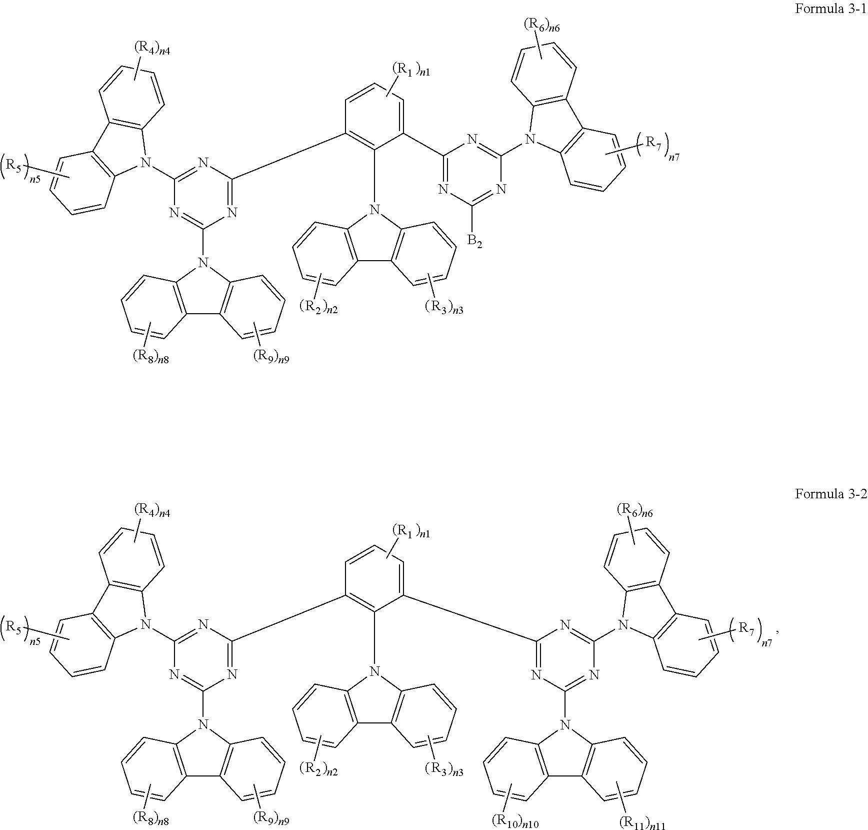
in Formula 3-1 and Formula 3-2,
R₄ to R₁₁ being each independently hydrogen, deuterium, a halogen, a cyano group, a substituted or unsubstituted silyl group, a substituted or unsubstituted germanium group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
n4 to n11 being each independently an integer of 0 to 4, and
R₁ to R₃, n1 to n3, and B₂ being each the same as defined in Formula 1.

4. The light emitting element of claim 1, wherein the nitrogen-containing compound represented by Formula 1 is represented by Formula 4-1 or Formula 4-2: 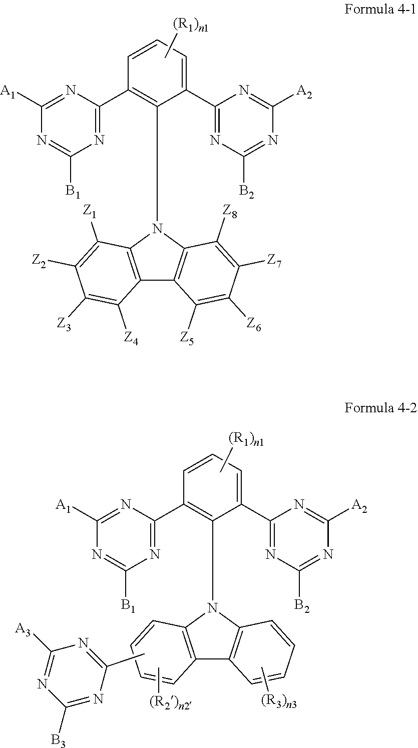
in Formula 4-1 and Formula 4-2,
Z₁ to Z₈ being each independently hydrogen or a substituted or unsubstituted aryl group of 6 to 30 ring-forming carbon atoms,
R_{2′} being hydrogen, deuterium, a halogen, a cyano group, a substituted or unsubstituted silyl group, a substituted or unsubstituted germanium group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
n2′ being an integer of 0 to 3,
A₃ and B₃ being each independently a substituted or unsubstituted aryl group of 6 to 30 ring-forming carbon atoms or a substituted or unsubstituted heteroaryl group of 2 to 30 ring-forming carbon atoms, and
R₁, R₃, n1, n3, A₁, A₂, B₁, and B₂ being each the same as defined in Formula 1.

5. The light emitting element of claim 1,
wherein at least one of A₁ or B₁, and at least one of A₂ or B₂ are each independently a substituted or unsubstituted carbazole group, and
the remainder selected from among A₁, B₁, A₂, and B₂ are each independently represented by one selected from among Formula A-1 to Formula A-4: 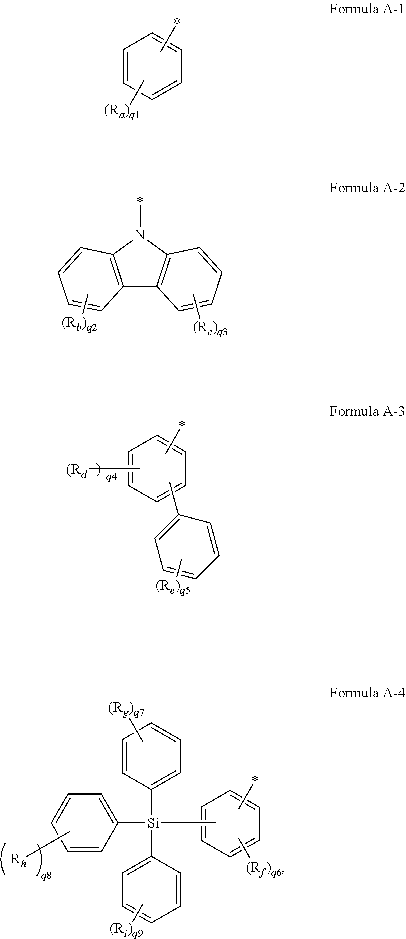
in Formula A-1 to Formula A-4,
Rₐ to Rᵢ being each independently hydrogen, deuterium, a halogen, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
q1, q5, and q7 to q9 being each independently an integer of 0 to 5, and
q2 to q4, and q6 being each independently an integer of 0 to 4.

6. The light emitting element of claim 1, wherein the nitrogen-containing compound is any one selected from among compounds in Compound Group 1:

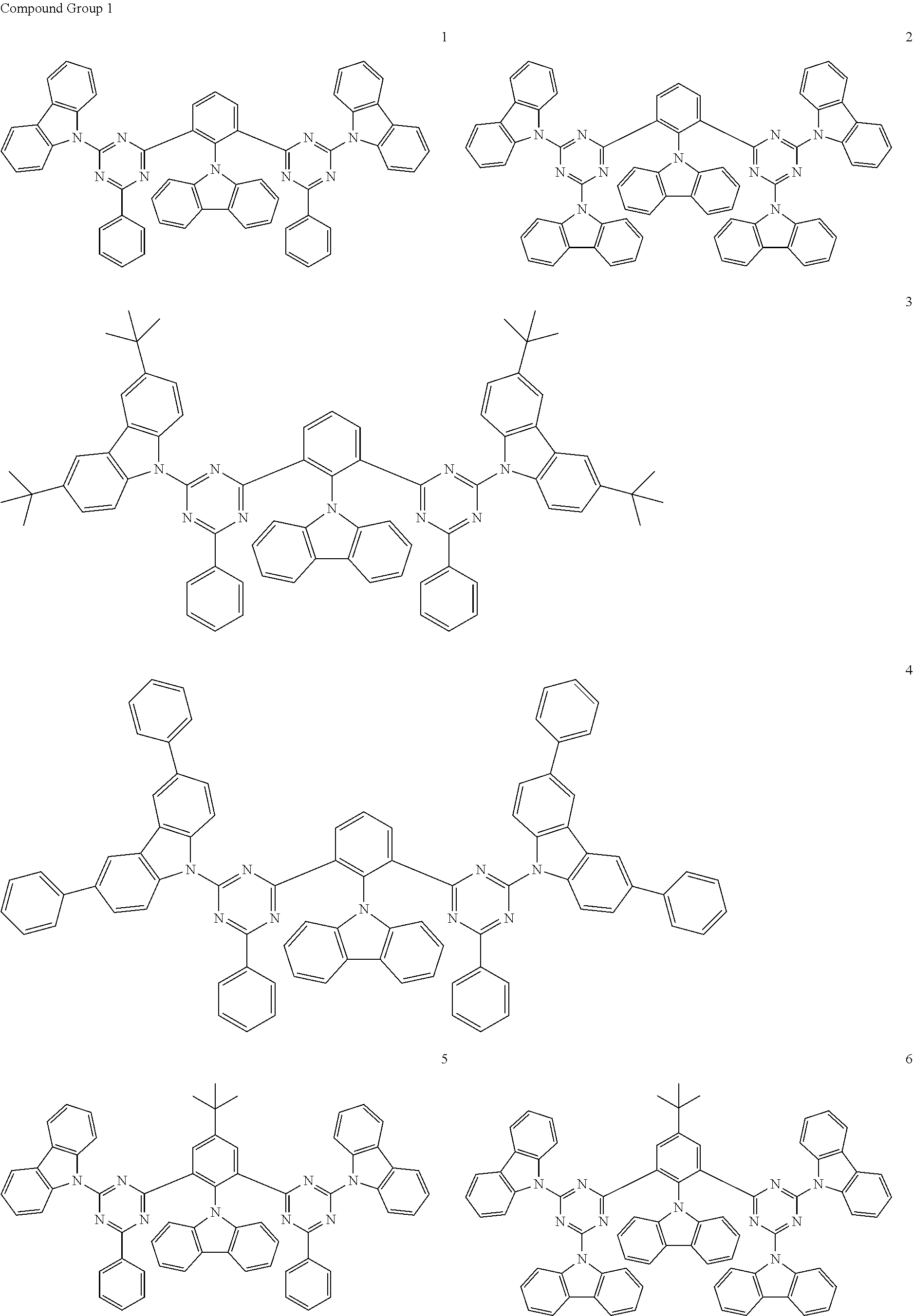

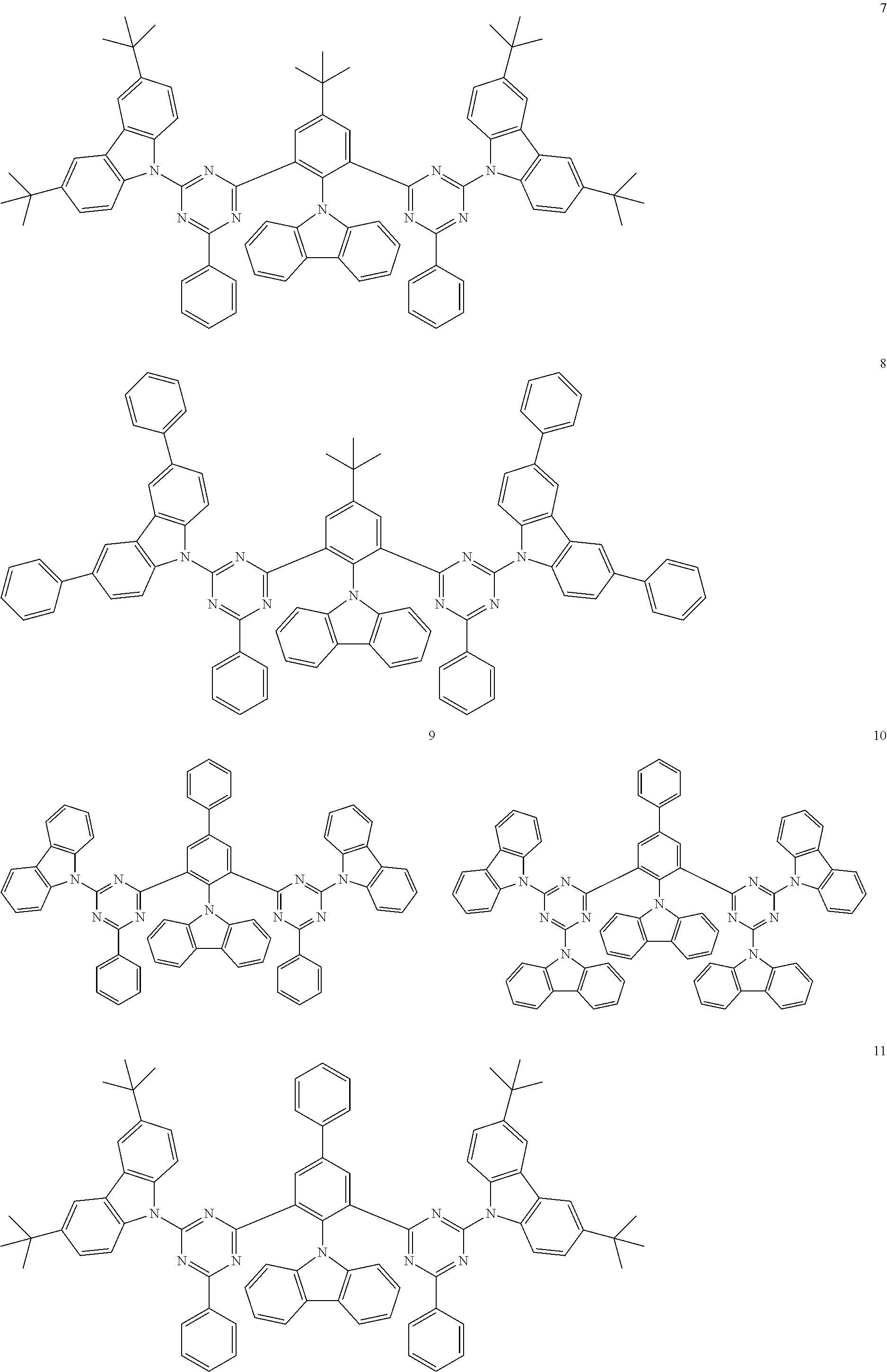

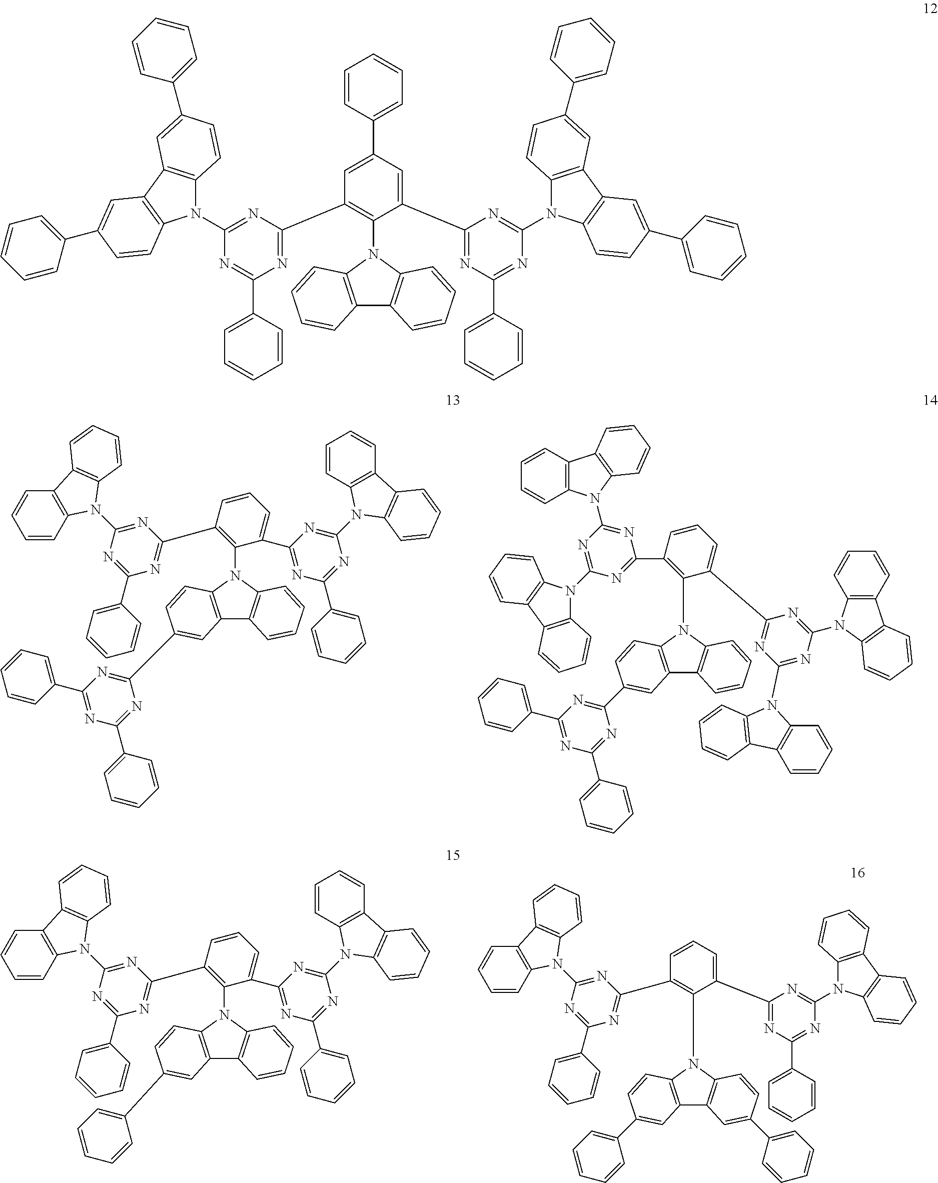

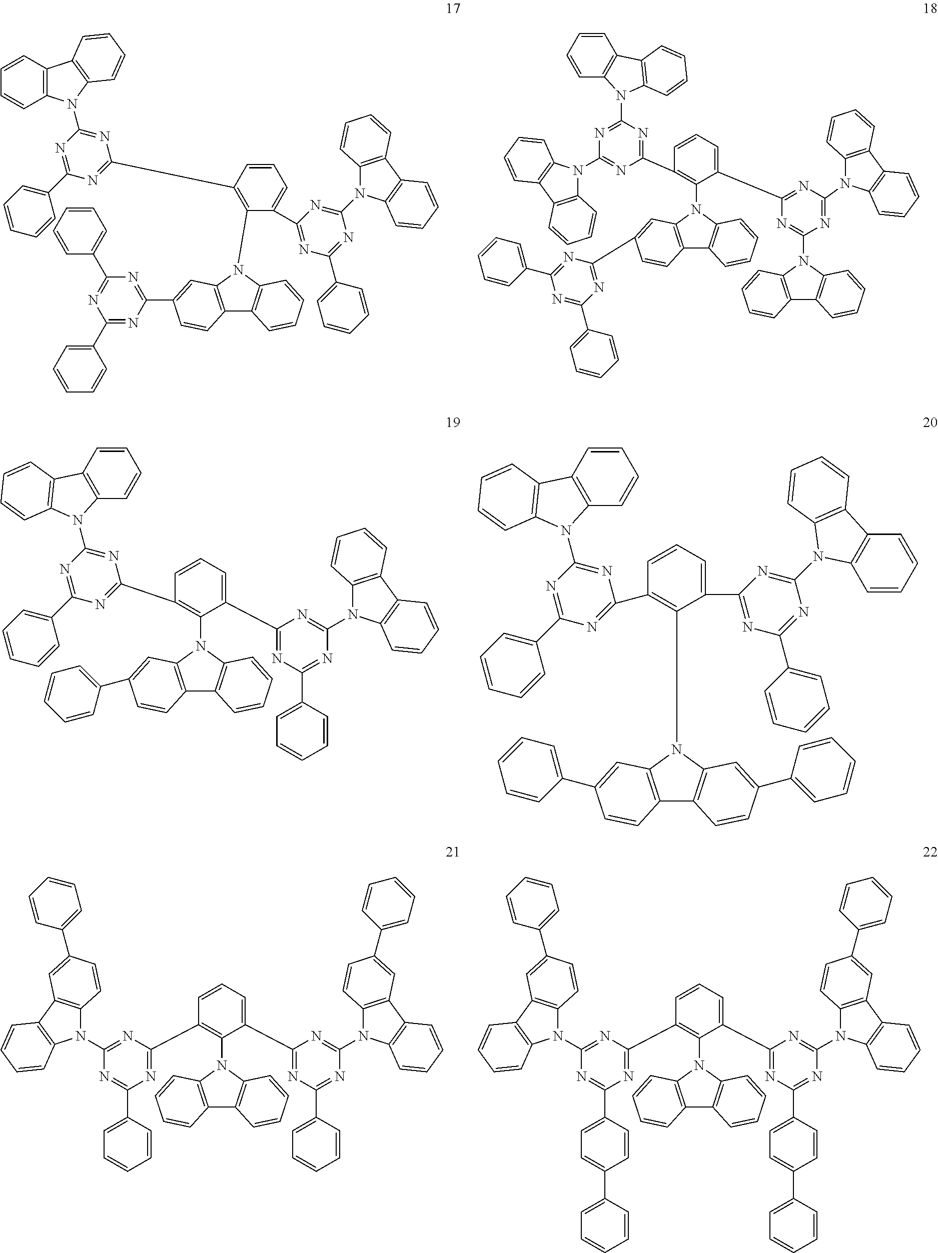

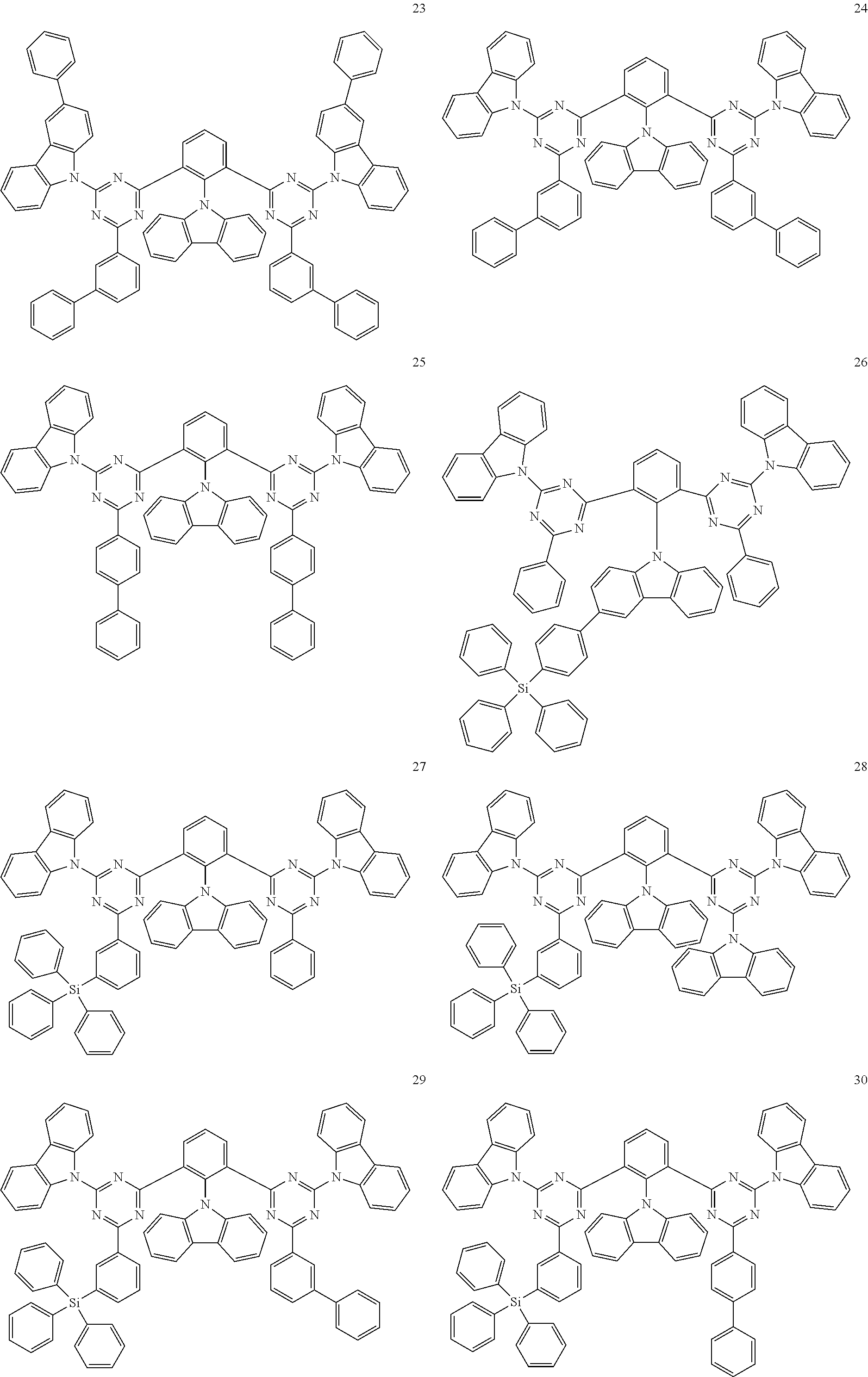

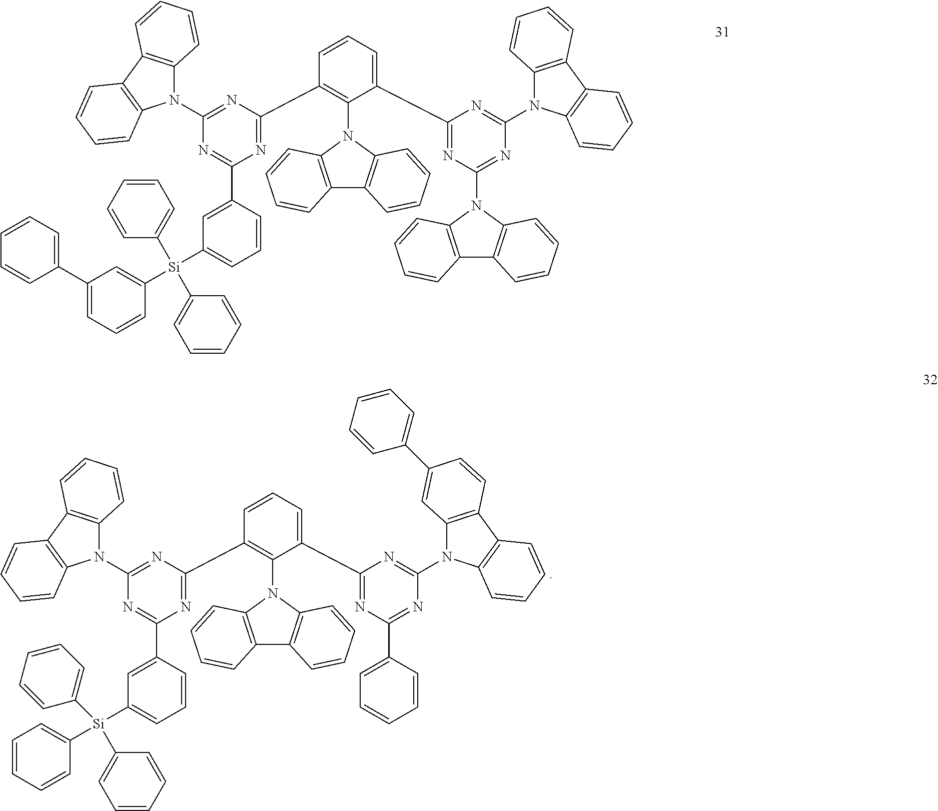 [SMILES: c1ccc(-c2cccc([Si](c3ccccc3)(c3ccccc3)c3cccc(-c4nc(-c5cccc(-c6nc(-n7c8ccccc8c8ccccc87)nc(-n7c8ccccc8c8ccccc87)n6)c5-n5c6ccccc6c6ccccc65)nc(-n5c6ccccc6c6ccccc65)n4)c3)c2)cc1 ; c1ccc(-c2ccc3c4ccccc4n(-c4nc(-c5ccccc5)nc(-c5cccc(-c6nc(-c7cccc([Si](c8ccccc8)(c8ccccc8)c8ccccc8)c7)nc(-n7c8ccccc8c8ccccc87)n6)c5-n5c6ccccc6c6ccccc65)n4)c3c2)cc1]

7. The light emitting element of claim 1, wherein the at least one functional layer comprises:
a hole transport region on the first electrode;
an emission layer on the hole transport region; and
an electron transport region on the emission layer.

8. The light emitting element of claim 7,
wherein the emission layer comprises a first host and a dopant, and
the first host comprises the nitrogen-containing compound.

9. The light emitting element of claim 8, wherein the emission layer is to emit thermally activated delayed fluorescence or phosphorescence.

10. The light emitting element of claim 8, wherein the dopant is represented by Formula D-1: 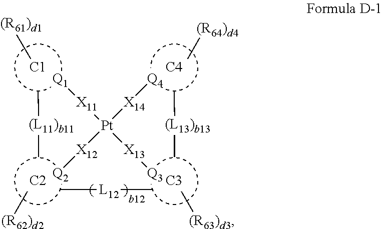
wherein, in Formula D-1,
Q₁ to Q₄ are each independently C or N,
C1 to C4 are each independently a substituted or unsubstituted hydrocarbon ring of 5 to 30 ring-forming carbon atoms, a substituted or unsubstituted aryl group of 6 to 30 ring-forming carbon atoms, or a substituted or unsubstituted heterocycle of 2 to 30 ring-forming carbon atoms,
X₁₁ to X₁₄ are each independently a direct linkage or *—O—*,
L₁₁ to L₁₃ are each independently a direct linkage, 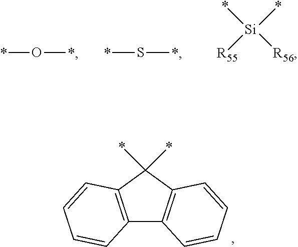
a substituted or unsubstituted alkylene group of 1 to 20 carbon atoms, a substituted or unsubstituted arylene group of 6 to 30 ring-forming carbon atoms, or a substituted or unsubstituted heteroarylene group of 2 to 30 ring-forming carbon atoms,
b11 to b13 are each independently 0 or 1,
R₆₁ to R₆₆ are each independently hydrogen, deuterium, a halogen, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbon atoms, a substituted or unsubstituted alkenyl group of 2 to 20 carbon atoms, a substituted or unsubstituted aryl group of 6 to 60 ring-forming carbon atoms, or a substituted or unsubstituted heteroaryl group of 2 to 60 ring-forming carbon atoms, and
d1 to d4 are each independently an integer of 0 to 4.

11. The light emitting element of claim 8,
wherein the emission layer further comprises a second host that is different from the first host, and
the second host is represented by Formula HT: 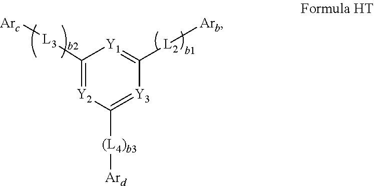
and
wherein, in Formula HT,
at least one selected from among Y₁ to Y₃ is N, and the remainder are CR₅₆,
R₅₆ is hydrogen, deuterium, a substituted or unsubstituted alkyl group of 1 to 20 carbon atoms, a substituted or unsubstituted aryl group of 6 to 60 ring-forming carbon atoms, or a substituted or unsubstituted heteroaryl group of 2 to 60 ring-forming carbon atoms,
b1 to b3 are each independently an integer of 0 to 10,
Ar_{b} to Ar_{d} are each independently hydrogen, deuterium, a substituted or unsubstituted alkyl group of 1 to 20 carbon atoms, a substituted or unsubstituted aryl group of 6 to 60 ring-forming carbon atoms, or a substituted or unsubstituted heteroaryl group of 2 to 60 ring-forming carbon atoms, and
L₂ to L₄ are each independently a direct linkage, a substituted or unsubstituted arylene group of 6 to 60 ring-forming carbon atoms, or a substituted or unsubstituted heteroarylene group of 2 to 60 ring-forming carbon atoms.

12. The light emitting element of claim 7,
wherein the electron transport region comprises:
an electron transport layer on the emission layer; and
an electron injection layer on the electron transport layer, and
the electron transport layer or the electron injection layer comprises the nitrogen-containing compound.

13. An electronic apparatus comprising:
a base layer;
a circuit layer on the base layer; and
a display element layer on the circuit layer and comprising a light emitting element,
wherein the light emitting element comprises a first electrode, a second electrode on the first electrode, and at least one functional layer between the first electrode and the second electrode and comprising a nitrogen-containing compound represented by Formula 1: 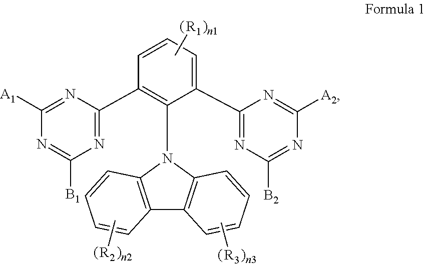
in Formula 1,
R₁ to R₃ being each independently hydrogen, deuterium, a halogen, a cyano group, a substituted or unsubstituted silyl group, a substituted or unsubstituted germanium group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
A₁, A₂, B₁, and B₂ being each independently a substituted or unsubstituted aryl group of 6 to 30 ring-forming carbon atoms or a substituted or unsubstituted heteroaryl group of 2 to 30 ring-forming carbon atoms,
at least one of A₁ or B₁, and at least one of A₂ or B₂ being each independently a substituted or unsubstituted carbazole group,
n1 being an integer of 0 to 3, and
n2 and n3 being each independently an integer of 0 to 4.

14. The electronic apparatus of claim 13,
wherein the light emitting element further comprises a capping layer on the second electrode, and
a refractive index of the capping layer with respect to light in a wavelength range of about 550 nm to about 660 nm is about 1.6 or more.

15. The electronic apparatus of claim 13,
wherein the electronic apparatus further comprises a light controlling layer on the display element layer and comprising a quantum dot,
the light emitting element is to emit first color light, and
the light controlling layer comprises:
a first light controlling part comprising a first quantum dot converting the first color light into second color light which is in a longer wavelength range than the first color light;
a second light controlling part comprising a second quantum dot converting the first color light into third color light which is in a longer wavelength range than the first color light and the second color light; and
a third light controlling part transmitting the first color light.

16. The electronic apparatus of claim 15,
wherein the electronic apparatus further comprises a color filter layer on the light controlling layer, and
the color filter layer comprises:
a first filter transmitting the second color light;
a second filter transmitting the third color light; and
a third filter transmitting the first color light.

17. The electronic apparatus of claim 13, wherein the electronic apparatus comprises one or more selected from among televisions, monitors, outside billboards, personal computers, laptop computers, personal digital terminals, display devices for automobiles, game consoles, portable electronic devices, Internet of Things devices, cameras, mobile phones, smartphones, tablet computers, mobile communication terminals, electronic notebooks, electronic books, portable multimedia players, navigation devices, ultra-mobile personal computers, smartwatches, watch phones, and head-mounted display devices.

18. A nitrogen-containing compound represented by Formula 1: 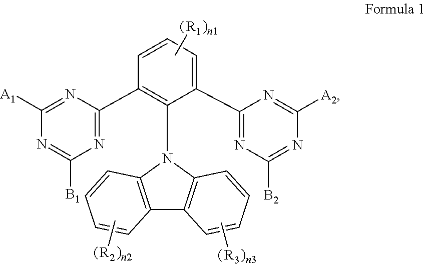
wherein, in Formula 1,
R₁ to R₃ are each independently hydrogen, deuterium, a halogen, a cyano group, a substituted or unsubstituted silyl group, a substituted or unsubstituted germanium group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
A₁, A₂, B₁, and B₂ are each independently a substituted or unsubstituted aryl group of 6 to 30 ring-forming carbon atoms or a substituted or unsubstituted heteroaryl group of 2 to 30 ring-forming carbon atoms,
at least one of A₁ or B₁, and at least one of A₂ or B₂ are each independently a substituted or unsubstituted carbazole group,
n1 is an integer of 0 to 3, and
n2 and n3 are each independently an integer of 0 to 4.

19. The nitrogen-containing compound of claim 18, wherein the nitrogen-containing compound represented by Formula 1 is represented by Formula 2: 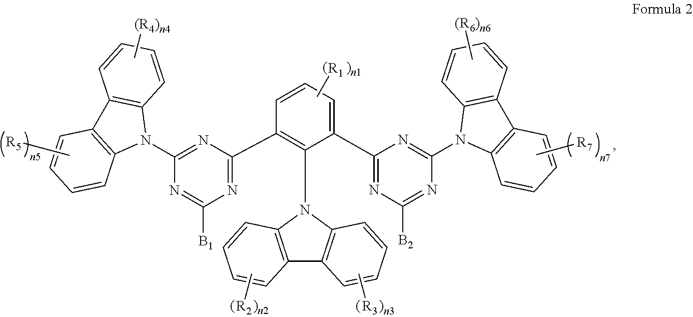
in Formula 2,
R₄ to R₇ being each independently hydrogen, deuterium, a halogen, a cyano group, a substituted or unsubstituted silyl group, a substituted or unsubstituted germanium group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
n4 to n7 being each independently an integer of 0 to 4, and
R₁ to R₃, n1 to n3, B₁, and B₂ being each the same as defined in Formula 1.

20. The nitrogen-containing compound of claim 18,
wherein at least one of A₁ or B₁, and at least one of A₂ or B₂ are each independently a substituted or unsubstituted carbazole group, and
the remainder selected from among A₁, B₁, A₂, and B₂ are each independently represented by one selected from among Formula A-1 to Formula A-4: 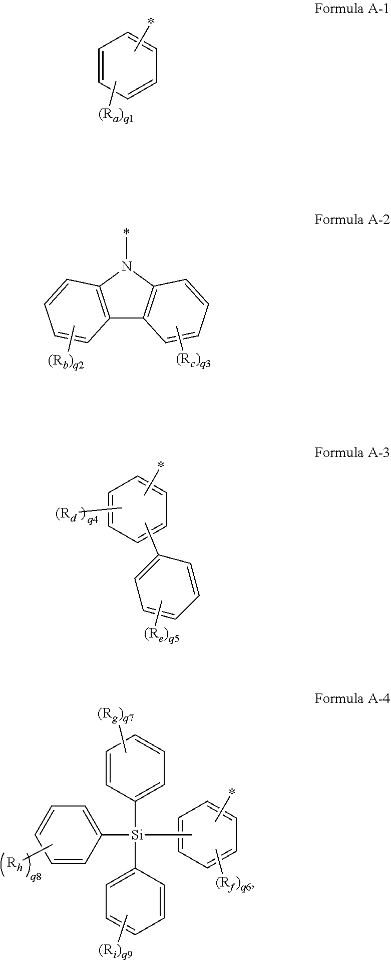
in Formula A-1 to Formula A-4,
Rₐ to Rᵢ being each independently hydrogen, deuterium, a halogen, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
q1, q5, and q7 to q9 being each independently an integer of 0 to 5, and
q2 to q4, and q6 being each independently an integer of 0 to 4.

21. The nitrogen-containing compound of claim 18, wherein the nitrogen-containing compound is any one selected from among compounds in Compound Group 1: 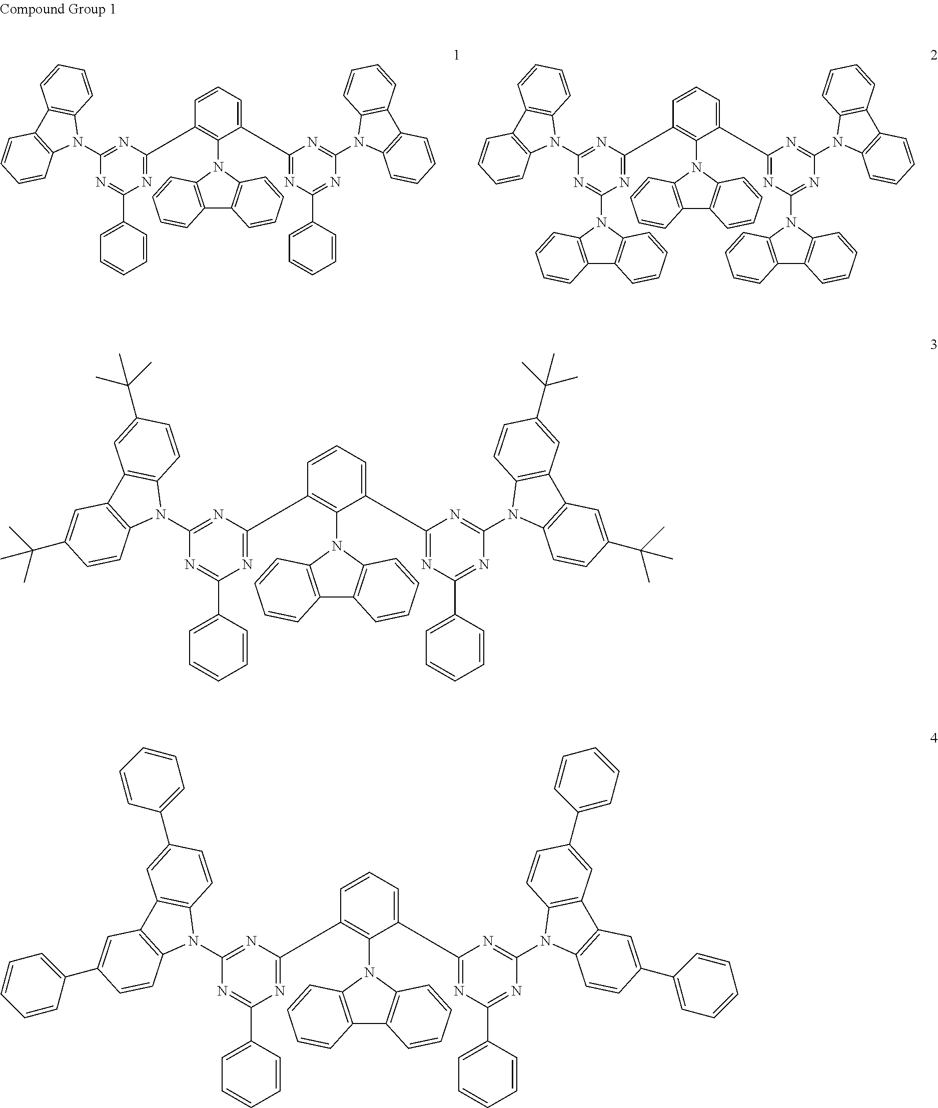 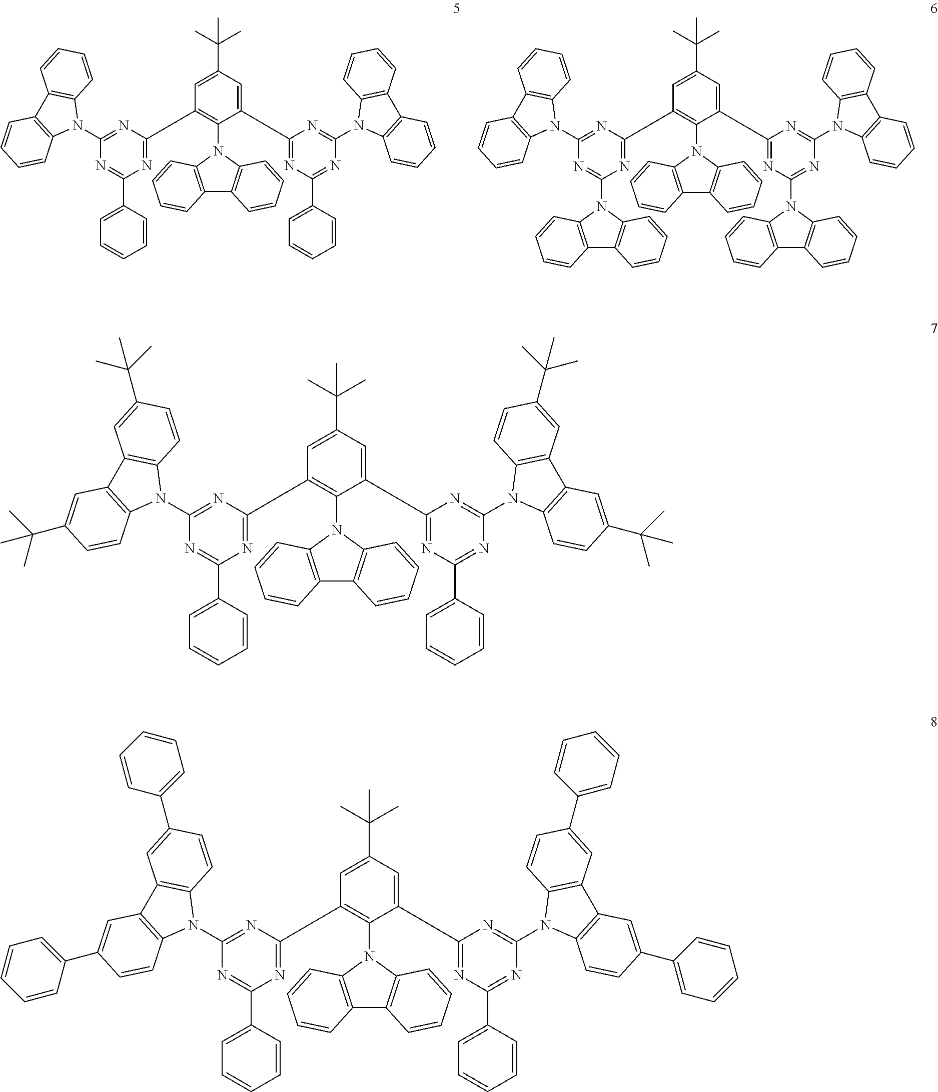 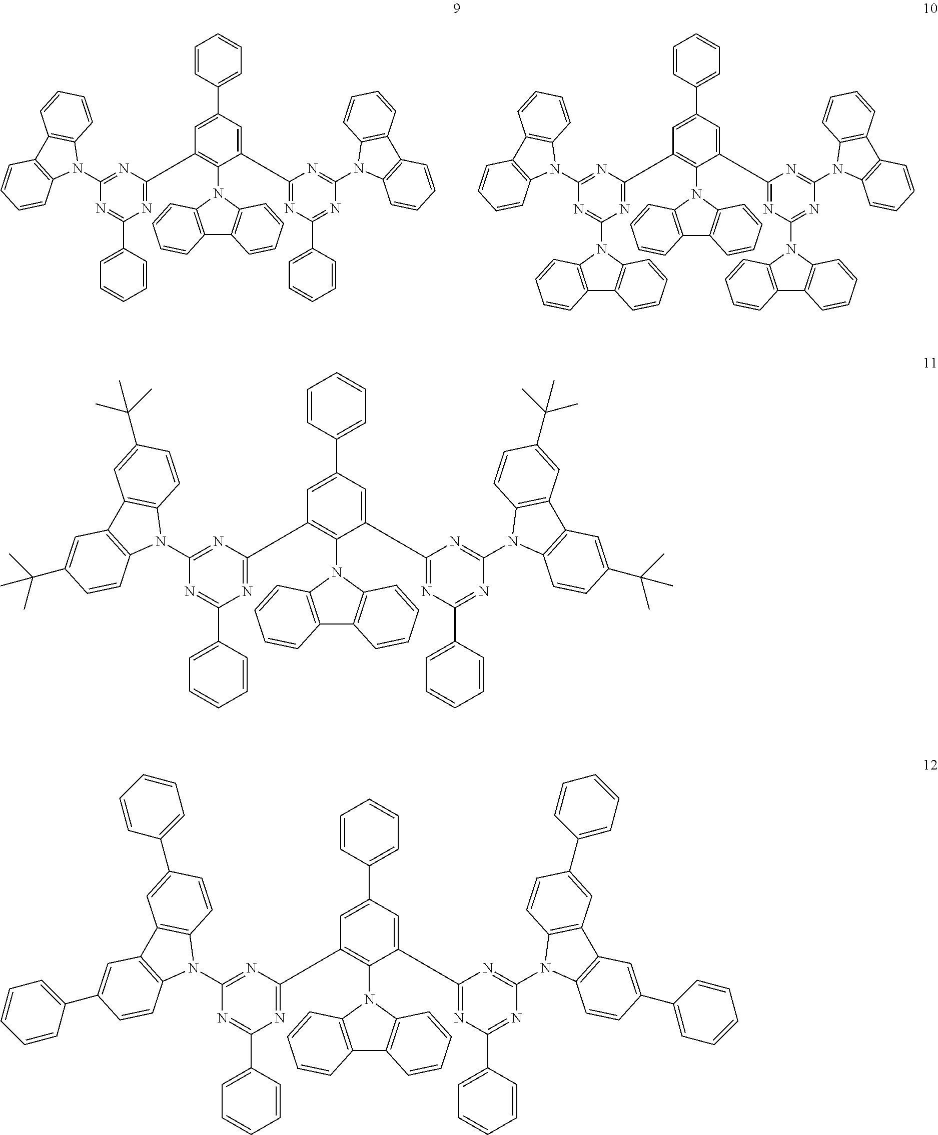 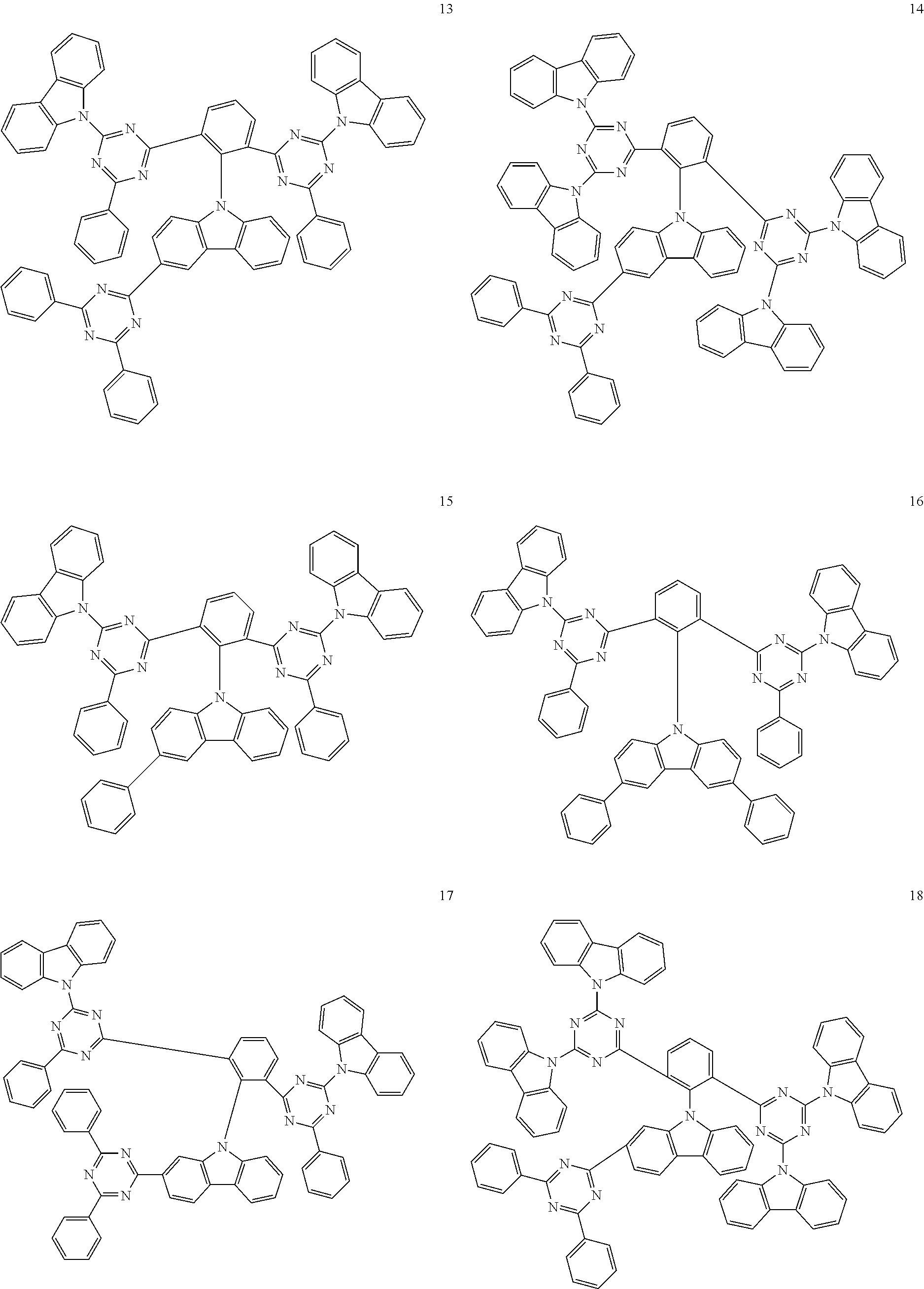 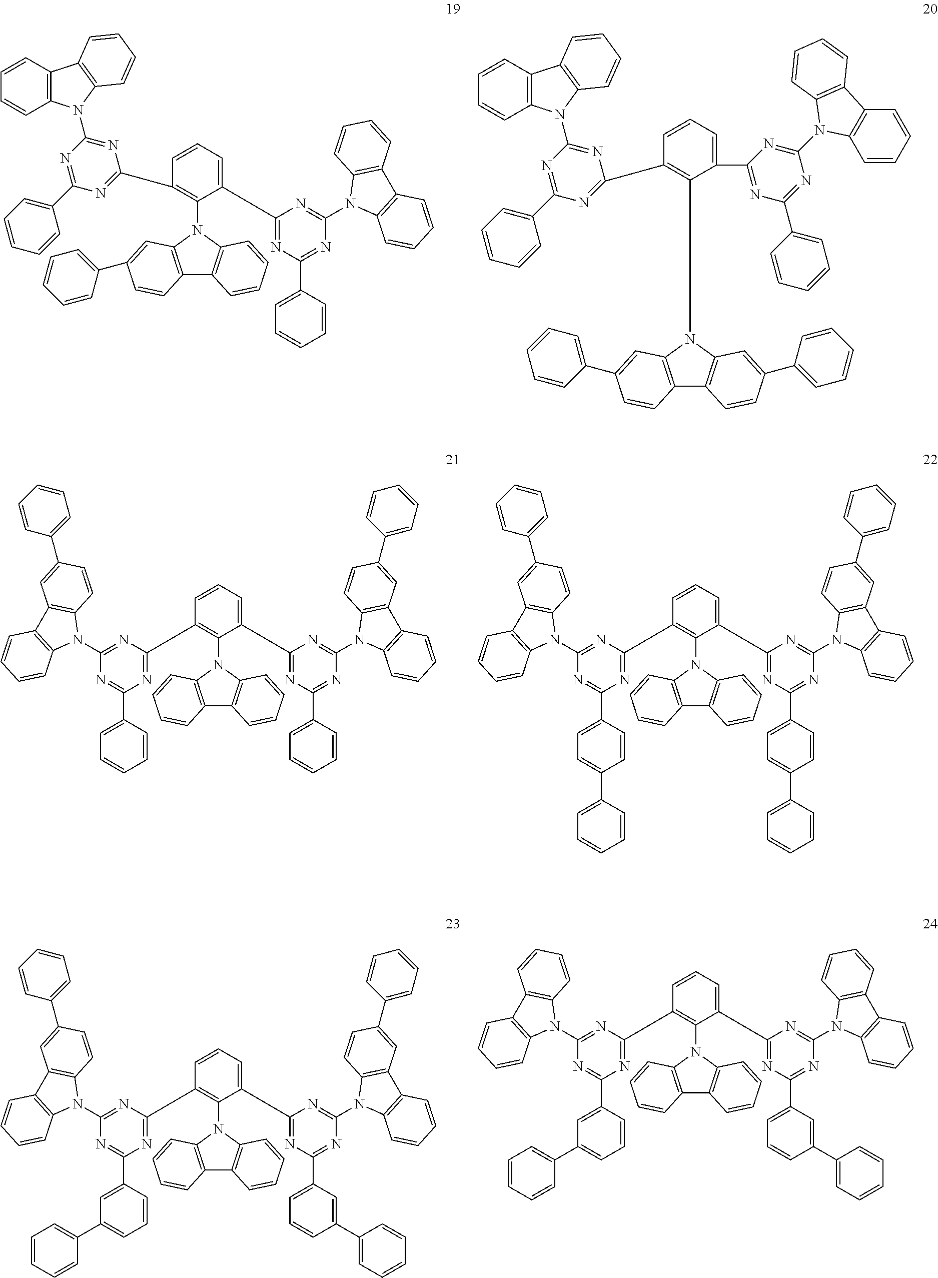 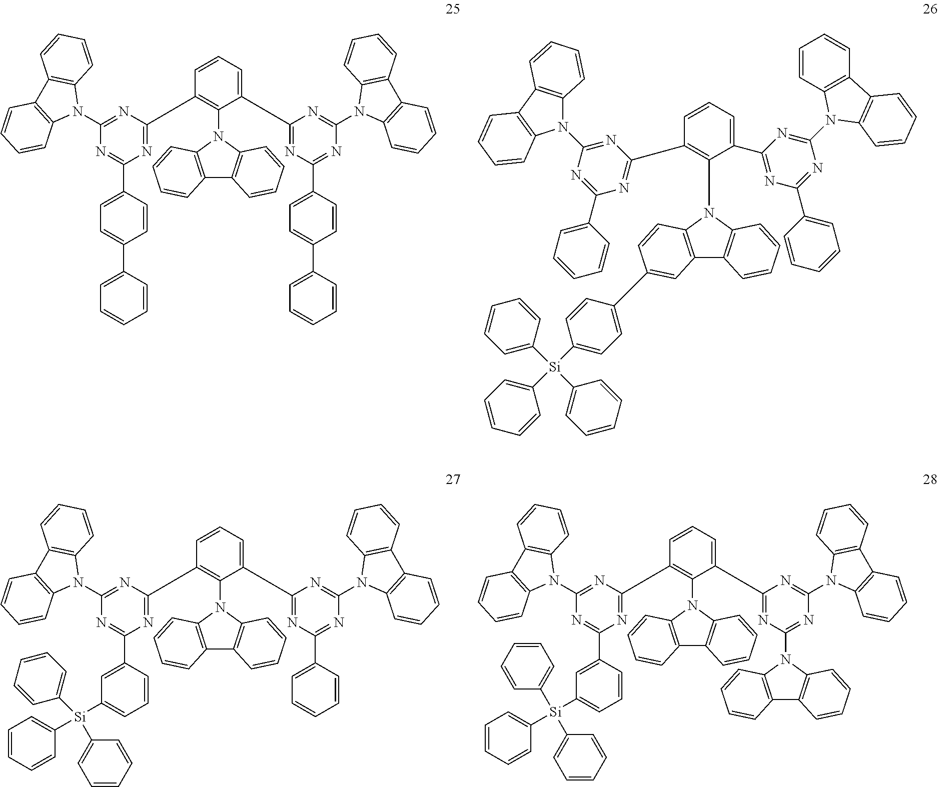 [chemical structure]
